(19) United States
(12) Patent Application Publication
(10) Pub. No.: US 2026/0048059 A1
(43) Pub. Date: Feb. 19, 2026

(54) USE OF SUBSTITUTED BENZOXAZOLE AND BENZOFURAN COMPOUNDS FOR THE TREATMENT AND PREVENTION OF DISEASES ASSOCIATED WITH CHRONIC FATIGUE, EXHAUSTION AND/OR EXERTIONAL INTOLERANC

(71) Applicants: Klaus WIRTH, Kriftel (DE); MITODICURE GMBH, Kriftel (DE)

(72) Inventor: Klaus WIRTH, Kriftel (DE)

(21) Appl. No.: 19/103,780

(22) PCT Filed: Aug. 16, 2023
(86) PCT No.: PCT/EP2023/072569

§ 371 Date: Feb. 13, 2025

(30) Foreign Application Priority Data
Aug. 18, 2022 (EP) 22191022.7
Mar. 8, 2023 (EP) 23160645.0

(51) Int. Cl. A61K 31/527; A61K 31/5386; A61P 21/00

(52) U.S. Cl. CPC A61K 31/527; A61K 31/5386; A61P 21/00

(57) ABSTRACT

The instant invention relates to the use of a substance with phosphodiesterase-7 inhibitory activity (PDE7 inhibitor), such as spiro[[1,3]oxazolo[5,4f-]quinazoline-9,1′-cyclohexan)-7-one derivatives, as active ingredient in a therapeutic agent with phosphodiesterase-7 inhibitory activity for the treatment and prevention of different diseases, syndromes, disease states, or conditions associated with chronic fatigue, exhaustion and/or exertional intolerance using PDE7 inhibitors alone or in combination with other therapeutic agents.

The instant invention relates to the use of a substance with phosphodiesterase-7 inhibitory activity (PDE7 inhibitor) as active ingredient in a therapeutic agent with phosphodiesterase-7 inhibitory activity for the treatment and prevention of different diseases, syndromes, disease states, or conditions associated with chronic fatigue, exhaustion and/or exertional intolerance using PDE7 inhibitors alone or in combination with other therapeutic agents. These diseases, syndromes, disease states, or conditions will subsumed by the term “diseases associated with chronic fatigue” and abbreviated as (DACF) include diseases diagnosed as Myalgic Encephalomyelitis/Chronic Fatigue Syndrome (ME/CFS), systemic exertional intolerance, exertional intolerance, Long COVID complaints, post-Covid-19 syndrome (PCS), post-acute Covid syndrome (PACS), Post-Acute Sequelae of SARS-CoV-2 Infection (PASC), post-vaccination syndrome (post-vac syndrome) after COVID-19 vaccinations (Long Vax) and vaccinations against other germs, virus and pathogenic agents, postinfectious fatigue after viral, bacterial, or fungal infections, in particular ME/CFS of non-infectious, non-inflammatory and non-immunological cause, cancer-related fatigue (wherein chronic fatigue and exhaustion are symptoms of or are associated with cancer), and fatigue associated with fibromyalgia, Ehlers-Danlos syndrome, Marfan syndrome, Gulf War illness and the autoimmune diseases Rheumatoid Arthritis, ANCA vasculitis (anti-neutrophil cytoplasmic antibodies (ANCA)-associated vasculitis) and Sjögren's syndrome, and other autoimmune diseases with fatigue and exhaustion as debilitating symptoms.

PDE7 inhibitors are known and treatments have been described in the context of inflammation, immunomodulation, neoplastic diseases related to the immune system, respiratory diseases, cardiovascular diseases (acute myocardial infarction, stroke), neurodegenerative disorders (multiple sclerosis, Alzheimer's disease, Parkinson's disease), other neurological disorders (spinal cord injury, schizophrenia) and weaning from different addictions, for example in U.S. Pat. Nos. 11,207,275, 8,846,654. The claim for treating neurodegenerative and neurological diseases is based on the assumption that PDE7 inhibition exerts anti-inflammatory effects and treats autoimmune mechanisms in the nervous system.

The pathophysiology of diseases associated with chronic fatigue (DACF) had been totally enigmatic so that no rationally based pharmacological or therapeutic strategies could be derived or developed. Therefore, no specific effective treatment exists due to a lack of understanding of these diseases and their pathophysiology.

The instant invention has been established by a unifying and comprehensive disease hypothesis elaborated by the instant inventor who found that DACF can be treated by stimulating the sodium-potassium ATPase (Na⁺/K⁺ ATPase) in skeletal muscle. The instant inventor found that a rise in cAMP (cyclic adenosine monophosphate) via PDE7-inhibition in skeletal muscle (myocytes) stimulates the sodium-potassium ATPase (Na⁺/K⁺ ATPase) to prevent unfavorable ionic changes in intracellular sodium (Na⁺) and subsequently intracellular and mitochondrial calcium (Ca²⁺), which play a strong pathophysiological role in the development and perpetuation of chronic fatigue, exhaustion and/or exertional intolerance. A second transporter equally important for the prevention of mitochondrial calcium overload is the mitochondrial sodium-calcium exchanger abbreviated as NCLX that exports calcium from the mitochondrium in exchange for sodium to limit and prevent mitochondrial calcium overload. The activity of the NCLX is equally stimulated by cAMP. Thus, a rise in cAMP by PDE7-inhibition has a synergistic action on two ion transporters that prevent cellular and mitochondrial calcium overload and damage. A rise in cAMP can be achieved by administering PDE7 inhibitors, which inhibit the hydrolysis of cAMP to raise its levels. Additionally, a rise in cAMP by PDE7-inhibition also occurs in blood vessels of skeletal muscles and of the brain and enhances organ blood flow for further improvements. Since the PDE7A variant has a much higher expression in skeletal muscle than the variant PDE7B, a PDE7-inhibitor is with a particularly strong inhibitory effect on PDE7A is preferred.

As of today, eleven families of phosphodiesterases (PDE) have been identified. They are distinguished by their primary structure, their substrate specificity and their sensitivity with regard to various effectors and inhibitors specific for PDEs. Each family is composed of one or more genes which are expressed in various tissues in the form of splicing variants. For example, PDE4, PDE7 and PDE8 specifically hydrolyse cAMP and PDE5, PDE6 and PDE9 specifically hydrolyse cGMP.

The family PDE7 is represented by the isoforms PDE7A with their variants PDE7A1, PDE7A2 and PDE7A3 and PDE7B with their variants PDE7B1, PDE7B2 and PDE7B3 originating from two distinct genes. PDE7 is highly selective for cAMP. The Michaelis constant Kₘ values of PDE7B for cAMP ranges from 0.13 μM to 0.2 μM. Kₘ values of PDE7A1 and PDE7A2 for cAMP are 0.2 μM and 0.1 μM, respectively. The catalytic part of PDE7B exhibits approximately 70% homology with that of PDE7A.

In conclusion, inhibition of PDE7 will only raise cAMP and the action of PDE7 inhibitors will to a great extent be restricted to tissues in which PDE7 is expressed.

The presence of PDE7 isoenzymes in skeletal muscle (in myocytes) is known, and there is a higher expression of PDE7A variant versus the PDE7B variant. Inhibition of PDE7 by appropriate inhibitors raises the concentration of the second messenger cAMP which is a key second messenger for appropriate functioning of skeletal muscle. Of additional importance for the treatment of DACF is the presence of PDE7 in blood vessels. Inhibition of PDE7 in blood vessels raises cAMP which has vasodilator actions and improves organ blood flow to improve the metabolic situation and performance. Altogether, raising cAMP in skeletal muscle (myocytes) and in the blood vessels supplying skeletal muscle and inter alia the brain will improve or cure DACF or prevent relapses or prevent chronic fatigue, exhaustion and/or exertional intolerance prophylactically after an infection. Due to the tissue distribution of PDE7, particularly its expression in skeletal muscle (myocytes), and its role in degrading cAMP in skeletal muscle (myocytes) and blood vessels of skeletal muscles and brain, PDE7 inhibitors can treat and prevent diseases of the DACF with minimal side effects.

Among the diseases associated with fatigue, exhaustion, and/or exertional intolerance, Myalgic Encephalomyelitis/Chronic Fatigue Syndrome (ME/CFS) is the most debilitating condition. Although ME/CFS was frequent affecting approximately 0.3% of the population even before Covid-19 it is not very well-known to most physicians. ME/CFS is characterized by profound fatigue, postexertional malaise (PEM), exertional intolerance, cognitive dysfunction, orthostatic intolerance including postural tachycardia (POTS) and associated with a number of different symptoms including muscle weakness and pain. Despite the fact that non-refreshing sleep is a typical symptom, too, and non-refreshing sleep may worsen the patient's situation, the sleep disturbance is not the initial cause for the profound fatigue and exhaustion, but a consequence of ME/CFS that may further worsen the condition.

Prior to the instant invention, no specific treatment had been available and current symptomatic treatments only provide some symptomatic relief and do not cure. Many patients with ME/CFS are bedridden or wheel chair bound; most are incapacitated for work. ME/CFS is often but not always triggered by infections with various viruses, like Epstein-Barr virus (EBV), enteroviruses, influenza virus, dengue fever and as a recent example Corona virus (SARS-CoV-2). Persisting or newly arising complaints after a past acute Corona-Virus infection are referred to as Long COVID or post-COVID-19 syndrome (PCS) or post-acute Covid syndrome (PACS) or post-Acute Sequelae of SARS-CoV-2 Infection (PASC). Studies into PCS show that there is broadest overlap in symptomatology and mechanisms between both conditions. A fraction of PCS patients develops the full picture of ME/CFS. With regard to symptoms and fall in cerebral blood flow in orthostatic tolerance testing, ME/CFS patients and ME/CFS patients with PCS showed indistinguishable symptomatology. The prevalence of ME/CFS is expected to strongly rise by Covid-19.

Hence, there is a strong demand for a drug or therapeutic agent which can be used for the treatment and prevention of chronic fatigue, exhaustion and/or exertional intolerance associated with different diseases, syndromes, disease states (DACF).

Clearly, there is a strong need in the art for new methods for treating and preventing chronic fatigue, exhaustion and/or extertional intolerance associated with different diseases, syndromes, disease states (DACF).

The present invention provides for a new pathway for treating and preventing DACF by the use of a substance with phosphodiesterase-7 inhibitory activity (PDE7 inhibitors) as active ingredient in a therapeutic agent with phosphodiesterase-7 inhibitory activity.

As used herein, the term “PDE7 inhibitor” includes chemical compounds, proteins or polypeptides, nucleic acids, ribozymes, DNAzymes, protein degraders, gene therapies, or other such agents, which directly or indirectly inhibit or block the phosphodiesterase 7 activity of the PDE7 protein (PDE7A, PDE7B) in a selective or non-selective way. In some cases, the agent may bind or interact directly with PDE7 protein. An agent that binds to PDE7 may act to inhibit or block the PDE7 activation by any suitable means, such as by inhibiting the binding of cAMP or substrate ligand with PDE7. In other cases, the PDE7 inhibitory agent may inhibit PDE7 activity indirectly, such as by decreasing expression of the PDE7 protein. In some cases, the PDE7 inhibitory agent may inhibit PDE7 activity by altering the cellular distribution of PDE7, for example, by interfering with the association between PDE7 and an intracellular anchoring protein.

As used herein, the term diseases associated with chronic fatigue includes different diseases, syndromes, disease states, or conditions associated with chronic fatigue, exhaustion and/or exertional intolerance. It includes Myalgic Encephalomyelitis/Chronic Fatigue Syndrome (ME/CFS), systemic exertional intolerance, exertional intolerance, Long COVID complaints, post-Covid-19 syndrome (PCS), post-acute Covid syndrome (PACS), Post-Acute Sequelae of SARS-CoV-2 Infection (PASC), post-vaccination syndrome (post-vac-syndrome) after Covid-19 vaccinations and vaccinations against other germs, virus and pathogenic agents postinfectious fatigue and exhaustion after viral, bacterial, or fungal infections, in particular ME/CFS of non-infectious, non-inflammatory and non-immunological cause. Furthermore, this term includes diseases in which chronic fatigue and exhaustion are symptoms of or associated with cancer (cancer-related fatigue), fibromyalgia, Ehlers-Danlos syndrome, Marfan syndrome, Gulf War illness, the autoimmune diseases Rheumatoid Arthritis, ANCA vasculitis and Sjögren's syndrome, and other autoimmune diseases with fatigue and exhaustion as debilitating symptoms. In the following these diseases, syndromes, disease states, syndromes or conditions are subsumed as diseases associated with chronic fatigue (DACF).

In a preferred embodiment of the instant invention the PDE7 inhibitory agent is a compound that is sufficiently potent to inhibit the enzymatic activity of PDE7 (PDE7A, PDE7B, or PDE7A and PDE7B) at an IC₅₀<50 μM, preferably less than or about 1 μM. Even more preferred, the PDE7 inhibitory agent is sufficiently potent to inhibit the enzymatic activity of PDE7 (PDE7A, PDE7B, or PDE7A and PDE7B) at an IC₅₀ of from about 0.1 to about 600 nM, in particular, the PDE7 inhibitory agent is potent to inhibit the enzymatic activity of PDE7 (PDE7A, PDE7B, or PDE7A and PDE7B) at an IC₅₀ of from about 0.2 to about 100 nM, preferrably at an IC₅₀ of from about 1 to about 100 nM.

In the instant invention, the PDE7 inhibitors stimulate the Na⁺/K⁺-ATPase and the NCLX in skeletal muscle to prevent unfavorable ionic changes that cause mitochondrial and energetic dysfunction in skeletal muscle. The harmful rise in sodium (Na⁺) and ensuing changes in calcium (Ca²⁺) are inhibited by stimulation of the Na⁺/K⁺-ATPase and the NCLX via PDE7-inhibition. PDE7-inhibition improves the energetic situation in skeletal muscle by a dual effect, by preventing the key ionic, metabolic and energetic disturbance in skeletal muscle caused by a rise in sodium (Na⁺) and by preventing subsequent unfavorable changes in cellular and mitochondrial calcium as well as by improving blood flow to the muscles. Furthermore, PDE7 inhibition ameliorates the energetic situation in the brain by raising cerebral blood flow to improve the metabolic situation and performance and it is able to reduce neuroinflammation in the brain by its effects via cAMP. Neuroinflammation is believed to play a pathophysiological role in the central nervous system disturbances causing autonomic dysfunction, impaired cognitions and other CNS symptoms.

The improvements of the energetic situation in skeletal muscle resulting from PDE7-inhibition prevent the release of vasoactive algesic (painful) mediators in skeletal muscles. Normally, these mediators are released to compensate for a poor metabolic situation and are physiologically meant to act only locally in the skeletal muscle. Since excessively produced in the disease state due to the very poor metabolic situation in the large muscle mass of the body, spillover into the systemic circulation occurs so that these mediators with algesic properties can reach and act on every organ in the body. These mediators cause many of the most different symptoms. Apart from fatigue and exhaustion symptoms associated include brain fog, impaired cognition, disturbed sleep, orthostatic intolerance, muscle weakness, pain, post-exertional malaise (PEM), which is an aggravation of the typical symptoms after small or moderate levels of exercise, and many other complaints.

By these combined beneficial effects, PDE7 inhibition improves the symptoms of DACF and potentially cures the diseases of DACF and prevents the relapses and prevents the diseases in a prophylactic manner.

Specific examples for PDE7 inhibitors used in the instant invention are the hereinafter described materials of the formula 1A, formula 1B, compound 1, compound 2, formula 2A, formula 2B, formula 2C, compound 3, formula 3, compound 4, formula 4A, formula 4B, formula 5, formula 6, formula 6A, formula 6B, formula 6C, formula 6D, formula 6E, formula 6F, formula 6G, formula 6H, formula 7A, formula 7B, formula 8, formula 8A, formula 9, formula 10, formula 11, formula 12, formula 13, formula 14, formula 15A, formula 15B, formula 16, formula 16A, formula 17A, formula 17B, formula 18, formula 19, formula 20, formula 21, formula 22, formula 23, formula 24, formula 25, formula 26, formula 27A, formula 27B, formula 27C, formula 27D, formula 28, formula 29, formula 30, formula 31, formula 32, formula 33, formula 34, formula 35, formula 35A, formula 36, formula 37, formula 38, formula 39, formula 40, formula 41, formula 42, formula 42A, formula 43A, formula 43B, formula 44, formula 44A, formula 44B, formula 45, formula 46, formula 47, formula 48, formula 49, formula 50, formula 51, formula 52, formula 53, formula 54, formula 55, formula 55A, formula 55B, formula 55C, formula 55D, formula 55E, formula 55F, formula 55G, formula 55H, formula 551, formula 55J, formula 56, formula 56B, formula 56C, formula 56D, formula 56E, formula 56F, formula 56G, formula 56H, formula 57, formula 57A, formula 57B, formula 57C, formula 57D, formula 57E, formula 58, formula 59, formula 60, formula 61, formula 62, formula 63, formula 63A, formula 64, dipyridamole, pentoxifylline, SUN11817, S14, S.14, and IBMX.

Methods of Treating and Preventing Diseases Associated with Chronic Fatigue (DACF) Using PDE7 Inhibitor(s):

Thus, the present invention includes methods of treating or preventing DACF, comprising administering one or more PDE7 inhibitors to a subject having DACF or at risk for developing DACF.

As used herein, unless the context makes clear otherwise, “treat” and similar word such as “treatment” or “treating” etc., is an approach for obtaining beneficial or desired results, including and preferably clinical results. Treatment can involve optionally either the reducing or amelioration of a disease or condition, e.g., DACF, or the delaying of the progression of the disease or condition, e.g., DACF.

As used herein, unless the context makes clear otherwise, “prevent,” and similar word such as “prevention,” “preventing” etc., is an approach for preventing the onset or recurrence of a disease or condition, (e.g., DACF) or preventing the occurrence, e.g., after a viral infection like with the Epstein-Barr virus, or recurrence of the symptoms of a disease or condition, or optionally an approach for delaying the onset or recurrence of a disease or condition or delaying the occurrence or recurrence of the symptoms of a disease or condition.

As used herein the term “PDE7” is used generically to refer to all translation products coded by transcripts of either or both of these two genes: PDE7A and/or PDE7B.

As used herein, an “effective amount” or a “therapeutically effective amount” of a substance, e.g., a PDE7 inhibitor, is that amount sufficient to affect a desired biological or psychological effect, such as beneficial results, including clinical results. For example, in the context of treating DACF using the methods of the present invention, an effective amount of a PDE7 inhibitor is that amount sufficient to treat and prevent DACF. Generally, a subject is provided with an effective amount of a PDE7 inhibitor.

### Pharmaceutical Compositions, Routes of Administration, Unit Dosage Forms, Kits:

The compositions of the present invention may be administered to a subject as a pharmaceutical composition or formulation. In particular embodiments, pharmaceutical compositions of the present invention may be in any form which allows for the composition to be administered to a subject. For example, the composition may be in the form of a solid, liquid or gas (aerosol). Typical routes of administration include, without limitation, oral, topical, parenteral, sublingual, rectal, vaginal, and intranasal. The term parenteral as used herein includes subcutaneous injections, intravenous, intramuscular, epidural, intrasternal injection or infusion techniques.

Pharmaceutical compositions used according to the present invention comprise a PDE7 inhibitor, another therapeutic agent, and a pharmaceutically acceptable diluent, excipient, or carrier.

“Pharmaceutically acceptable carriers” for therapeutic use are well known in the pharmaceutical art. For example, sterile saline and phosphate buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

Pharmaceutical compositions of the invention are generally formulated so as to allow the active ingredients contained therein to be bioavailable upon administration of the composition to a subject. Compositions that will be administered to a subject may take the form of one or more dosage units, where for example, a tablet, capsule or cachet may be a single dosage unit, and a container comprising a combination of agents according to the present invention in aerosol form may hold a plurality of dosage units. In particular embodiments, the composition comprising a PDE7 inhibitor and eventually another therapeutic agent is administered in one or more doses of a tablet formulation, typically for oral administration. The tablet formulation may be, e.g., an immediate release formulation, a controlled-release formulation, or an extended-release formulation. In one embodiment, a tablet formulation comprises an effective amount of a composition comprising a PDE7 inhibitor and eventually another therapeutic agent. In particular embodiments, a tablet comprises about 1, 5, 10, 20, 30, 50 100, 150, 200, 250, or 300 mg of a PDE7 inhibitor, and eventually about 1, 5, 10, 20, 30, 50 100, 150, 200, 250, or 300 mg of another therapeutic agent if used in combination.

By way of example, a unit administration form of a compound in accordance with the invention in the form of a tablet can comprise the following components: Compound with formula 55E 50 mg, mannitol 223.75 mg, croscarmellose sodium 6 mg, maize starch 15 mg, hydroxypropylmethylcellulose 2.25 mg, and magnesium stearate 3 mg.

Alternatively, in accordance with the invention a capsule formulation can comprise, by way of example, 50 mg of a compound with formula 55E 50 and pharmaceutically acceptable excipients, wherein said excipients can comprise one or more selected from the group consisting of disintegrators, fillers, and lubricants and can comprise an effective amount of binder. Preferably, the PDE7 inhibitor is provided to a subject in an amount in the range of 0.1-1000 mg/day, 1-1000 mg/day, 10-100 mg/day, or 25-50 mg/day.

Certain combinations of PDE7 inhibitors and other therapeutic agents may not be readily adaptable to coformulation. For example, one of the agents may be more amenable to intravenous administration, while another of the agents may be more amenable to oral administration. Or, the serum half-life of the two agents may be such that one must be administered more frequently than the other. Accordingly, the present invention contemplates kits comprising one or more unit dosage forms of a PDE7 inhibitor and one or more unit dosage forms of another therapeutic agent, such that the two unit dosage forms may be provided to a subject in a therapeutically effective manner.

### PDE7 Proteins and Inhibitory Agents

Cyclic nucleotide phosphodiesterase type 7 (PDE7) is identified as a unique family based on its primary amino acid sequence and distinct enzymatic activity. The PDE genes identified as PDE7 (PDE7A and PDE7B), code for cAMP-specific PDEs. The biochemical and pharmacological characterization of PDE7 shows that PDE7 is a high-affinity cAMP-specific PDE-isoenzyme that does not change levels of cGMP. PDE7 is not inhibited by selective inhibitors of other PDE-isoenzymes. The PDE7 enzyme selectively hydrolyzes cAMP and is characterized as an enzyme that is not inhibited by rolipram, a selective inhibitor of PDE4, which is another distinct, cAMP-specific PDE family. Two sub-types have been identified within the PDE7 family, PDE7A and PDE7B. PDE7A and
PDE7B are encoded by two separate genes located on chromosomes 8, g13-q22 and 6q23-q24, respectively. The two gene products exhibit 70% amino acid sequence identity in their C-terminal catalytic domains.

PDE7A (Uniprot ID: Q13946) has three splice variants (PDE7A1, PDE7A2 and PDE7A3) in humans; these variants are generated via alternative splicing at the N- or C-termini. PDE7A1 and PDE7A2 display 97% sequence identity and vary in their N-terminal regions, while PDE7A3 is a C-terminal variant of PDE7A1 but is a shorter protein that still displays 99% sequence identity with PDE7A1. The nucleotide sequence of PDE7A, transcript variant 1, is accessible in public databases. Human PDE7A1 protein has 482 amino acids. The nucleotide sequence of PDE7A, transcript variant 2, is accessible in public databases. Human PDE7A2 protein has 456 amino acids. Human PDE7A3 protein has 424 amino acids. The nucleotide sequence of PDE7A, transcript variant 3, is accessible in public databases. The PDE7A protein has a region of about 270 amino acids at the carboxy terminal end that displays significant similarity (˜23% homology) to the analogous regions of other cAMP-hydrolyzing PDEs. This region serves as the catalytic domain. The amino-terminal region of this protein is divergent from that of other PDEs and presumably mediates the distinctive and regulatory properties unique to this enzyme family. In skeletal muscle PDE7 is the predominant PDE isoenzyme. The expression of variant PDE7A is much higher than that of the variant PDE7B.

Three splice variants of PDE7B have been reported: PDE7B1, PDE7B2 and PDE7B3 with unique N-terminal sequences. The protein sequence of human PDE7B (Uniprot ID: Q9NP56) is accessible in public databases. Similar to the PDE7A protein, the amino-terminal region of PDE7B protein is divergent and presumably accounts for the distinctive and regulatory properties unique to the individual PDE families. The PDE7B protein shows homology to other cAMP-dependent PDEs (^{˜}23%) within the catalytic domain. The PDE7B polypeptide is 59% homologous to PDE7A. PDE7 is also uniquely localized in mammalian subjects relative to other PDE families. PDE7A expression has been detected in the majority of tissues analyzed, including the skeletal muscle, heart, kidney, and spleen. PDE7B expression has been detected in the: brain, peripheral blood mononuclear cells, liver, heart, kidney, small intestine, and skeletal muscle. In skeletal muscle PDE7A expression is much higher than that of the variant PDE7B while the latter shows higher expression in the brain.

In the practice of the methods of the invention, representative PDE7 inhibitory agents that inhibit the phosphodiesterase activity of PDE7 include: Molecules that bind to PDE7 and inhibit the enzyme activity of PDE7 (such as small molecule inhibitors or blocking peptides or proteins/protein fragments/fusion proteins that bind to PDE7 and reduce enzymatic activity), molecules that decrease the expression of PDE7 at the transcriptional and/or translational level (such as PDE7 antisense nucleic acid molecules, PDE7 specific RNAi molecules and PDE7 ribozymes, DNAzymes), and PDE7-directed gene therapies, thereby preventing PDE7 from cleaving cAMP. The PDE7 inhibitory agents can be used alone as a primary therapy or in combination with other therapeutics as an adjuvant therapy to enhance the therapeutic benefits, as discussed here.

The inhibition of PDE7 is characterized by at least one of the following changes that occur as a result of administration of a PDE7 inhibitory agent in accordance with the methods of the invention: the inhibition of PDE7-dependent enzymatic cleavage of the 3′-phosphodiester bond in cAMP to form 5′-adenosine monophosphate (5′-AMP), a reduction in the gene or protein expression level of PDE7, measured, for example, by gene expression analysis (e.g., RT-PCR analysis) or protein analysis (e.g., Western blot).

In some embodiments, a PDE7 inhibitory agent is a molecule or composition that inhibits the expression of PDE7A, PDE7B, or both PDE7A and PDE7B, such as an antisense or small inhibitory nucleotide (e.g., siRNA) that specifically hybridizes with the cellular mRNA and/or genomic DNA corresponding to the gene(s) of the target PDE7 so as to inhibit their transcription and/or translation, or a ribozyme that specifically cleaves the mRNA of a target PDE7.

### Potency of PDE7 Inhibitory Agents:

In one embodiment, a PDE7 inhibitory agent useful in the methods of the invention is a compound that is sufficiently potent to inhibit the enzymatic activity of PDE7 (PDE7A, PDE7B, or PDE7A and PDE7B) at an IC₅₀<50 μM, preferably less than or about 1 μM. In one embodiment, the PDE7 inhibitory agent is sufficiently potent to inhibit the enzymatic activity of PDE7 (PDE7A, PDE7B, or PDE7A and PDE7B) at an IC₅₀ of from about 0.1 to about 600 nM. In one embodiment, the PDE7 inhibitory agent is potent to inhibit the enzymatic activity of PDE7 (PDE7A, PDE7B, or PDE7A and PDE7B) at an IC₅₀ of from about 0.2 to about 100 nM, preferrably at an IC₅₀ of from about 1 to about 100 nM.

Representative methods for determining the IC₅₀ for a PDE7 (PDE7A or PDE7B) inhibitory agent are well known in the art, such as the Scintillation Proximity Assay (SPA).

### PDE7A or PDE7B Selective Inhibitory Agents:

In one embodiment, the PDE7 inhibitor useful in the method of the invention is a PDE7A inhibitory agent. In one embodiment, the PDE7A inhibitory agent is potent to inhibit the enzymatic activity of PDE7A at an IC₅₀ of from about 0.1 to about 600 nM.

In some embodiments, the PDE7 inhibitory agent exhibits isozyme-selective activity against PDE7A. A PDE7A selective inhibitory agent reduces PDE7A activity at least two-fold more than PDE7B activity, more preferably at least 10-fold, at least 20-fold, at least 50-fold, or at least 100-fold. In some embodiments, the PDE7A inhibitory agent is an inhibitory agent that is at least 10-fold (such as at least 20-fold, or at least 50-fold or at least 100-fold) more selective for inhibiting PDE 7A activity than for the enzyme activity of any other PDE (PDE1-6, 7B, and 8-11).

In one embodiment, the PDE7B inhibitory agent is potent to inhibit the enzymatic activity of PDE7B at an IC₅₀ of from about 0.1 to about 600 nM.

In some embodiments, the PDE7 inhibitor exhibits isozyme-selective activity against PDE7B. A PDE7B selective inhibitory agent reduces PDE7B activity at least two-fold more than PDE7A activity, more preferably at least 10-fold, at least 20-fold, at least 50-fold, or at least 100-fold. In some embodiments, the PDE7B inhibitory agent is an inhibitory agent that is at least 10-fold (such as at least 20-fold, or at least 50-fold or at least 100-fold) more selective for inhibiting PDE7B activity than for the enzyme activity of any other PDE (PDE1-6, 7A, and 8-11).

### Types of PDE7 Inhibitory Agents:

The PDE7 inhibitory agent can be any type of agent including, but not limited to, a chemical compound, a protein or polypeptide, a peptidomimetic, a nucleic acid molecule, a ribozyme, a DNAzyme, a protein degrader, or a gene therapy. In some embodiments, PDE7 inhibitory agents are small molecule inhibitors including natural and synthetic substances that have a low molecular weight, such as, for example, peptides, peptidomimetics and nonpeptide inhibitors such as chemical compounds.

### Chemical Compounds:

The PDE7 inhibitors useful in the methods of the invention include agents that are administered by a conventional route (e.g., oral, intramuscular, subcutaneous, transdermal, transbuccal, intravenous, etc.) into the bloodstream and are ultimately transported through the vascular system to inhibit PDE7 in skeletal muscles and the vasculature.

The following is a description of exemplary PDE7 inhibitors useful in the methods of the invention. They can be orthosteric, allosteric or other inhibitors. If it is not explicitly defined later, the following terms mean:
i) aryl: a functional group derived from an aromatic ring or ring system with or without hetero atoms when one hydrogen is removed from such ring structure,
ii) alkyl: in a broad definition the hydrocarbon group formed when a hydrogen atom is removed from an alkane, alkene, or alkyne group,
iii) alkoxy: functional group containing an alkyl group bonded to oxygen,
iv) acyl: functional group with the formula R—C═O and R being aryl, alkyl or other structures.

PDE7 inhibitors useful in the methods of the invention are selected from those compounds generally or specifically disclosed in U.S. Pat. No. 11,685,745 and WO 2019/014305 each expressly incorporated by reference herein in its entirety.

In one embodiment, PDE7 inhibitors useful in the methods of the invention have the formula:

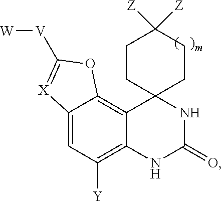

(1), ((1) also referred to as spiro derivatives),
or pharmaceutically acceptable salt thereof, wherein, X is —CH or —N; Y is selected from the group consisting of: —H, halo, and —C₁₋₄alkyl; each Z is independently selected from the group consisting of: —H, -halo, and —C₁₋₄alkyl; V is selected from the group consisting of: a bond, —(CH₂)ₘ—, —(CH₂)ₘO(CH₂)ₙ—, —(CH₂)ₘO(CH₂)ₙC(O)—, —(CH₂)_{M}N[(CH₂)_{N}R^{1A}]—, —(CH₂)ₘC(O)O—, —(CH₂)ₘC(O)—, —(CH₂)ₘC(O)N[(CH₂)ₙR^{1A}]—, -L¹-L²-, -L¹-L²-L³-, -L¹-L²-L³-L⁴-, and -L¹-L⁵; each m is independently 0, 1, 2 or 3; each n is independently 0, 1, 2 or 3; L¹ is —C(O)(CH₂)ₘ—, —[C(R^{1A})₂]ₘ— or —(CH₂)ₘ—; L² is —N[(CH₂)ₙR^{1A}]— or —N((CH₂)ₙR^{1A})(CH₂)ₚ—; L³ is selected from the group consisting of —(CH₂)ₘ—C₃₋₇cycloalkyl, —[C((CH₂)ₙR^{1B})₂]ₘ[C((CH₂)ₙR^{1C})₂]ₙ—, —[N[(CH₂)ₘR^{1A}](CH₂)ₙ]—, —[(CH₂)ₘN((CH₂)ₙR^{1A})(CH₂)ₚ]—, —[(CH₂)ₘC(O)N((CH₂)ₙR^{1B})(CH₂)ₚ]—, —(CH₂)ₘC(O)—, —[C(R^{1A})₂]ₘ— and —[(CH₂)ₚO]—; L⁴ is selected from the group consisting of —(CH₂)ₘC(O)—, —(CH₂)ₘO—, —C₁₋₆alkyl, —C₃₋₇cycloalkyl, heteroaryl, aryl, 3-10 membered heterocycloalkyl, and —CH(OH)—, said —C₁₋₆alkyl, —C₃₋₇cycloalkyl, heteroaryl, aryl, and 3-10 membered heterocycloalkyl each optionally substituted with one to four R^{2A}; L⁵ is selected from the group consisting of —C(O)(CH₂)ₘN[(CH₂)ₙR^{1B}]—, —C(O)(CH₂)ₘ—, —CH(OH)— and —(CH₂)ₘC(O)O—; each p is independently 0, 1, 2 or 3; each R^{1A} is independently selected from the group consisting of: —H, —OH, —CN, halo, —C₁₋₆alkyl, —C₂₋₆alkenyl, —C₂₋₆alkynyl, —C₃₋₇cycloalkyl, aryl, heteroaryl, 3-15 membered heterocycloalkyl, —C₁₋₆haloalkyl, and —C₁₋₆alkoxy, said —C₃₋₇cycloalkyl, aryl, heteroaryl, 3-15 membered heterocycloalkyl, —C₁₋₆haloalkyl, and —C₁₋₆alkoxy each optionally substituted with one to four R^{2A}; each R^{1B} is independently selected from the group consisting of: —H, —OH, —CN, halo, —C₁₋₆alkyl, —C₂₋₆alkenyl, —C₂₋₆alkynyl, —C₃₋₇cycloalkyl, aryl, heteroaryl, 3-15 membered heterocycloalkyl, —C₁₋₆haloalkyl, and —C₁₋₆alkoxy, said —C₃₋₇cycloalkyl, aryl, heteroaryl, 3-15 membered heterocycloalkyl, —C₁₋₆haloalkyl, and —C₁₋₆alkoxy each optionally substituted with one to four R^{2A}; each R^{1C} is independently selected from the group consisting of: —H, —OH, —CN, halo, —C₁₋₆alkyl, —C₂₋₆alkenyl, —C₂₋₆alkynyl, —C₃₋₇cycloalkyl, aryl, heteroaryl, 3-15 membered heterocycloalkyl, —C₁₋₆haloalkyl, and —C₁₋₆alkoxy, said —C₃₋₇cycloalkyl, aryl, heteroaryl, 3-15 membered heterocycloalkyl, —C₁₋₆haloalkyl, and —C₁₋₆alkoxy each optionally substituted with one to four R^{2A}; W is selected from the group consisting of: —H, -halo, —OH, —CN, —C₁₋₆alkyl, —C₁₋₆haloalkyl, aryl, heteroaryl, 3-15 membered heterocycloalkyl, —C₃₋₇cycloalkyl, —SO₂C₁₋₆alkyl, —(CH₂)ₘR^{1A}, —(CH₂)ₘN(R^{1B})₂, —(CH₂)ₘO(CH₂)ₙR^{1A}, —(CH₂)ₘS(CH₂)ₙR^{1A}, —(CH₂)ₘC(O)C₁₋₆alkyl, —(CH₂)ₘC(O)heterocycloalkyl, —(CH₂)ₘC(O)OH, —(CH₂)ₘC(O)OC₁₋₆alkyl, —(CH₂)ₘC(O)NH₂, —(CH₂)ₘC(O)N(C₁₋₄alkyl)₂, and —(CH₂)ₘC(O)NH(C₁₋₄alkyl), said aryl, heteroaryl, 3-15 membered heterocycloalkyl, and —C₃₋₇cycloalkyl, each optionally substituted with one to five R^{3A}; each R^{2A} is independently selected from the group consisting of: halo, —CN, ═O, —OH, —SO₂C₁₋₆alkyl, —C₁₋₆alkyl, —C₁₋₆alkoxy, —C₁₋₆haloalkyl, aryl, heteroaryl, 3-15 membered heterocycloalkyl, —C₃₋₇cycloalkyl, —(CH₂)ₘN(C₁₋₄alkyl)₂, —OCH₂(CH₂)ₘR^{2AA}, —CH₂(CH₂)ₘR^{2AA}, —C₁₋₆alkyl-OH, —C₁₋₆haloalkylOH, —C₁₋₆haloalkyl-C₃₋₇cycloalkyl, —C₂₋₆alkenyl, —C₂₋₆alkynyl, —C(O)C(CH₃)₃, —OC₃₋₇cycloalkyl, —C(O)C₁₋₆alkyl, —C(O)aryl, —C(O)heterocycloalkyl, —C(O)OC₁₋₆alkyl, —C(O)R^{2A}, —NHC(O)R^{2AA}, —C(O)NH(C₁₋₄alkyl), —C(O)N(C₁₋₄alkyl)₂, —CH₂CH(OH)C₃₋₇cycloalkyl, —C(CH₃)₂OH, —N(R^{2AA})₂, —C(CH₃)₂CH₂OCH₃, —CH(CH₃)C(O)N(C₁₋₄alkyl)₂, and —CH₂CF₂C₃₋₇cycloalkyl, said aryl, heteroaryl, 3-15 membered heterocycloalkyl, and —C₃₋₇cycloalkyl, each optionally substituted with one to five substituents each independently selected from the group consisting of: —CN, ═O, —OH, —SO₂C₁₋₆alkyl, —C₁₋₆alkyl, —C₁₋₆alkoxy, —C₁₋₆haloalkyl, aryl, heteroaryl, 3-15 membered heterocycloalkyl, —C₃₋₇cycloalkyl, —N(C₁₋₄alkyl)₂, —NH(C₁₋₄alkyl), —C₂₋₆alkenyl, —C₂₋₆alkynyl, —C(O)C(CH₃)₃, —OC₃₋₇cycloalkyl, —C(O)C₁₋₆alkyl, —C(O)aryl, —C(O)heterocycloalkyl, —C(O)OC₁₋₆alkyl, —C(O)N(C₁₋₄alkyl)₂, —CH₂CH(OH)C₃₋₇cycloalkyl, and —C(CH₃)₂OH; each R^{3A} is independently selected from the group consisting of: halo, —CN, ═O, —OH, —SO₂C₁₋₆alkyl, —C₁₋₆alkyl, —C₁₋₆alkoxy, —C₁₋₆haloalkyl, aryl, heteroaryl, 3-15 membered heterocycloalkyl, —C₃₋₇cycloalkyl, —(CH₂)ₘN(C₁₋₄alkyl)₂, —OCH₂(CH₂)ₘR^{3AA}, —CH₂(CH₂)ₘR^{3AA}, —C₁₋₆alkyl-OH, —C₁₋₆haloalkylOH, —C₁₋₆haloalkyl-C₃₋₇cycloalkyl, —C₂₋₆alkenyl, —C₂₋₆alkynyl, —C(O)C(CH₃)₃, —OC₃₋₇cycloalkyl, —C(O)C₁₋₆alkyl, —C(O)aryl, —C(O)heterocycloalkyl, —C(O)OC₁₋₆alkyl, —C(O)R^{3AA}, —NHC(O)R^{3AA}, —C(O)NH(C₁₋₄alkyl), —C(O)N(C₁₋₄alkyl)₂, —CH₂CH(OH)C₃₋₇cycloalkyl, —C(CH₃)₂OH, —N(R^{3AA})₂, —C(CH₃)₂CH₂OCH₃, —CH(CH₃)C(O)N(C₁₋₄alkyl)₂, and —CH₂CF₂C₃₋₇cycloalkyl, said aryl, heteroaryl, 3-15 membered heterocycloalkyl, and —C₃₋₇cycloalkyl, each optionally substituted with one to five substituents each independently selected from the group consisting of: —CN, ═O, —OH, —SO₂C₁₋₆alkyl, —C₁₋₆alkyl, —C₁₋₆alkoxy, —C₁₋₆haloalkyl, aryl, heteroaryl, 3-15 membered heterocycloalkyl, —C₃₋₇cycloalkyl, —N(C₁₋₄alkyl)₂, —NH(C₁₋₄alkyl), —C₂₋₆alkenyl, —C₂₋₆alkynyl, —C(O)C(CH₃)₃, —OC₃₋₇cycloalkyl, —C(O)C₁₋₆alkyl, —C(O)aryl, —C(O)heterocycloalkyl, —C(O)OC₁₋₆alkyl, —C(O)N(C₁₋₄alkyl)₂, —CH₂CH(OH)C₃₋₇cycloalkyl, and —C(CH₃)₂OH; each R^{2AA} is independently selected from the group consisting of: —H, —OH, —SO₂C₁₋₆alkyl, halo, —CN, —C₁₋₆alkoxy, —C₁₋₆haloalkyl, —N(C₁₋₄alkyl)₂, —NH(C₁₋₄alkyl), —NH₂, aryl, heteroaryl, 3-15 membered heterocycloalkyl, —C₂₋₆alkenyl, —C₂₋₆alkynyl, —C₃₋₇cycloalkyl, —C(O)C₁₋₆alkyl, —C(O)heterocycloalkyl, —C(O)OC₁₋₆alkyl, —C(O)N(C₁₋₄alkyl)₂, and —C(O)aryl; and each R^{3AA} is independently selected from the group consisting of: —H, —OH, —SO₂C₁₋₆alkyl, halo, —CN, —C₁₋₆alkoxy, —C₁₋₆haloalkyl, —N(C₁₋₄alkyl)₂, —NH(C₁₋₄alkyl), —NH₂, aryl, heteroaryl, 3-15 membered heterocycloalkyl, —C₂₋₆alkenyl, —C₂₋₆alkynyl, —C₃₋₇cycloalkyl, —C(O)C₁₋₆alkyl, —C(O)heterocycloalkyl, —C(O)OC₁₋₆alkyl, —C(O)N(C₁₋₄alkyl)₂, and —C(O)aryl.

In one embodiment, PDE7 inhibitors useful in the methods of the invention have the formula:

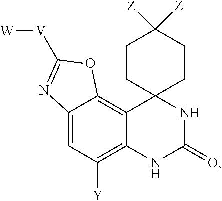

(1A), ((1A) also referred to as benzoxazole derivatives),
or pharmaceutically acceptable salt thereof, wherein, Y is selected from the group consisting of: —H, —F, —Cl, —Br, and —C₁₋₄alkyl; Z is selected from the group consisting of: —H, —F, and —C₁₋₄alkyl; V is selected from the group consisting of: a bond, —CH₂—, —C(O)—, and —NR^{a}—, where R^{a} is —H, —C₁₋₆alkyl, —C₁₋₆haloalkyl, —C₃₋₇cycloalkyl, or —CH₂CH₂OC₁₋₆alkyl; and W is selected from the group consisting of: —H, -halo, —C₁₋₆alkyl, —C₁₋₆haloalkyl, —CH₂OC₁₋₅alkyl, —CH₂OH, —CH₂CH₂OH, —CH₂CH₂OCH₃, —C(O)C₁₋₆alkyl, —C(O)C₃₋₇cycloalkyl, —C(O)OC₁₋₄alkyl, —C₃₋₇cycloalkyl, said —C₃₋₇cycloalkyl optionally substituted with one or more -halo or —C₁₋₆alkyl, —C₁₋₆aryl, —C₁₋₆heteroaryl, said aryl or heteroaryl optionally substituted, —OH, —OC₁₋₆alkyl, —OCH₂CH₂OCH₃, —OCH₂C(O)N(CH₃)₂, —O-tetrahydropyranyl, —Opiperidinyl, optionally substituted heterocycloalkyl, and —N(R^{b})₂, where each R^{b} is independently selected from the group consisting of: —H, —C₁₋₆alkyl, —C₁₋₆alkyl-OH, —C₁₋₆halo alkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)heterocycloalkyl, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c}, and —(CH₂)ₙ—R^{d}, where R^{c} is —C₂₋₆alkynyl, —C₃₋₇cycloalkyl, —OH, —O—C₁₋₆alkyl, —OC(CH₃)₂, —O—C₁₋₆halo alkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, where R^{d} is —C₃₋₇cycloalkyl, —C₃₋₇halocycloalkyl, heterocycloalkyl, aryl, or heteroaryl, each optionally substituted with up to 4 substituents each independently selected from the group consisting of: halo, —C₁₋₆alkyl, —C₁₋₆halo alkyl, —C₃₋₇cycloalkyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, phenyl, or benzyl, where n is 0, 1, 2 or 3; or, alternatively, both R^{b} come together to form a 4-15-membered, monocyclic, bicyclic or tricyclic ring, optionally containing up to 3 additional heteroatoms each independently selected from O (oxygen), N (nitrogen) and S (sulfur), said 4-15-membered monocyclic, bicyclic or tricyclic ring optionally substituted with one to 4 R^{b1} where each R^{b1} is independently selected from the group consisting of halo, —C₁₋₆alkyl, —C₁₋₆haloalkyl, —C₁₋₆haloalkyl-C₃₋₇cycloalkyl, —C₃₋₇cycloalkyl, bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆halo alkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole, or —C(O)phenyl.

In one embodiment, PDE7 inhibitors useful in the methods of the invention have the formula:

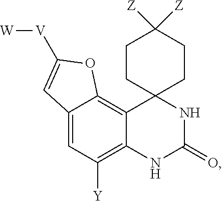

(1B), ((1B) also referred to as benzofuran derivatives),
or pharmaceutically acceptable salt thereof, wherein, Y is selected from the group consisting of: —H, —F, —Cl, —Br, and —C₁₋₄alkyl; Z is selected from the group consisting of: —H, —F and —CH₃; V is selected from the group consisting of: a bond, —CH₂—, —C(O)—, and —NR^{a}—, where R^{a} is —H, —C₁₋₆alkyl, —C₁₋₆haloalkyl, —C₃₋₇cycloalkyl, and —CH₂CH₂OC₁₋₆alkyl; and W is selected from the group consisting of: —H, halo, —C₁₋₆alkyl, —C₁₋₆haloalkyl, —CH₂OC₁₋₅alkyl, —CH₂OH, —CH₂CH₂OH, —CH₂CH₂OCH₃, —C(O)C₁₋₆alkyl, —C(O)C₃₋₇cycloalkyl, —C(O)OC₁₋₄alkyl, —C₃₋₇cycloalkyl, said —C₃₋₇cycloalkyl optionally substituted with one or more halo or —C₁₋₆alkyl, —C₁₋₆aryl, —C₁₋₆heteroaryl, said aryl or heteroaryl optionally substituted, —OH, —OC₁₋₆alkyl, —OCH₂CH₂OCH₃, —OCH₂C(O)N(CH₃)₂, —O-tetrahydropyranyl, —Opiperidinyl, optionally substituted heterocycloalkyl, and —N(R^{b})₂, where each R^{b} is independently selected from the group consisting of: —H, —C₁₋₆alkyl, —C₁₋₆alkyl-OH, —C₁₋₆halo alkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)heterocycloalkyl, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c}, and —(CH₂)ₙ—R^{d}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, where R^{d} is cyclopropyl, cyclobutyl, cyclopentyl, cyclohexyl, bicyclo[1.1.1]pentane, bicyclo[3.1.0]hexane, —C₃₋₇halocycloalkyl, oxetane, pyrrolidine, pyrrolidinone, piperidine, piperidinone, tetrahydropyranyl, tetrahydrofuranyl, oxepane, morpholine, 3-azabicyclo[3.1.0]hexane, tetrahydrothiophene-1,1-dioxide, tetrahydro-2H-thiopyran-1,1-dioxide, thiomorpholine-1,1-dioxide, tetrahydrothiophene 1,1-dioxide, 1,3-dihydroisobenzofuran, benzofuran, indoline, benzodioxine, benzodioxole, phenyl, benzyl, —CH₂CH₂phenyl, —CH₂CH₂CH₂phenyl, furan, pyrrole, pyrazole, imidazole, triazole, isoxazole, oxazole, thiazole, pyridine, pyrimidine, thiophene, pyrrolopyridine, or benzimidazole, said R^{d} optionally substituted with up to 4 substituents each independently selected from the group consisting of: halo, —C₁₋₆alkyl, —C₁₋₆haloalkyl, -cyclopropyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, phenyl, or benzyl, where n is 0, 1, 2 or 3; or, alternatively, both R^{b} come together to form a monocyclic, bicyclic or tricyclic ring selected from the group consisting of: azetidine, pyrrole, pyrrolidine, pyrrolidinone, piperazine, piperazinone, piperidine, piperidinone, azepane, morpholine, 1-oxa-8-azaspiro[4.5]decan-3-one, 2-oxa-5-azabicyclo[2.2.1]heptane, 2-oxa-7-azaspiro[3.5]nonane, 2-oxa-8-azaspiro[4.5]decane, 2-oxa-8-azaspiro[4.5]decan-1-one, -6-oxa-9-azaspiro[4.5]decane, 7-oxa-2-azaspiro[3.5]nonane, octahydropyrrolo[3,4-c]pyrrole, octahydropyrrolo[1,2-a]pyrazine, oxaazabicyclo[2.2.1]heptane, thiomorpholine-1,1-dioxide, diazepane, 2,3,4,5-tetrahydropyrido[2,3-f][1,4]oxazepine, 5,6,7,8-tetrahydro[1,2,4]triazolo[4,3-a]pyrazine, 5,6,7,8-tetrahydroimidazo[1,2-a]pyrazine, 5,6,7,8-tetrahydropyrido[3,4-d]pyridazin-1(2H)-one, azaspiro[2.5]octane, azabicyclo[3.1.0]hexane, azabicyclo[4.1.0]heptane, diazaspiro[5.5]undecane, tetrahydroimidazopyrazine, dihydropyrrolopyridine, spiro[chroman-2,3′-pyrrolidin]-4-one, Spiro[isochroman-1,4′-piperidine], 6′,7′-dihydro-5′H-spiro[piperidine-4,4′-thieno[3,2-c]pyridine], and 5′,6′-dihydro-4′H-spiro[piperidine-4,7′-thieno[2,3-c]pyridine]), each optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention have the formula:

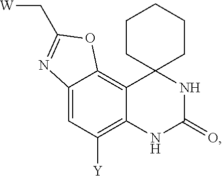

(1C), ((1C) also referred to as methylene-substituted 7,8-dihydro-6H-spiro[[1,3]oxazolo[5,4-f]quinazoline-9,1′-cyclohexane]-7-one derivatives),
or a pharmaceutically acceptable salt thereof, wherein, Y is selected from the group consisting of: —H, —F, —Cl, —Br, and —CH₃; and W is —N(R^{b})₂, where each R^{b} is independently selected from the group consisting of: —H, —C₁₋₆alkyl, —C₁₋₆alkylOH, —C₁₋₆haloalkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)heterocycloalkyl, —CH₂C(O)OC₁₋₆alkyl, —CH₂C(O)N(C₁₋₆alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂₋₁₂c and —(CH₂)ₙ—R^{d}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, where R^{d} is —C₃₋₇cycloalkyl, —C₃₋₇halocycloalkyl, heterocycloalkyl, aryl, or -heteroaryl, each optionally substituted with up to 4 substituents each independently selected from the group consisting of: halo, —C₁₋₆alkyl, —C₁₋₆haloalkyl, —C₃₋₇cycloalkyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, morpholine, phenyl, or benzyl, where n is 0, 1, 2 or 3; or, alternatively, both R^{b} come together to form a 4-15-membered, monocyclic, bicyclic or tricyclic ring, optionally containing up to 3 additional heteroatoms each independently selected from O (oxygen), N (nitrogen) and S (sulfur), said 4-15-membered monocyclic, bicyclic or tricyclic ring optionally substituted with up to 4 substituents each independently selected from the group consisting of: halo, —C₁₋₆alkyl, —C₁₋₆halo alkyl, —C₁₋₆haloalkyl-C₃₋₇cycloalkyl, —C₃₋₇cycloalkyl, -bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention have the formula:

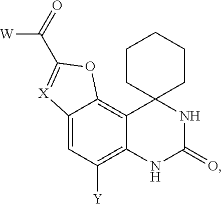

(1D), ((1D) also referred to as acylated 7,8-dihydro-6H-spiro[[1,3]oxazolo[5,4-f]quinazoline-9,1′-cyclohexane]-7-one derivatives),
or a pharmaceutically acceptable salt thereof, wherein, Y is selected from the group consisting of: —H, —F, —Cl, —Br, and —CH₃; and W is —N(R^{b})₂, where each R^{b} is independently selected from the group consisting of: —H, —C₁₋₆alkyl, —C₁₋₆alkyl-OH, —C₁₋₆halo alkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)heterocycloalkyl, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂₋₁₂c and —(CH₂)ₙ—R^{d}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, where R^{d} is —C₃₋₇cycloalkyl, —C₃₋₇halocycloalkyl, heterocycloalkyl, aryl, or heteroaryl, each optionally substituted with up to 4 substituents each independently selected from the group consisting of: halo, —C₁₋₆alkyl, —C₁₋₆halo alkyl, —C₃₋₇cycloalkyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, morpholine, aryl, phenyl, or benzyl, where n is 0, 1, 2 or 3; or, alternatively, both R^{b} come together to form a 4-15-membered, monocyclic, bicyclic or tricyclic ring, optionally containing up to 3 additional heteroatoms each independently selected from O (oxygen), N (nitrogen) and S (sulfur), said 4-15-membered monocyclic, bicyclic or tricyclic ring optionally substituted with up to 4 substituents each independently selected from the group consisting of: halo, —C₁₋₆alkyl, —C₁₋₆halo alkyl, —C₁₋₆haloalkyl-C₃₋₇cycloalkyl, —C₃₋₇cycloalkyl, bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention have the formula:

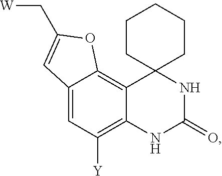

(1E), ((1E) also referred to as methylene-substituted 7,8-dihydro-6H-spiro[[1,3]furo[5,4-f]quinazoline-9,1′-cyclohexane]-7-one derivatives),
or pharmaceutically acceptable salt thereof, wherein, Y is selected from the group consisting of: —H, —F, —Cl, —Br, and —CH₃; and W is —N(R^{b})₂, where each R^{b} is independently selected from the group consisting of: —H, —C₁₋₆alkyl, —C₁₋₆alkyl-OH, —C₁₋₆halo alkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)heterocycloalkyl, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c}, and —(CH₂)ₙ—R^{d}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, where R^{d} is —C₃₋₇cycloalkyl, —C₃₋₇halocycloalkyl, heterocycloalkyl, aryl, or heteroaryl, each optionally substituted with up to 4 substituents each independently selected from the group consisting of: halo, —C₁₋₆alkyl, —C₁₋₆halo alkyl, —C₃₋₇cycloalkyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, phenyl, or benzyl, where n is 0, 1, 2 or 3; or, alternatively, both R^{b} come together to form a 4-15-membered, monocyclic, bicyclic or tricyclic ring, optionally containing up to 3 additional heteroatoms each independently selected from O (oxygen), N (nitrogen) and S (sulfur), said 4-15-membered monocyclic, bicyclic or tricyclic ring optionally substituted with up to 4 substituents each independently selected from the group consisting of: halo, —C₁₋₆alkyl, —C₁₋₆halo alkyl, —C₁₋₆haloalkyl-C₃₋₇cycloalkyl, —C₃₋₇cycloalkyl, bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention have the formula:

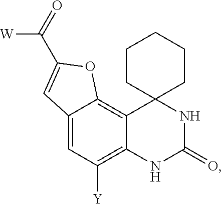

(1F), ((1F) also referred to as acylated 7,8-dihydro-6H-spiro[[1,3]furo[5,4-f]quinazoline-9,1′-cyclohexane]-7-one derivatives),
or pharmaceutically acceptable salt thereof, wherein, Y is selected from the group consisting of: —H, —F, —Cl, —Br, and —CH₃; and W is —C₁₋₆aryl, —C₁₋₆heteroaryl, said aryl or heteroaryl optionally substituted, or —N(R^{b})₂, where each R^{b} is independently selected from the group consisting of: —H, —C₁₋₆alkyl, —C₁₋₆alkyl-OH, —C₁₋₆halo alkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)heterocycloalkyl, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c}, and —(CH₂)ₙ—R^{d}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, where R^{d} is —C₃₋₇cycloalkyl, —C₃₋₇halocycloalkyl, heterocycloalkyl, aryl, or heteroaryl, each optionally substituted with up to 4 substituents each independently selected from the group consisting of: halo, —C₁₋₆alkyl, —C₁₋₆haloalkyl, —C₃₋₇cycloalkyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, morpholine, phenyl, or benzyl, where n is 0, 1, 2 or 3; or, alternatively, both R^{b} come together to form a 4-15-membered, monocyclic, bicyclic or tricyclic ring, optionally containing up to 3 additional heteroatoms each independently selected from O (oxygen), N (nitrogen) and S (sulfur), said 4-15-membered monocyclic, bicyclic or tricyclic ring optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, -bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention have the formula (1) or are a pharmaceutically acceptable salt thereof, wherein W is selected from the group consisting of: azetidine, pyrrole, pyrrolidine, pyrrolidinone, piperazine, piperazinone, piperidine, piperidinone, azepane, morpholine, 1-oxa-8-azaspiro[4.5]decan-3-one, 2-oxa-5-azabicyclo[2.2.1]heptane, 2-oxa-7-azaspiro[3.5]nonane, -2-oxa-8-azaspiro[4.5]decane, 2-oxa-8-azaspiro[4.5]decan-1-one, 6-oxa-9-azaspiro[4.5]decane, 7-oxa-2-azaspiro[3.5]nonane, octahydropyrrolo[3,4-c]pyrrole, octahydropyrrolo[1,2-a]pyrazine, oxaazabicyclo[2.2.1]heptane, thiomorpholine-1,1-dioxide, diazepane, 2,3,4,5-tetrahydropyrido[2,3-f][1,4]oxazepine, 5,6,7,8-tetrahydro[1,2,4]triazolo[4,3-a]pyrazine, 5,6,7,8-tetrahydroimidazo [1,2-a]pyrazine, 5,6,7,8-tetrahydropyrido[3,4-d]pyridazin-1(2H)-one, azaspiro[2.5]octane, azabicyclo[3.1.0]hexane, azabicyclo[4.1.0]heptane, diazaspiro[5.5]undecane, tetrahydroimidazopyrazine, dihydropyrrolopyridine, spiro[chroman-2,3′-pyrrolidin]-4-one, spiro[isochroman-1,4′-piperidine], 6′,7′-dihydro-5′H-spiro[piperidine-4,4′-thieno[3,2-c]pyridine], and 5′,6′-dihydro-4′H-spiro[piperidine-4,7′-thieno[2,3-c]pyridine], each optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, -bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention have the formula (1) or are a pharmaceutically acceptable salt thereof, wherein W is halo, —C₁₋₆alkyl, —C₁₋₆haloalkyl or —C₃₋₇cycloalkyl; said —C₃₋₇cycloalkyl optionally substituted with one or more halo or —C₁₋₆alkyl.

In one embodiment, PDE7 inhibitors useful in the methods of the invention have the formula (1) or are a pharmaceutically acceptable salt thereof, wherein W is selected from the group consisting of: halo, —C₁₋₆alkyl, —C₁₋₆haloalkyl, —CH₂OC₁₋₅alkyl, —CH₂OH, —CH₂CH₂OH, —CH₂CH₂OCH₃, —C(O)C₁₋₆alkyl, —C(O)C₃₋₇cycloalkyl, —C(O)OC₁₋₄alkyl, —C₃₋₇cycloalkyl, said —C₃₋₇cycloalkyl optionally substituted with one or more halo or —C₁₋₆alkyl, optionally substituted —C₁₋₆aryl, optionally substituted —C₁₋₆heteroaryl, —OH, —OC₁₋₆alkyl, —OCH₂CH₂OCH₃, —OCH₂C(O)N(CH₃)₂, —O-tetrahydropyranyl, —Opiperidinyl, and —N(R^{b})₂, where each R bis independently selected from the group consisting of: —H, —C₁₋₆alkyl, —C₁₋₆alkyl-OH, —C₁₋₆haloalkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)heterocycloalkyl, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c} where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, and —(CH₂)ₙ—R^{d}, where R^{d} is —C₃₋₇cycloalkyl, —C₃₋₇halocycloalkyl, heterocycloalkyl, aryl, or heteroaryl, each optionally substituted with up to 4 substituents each independently selected from the group consisting of: halo, —C₁₋₆alkyl, —C₁₋₆haloalkyl, —C₃₋₇cycloalkyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, phenyl, or benzyl, where n is 0, 1, 2 or 3; or, alternatively, both R^{b} come together to form a 4-15-membered, monocyclic, bicyclic or tricyclic ring, optionally containing up to 3 additional heteroatoms each independently selected from O (oxygen), N (nitrogen) and S (sulfur), said 4-15-membered monocyclic, bicyclic or tricyclic ring optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, -bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention have the formula (1) or are a pharmaceutically acceptable salt thereof, wherein W is selected from the group consisting of: halo, —C₁₋₆alkyl, —C₁₋₆haloalkyl, —CH₂OC₁₋₅alkyl, —CH₂OH, —CH₂CH₂OH, —CH₂CH₂OCH₃, —C(O)C₁₋₆alkyl, —C(O)C₃₋₇cycloalkyl, —C(O)OC₁₋₄alkyl, —C₃₋₇cycloalkyl, said —C₃₋₇cycloalkyl optionally substituted with one or more halo or —C₁₋₆alkyl, —C₁₋₆aryl, —C₁₋₆heteroaryl, said aryl or heteroaryl optionally substituted, —OH, —OC₁₋₆alkyl, —OCH₂CH₂OCH₃, —OCH₂C(O)N(CH₃)₂, —O-tetrahydropyranyl, —Opiperidinyl, and —N(R^{b})₂, where each R^{b} is independently selected from the group consisting of: —H, —C₁₋₆alkyl, —C₁₋₆alkyl-OH, —C₁₋₆haloalkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)piperidine, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R_{c}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, and —(CH₂)ₙ—R^{d}, where R^{d} is cyclopropyl, cyclobutyl, cyclopentyl, cyclohexyl, bicyclo[1.1.1]pentane, bicyclo[3.1.0]hexane, —C₃₋₇halocycloalkyl, oxetane, pyrrolidine, pyrrolidinone, piperidine, piperidinone, tetrahydropyranyl, tetrahydrofuranyl, oxepane, morpholine, 3-azabicyclo[3.1.0]hexane, tetrahydrothiophene-1,1-dioxide, tetrahydro-2H-thiopyran-1,1-dioxide, thiomorpholine-1,1-dioxide, tetrahydrothiophene-1,1-dioxide, 1,3-dihydroisobenzofuran, benzofuran, indoline, benzodioxine, benzodioxole, phenyl, benzyl, —CH₂CH₂phenyl, —CH₂CH₂CH₂phenyl, furan, pyrrole, pyrazole, imidazole, triazole, isoxazole, oxazole, thiazole, pyridine, pyrimidine, thiophene, pyrrolopyridine, benzimidazole, said R^{d} optionally substituted with up to 4 substituents each independently selected from the group consisting of: —F, —C₁₋₆alkyl, —C₁₋₆haloalkyl, cyclopropyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, morpholine, phenyl, pyrrolidinone, hydroxypiperidine, benzyl, imidazole, pyridine, where n is 0, 1, 2 or 3; or, alternatively, both R^{b} come together to form a monocyclic, bicyclic or tricyclic ring selected from the group consisting of: azetidine, pyrrole, pyrrolidine, pyrrolidinone, piperazine, piperazinone, piperidine, piperidinone, azepane, morpholine, 1-oxa-8-azaspiro[4.5]decan-3-one, 2-oxa-5-azabicyclo[2.2.1]heptane, 2-oxa-7-azaspiro[3.5]nonane, 2-oxa-8-azaspiro[4.5]decane, 2-oxa-8-azaspiro[4.5]decan-1-one, 6-oxa-9-azaspiro[4.5]decane, 7-oxa-2-azaspiro[3.5]nonane, octahydropyrrolo[3,4-c]pyrrole, octahydropyrrolo[1,2-a]pyrazine, oxaazabicyclo[2.2.1]heptane, thiomorpholine-1,1-dioxide, diazepane, 2,3,4,5-tetrahydropyrido[2,3-f][1,4]oxazepine, 5,6,7,8-tetrahydro[1,2,4]triazolo[4,3-a]pyrazine, 5,6,7,8-tetrahydroimidazo [1,2-a]pyrazine, 5,6,7,8-tetrahydropyrido[3,4-d]pyridazin-1(2H)-one, azaspiro[2.5]octane, azabicyclo[3.1.0]hexane, azabicyclo[4.1.0]heptane, diazaspiro[5.5]undecane, tetrahydroimidazopyrazine, dihydropyrrolopyridine, spiro[chroman-2,3′-pyrrolidin]-4-one, spiro[isochroman-1,4′-piperidine], 6′,7′-dihydro-5′H-spiro[piperidine-4,4′-thieno[3,2-c]pyridine], and 5′,6′-dihydro-4′H-spiro[piperidine-4,7′-thieno[2,3-c]pyridine], said 4-15-membered monocyclic, bicyclic or tricyclic ring optionally substituted with up to 4 substituents each independently selected from the group consisting of: halo, —C₁₋₆alkyl, —Ci-6haloalkyl, —C₁₋₆haloalkyl-C₃₋₇cycloalkyl, —C₃₋₇cycloalkyl, bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention have the formula (1) or are a pharmaceutically acceptable salt thereof, wherein W is —N(R^{b})₂, where each R^{b} is independently selected from the group consisting of: —H, —C₁₋₆alkyl, —C₁₋₆alkyl-OH, —C₁₋₆haloalkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)piperidine, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c} where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, and —(CH₂)ₙ—R^{d}, where R^{d} is cyclopropyl, cyclobutyl, cyclopentyl, cyclohexyl, bicyclo[1.1.1]pentane, bicyclo[3.1.0]hexane), —C₃₋₇halocycloalkyl, oxetane, pyrrolidine, pyrrolidinone, piperidine, piperidinone, tetrahydropyranyl, tetrahydrofuranyl, oxepane, morpholine, 3-azabicyclo[3.1.0]hexane, tetrahydrothiophene-1,1-dioxide, tetrahydro-2H-thiopyran-1,1-dioxide, thiomorpholine-1,1-dioxide, tetrahydrothiophene-1,1-dioxide, 1,3-dihydroisobenzofuran, benzofuran, indoline, benzodioxine, benzodioxole, phenyl, benzyl, —CH₂CH₂phenyl, —CH₂CH₂CH₂phenyl, furan, pyrrole, pyrazole, imidazole, triazole, isoxazole, oxazole, thiazole, pyridine, pyrimidine, thiophene, pyrrolopyridine, benzimidazole, said R^{d} optionally substituted with up to 4 substituents each independently selected from the group consisting of: —F, —C₁₋₆alkyl, —C₁₋₆haloalkyl, cyclopropyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, or —N(CH₃)₂, —N(CH₃)benzyl, heteroaryl, heterocycloalkyl, morpholine, phenyl, pyrrolidinone, hydroxypiperidine, benzyl, imidazole, pyridine, where n is 0, 1, 2 or 3.

In one embodiment, PDE7 inhibitors useful in the methods of the invention have the formula (1) or are a pharmaceutically acceptable salt thereof, wherein W is —N(R^{b})₂, where both R^{b} come together to form a monocyclic, bicyclic or tricyclic ring selected from the group consisting of: azetidine, pyrrole, pyrrolidine, pyrrolidinone, piperazine, piperazinone, piperidine, piperidinone, azepane, morpholine, 1-oxa-8-azaspiro[4.5]decan-3-one, 2-oxa-5-azabicyclo[2.2.1]heptane, 2-oxa-7-azaspiro[3.5]nonane, -2-oxa-8-azaspiro[4.5]decane, 2-oxa-8-azaspiro[4.5]decan-1-one, 6-oxa-9-azaspiro[4.5]decane, 7-oxa-2-azaspiro[3.5]nonane, octahydropyrrolo[3,4-c]pyrrole, octahydropyrrolo[1,2-a]pyrazine, oxaazabicyclo[2.2.1]heptane, thiomorpholine-1,1-dioxide, diazepane, 2,3,4,5-tetrahydropyrido[2,3-f][1,4]oxazepine, 5,6,7,8-tetrahydro[1,2,4]triazolo[4,3-a]pyrazine, 5,6,7,8-tetrahydroimidazo[1,2-c]pyrazine, 5,6,7,8-tetrahydropyrido[3,4-d]pyridazin-1(2H)-one, azaspiro[2.5]octane, azabicyclo[3.1.0]hexane, azabicyclo[4.1.0]heptane, diazaspiro[5.5]undecane, tetrahydroimidazopyrazine, dihydropyrrolopyridine, spiro[chroman-2,3′-pyrrolidin]-4-one, spiro[isochroman-1,4′-piperidine], 6′,7′-dihydro-5′H-spiro[piperidine-4,4′-thieno]3,2-c[pyridine], and 5′,6′-dihydro-4′H-spiro[piperidine-4,7′-thieno]2,3-c[pyridine], said monocyclic, bicyclic or tricyclic ring optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, -bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention have the formula (1) or are a pharmaceutically acceptable salt thereof, wherein W is selected from the group consisting of: —C₁₋₆alkyl, —C₁₋₆haloalkyl, —C₃₋₇cycloalkyl, —C(O)C₁₋₆alkyl, —C(O)C₃₋₇cycloalkyl, and —CH₂CH₂OCH₃.

In one embodiment, PDE7 inhibitors useful in the methods of the invention have the formula (1) or are a pharmaceutically acceptable salt thereof, wherein V is a bond and W is halo, —C₁₋₃alkyl, —C₁₋₃haloalkyl or —C₃₋₅cycloalkyl, said —C₃₋₅cycloalkyl optionally substituted with one or more halo or —C₁₋₃alkyl.

In one embodiment, PDE7 inhibitors useful in the methods of the invention have the formula (1) or are a pharmaceutically acceptable salt thereof, wherein V is —CH₂— and W is: —N(R^{b})₂, where each R^{b} is independently selected from the group consisting of: —H, —C₁₋₆alkyl, —C₁₋₆alkyl-OH, —C₁₋₆haloalkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)heterocycloalkyl, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, and —(CH₂)ₙ—R^{d}, where R^{d} is —C₃₋₇cycloalkyl, —C₃₋₇halocycloalkyl, heterocycloalkyl, aryl, or heteroaryl, each optionally substituted with up to 4 substituents each independently selected from the group consisting of: halo, —C₁₋₆alkyl, —C₁₋₆haloalkyl, —C₃₋₇cycloalkyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, phenyl, or benzyl, where n is 0, 1, 2 or 3; or, alternatively, both R^{b} come together to form a 4-15-membered, monocyclic, bicyclic or tricyclic ring, optionally containing up to 3 additional heteroatoms each independently selected from O (oxygen), N (nitrogen) and S (sulfur), said 4-15-membered monocyclic, bicyclic or tricyclic ring optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl. In a more specific embodiment such a PDE7 inhibitors useful in the methods of the invention or a pharmaceutically acceptable salt thereof, wherein W is selected from the group consisting of: halo, —C₁₋₆alkyl, —C₁₋₆haloalkyl, —CH₂OC₁₋₅alkyl, —CH₂OH, —CH₂CH₂OH, —CH₂CH₂OCH₃, —C(O)C₁₋₆alkyl, —C(O)C₃₋₇cycloalkyl, —C(O)OC₁₋₄alkyl, —C₃₋₇cycloalkyl, said —C₃₋₇cycloalkyl optionally substituted with one or more halo or —C₁₋₆alkyl, optionally substituted —C₁₋₆aryl, optionally substituted —C₁₋₆heteroaryl, —OH, —OC₁₋₆alkyl, —OCH₂CH₂OCH₃, —OCH₂C(O)N(CH₃)₂, —O-tetrahydropyranyl, —Opiperidinyl, and —N(R^{b})₂, where each R^{b} is independently selected from the group consisting of: —H, —C₁₋₆alkyl, —C₁₋₆alkyl-OH, —C₁₋₆haloalkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)piperidine, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂₋₁₂c, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, and —(CH₂)ₙ—R^{d}, where R^{d} is cyclopropyl, cyclobutyl, cyclopentyl, cyclohexyl, bicyclo[1.1.1]pentane, bicyclo[3.1.0]hexane, —C₃₋₇halocycloalkyl, oxetane, pyrrolidine, pyrrolidinone, piperidine, piperidinone, tetrahydropyranyl, tetrahydrofuranyl, oxepane, morpholine, 3-azabicyclo[3.1.0]hexane, tetrahydrothiophene-1,1-dioxide, tetrahydro-2H-thiopyran-1,1-dioxide, thiomorpholine-1,1-dioxide, tetrahydrothiophene-1,1-dioxide, 1,3-dihydroisobenzofuran, benzofuran, indoline, benzodioxine, benzodioxole, phenyl, benzyl, —CH₂CH₂phenyl, —CH₂CH₂CH₂phenyl, furan, pyrrole, pyrazole, imidazole, triazole, isoxazole, oxazole, thiazole, pyridine, pyrimidine, thiophene, pyrrolopyridine, benzimidazole, said R^{d} optionally substituted with up to 4 substituents each independently selected from the group consisting of: —F, —C₁₋₆alkyl, —C₁₋₆haloalkyl, cyclopropyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, morpholine, phenyl, pyrrolidinone, hydroxypiperidine, phenyl, benzyl, imidazole, pyridine, where n is 0, 1, 2 or 3; or, alternatively, both R^{b} come together to form a monocyclic, bicyclic or tricyclic ring selected from the group consisting of: azetidine, pyrrole, pyrrolidine, pyrrolidinone, piperazine, piperazinone, piperidine, piperidinone, azepane, morpholine, 1-oxa-8-azaspiro[4.5]decan-3-one, 2-oxa-5-azabicyclo[2.2.1]heptane, 2-oxa-7-azaspiro[3.5]nonane, 2-oxa-8-azaspiro[4.5]decane, 2-oxa-8-azaspiro[4.5]decan-1-one, 6-oxa-9-azaspiro[4.5]decane, 7-oxa-2-azaspiro[3.5]nonane, octahydropyrrolo[3,4-c]pyrrole, octahydropyrrolo[1,2-a]pyrazine, oxaazabicyclo[2.2.1]heptane, thiomorpholine-1,1-dioxide, diazepane, 2,3,4,5-tetrahydropyrido[2,3-f][1,4]oxazepine, 5,6,7,8-tetrahydro[1,2,4]triazolo[4,3-a]pyrazine, 5,6,7,8-tetrahydroimidazo [1,2-a]pyrazine, 5,6,7,8-tetrahydropyrido[3,4-d]pyridazin-1(2H)-one, azaspiro[2.5]octane, azabicyclo[3.1.0]hexane, azabicyclo[4.1.0]heptane, diazaspiro[5.5]undecane, tetrahydroimidazopyrazine, dihydropyrrolopyridine, spiro[chroman-2,3′-pyrrolidin]-4-one, spiro[isochroman-1,4′-piperidine], 6′,7′-dihydro-5′H-spiro[piperidine-4,4′-thieno[3,2-c]pyridine], 5′,6′-dihydro-4′H-spiro[piperidine-4,7′-thieno[2,3-c]pyridine], said monocyclic, bicyclic or tricyclic ring optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, bicyclo[2.2.1]heptane, —C₁₋₆ alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole and —C(O)phenyl.

In another more specific embodiment such a PDE7 inhibitors useful in the methods of the invention or a pharmaceutically acceptable salt thereof, wherein W is —N(R^{b})₂, where each R b is independently selected from the group consisting of: —H, —C₁₋₆alkyl, —C₁₋₆alkyl-OH, —C₁₋₆haloalkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)piperidine, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, and —(CH₂)ₙ—R^{d}, where R^{d} is cyclopropyl, cyclobutyl, cyclopentyl, cyclohexyl, bicyclo[1.1.1]pentane, bicyclo[3.1.0]hexane, —C₃₋₇halocycloalkyl, oxetane, pyrrolidine, pyrrolidinone, piperidine, piperidinone, tetrahydropyranyl, tetrahydrofuranyl, oxepane, morpholine, 3-azabicyclo[3.1.0]hexane, tetrahydrothiophene-1,1-dioxide, tetrahydro-2H-thiopyran-1,1-dioxide, thiomorpholine-1,1-dioxide, tetrahydrothiophene-1,1-dioxide, 1,3-dihydroisobenzofuran, benzofuran, indoline, benzodioxine, benzodioxole, phenyl, benzyl, —CH₂CH₂phenyl, —CH₂CH₂CH₂phenyl, furan, pyrrole, pyrazole, imidazole, triazole, isoxazole, oxazole, thiazole, pyridine, pyrimidine, thiophene, pyrrolopyridine, benzimidazole, said R^{d} optionally substituted with up to 4 substituents each independently selected from the group consisting of: —F, —C₁₋₆alkyl, —C₁₋₆haloalkyl, cyclopropyl, —OH, —OC₁₋₆alkyl, —O—C₁₋₆haloalkyl, —O— phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, morpholine, pyrrolidinone, hydroxypiperidine, phenyl, benzyl, imidazole, or pyridine, where n is 0, 1, 2 or 3. In another more specific embodiment such a PDE7 inhibitors useful in the methods of the invention or a pharmaceutically acceptable salt thereof, wherein W is —N(R^{b})₂, where both R^{b} come together to form a monocyclic, bicyclic or tricyclic ring selected from the group consisting of: azetidine, pyrrole, pyrrolidine, pyrrolidinone, piperazine, piperazinone, piperidine, piperidinone, azepane, morpholine, 1-oxa-8-azaspiro[4.5]decan-3-one, 2-oxa-5-azabicyclo[2.2.1]heptane, 2-oxa-7-azaspiro[3.5]nonane, 2-oxa-8-azaspiro[4.5]decane, 2-oxa-8-azaspiro[4.5]decan-1-one, 6-oxa-9-azaspiro[4.5]decane, 7-oxa-2-azaspiro[3.5]nonane, octahydropyrrolo[3,4-c]pyrrole, octahydropyrrolo[1,2-a]pyrazine, oxaazabicyclo[2.2.1]heptane, thiomorpholine-1,1-dioxide, diazepane, 2,3,4,5-tetrahydropyrido[2,3-f][1,4]oxazepine, 5,6,7,8-tetrahydro[1,2,4]triazolo[4,3-a]pyrazine, 5,6,7,8-tetrahydroimidazo [1,2-a]pyrazine, 5,6,7,8-tetrahydropyrido[3,4-d]pyridazin-1(2H)-one, azaspiro[2.5]octane, azabicyclo[3.1.0]hexane, azabicyclo[4.1.0]heptane, diazaspiro[5.5]undecane, tetrahydroimidazopyrazine, dihydropyrrolopyridine, spiro[chroman-2,3′-pyrrolidin]-4-one, spiro[isochroman-1,4′-piperidine], 6′,7′-dihydro-5′H-spiro[piperidine-4,4′-thieno[3,2-c]pyridine], and 5′,6′-dihydro-4′H-spiro[piperidine-4,7′-thieno[2,3-c]pyridine], said monocyclic, bicyclic or tricyclic ring optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole, or —C(O)phenyl.

In one embodiment, PDE7 inhibitors useful in the methods of the invention have the formula (1) or are a pharmaceutically acceptable salt thereof, wherein W is selected from the group consisting of: halo, —C₁₋₆alkyl, —C₁₋₆haloalkyl, —CH₂OC₁₋₅alkyl, —CH₂OH, —CH₂CH₂OH, —CH₂CH₂OCH₃, —C(O)C₁₋₆alkyl, —C(O)C₃₋₇cycloalkyl, —C(O)OC₁₋₄alkyl, —C₃₋₇cycloalkyl, said —C₃₋₇cycloalkyl optionally substituted with one or more halo or —C₁₋₆alkyl, optionally substituted —C₁₋₆aryl, optionally substituted —C₁₋₆heteroaryl, —OH, —OC₁₋₆alkyl, —OCH₂CH₂OCH₃, —OCH₂C(O)N(CH₃)₂, —O— tetrahydropyranyl, —Opiperidinyl, and —N(R^{b})₂, where each R^{b} is independently selected from the group consisting of: —H, —C₁₋₆alkyl, —C₁₋₆alkyl-OH, —C₁₋₆haloalkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)heterocycloalkyl, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, and —(CH₂)ₙ—R^{d}, where R^{d} is —C₃₋₇cycloalkyl, —C₃₋₇halocycloalkyl, heterocycloalkyl, aryl, and heteroaryl, each optionally substituted with up to 4 substituents each independently selected from the group consisting of: halo, —C₁₋₆alkyl, —C₁₋₆haloalkyl, —C₃₋₇cycloalkyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, phenyl, or benzyl, where n is 0, 1, 2 or 3; or, alternatively, both R^{b} come together to form a 4-15-membered, monocyclic, bicyclic or tricyclic ring, optionally containing up to 3 additional heteroatoms each independently selected from O (oxygen), N (nitrogen) and S (sulfur), said 4-15-membered monocyclic, bicyclic or tricyclic ring optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, -bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole, or —C(O)phenyl. In a more specific embodiment such a PDE7 inhibitors useful in the methods of the invention or a pharmaceutically acceptable salt thereof, wherein W is selected from the group consisting of: halo, —C₁₋₆alkyl, —C₁₋₆haloalkyl, —CH₂OC₁₋₅alkyl, —CH₂OH, —CH₂CH₂OH, —CH₂CH₂OCH₃, —C(O)C₁₋₆alkyl, —C(O)C₃₋₇cycloalkyl, —C(O)OC₁₋₄alkyl, —C₃₋₇cycloalkyl, said —C₃₋₇cycloalkyl optionally substituted with one or more halo or —C₁₋₆alkyl, optionally substituted —C₁₋₆aryl, optionally substituted —C₁₋₆heteroaryl, —OH, —OC₁₋₆alkyl, —OCH₂CH₂OCH₃, —OCH₂C(O)N(CH₃)₂, —O-tetrahydropyranyl, —Opiperidinyl, and —N(R^{b})₂ where each R^{b} is independently selected from the group consisting of: —H, —C₁₋₆alkyl, —C₁₋₆alkyl-OH, —C₁₋₆halo alkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)piperidine, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c}, where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, and —(CH₂)ₙ—R^{d}, where R^{d} is cyclopropyl, cyclobutyl, cyclopentyl, cyclohexyl, bicyclo[1.1.1]pentane, bicyclo[3.1.0]hexane), —C₃₋₇halocycloalkyl, oxetane, pyrrolidine, pyrrolidinone, piperidine, piperidinone, tetrahydropyranyl, tetrahydrofuranyl, oxepane, morpholine, 3-azabicyclo[3.1.0]hexane, tetrahydrothiophene-1,1-dioxide, tetrahydro-2H-thiopyran-1,1-dioxide, thiomorpholine-1,1-dioxide, tetrahydrothiophene-1,1-dioxide, 1,3-dihydroisobenzofuran, benzofuran, indoline, benzodioxine, benzodioxole, phenyl, benzyl, —CH₂CH₂phenyl, —CH₂CH₂CH₂phenyl, furan, pyrrole, pyrazole, imidazole, triazole, isoxazole, oxazole, thiazole, pyridine, pyrimidine, thiophene, pyrrolopyridine, benzimidazole, said R^{d} optionally substituted with up to 4 substituents each independently selected from the group consisting of: —F, —C₁₋₆alkyl, —C₁₋₆haloalkyl, cyclopropyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, morpholine, pyrrolidinone, hydroxypiperidine, phenyl, benzyl, imidazole, or pyridine, where n is 0, 1, 2 or 3; or, alternatively, both R^{b} come together to form a monocyclic, bicyclic or tricyclic ring selected from the group consisting of: azetidine, pyrrole, pyrrolidine, pyrrolidinone, piperazine, piperazinone, piperidine, piperidinone, azepane, morpholine, 1-oxa-8-azaspiro[4.5]decan-3-one, 2-oxa-5-azabicyclo[2.2.1]heptane, 2-oxa-7-azaspiro[3.5]nonane, 2-oxa-8-azaspiro[4.5]decane, 2-oxa-8-azaspiro[4.5]decan-1-one, 6-oxa-9-azaspiro[4.5]decane, 7-oxa-2-azaspiro[3.5]nonane, octahydropyrrolo[3,4-c]pyrrole, octahydropyrrolo[1,2-a]pyrazine, oxaazabicyclo[2.2.1]heptane, thiomorpholine-1,1-dioxide, diazepane, 2,3,4,5-tetrahydropyrido[2,3-f][1,4]oxazepine, 5,6,7,8-tetrahydro[1,2,4]triazolo[4,3-a]pyrazine, 5,6,7,8-tetrahydroimidazo[1,2-a]pyrazine, 5,6,7,8-tetrahydropyrido[3,4-d]pyridazin-1 (2H)-one, azaspiro[2.5]octane, azabicyclo[3.1.0]hexane, azabicyclo[4.1.0]heptane, diazaspiro[5.5]undecane, tetrahydroimidazopyrazine, dihydropyrrolopyridine, spiro[chroman-2,3′-pyrrolidin]-4-one, spiro[isochroman-1,4′-piperidine], 6′,7′-dihydro-5′H-spiro[piperidine-4,4′-thieno[3,2-c]pyridine], 5′,6′-dihydro-4′H-spiro[piperidine-4,7′-thieno[2,3-c]pyridine], said monocyclic, bicyclic or tricyclic ring optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole and —C(O)phenyl.

In another more specific embodiment such a PDE7 inhibitors useful in the methods of the invention or a pharmaceutically acceptable salt thereof, wherein W is —N(R^{b})₂, where each R^{b} is independently selected from the group consisting of: —H, —C₁₋₆alkyl, —C₁₋₆alkyl-OH, —C₁₋₆haloalkyl, —C₁₋₆haloalkylOH, —C₁₋₆haloalkyl-C₃₋₇cycloalkyl, —C₃₋₇cycloalkyl, —CH₂-alkenyl, —CH₂-alkynyl, —CH₂C₃₋₇cycloalkyl, —CH₂C(O)C(CH₃)₃, —CH₂C(O)C₁₋₆alkyl, —CH₂C(O)piperidine, —CH₂C(O)OC₁₋₆alkyl, —CH₂C(O)N(C₁₋₄alkyl)₂, —CH₂CH(OH)C₃₋₇cycloalkyl, —C(CH₃)₂OH, —C(CH₃)₂CH₂OCH₃, —CH(CH₃)C(O)N(C₁₋₄alkyl)₂, —CH₂CF₂C₃₋₇cycloalkyl, —CH₂CH₂—R^{c} where R^{c} is —C₂₋₆alkynyl, —C₃₋₇cycloalkyl, —OH, —O—C₁₋₆alkyl, —OC(CH₃)₂, —O—C₁₋₆haloalkyl, —Ophenyl, —Opyridyl, —CH₂N(CH₃)₂, —C(O)NHCH₃, —C(O)N(CH₃)₂, —CN, —NHCH₃, —N(CH₃)₂, —NHC(O)CH₃, or —SO₂CH₃, and —(CH₂)ₙ—R^{d}, where R^{d} is cyclopropyl, cyclobutyl, cyclopentyl, cyclohexyl, bicyclo[1.1.1]pentane, bicyclo[3.1.0]hexane, —C₃₋₇halocycloalkyl, oxetane, pyrrolidine, pyrrolidinone, piperidine, piperidinone, tetrahydropyranyl, tetrahydrofuranyl, oxepane, morpholine, 3-azabicyclo[3.1.0]hexane, tetrahydrothiophene-1,1-dioxide, tetrahydro-2H-thiopyran-1,1-dioxide, thiomorpholine-1,1-dioxide, tetrahydrothiophene-1,1-dioxide, 1,3-dihydroisobenzofuran, benzofuran, indoline, benzodioxine, benzodioxole, phenyl, benzyl, —CH₂CH₂phenyl, —CH₂CH₂CH₂phenyl, furan, pyrrole, pyrazole, imidazole, triazole, isoxazole, oxazole, thiazole, pyridine, pyrimidine, thiophene, pyrrolopyridine, benzimidazole, said R^{d} optionally substituted with up to 4 substituents each independently selected from the group consisting of: —F, —C₁₋₆alkyl, —C₁₋₆haloalkyl, cyclopropyl, —OH, —OC₁₋₆alkyl, —O—C₁₋₆haloalkyl, —O-phenyl, —O-pyridyl, —CH₂OH, —CH₂OCH₃, —CH₂OCH₂CH₃, —CN, —N(CH₃)₂, —N(CH₃)benzyl, heteroaryl, heterocycloalkyl, morpholine, pyrrolidinone, hydroxypiperidine, phenyl, benzyl, imidazole, or pyridine, where n is 0, 1, 2 or 3. In another more specific embodiment such a PDE7 inhibitors useful in the methods of the invention or a pharmaceutically acceptable salt thereof, wherein W is —N(R^{b})₂, where both R^{b} come together to form a monocyclic, bicyclic or tricyclic ring selected from the group consisting of: azetidine, pyrrole, pyrrolidine, pyrrolidinone, piperazine, piperazinone, piperidine, piperidinone, azepane, morpholine, 1-oxa-8-azaspiro[4.5]decan-3-one, 2-oxa-5-azabicyclo[2.2.1]heptane, 2-oxa-7-azaspiro[3.5]nonane, 2-oxa-8-azaspiro[4.5]decane, 2-oxa-8-azaspiro[4.5]decan-1-one, 6-oxa-9-azaspiro[4.5]decane, 7-oxa-2-azaspiro[3.5]nonane, octahydropyrrolo[3,4-c]pyrrole, octahydropyrrolo[1,2-a]pyrazine, oxaazabicyclo[2.2.1]heptane, thiomorpholine-1,1-dioxide, diazepane, 2,3,4,5-tetrahydropyrido[2,3-f][1,4]oxazepine, 5,6,7,8-tetrahydro[1,2,4]triazolo[4,3-a]pyrazine, 5,6,7,8-tetrahydroimidazo[1,2-a]pyrazine, 5,6,7,8-tetrahydropyrido[3,4-d]pyridazin-1(2H)-one, azaspiro[2.5]octane, azabicyclo[3.1.0]hexane, azabicyclo[4.1.0]heptane, diazaspiro[5.5]undecane, tetrahydroimidazopyrazine, dihydropyrrolopyridine, spiro[chroman-2,3′-pyrrolidin]-4-one, spiro[isochroman-1,4′-piperidine], 6′,7′-dihydro-5′H-spiro[piperidine-4,4′-thieno[3,2-c]pyridine], and 5′,6′-dihydro-4′H-spiro[piperidine-4,7′-thieno[2,3-c]pyridine], said 4-15-membered monocyclic, bicyclic or tricyclic ring optionally substituted with up to 4 substituents each independently selected from the group consisting of: halo, —C₁₋₆alkyl, —C₁₋₆haloalkyl, —C₁₋₆haloalkyl-C₃₋₇cycloalkyl, —C₃₋₇cycloalkyl, bicyclo[2.2.1]heptane, —C₁₋₆alkyl-OH, —C(CH₃)₂OH, —CH₂OC₁₋₆alkyl, —CH₂C(O)OCH₂CH₃, —CH₂CH₂OC₁₋₆alkyl, —CH₂CH₂OC₁₋₆haloalkyl, —CH₂CH₂C(O)OCH₃, —OH, —OC₁₋₆alkyl, —OCH₂—C₃₋₇cycloalkyl, —OCH₂CH₂OCH₃, ═O, —CH₂N(CH₃)₂, —CH₂CH₂N(CH₃)₂, —C(O)C₁₋₆alkyl, —C(O)H, —C(O)OH, —C(O)OC₁₋₆alkyl, —C(O)CH₃, —C(O)NH₂, —C(O)NHCH₃, —C(O)N(CH₃)₂, —CN, —NH₂, —NHC(O)CH₃, —N(CH₃)₂, —SO₂CH₃, oxetane, pyrrolidine, morpholine, furan, thiazole, pyridyl, phenyl, thiophene, —CH₂imidazole, —CH₂furan, —CH₂-thiophene, —CH₂pyridyl, benzyl, —C(O)-tetrahydrofuran, —C(O)pyrrole or —C(O)phenyl.

In one embodiment, PDE7 inhibitors useful in the methods of the invention are selected from the group consisting of: methyl 5-chloro-7-oxo-7,8-dihydro-6H-spiro[[1,3]oxazolo[5,4-f]quinazoline-9,1′-cyclohexane]-2-carboxylate; 5-chloro-7-oxo-7,8-dihydro-6H-spiro[[1,3]oxazolo[5,4-f]quinazoline-9,1′-cyclohexane]-2-carboxylic acid; 5-chloro-2-(chloromethyl)-7,8-dihydro-6H-spiro[[1,3]oxazolo[5,4-f]quinazoline-9,1′-cyclohexane]-7-one; 5-chloro-2-{[(2-methoxyethyl)amino]methyl}-7,8-dihydro-6H-spiro[[1,3]oxazolo[5,4-f]quinazoline-9,1′-cyclohexane]-7-one; 5-chloro-2-[(2-methoxyethoxy)methyl]-7,8-dihydro-6H-spiro[[1,3]oxazolo[5,4-f]quinazoline-9,1′-cyclohexane]-7-one; ethyl 2-{5-chloro-7-oxo-7,8-dihydro-6H-spiro[[1,3]oxazolo[5,4f]quinazoline-9,1′-cyclohexane]-2-yl}acetate; 5-chloro-2-(4-methoxypiperidine-1-carbonyl)-7,8-dihydro-6H-spiro[[1,3]oxazolo[5,4-f]quinazoline-9,1′-cyclohexane]-7-one; 5-fluoro-2-[(3-methoxyazetidin-1-yl)methyl]-7,8-dihydro-6H-spiro[[1,3]oxazolo[5,4-f]quinazoline-9,1′-cyclohexane]-7-one; methyl 5-chloro-4′,4′-difluoro-7-oxo-7,8-dihydro-6H-spiro[[1,3]oxazolo[5,4-f]quinazoline-9,1′-cyclohexane]-2-carboxylate; 5-chloro-4′,4′-difluoro-2-[(4-methylpiperazin-1-yl)methyl]-7,8-dihydro-6H-spiro[[1,3]oxazolo[5,4-f]quinazoline-9,1′-cyclohexane]-7-one; 5-chloro-4′,4′-difluoro-2-methyl-7,8-dihydro-6H-spiro[[1,3]oxazolo[5,4-f]quinazoline-9,1′-cyclohexane]-7-one; 5-chloro-2,4′,4′-trimethyl-7,8-dihydro-6H-spiro[[1,3]oxazolo[5,4-f]quinazoline-9,1′-cyclohexane]-7-one; 5-chloro-7,8-dihydro-6H-spiro[[1,3]oxazolo[5,4-f]quinazoline-9,1′-cyclohexane]-7-one; 5-chloro-2-methyl-7,8-dihydro-6H-spiro[[1,3]oxazolo[5,4-f]quinazoline-9,1′-cyclohexane]-7-one; 5-chloro-2-ethyl-7,8-dihydro-6H-spiro[[1,3]oxazolo[5,4-f]quinazoline-9,1′-cyclohexane]-7-one; 5-chloro-2-propyl-7,8-dihydro-6H-spiro[[1,3]oxazolo[5,4-f]quinazoline-9,1′-cyclohexane]-7-one; 5-chloro-2-(propan-2-yl)-7,8-dihydro-6H-spiro[[1,3]oxazolo[5,4-f]quinazoline-9,1′-cyclohexane]-7-one; 5-chloro-2-cyclopropyl-7,8-dihydro-6H-spiro[[1,3]oxazolo[5,4-f]quinazoline-9,1′-cyclohexane]-7-one; 5-chloro-2-(methoxymethyl)-7,8-dihydro-6H-spiro[[1,3]oxazolo[5,4-f]quinazoline-9,1′-cyclohexane]-7-one; 5-chloro-2-(hydroxymethyl)-7,8-dihydro-6H-spiro[[1,3]oxazolo[5,4-f]quinazoline-9,1′-cyclohexane]-7-one; 5-chloro-2-(2-methoxyethyl)-7,8-dihydro-6H-spiro[[1,3]oxazolo[5,4-f]quinazoline-9,1′-cyclohexane]-7-one; 5-chloro-2-(2-hydroxyethyl)-7,8-dihydro-6H-spiro[[1,3]oxazolo[5,4-f]quinazoline-9,1′-cyclohexane]-7-one; N-[5-chloro-7-oxo-7,8-dihydro-6H-spiro[[1,3]oxazolo[5,4-f]quinazoline-9,1′-cyclohexane]-2-yl]acetamide; 2-[bis(2-methoxyethyl)amino]-5-chloro-7,8-dihydro-6H-spiro[[1,3]oxazolo[5,4-f]quinazoline-9,1′-cyclohexane]-7-one; 5-chloro-7-oxo-7,8-dihydro-6H-spiro[[1,3]oxazolo[5,4-f]quinazoline-9,1′-cyclohexane]-2-carboxamide; 5-chloro-2-[(dimethylamino)methyl]-7,8-dihydro-6H-spiro[[1,3]oxazolo[5,4-f]quinazoline-9,1′-cyclohexane]-7-one; 5-chloro-2-[(4-methylpiperazin-1-yl)methyl]-7,8-dihydro-6H-spiro[[1,3]oxazolo[5,4-f]quinazoline-9,1′-cyclohexane]-7-one; 5-chloro-2-(morpholin-4-ylmethyl)-7,8-dihydro-6H-spiro[[1,3]oxazolo[5,4-f]quinazoline-9,1′-cyclohexane]-7-one; 5-chloro-2-[(4-hydroxypiperidin-1-yl)methyl]-7,8-dihydro-6H-spiro[[1,3]oxazolo[5,4-f]quinazoline-9,1′-cyclohexane]-7-one; 5-chloro-2-[(methylamino)methyl]-7,8-dihydro-6H-spiro[[1,3]oxazolo[5,4-f]quinazoline-9,1′-cyclohexane]-7-one; 5-chloro-2-[(cyclopropylamino)methyl]-7,8-dihydro-6H-spiro[[1,3]oxazolo[5,4-f]quinazoline-9,1′-cyclohexane]-7-one; 5-chloro-2-(piperazin-1-ylmethyl)-7,8-dihydro-6H-spiro[[1,3]oxazolo[5,4-f]quinazoline-9,1′-cyclohexane]-7-one; 5-chloro-2-(pyrrolidin-1-ylmethyl)-7,8-dihydro-6H-spiro[[1,3]oxazolo[5,4-f]quinazoline-9,1′-cyclohexane]-7-one; 5-chloro-2-{[(propan-2-yl)amino]methyl}-7,8-dihydro-6H-spiro[[1,3]oxazolo[5,4-f]quinazoline-9,1′-cyclohexane]-7-one; 5-chloro-2-[(ethylamino)methyl]-7,8-dihydro-6H-spiro[[1,3]oxazolo[5,4-f]quinazoline-9,1′-cyclohexane]-7-one; 5-chloro-2-{[(2-hydroxyethyl)amino]methyl}-7,8-dihydro-6H-spiro[[1,3]oxazolo[5,4-f]quinazoline-9,1′-cyclohexane]-7-one; 5-chloro-2-[(4,4-difluoropiperidin-1-yl)methyl]-7,8-dihydro-6H-spiro[[1,3]oxazolo[5,4-f]quinazoline-9,1′-cyclohexane]-7-one; 5-chloro-2-{[(2,2,2-trifluoroethyl)amino]methyl}-7,8-dihydro-6H-spiro[[1,3]oxazolo[5,4-f]quinazoline-9,1′-cyclohexane]-7-one; 5-chloro-2-{[(2-hydroxyethyl)(methyl)amino]methyl}-7,8-dihydro-6H-spiro[[1,3]oxazolo[5,4-f]quinazoline-9,1′-cyclohexane]-7-one; 5-chloro-2-[(3,3-difluoroazetidin-1-yl)methyl]-7,8-dihydro-6H-spiro[[1,3]oxazolo[5,4-f]quinazoline-9,1′-cyclohexane]-7-one; 5-chloro-2-[(3,3-difluoropyrrolidin-1-yl)methyl]-7,8-dihydro-6H-spiro[[1,3]oxazolo[5,4-f]quinazoline-9,1′-cyclohexane]-7-one; 2-[(4-acetylpiperazin-1-yl)methyl]-5-chloro-7,8-dihydro-6H-spiro[[1,3]oxazolo[5,4-f]quinazoline-9,1′-cyclohexane]-7-one; 5-chloro-2-[(4-propanoylpiperazin-1-yl)methyl]-7,8-dihydro-6H-spiro[[1,3]oxazolo[5,4-f]quinazoline-9,1′-cyclohexane]-7-one; 5-chloro-2-{2-oxa-8-azaspiro[4.5]decan-8-ylmethyl}-7,8-dihydro-6H-spiro[[1,3]oxazolo[5,4-f]quinazoline-9,1′-cyclohexane]-7-one; 5-chloro-2-{[4-(oxetan-3-yl)piperazin-1-yl]methyl}-7,8-dihydro-6H-spiro[[1,3]oxazolo[5,4-f]quinazoline-9,1′-cyclohexane]-7-one; 2-({5-chloro-7-oxo-7,8-dihydro-6H-spiro[[1,3]oxazolo[5,4-f]quinazoline-9,1′-cyclohexane]-2-ylmethyl}amino)-N,N-dimethylacetamide; tert-butyl 2-[({5-chloro-7-oxo-7,8-dihydro-6H-spiro[[1,3]oxazolo[5,4-f]quinazoline-9,1′-cyclohexane]-2-ylmethyl}amino)methyl]piperidine-1-carboxylate; 5-chloro-2-({[(3,5-difluoropyridin-2-yl)methyl]amino}methyl)-7,8-dihydro-6H-spiro[[1,3]oxazolo[5,4-f]quinazoline-9,1′-cyclohexane]-7-one; 5-chloro-2-{[(piperidin-2-ylmethyl)amino]methyl}-7,8-dihydro-6H-spiro[[1,3]oxazolo[5,4-f]quinazoline-9,1′-cyclohexane]-7-one; 5-chloro-2-({[2-oxo-2-(piperidin-1-yl)ethyl]amino}methyl)-7,8-dihydro-6H-spiro[[1,3]oxazolo[5,4-f]quinazoline-9,1′-cyclohexane]-7-one; 2-({5-chloro-7-oxo-7,8-dihydro-6H-spiro[[1,3]oxazolo[5,4-f]quinazoline-9,1′-cyclohexane]-2-ylmethyl}amino)-N-ethyl-N-methylacetamide; 5-chloro-2-[(4-methyl-3-oxopiperazin-1-yl)methyl]-7,8-dihydro-6H-spiro[[1,3]oxazolo[5,4-f]quinazoline-9,1′-cyclohexane]-7-one; 5-chloro-2-[(4-ethyl-3-oxopiperazin-1-yl)methyl]-7,8-dihydro-6H-spiro[[1,3]oxazolo[5,4-f]quinazoline-9,1′-cyclohexane]-7-one; 5-chloro-2-{[(pyridin-2-ylmethyl)amino]methyl}-7,8-dihydro-6H-spiro[[1,3]oxazolo[5,4-f]quinazoline-9,1′-cyclohexane]-7-one; 5-chloro-2-{[(pyrimidin-2-ylmethyl)amino]methyl}-7,8-dihydro-6H-spiro[[1,3]oxazolo[5,4-f]quinazoline-9,1′-cyclohexane]-7-one; 5-chloro-2-({[(5-methoxypyridin-3-yl)methyl]amino}methyl)-7,8-dihydro-6H-spiro[[1,3]oxazolo[5,4-f]quinazoline-9,1′-cyclohexane]-7-one; 5-chloro-2-[(2,4-dimethyl-3-oxopiperazin-1-yl)methyl]-7,8-dihydro-6H-spiro[[1,3]oxazolo[5,4-f]quinazoline-9,1′-cyclohexane]-7-one; ethyl 1-{5-chloro-7-oxo-7,8-dihydro-6H-spiro[[1,3]oxazolo[5,4-f]quinazoline-9,1′-cyclohexane]-2-ylmethyl}piperidine-4-carboxylate; 5-chloro-2-({1-oxo-2-oxa-8-azaspiro[4.5]decan-8-yl}methyl)-7,8-dihydro-6H-spiro[[1,3]oxazolo[5,4-f]quinazoline-9,1′-cyclohexane]-7-one; 2-[(4-acetylpiperidin-1-yl)methyl]-5-chloro-7,8-dihydro-6H-spiro[[1,3]oxazolo[5,4-f]quinazoline-9,1′-cyclohexane]-7-one; methyl 1-{5-chloro-7-oxo-7,8-dihydro-6H-spiro[[1,3]oxazolo[5,4-f]quinazoline-9,1′-cyclohexane]-2-ylmethyl}piperidine-4-carboxylate; 5-chloro-2-({[(5-fluoropyridin-2-yl)methyl]amino}methyl)-7,8-dihydro-6H-spiro[[1,3]oxazolo[5,4-f]quinazoline-9,1′-cyclohexane]-7-one; 5-chloro-2-{[3-(2-methylpropoxy)azetidin-1-yl]methyl}-7,8-dihydro-6H-spiro[[1,3]oxazolo[5,4-f]quinazoline-9,1′-cyclohexane]-7-one; 5-chloro-2-[(4-ethoxypiperidin-1-yl)methyl]-7,8-dihydro-6H-spiro[[1,3]oxazolo[5,4-f]quinazoline-9,1′-cyclohexane]-7-one; 5-chloro-2-{[4-(cyclopropylmethoxy)piperidin-1-yl]methyl}-7,8-dihydro-6H-spiro[[1,3]oxazolo[5,4-f]quinazoline-9,1′-cyclohexane]-7-one; 5-chloro-2-{[4-hydroxy-4-(trifluoromethyl)piperidin-1-yl]methyl}-7,8-dihydro-6H-spiro[[1,3]oxazolo[5,4-f]quinazoline-9,1′-cyclohexane]-7-one; 5-chloro-2-{[4-(propan-2-yloxy)piperidin-1-yl]methyl}-7,8-dihydro-6H-spiro[[1,3]oxazolo[5,4-f]quinazoline-9,1′-cyclohexane]-7-one; 5-chloro-2-[(3-fluoroazetidin-1-yl)methyl]-7,8-dihydro-6H-spiro[[1,3]oxazolo[5,4-f]quinazoline-9,1′-cyclohexane]-7-one; 5-chloro-2-[(3-methoxyazetidin-1-yl)methyl]-7,8-dihydro-6H-spiro[[1,3]oxazolo[5,4-f]quinazoline-9,1′-cyclohexane]-7-one; 5-chloro-2-{[3-(methoxymethyl)azetidin-1-yl]methyl}-7,8-dihydro-6H-spiro[[1,3]oxazolo[5,4-f]quinazoline-9,1′-cyclohexane]-7-one; 5-chloro-2-{7-oxa-2-azaspiro[3.5]nonan-2-ylmethyl}-7,8-dihydro-6H-spiro[[1,3]oxazolo[5,4-f]quinazoline-9,1′-cyclohexane]-7-one; 5-chloro-2-[(4-methoxy-4-methylpiperidin-1-yl)methyl]-7,8-dihydro-6H-spiro[[1,3]oxazolo[5,4-f]quinazoline-9,1′-cyclohexane]-7-one; 5-chloro-2-({3-oxo-1-oxa-8-azaspiro[4.5]decan-8-yl}methyl)-7,8-dihydro-6H-spiro[[1,3]oxazolo[5,4-f]quinazoline-9,1′-cyclohexane]-7-one; 2-({5-chloro-7-oxo-7,8-dihydro-6H-spiro[[1,3]oxazolo[5,4-f]quinazoline-9,1′-cyclohexane]-2-ylmethyl}(methyl)amino)-N,N-dimethylacetamide; 5-chloro-2-({[(6-methylpyridin-3-yl)methyl]amino}methyl)-7,8-dihydro-6H-spiro[[1,3]oxazolo[5,4-f]quinazoline-9,1′-cyclohexane]-7-one; 5-chloro-2-{[methyl(oxan-4-ylmethyl)amino]methyl}-7,8-dihydro-6H-spiro[[1,3]oxazolo[5,4-f]quinazoline-9,1′-cyclohexane]-7-one; 5-chloro-2-({methyl[(3-methyloxetan-3-yl)methyl]amino}methyl)-7,8-dihydro-6H-spiro[[1,3]oxazolo[5,4-f]quinazoline-9,1′-cyclohexane]-7-one; 5-chloro-2-({[(3-fluorooxetan-3-yl)methyl]amino}methyl)-7,8-dihydro-6H-spiro[[1,3]oxazolo[5,4-f]quinazoline-9,1′-cyclohexane]-7-one; 5-chloro-2-{[(oxan-4-ylmethyl)amino]methyl}-7,8-dihydro-6H-spiro[[1,3]oxazolo[5,4-f]quinazoline-9,1′-cyclohexane]-7-one; 5-chloro-2-{2-oxa-7-azaspiro[3.5]nonan-7-ylmethyl}-7,8-dihydro-6H-spiro[[1,3]oxazolo[5,4-f]quinazoline-9,1′-cyclohexane]-7-one; 5-chloro-2-({[(3-methyloxetan-3-yl)methyl]amino}methyl)-7,8-dihydro-6H-spiro[[1,3]oxazolo[5,4-f]quinazoline-9,1′-cyclohexane]-7-one; 5-chloro-2-{2-oxa-5-azabicyclo[2.2.1]heptan-5-ylmethyl}-7,8-dihydro-6H-spiro[[1,3]oxazolo[5,4-f]quinazoline-9,1′-cyclohexane]-7-one; 5-chloro-2-{[methyl(2,2,2-trifluoroethyl)amino]methyl}-7,8-dihydro-6H-spiro[[1,3]oxazolo[5,4-f]quinazoline-9,1′-cyclohexane]-7-one; 5-chloro-2-{[(oxetan-3-ylmethyl)amino]methyl}-7,8-dihydro-6H-spiro[[1,3]oxazolo[5,4-f]quinazoline-9,1′-cyclohexane]-7-one; 5-chloro-2-[(4-oxopiperidin-1-yl)methyl]-7,8-dihydro-6H-spiro[[1,3]oxazolo[5,4-f]quinazoline-9,1′-cyclohexane]-7-one; 5-chloro-2-({[(2-methoxyphenyl)methyl]amino}methyl)-7,8-dihydro-6H-spiro[[1,3]oxazolo[5,4-f]quinazoline-9,1′-cyclohexane]-7-one; 5-chloro-2-({[(2,4-dimethoxyphenyl)methyl]amino}methyl)-7,8-dihydro-6H-spiro[[1,3]oxazolo[5,4-f]quinazoline-9,1′-cyclohexane]-7-one; 5-chloro-2-{[4-(propan-2-yl)piperazin-1-yl]methyl}-7,8-dihydro-6H-spiro[[1,3]oxazolo[5,4-f]quinazoline-9,1′-cyclohexane]-7-one; 5-chloro-2-[(3,3,4-trimethylpiperazin-1-yl)methyl]-7,8-dihydro-6H-spiro[[1,3]oxazolo[5,4-f]quinazoline-9,1′-cyclohexane]-7-one; 5-chloro-2-{[2-(hydroxymethyl)piperidin-1-yl]methyl}-7,8-dihydro-6H-spiro[[1,3]oxazolo[5,4-f]quinazoline-9,1′-cyclohexane]-7-one; 5-chloro-2-({[1-(hydroxymethyl)cyclopentyl]amino}methyl)-7,8-dihydro-6H-spiro[[1,3]oxazolo[5,4-f]quinazoline-9,1′-cyclohexane]-7-one; 5-chloro-2-{[(2R)-2-(hydroxymethyl)pyrrolidin-1-yl]methyl}-7,8-dihydro-6H-spiro[[1,3]oxazolo[5,4-f]quinazoline-9,1′-cyclohexane]-7-one; 5-chloro-2-[(2-methylpiperidin-1-yl)methyl]-7,8-dihydro-6H-spiro[[1,3]oxazolo[5,4-f]quinazoline-9,1′-cyclohexane]-7-one; 5-chloro-2-{[2-(2-methylpropyl)morpholin-4-yl]methyl}-7,8-dihydro-6H-spiro[[1,3]oxazolo[5,4-f]quinazoline-9,1′-cyclohexane]-7-one; 5-chloro-2-{[(2-cyclobutyl-2,2-difluoroethyl)amino]methyl}-7,8-dihydro-6H-spiro[[1,3]oxazolo[5,4-f]quinazoline-9,1′-cyclohexane]-7-one; 5-chloro-2-[(3-ethylmorpholin-4-yl)methyl]-7,8-dihydro-6H-spiro[[1,3]oxazolo[5,4-f]quinazoline-9,1′-cyclohexane]-7-one; 5-chloro-2-{[(2-cyclohexyl-2-hydroxyethyl)amino]methyl}-7,8-dihydro-6H-spiro[[1,3]oxazolo[5,4-f]quinazoline-9,1′-cyclohexane]-7-one; 5-chloro-2-{6-oxa-9-azaspiro[4.5]decan-9-ylmethyl}-7,8-dihydro-6H-spiro[[1,3]oxazolo[5,4-f]quinazoline-9,1′-cyclohexane]-7-one; 5-chloro-2-({[1-(oxan-2-yl)ethyl]amino}methyl)-7,8-dihydro-6H-spiro[[1,3]oxazolo[5,4-f]quinazoline-9,1′-cyclohexane]-7-one; 5-chloro-2-{[(oxan-2-ylmethyl)amino]methyl}-7,8-dihydro-6H-spiro[[1,3]oxazolo[5,4-f]quinazoline-9,1′-cyclohexane]-7-one; 5-chloro-2-{[(3-methyl-2-oxobutyl)amino]methyl}-7,8-dihydro-6H-spiro[[1,3]oxazolo[5,4-f]quinazoline-9,1′-cyclohexane]-7-one; 5-chloro-2-{[(4,4-difluorocyclohexyl)amino]methyl}-7,8-dihydro-6H-spiro[[1,3]oxazolo[5,4-f]quinazoline-9,1′-cyclohexane]-7-one; 5-chloro-2-({[(1r,4r)-4-methoxycyclohexyl]amino}methyl)-7,8-dihydro-6H-spiro[[1,3]oxazolo[5,4-f]quinazoline-9,1′-cyclohexane]-7-one; 5-chloro-2-[(1R,4R)-2-oxa-5-azabicyclo[2.2.1]heptan-5-ylmethyl]-7,8-dihydro-6H-spiro[[1,3]oxazolo[5,4-f]quinazoline-9,1′-cyclohexane]-7-one; 5-chloro-2-[(1S,4S)-2-oxa-5-azabicyclo[2.2.1]heptan-5-ylmethyl]-7,8-dihydro-6H-spiro[[1,3]oxazolo[5,4-f]quinazoline-9,1′-cyclohexane]-7-one; 5-chloro-2-[(3-methoxy-3-methylazetidin-1-yl)methyl]-7,8-dihydro-6H-spiro[[1,3]oxazolo[5,4-f]quinazoline-9,1′-cyclohexane]-7-one; 5-chloro-2-[(4-methoxypiperidin-1-yl)methyl]-7,8-dihydro-6H-spiro[[1,3]oxazolo[5,4-f]quinazoline-9,1′-cyclohexane]-7-one; 5-chloro-2-{[4-(dimethylamino)piperidin-1-yl]methyl}-7,8-dihydro-6H-spiro[[1,3]oxazolo[5,4-f]quinazoline-9,1′-cyclohexane]-7-one; 5-chloro-2-({5-methyl-octahydropyrrolo[3,4-c]pyrrol-2-yl}methyl)-7,8-dihydro-6H-spiro[[1,3]oxazolo[5,4-f]quinazoline-9,1′-cyclohexane]-7-one; 5-chloro-2-{[(2R)-2-(methoxymethyl)pyrrolidin-1-yl]methyl}-7,8-dihydro-6H-spiro[[1,3]oxazolo[5,4-f]quinazoline-9,1′-cyclohexane]-7-one; 5-chloro-2-{[3-(hydroxymethyl)pyrrolidin-1-yl]methyl}-7,8-dihydro-6H-spiro[[1,3]oxazolo[5,4-f]quinazoline-9,1′-cyclohexane]-7-one; 5-chloro-2-{[(3R)-3-methoxypiperidin-1-yl]methyl}-7,8-dihydro-6H-spiro[[1,3]oxazolo[5,4-f]quinazoline-9,1′-cyclohexane]-7-one; 5-chloro-2-{[(3S)-3-methoxypiperidin-1-yl]methyl}-7,8-dihydro-6H-spiro[[1,3]oxazolo[5,4-f]quinazoline-9,1′-cyclohexane]-7-one; 5-chloro-2-{[(3R)-3-ethoxypyrrolidin-1-yl]methyl}-7,8-dihydro-6H-spiro[[1,3]oxazolo[5,4-f]quinazoline-9,1′-cyclohexane]-7-one; 5-chloro-2-{[(3R)-3-(2-hydroxypropan-2-yl)pyrrolidin-1-yl]methyl}-7,8-dihydro-6H-spiro[[1,3]oxazolo[5,4-f]quinazoline-9,1′-cyclohexane]-7-one; 5-chloro-2-{[(3R)-3-hydroxypyrrolidin-1-yl]methyl}-7,8-dihydro-6H-spiro[[1,3]oxazolo[5,4-f]quinazoline-9,1′-cyclohexane]-7-one; 5-chloro-2-({[2-(dimethylamino)ethyl](methyl)amino}methyl)-7,8-dihydro-6H-spiro[[1,3]oxazolo[5,4-f]quinazoline-9,1′-cyclohexane]-7-one; 3-({5-chloro-7-oxo-7,8-dihydro-6H-spiro[[1,3]oxazolo[5,4-f]quinazoline-9,1′-cyclohexane]-2-ylmethyl}(methyl)amino)-N,N-dimethylpropanamide; 5-chloro-2-({methyl[2-(morpholin-4-yl)ethyl]amino}methyl)-7,8-dihydro-6H-spiro[[1,3]oxazolo[5,4-f]quinazoline-9,1′-cyclohexane]-7-one; 5-chloro-2-[(3-methoxypyrrolidin-1-yl)methyl]-7,8-dihydro-6H-spiro[[1,3]oxazolo[5,4-f]quinazoline-9,1′-cyclohexane]-7-one; 5-chloro-2-{[4-(2,2,2-trifluoroethyl)piperazin-1-yl]methyl}-7,8-dihydro-6H-spiro[[1,3]oxazolo[5,4-f]quinazoline-9,1′-cyclohexane]-7-one; methyl 2-({5-chloro-7-oxo-7,8-dihydro-6H-spiro[[1,3]oxazolo[5,4-f]quinazoline-9,1′-cyclohexane]-2-ylmethyl}(methyl)amino)acetate; 2-[(8aR)-octahydropyrrolo[1,2-a]piperazin-2-ylmethyl]-5-chloro-7,8-dihydro-6H-spiro[[1,3]oxazolo[5,4-f]quinazoline-9,1′-cyclohexane]-7-one; 5-chloro-2-{[methyl(oxan-4-yl)amino]methyl}-7,8-dihydro-6H-spiro[[1,3]oxazolo[5,4-f]quinazoline-9,1′-cyclohexane]-7-one; methyl 1-{5-chloro-7-oxo-7,8-dihydro-6H-spiro[[1,3]oxazolo[5,4-f]quinazoline-9,1′-cyclohexane]-2-ylmethyl}piperidine-3-carboxylate; methyl 2-({5-chloro-7-oxo-7,8-dihydro-6H-spiro[[1,3]oxazolo[5,4-f]quinazoline-9,1′-cyclohexane]-2-ylmethyl}amino)acetate; 5-chloro-2-{[(3S)-3-methoxypyrrolidin-1-yl]methyl}-7,8-dihydro-6H-spiro[[1,3]oxazolo[5,4-f]quinazoline-9,1′-cyclohexane]-7-one; 5-chloro-2-{[(oxan-3-yl)amino]methyl}-7,8-dihydro-6H-spiro[[1,3]oxazolo[5,4-f]quinazoline-9,1′-cyclohexane]-7-one; 5-chloro-2-{[(3R)-3-methoxypyrrolidin-1-yl]methyl}-7,8-dihydro-6H-spiro[[1,3]oxazolo[5,4-f]quinazoline-9,1′-cyclohexane]-7-one; 5-chloro-2-{[(3S)-3-(methoxymethyl)pyrrolidin-1-yl]methyl}-7,8-dihydro-6H-spiro[[1,3]oxazolo[5,4-f]quinazoline-9,1′-cyclohexane]-7-one; 5-chloro-2-{[(3R)-3-(methoxymethyl)pyrrolidin-1-yl]methyl}-7,8-dihydro-6H-spiro[[1,3]oxazolo[5,4-f]quinazoline-9,1′-cyclohexane]-7-one; methyl (3R)-1-{5-chloro-7-oxo-7,8-dihydro-6H-spiro[[1,3]oxazolo[5,4-f]quinazoline-9,1′-cyclohexane]-2-ylmethyl}piperidine-3-carboxylate; 5-chloro-2-({[2-(trifluoromethoxy)ethyl]amino}methyl)-7,8-dihydro-6H-spiro[[1,3]oxazolo[5,4-f]quinazoline-9,1′-cyclohexane]-7-one; 5-chloro-2-[(4-ethylpiperazin-1-yl)methyl]-7,8-dihydro-6H-spiro[[1,3]oxazolo[5,4-f]quinazoline-9,1′-cyclohexane]-7-one; 5-chloro-2-{[3-(dimethylamino)pyrrolidin-1-yl]methyl}-7,8-dihydro-6H-spiro[[1,3]oxazolo[5,4-f]quinazoline-9,1′-cyclohexane]-7-one; 2-({5-chloro-7-oxo-7,8-dihydro-6H-spiro[[1,3]oxazolo[5,4-f]quinazoline-9,1′-cyclohexane]-2-ylmethyl}amino)-N,N-diethylacetamide; (2S)-2-({5-chloro-7-oxo-7,8-dihydro-6H-spiro[[1,3]oxazolo[5,4-f]quinazoline-9,1′-cyclohexane]-2-ylmethyl}amino)-N,N-dimethylpropanamide; (2R)-2-({5-chloro-7-oxo-7,8-dihydro-6H-spiro[[1,3]oxazolo[5,4-f]quinazoline-9,1′-cyclohexane]-2-ylmethyl}amino)-N,N-dimethylpropanamide; 5-chloro-2-({[2-oxo-2-(pyrrolidin-1-yl)ethyl]amino}methyl)-7,8-dihydro-6H-spiro[[1,3]oxazolo[5,4-f]quinazoline-9,1′-cyclohexane]-7-one; 5-chloro-2-{[(oxan-4-yl)amino]methyl}-7,8-dihydro-6H-spiro[[1,3]oxazolo[5,4-f]quinazoline-9,1′-cyclohexane]-7-one; 5-chloro-2-[({3-oxabicyclo[3.1.0]hexan-6-yl}amino)methyl]-7,8-dihydro-6H-spiro[[1,3]oxazolo[5,4-f]quinazoline-9,1′-cyclohexane]-7-one; 5-chloro-2-{[(oxepan-4-yl)amino]methyl}-7,8-dihydro-6H-spiro[[1,3]oxazolo[5,4-f]quinazoline-9,1′-cyclohexane]-7-one; 5-chloro-2-{[(oxolan-3-ylmethyl)amino]methyl}-7,8-dihydro-6H-spiro[[1,3]oxazolo[5,4-f]quinazoline-9,1′-cyclohexane]-7-one; 5-chloro-2-{[(3,3-difluoro-2-hydroxypropyl)amino]methyl}-7,8-dihydro-6H-spiro[[1,3]oxazolo[5,4-f]quinazoline-9,1′-cyclohexane]-7-one; 5-chloro-2-{[(4-cyclopropyloxan-4-yl)amino]methyl}-7,8-dihydro-6H-spiro[[1,3]oxazolo[5,4-f]quinazoline-9,1′-cyclohexane]-7-one; 5-chloro-2-{[(2,6-dimethyloxan-4-yl)amino]methyl}-7,8-dihydro-6H-spiro[[1,3]oxazolo[5,4-f]quinazoline-9,1′-cyclohexane]-7-one; 5-chloro-2-({[(3S)-oxan-3-yl]amino}methyl)-7,8-dihydro-6H-spiro[[1,3]oxazolo[5,4-f]quinazoline-9,1′-cyclohexane]-7-one; 5-chloro-2-({[(3S)-oxolan-3-yl]amino}methyl)-7,8-dihydro-6H-spiro[[1,3]oxazolo[5,4-f]quinazoline-9,1′-cyclohexane]-7-one; 5-chloro-2-{[(3-methyloxolan-3-yl)amino]methyl}-7,8-dihydro-6H-spiro[[1,3]oxazolo[5,4-f]quinazoline-9,1′-cyclohexane]-7-one; 5-chloro-2-({[(3R)-oxolan-3-yl]amino}methyl)-7,8-dihydro-6H-spiro[[1,3]oxazolo[5,4-f]quinazoline-9,1′-cyclohexane]-7-one; 5-chloro-2-{[4-(2-methoxyethoxy)piperidin-1-yl]methyl}-7,8-dihydro-6H-spiro[[1,3]oxazolo[5,4-f]quinazoline-9,1′-cyclohexane]-7-one; 2-[(8aS)-octahydropyrrolo[1,2-a]piperazin-2-ylmethyl]-5-chloro-7,8-dihydro-6H-spiro[[1,3]oxazolo[5,4-f]quinazoline-9,1′-cyclohexane]-7-one; methyl (3R)-1-{5-chloro-7-oxo-7,8-dihydro-6H-spiro[[1,3]oxazolo[5,4-f]quinazoline-9,1′-cyclohexane]-2-ylmethyl}pyrrolidine-3-carboxylate; 5-chloro-2-{[4-(2,2-difluoroethyl)piperazin-1-yl]methyl}-7,8-dihydro-6H-spiro[[1,3]oxazolo[5,4-f]quinazoline-9,1′-cyclohexane]-7-one; 5-chloro-2-({[(3-methyloxolan-3-yl)methyl]amino}methyl)-7,8-dihydro-6H-spiro[[1,3]oxazolo[5,4-f]quinazoline-9,1′-cyclohexane]-7-one; 5-chloro-2-({[(3R)-oxan-3-yl]amino}methyl)-7,8-dihydro-6H-spiro[[1,3]oxazolo[5,4-f]quinazoline-9,1′-cyclohexane]-7-one; 5-chloro-2-({[(3R)-oxolan-3-ylmethyl]amino}methyl)-7,8-dihydro-6H-spiro[[1,3]oxazolo[5,4-f]quinazoline-9,1′-cyclohexane]-7-one; 5-chloro-2-{[(3-fluorooxan-4-yl)amino]methyl}-7,8-dihydro-6H-spiro[[1,3]oxazolo[5,4-f]quinazoline-9,1′-cyclohexane]-7-one; 5-chloro-2-{[(2,2-difluoro-3-hydroxypropyl)amino]methyl}-7,8-dihydro-6H-spiro[[1,3]oxazolo[5,4-f]quinazoline-9,1′-cyclohexane]-7-one; 5-chloro-2-{[(2,2-difluorocyclopentyl)amino]methyl}-7,8-dihydro-6H-spiro[[1,3]oxazolo[5,4-f]quinazoline-9,1′-cyclohexane]-7-one; 5-chloro-2-({[4-(methoxymethyl)oxan-4-yl]amino}methyl)-7,8-dihydro-6H-spiro[[1,3]oxazolo[5,4-f]quinazoline-9,1′-cyclohexane]-7-one; 5-chloro-2-({[(3S)-oxolan-3-ylmethyl]amino}methyl)-7,8-dihydro-6H-spiro[[1,3]oxazolo[5,4-f]quinazoline-9,1′-cyclohexane]-7-one; 5-chloro-4′,4′-difluoro-2-[(1S,4S)-2-oxa-5-azabicyclo[2.2.1]heptan-5-ylmethyl]-7,8-dihydro-6H-spiro[[1,3]oxazolo[5,4-f]quinazoline-9,1′-cyclohexane]-7-one; 5-chloro-4′,4′-difluoro-2-({[(3S)-oxolan-3-ylmethyl]amino}methyl)-7,8-dihydro-6H-spiro[[1,3]oxazolo[5,4-f]quinazoline-9,1′-cyclohexane]-7-one; 5-chloro-2-{[(1,3-oxazol-2-ylmethyl)amino]methyl}-7,8-dihydro-6H-spiro[[1,3]oxazolo[5,4-f]quinazoline-9,1′-cyclohexane]-7-one; 5-chloro-2-{[(1,3-oxazol-5-ylmethyl)amino]methyl}-7,8-dihydro-6H-spiro[[1,3]oxazolo[5,4-f]quinazoline-9,1′-cyclohexane]-7-one; 5-chloro-2-({[2-(1,2-oxazol-3-yl)ethyl]amino}methyl)-7,8-dihydro-6H-spiro[[1,3]oxazolo[5,4-f]quinazoline-9,1′-cyclohexane]-7-one; 5-chloro-2-{[(1,3-oxazol-4-ylmethyl)amino]methyl}-7,8-dihydro-6H-spiro[[1,3]oxazolo[5,4-f]quinazoline-9,1′-cyclohexane]-7-one; 5-chloro-2-({[(1-methyl-1H-pyrazol-3-yl)methyl]amino}methyl)-7,8-dihydro-6H-spiro[[1,3]oxazolo[5,4-f]quinazoline-9,1′-cyclohexane]-7-one; 5-chloro-2-{[(1,2-oxazol-5-ylmethyl)amino]methyl}-7,8-dihydro-6H-spiro[[1,3]oxazolo[5,4-f]quinazoline-9,1′-cyclohexane]-7-one; 2-{5-chloro-7-oxo-7,8-dihydro-6H-spiro[[1,3]oxazolo[5,4-f]quinazoline-9,1′-cyclohexane]-2-ylmethoxy}-N,N-dimethylacetamide; 5-chloro-2-[(oxan-4-yloxy)methyl]-7,8-dihydro-6H-spiro[[1,3]oxazolo[5,4-f]quinazoline-9,1′-cyclohexane]-7-one; 5-chloro-2-{[(1-methylpiperidin-4-yl)oxy]methyl}-7,8-dihydro-6H-spiro[[1,3]oxazolo[5,4-f]quinazoline-9,1′-cyclohexane]-7-one; 5-chloro-N-[2-(dimethylamino)ethyl]-7-oxo-7,8-dihydro-6H-spiro[[1,3]oxazolo[5,4-f]quinazoline-9,1′-cyclohexane]-2-carboxamide; 5-chloro-2-(morpholine-4-carbonyl)-7,8-dihydro-6H-spiro[[1,3]oxazolo[5,4-f]quinazoline-9,1′-cyclohexane]-7-one; 5-chloro-N,N-dimethyl-7-oxo-7,8-dihydro-6H-spiro[[1,3]oxazolo[5,4-f]quinazoline-9,1′-cyclohexane]-2-carboxamide; 5-chloro-2-(4-methylpiperazine-1-carbonyl)-7,8-dihydro-6H-spiro[[1,3]oxazolo[5,4-f]quinazoline-9,1′-cyclohexane]-7-one; 5-chloro-N-(2-methoxyethyl)-7-oxo-7,8-dihydro-6H-spiro[[1,3]oxazolo[5,4-f]quinazoline-9,1′-cyclohexane]-2-carboxamide; 5-chloro-2-[(1S,4S)-2-oxa-5-azabicyclo[2.2.1]heptane-5-carbonyl]-7,8-dihydro-6H-spiro[[1,3]oxazolo[5,4-f]quinazoline-9,1′-cyclohexane]-7-one; 5-chloro-N-(2-methoxyethyl)-N-methyl-7-oxo-7,8-dihydro-6H-spiro[[1,3]oxazolo[5,4-f]quinazoline-9,1′-cyclohexane]-2-carboxamide; 5-chloro-N-methyl-N-(oxan-4-ylmethyl)-7-oxo-7,8-dihydro-6H-spiro[[1,3]oxazolo[5,4-f]quinazoline-9,1′-cyclohexane]-2-carboxamide; 5-chloro-2-[(3R)-3-ethoxypyrrolidine-1-carbonyl]-7,8-dihydro-6H-spiro[[1,3]oxazolo[5,4-f]quinazoline-9,1′-cyclohexane]-7-one; 2-{1-5-chloro-7-oxo-7,8-dihydro-6H-spiro[[1,3]oxazolo[5,4-f]quinazoline-9,1′-cyclohexane]-2-yl-N-methylformamido}-N,N-dimethylacetamide; 3-{1-5-chloro-7-oxo-7,8-dihydro-6H-spiro[[1,3]oxazolo[5,4-f]quinazoline-9,1′-cyclohexane]-2-yl-N-methylformamido}-N,N-dimethylpropanamide; 5-chloro-2-[(2S)-2-(methoxymethyl)pyrrolidine-1-carbonyl]-7,8-dihydro-6H-spiro[[1,3]oxazolo[5,4-f]quinazoline-9,1′-cyclohexane]-7-one; methyl 2-{1-5-chloro-7-oxo-7,8-dihydro-6H-spiro[[1,3]oxazolo[5,4-f]quinazoline-9,1′-cyclohexane]-2-yl-N-methylformamido}acetate; 5-chloro-N-methyl-N-[2-(methylamino)ethyl]-7-oxo-7,8-dihydro-6H-spiro[[1,3]oxazolo[5,4-f]quinazoline-9,1′-cyclohexane]-2-carboxamide; 5-chloro-N-[2-(dimethylamino)ethyl]-N-methyl-7-oxo-7,8-dihydro-6H-spiro[[1,3]oxazolo[5,4-f]quinazoline-9,1′-cyclohexane]-2-carboxamide; 5-chloro-2-[4-(dimethylamino)piperidine-1-carbonyl]-7,8-dihydro-6H-spiro[[1,3]oxazolo[5,4-f]quinazoline-9,1′-cyclohexane]-7-one; 5-chloro-2-[4-(2,2,2-trifluoroethyl)piperazine-1-carbonyl]-7,8-dihydro-6H-spiro[[1,3]oxazolo[5,4-f]quinazoline-9,1′-cyclohexane]-7-one; 5-chloro-2-[4-(2-methoxyethyl)piperazine-1-carbonyl]-7,8-dihydro-6H-spiro[[1,3]oxazolo[5,4-f]quinazoline-9,1′-cyclohexane]-7-one; 2-[(8aR)-octahydropyrrolo[1,2-a]piperazine-2-carbonyl]-5-chloro-7,8-dihydro-6H-spiro[[1,3]oxazolo[5,4-f]quinazoline-9,1′-cyclohexane]-7-one; 5-chloro-N-methyl-N-(oxan-4-yl)-7-oxo-7,8-dihydro-6H-spiro[[1,3]oxazolo[5,4-f]quinazoline-9,1′-cyclohexane]-2-carboxamide; 5-chloro-2-(3-methoxypyrrolidine-1-carbonyl)-7,8-dihydro-6H-spiro[[1,3]oxazolo[5,4-f]quinazoline-9,1′-cyclohexane]-7-one; 5-chloro-2-[(3R)-3-methoxypiperidine-1-carbonyl]-7,8-dihydro-6H-spiro[[1,3]oxazolo[5,4-f]quinazoline-9,1′-cyclohexane]-7-one; 5-chloro-N-(oxan-3-yl)-7-oxo-7,8-dihydro-6H-spiro[[1,3]oxazolo[5,4-f]quinazoline-9,1′-cyclohexane]-2-carboxamide; 5-chloro-2-[(3S)-3-methoxypyrrolidine-1-carbonyl]-7,8-dihydro-6H-spiro[[1,3]oxazolo[5,4-f]quinazoline-9,1′-cyclohexane]-7-one; 5-chloro-2-[(3R)-3-(methoxymethyl)pyrrolidine-1-carbonyl]-7,8-dihydro-6H-spiro[[1,3]oxazolo[5,4-f]quinazoline-9,1′-cyclohexane]-7-one; 5-chloro-2-[(3S)-3-(methoxymethyl)pyrrolidine-1-carbonyl]-7,8-dihydro-6H-spiro[[1,3]oxazolo[5,4-f]quinazoline-9,1′-cyclohexane]-7-one; 5-chloro-7-oxo-N-[2-(trifluoromethoxy)ethyl]-7,8-dihydro-6H-spiro[[1,3]oxazolo[5,4-f]quinazoline-9,1′-cyclohexane]-2-carboxamide; 5-chloro-2-[(3R)-3-methoxypyrrolidine-1-carbonyl]-7,8-dihydro-6H-spiro[[1,3]oxazolo[5,4-f]quinazoline-9,1′-cyclohexane]-7-one; 5-chloro-2-(4-ethylpiperazine-1-carbonyl)-7,8-dihydro-6H-spiro[[1,3]oxazolo[5,4-f]quinazoline-9,1′-cyclohexane]-7-one; 5-chloro-2-[4-(propan-2-yl)piperazine-1-carbonyl]-7,8-dihydro-6H-spiro[[1,3]oxazolo[5,4-f]quinazoline-9,1′-cyclohexane]-7-one; 5-chloro-2-[4-(2,2-difluoroethyl)piperazine-1-carbonyl]-7,8-dihydro-6H-spiro[[1,3]oxazolo[5,4-f]quinazoline-9,1′-cyclohexane]-7-one; 2-[(8aS)-octahydropyrrolo[1,2-a]piperazine-2-carbonyl]-5-chloro-7,8-dihydro-6H-spiro[[1,3]oxazolo[5,4-f]quinazoline-9,1′-cyclohexane]-7-one; 5-chloro-2-(piperazine-1-carbonyl)-7,8-dihydro-6H-spiro[[1,3]oxazolo[5,4-f]quinazoline-9,1′-cyclohexane]-7-one; 5-chloro-7-oxo-N-[(3R)-oxolan-3-ylmethyl]-7,8-dihydro-6H-spiro[[1,3]oxazolo[5,4-f]quinazoline-9,1′-cyclohexane]-2-carboxamide; 5-chloro-2-[4-(2-fluoroethyl)piperazine-1-carbonyl]-7,8-dihydro-6H-spiro[[1,3]oxazolo[5,4-f]quinazoline-9,1′-cyclohexane]-7-one; 5-chloro-N-(2,2-difluorocyclopentyl)-7-oxo-7,8-dihydro-6H-spiro[[1,3]oxazolo[5,4-f]quinazoline-9,1′-cyclohexane]-2-carboxamide; 5-chloro-N-[4-(methoxymethyl)oxan-4-yl]-7-oxo-7,8-dihydro-6H-spiro[[1,3]oxazolo[5,4-f]quinazoline-9,1′-cyclohexane]-2-carboxamide; 5-chloro-N-(3-fluorooxan-4-yl)-7-oxo-7,8-dihydro-6H-spiro[[1,3]oxazolo[5,4-f]quinazoline-9,1′-cyclohexane]-2-carboxamide; 5-chloro-N-(2,2-difluoro-3-hydroxypropyl)-7-oxo-7,8-dihydro-6H-spiro[[1,3]oxazolo[5,4-f]quinazoline-9,1′-cyclohexane]-2-carboxamide; 5-chloro-7-oxo-N-[(3S)-oxolan-3-ylmethyl]-7,8-dihydro-6H-spiro[[1,3]oxazolo[5,4-f]quinazoline-9,1′-cyclohexane]-2-carboxamide; 5-chloro-7-oxo-N-[(3S)-oxolan-3-yl]-7,8-dihydro-6H-spiro[[1,3]oxazolo[5,4-f]quinazoline-9,1′-cyclohexane]-2-carboxamide; 5-chloro-7-oxo-N-[(3R)-oxolan-3-yl]-7,8-dihydro-6H-spiro[[1,3]oxazolo[5,4-f]quinazoline-9,1′-cyclohexane]-2-carboxamide; 2-[(8aS)-octahydropyrrolo[1,2-a]piperazine-2-carbonyl]-5-chloro-4′,4′-difluoro-7,8-dihydro-6H-spiro[[1,3]oxazolo[5,4-f]quinazoline-9,1′-cyclohexane]-7-one; 2-[(4,4-difluoropiperidin-1-yl)methyl]-5-fluoro-7,8-dihydro-6H-spiro[[1,3]oxazolo[5,4-f]quinazoline-9,1′-cyclohexane]-7-one; 5-fluoro-2-{[(2-methoxyethyl)amino]methyl}-7,8-dihydro-6H-spiro[[1,3]oxazolo[5,4-f]quinazoline-9,1′-cyclohexane]-7-one; 2-({5-fluoro-7-oxo-7,8-dihydro-6H-spiro[[1,3]oxazolo[5,4-f]quinazoline-9,1′-cyclohexane]-2-ylmethyl}(methyl)amino)-N,N-dimethylacetamide; 2-({5-fluoro-7-oxo-7,8-dihydro-6H-spiro[[1,3]oxazolo[5,4-f]quinazoline-9,1′-cyclohexane]-2-ylmethyl}amino)-N,N-dimethylacetamide; 5-chloro-4′,4′-difluoro-2-({[(3S)-oxan-3-yl]amino}methyl)-7,8-dihydro-6H-spiro[[1,3]oxazolo[5,4-f]quinazoline-9,1′-cyclohexane]-7-one; 5-chloro-4′,4′-difluoro-2-({[(3R)-oxolan-3-ylmethyl]amino}methyl)-7,8-dihydro-6H-spiro[[1,3]oxazolo[5,4-f]quinazoline-9,1′-cyclohexane]-7-one; 5-chloro-4′,4′-difluoro-2-({[(3S)-oxolan-3-yl]amino}methyl)-7,8-dihydro-6H-spiro[[1,3]oxazolo[5,4-f]quinazoline-9,1′-cyclohexane]-7-one; 5-chloro-4′,4′-difluoro-2-({[(3R)-oxan-3-yl]amino}methyl)-7,8-dihydro-6H-spiro[[1,3]oxazolo[5,4-f]quinazoline-9,1′-cyclohexane]-7-one; and pharmaceutically acceptable salts thereof; or
a compound selected from the group consisting of methyl 5-chloro-7-oxo-7,8-dihydro-6H-spiro[[1,3]oxazolo[5,4-f]quinazoline-9,1′-cyclohexane]-2-carboxylate; 5-chloro-7-oxo-7,8-dihydro-6H-spiro[[1,3]oxazolo[5,4-f]quinazoline-9,1′-cyclohexane]-2-carboxamide; 5-chloro-N-[2-(dimethylamino)ethyl]-7-oxo-7,8-dihydro-6H-spiro[[1,3]oxazolo[5,4-f]quinazoline-9,1′-cyclohexane]-2-carboxamide; 5-chloro-2-(morpholine-4-carbonyl)-7,8-dihydro-6H-spiro[[1,3]oxazolo[5,4-f]quinazoline-9,1′-cyclohexane]-7-one; 5-chloro-7-oxo-7,8-dihydro-6H-spiro[[1,3]oxazolo[5,4-f]quinazoline-9,1′-cyclohexane]-2-carboxylic acid; 5-chloro-N,N-dimethyl-7-oxo-7,8-dihydro-6H-spiro[[1,3]oxazolo[5,4-f]quinazoline-9,1′-cyclohexane]-2-carboxamide; 5-chloro-2-(4-methylpiperazine-1-carbonyl)-7,8-dihydro-6H-spiro[[1,3]oxazolo[5,4-f]quinazoline-9,1′-cyclohexane]-7-one; 5-chloro-2-(4-methoxypiperidine-1-carbonyl)-7,8-dihydro-6H-spiro[[1,3]oxazolo[5,4-f]quinazoline-9,1′-cyclohexane]-7-one; 5-chloro-N-(2-methoxyethyl)-7-oxo-7,8-dihydro-6H-spiro[[1,3]oxazolo[5,4-f]quinazoline-9,1′-cyclohexane]-2-carboxamide; 5-chloro-2-[(1 S,4S)-2-oxa-5-azabicyclo[2.2.1]heptane-5-carbonyl]-7,8-dihydro-6H-spiro[[1,3]oxazolo[5,4-f]quinazoline-9,1′-cyclohexane]-7-one; 5-chloro-N-(2-methoxyethyl)-N-methyl-7-oxo-7,8-dihydro-6H-spiro[[1,3]oxazolo[5,4-f]quinazoline-9,1′-cyclohexane]-2-carboxamide; 5-chloro-N-methyl-N-(oxan-4-ylmethyl)-7-oxo-7,8-dihydro-6H-spiro[[1,3]oxazolo[5,4-f]quinazoline-9,1′-cyclohexane]-2-carboxamide; 5-chloro-2-[(3R)-3-ethoxypyrrolidine-1-carbonyl]-7,8-dihydro-6H-spiro[[1,3]oxazolo[5,4-f]quinazoline-9,1′-cyclohexane]-7-one; 2-{1-5-chloro-7-oxo-7,8-dihydro-6H-spiro[[1,3]oxazolo[5,4-f]quinazoline-9,1′-cyclohexane]-2-yl-N-methylformamido}-N,N-dimethylacetamide; 3-{1-5-chloro-7-oxo-7,8-dihydro-6H-spiro[[1,3]oxazolo[5,4-f]quinazoline-9,1′-cyclohexane]-2-yl-N-methylformamido}-N,N-dimethylpropanamide; 5-chloro-2-[(2S)-2-(methoxymethyl)pyrrolidine-1-carbonyl]-7,8-dihydro-6H-spiro[[1,3]oxazolo[5,4-f]quinazoline-9,1′-cyclohexane]-7-one; methyl 2-{1-5-chloro-7-oxo-7,8-dihydro-6H-spiro[[1,3]oxazolo[5,4-f]quinazoline-9,1′-cyclohexane]-2-yl-N-methylformamido}acetate; 5-chloro-N-methyl-N-[2-(methylamino)ethyl]-7-oxo-7,8-dihydro-6H-spiro[[1,3]oxazolo[5,4-f]quinazoline-9,1′-cyclohexane]-2-carboxamide; 5-chloro-N-[2-(dimethylamino)ethyl]-N-methyl-7-oxo-7,8-dihydro-6H-spiro[[1,3]oxazolo[5,4-f]quinazoline-9,1′-cyclohexane]-2-carboxamide; 5-chloro-2-[4-(dimethylamino)piperidine-1-carbonyl]-7,8-dihydro-6H-spiro[[1,3]oxazolo[5,4-f]quinazoline-9,1′-cyclohexane]-7-one; 5-chloro-2-[4-(2,2,2-trifluoroethyl)piperazine-1-carbonyl]-7,8-dihydro-6H-spiro[[1,3]oxazolo[5,4-f]quinazoline-9,1′-cyclohexane]-7-one; 5-chloro-2-[4-(2-methoxyethyl)piperazine-1-carbonyl]-7,8-dihydro-6H-spiro[[1,3]oxazolo[5,4-f]quinazoline-9,1′-cyclohexane]-7-one; 2-[(8aR)-octahydropyrrolo[1,2-a]piperazine-2-carbonyl]-5-chloro-7,8-dihydro-6H-spiro[[1,3]oxazolo[5,4-f]quinazoline-9,1′-cyclohexane]-7-one; 5-chloro-N-methyl-N-(oxan-4-yl)-7-oxo-7,8-dihydro-6H-spiro[[1,3]oxazolo[5,4-f]quinazoline-9,1′-cyclohexane]-2-carboxamide; 5-chloro-2-(3-methoxypyrrolidine-1-carbonyl)-7,8-dihydro-6H-spiro[[1,3]oxazolo[5,4-f]quinazoline-9,1′-cyclohexane]-7-one; 5-chloro-2-[(3R)-3-methoxypiperidine-1-carbonyl]-7,8-dihydro-6H-spiro[[1,3]oxazolo[5,4-f]quinazoline-9,1′-cyclohexane]-7-one; 5-chloro-N-(oxan-3-yl)-7-oxo-7,8-dihydro-6H-spiro[[1,3]oxazolo[5,4-f]quinazoline-9,1′-cyclohexane]-2-carboxamide; 5-chloro-2-[(3S)-3-methoxypyrrolidine-1-carbonyl]-7,8-dihydro-6H-spiro[[1,3]oxazolo[5,4-f]quinazoline-9,1′-cyclohexane]-7-one; 5-chloro-2-[(3R)-3-(methoxymethyl)pyrrolidine-1-carbonyl]-7,8-dihydro-6H-spiro[[1,3]oxazolo[5,4-f]quinazoline-9,1′-cyclohexane]-7-one; 5-chloro-2-[(3S)-3-(methoxymethyl)pyrrolidine-1-carbonyl]-7,8-dihydro-6H-spiro[[1,3]oxazolo[5,4-f]quinazoline-9,1′-cyclohexane]-7-one; 5-chloro-7-oxo-N-[2-(trifluoromethoxy)ethyl]-7,8-dihydro-6H-spiro[[1,3]oxazolo[5,4-f]quinazoline-9,1′-cyclohexane]-2-carboxamide; 5-chloro-2-[(3R)-3-methoxypyrrolidine-1-carbonyl]-7,8-dihydro-6H-spiro[[1,3]oxazolo[5,4-f]quinazoline-9,1′-cyclohexane]-7-one; 5-chloro-2-(4-ethylpiperazine-1-carbonyl)-7,8-dihydro-6H-spiro[[1,3]oxazolo[5,4-f]quinazoline-9,1′-cyclohexane]-7-one; 5-chloro-2-[4-(propan-2-yl)piperazine-1-carbonyl]-7,8-dihydro-6H-spiro[[1,3]oxazolo[5,4-f]quinazoline-9,1′-cyclohexane]-7-one; 5-chloro-2-[4-(2,2-difluoroethyl)piperazine-1-carbonyl]-7,8-dihydro-6H-spiro[[1,3]oxazolo[5,4-f]quinazoline-9,1′-cyclohexane]-7-one; 2-[(8aS)-octahydropyrrolo[1,2-a]piperazine-2-carbonyl]-5-chloro-7,8-dihydro-6H-spiro[[1,3]oxazolo[5,4-f]quinazoline-9,1′-cyclohexane]-7-one; 5-chloro-2-(piperazine-1-carbonyl)-7,8-dihydro-6H-spiro[[1,3]oxazolo[5,4-f]quinazoline-9,1′-cyclohexane]-7-one; 5-chloro-7-oxo-N-[(3R)-oxolan-3-ylmethyl]-7,8-dihydro-6H-spiro[[1,3]oxazolo[5,4-f]quinazoline-9,1′-cyclohexane]-2-carboxamide; 5-chloro-2-[4-(2-fluoroethyl)piperazine-1-carbonyl]-7,8-dihydro-6H-spiro[[1,3]oxazolo[5,4-f]quinazoline-9,1′-cyclohexane]-7-one; 5-chloro-N-(2,2-difluorocyclopentyl)-7-oxo-7,8-dihydro-6H-spiro[[1,3]oxazolo[5,4-f]quinazoline-9,1′-cyclohexane]-2-carboxamide; 5-chloro-N-[4-(methoxymethyl)oxan-4-yl]-7-oxo-7,8-dihydro-6H-spiro[[1,3]oxazolo[5,4-f]quinazoline-9,1′-cyclohexane]-2-carboxamide; 5-chloro-N-(3-fluorooxan-4-yl)-7-oxo-7,8-dihydro-6H-spiro[[1,3]oxazolo[5,4-f]quinazoline-9,1′-cyclohexane]-2-carboxamide; 5-chloro-N-(2,2-difluoro-3-hydroxypropyl)-7-oxo-7,8-dihydro-6H-spiro[[1,3]oxazolo[5,4-f]quinazoline-9,1′-cyclohexane]-2-carboxamide; 5-chloro-7-oxo-N-[(3S)-oxolan-3-ylmethyl]-7,8-dihydro-6H-spiro[[1,3]oxazolo[5,4-f]quinazoline-9,1′-cyclohexane]-2-carboxamide; 5-chloro-7-oxo-N-[(3S)-oxolan-3-yl]-7,8-dihydro-6H-spiro[[1,3]oxazolo[5,4-f]quinazoline-9,1′-cyclohexane]-2-carboxamide; 5-chloro-7-oxo-N-[(3R)-oxolan-3-yl]-7,8-dihydro-6H-spiro[[1,3]oxazolo[5,4-f]quinazoline-9,1′-cyclohexane]-2-carboxamide; methyl 5-chloro-4′,4′-difluoro-7-oxo-7,8-dihydro-6H-spiro[[1,3]oxazolo[5,4-f]quinazoline-9,1′-cyclohexane]-2-carboxylate; 2-[(8aS)-octahydropyrrolo[1,2-a]piperazine-2-carbonyl]-5-chloro-4′,4′-difluoro-7,8-dihydro-6H-spiro[[1,3]oxazolo[5,4-f]quinazoline-9,1′-cyclohexane]-7-one; and pharmaceutically acceptable salts thereof; or
a compound selected from the group consisting of: 5-chloro-2-methyl-7,8-dihydro-6H-spiro[[1,3]oxazolo[5,4-f]quinazoline-9,1′-cyclohexan]-7-one; 5-chloro-2-(chloromethyl)-7,8-dihydro-6H-spiro[[1,3]oxazolo[5,4-f]quinazoline-9,1′-cyclohexan]-7-one; 5-chloro-2-(methoxymethyl)-7,8-dihydro-6H-spiro[[1,3]oxazolo[5,4-f]quinazoline-9,1′-cyclohexan]-7-one; 5-chloro-2-(hydroxymethyl)-7,8-dihydro-6H-spiro[[1,3]oxazolo[5,4-f]quinazoline-9,1′-cyclohexan]-7-one; 5-chloro-2-propyl-7,8-dihydro-6H-spiro[[1,3]oxazolo[5,4-f]quinazoline-9,1′-cyclohexan]-7-one; 5-chloro-2-ethyl-7,8-dihydro-6H-spiro[[1,3]oxazolo[5,4-f]quinazoline-9,1′-cyclohexan]-7-one; 5-chloro-2-[(dimethylamino)methyl]-7,8-dihydro-6H-spiro[[1,3]oxazolo[5,4-f]quinazoline-9,1′-cyclohexan]-7-one; 5-chloro-2-[(4-methylpiperazin-1-yl)methyl]-7,8-dihydro-6H-spiro[[1,3]oxazolo[5,4-f]quinazoline-9,1′-cyclohexan]-7-one; 5-chloro-2-[(morpholin-4-yl)methyl]-7,8-dihydro-6H-spiro[[1,3]oxazolo[5,4-f]quinazoline-9,1′-cyclohexan]-7-one; 5-chloro-2-[(4-hydroxypiperidin-1-yl)methyl]-7,8-dihydro-6H-spiro[[1,3]oxazolo[5,4-f]quinazoline-9,1′-cyclohexan]-7-one; 5-chloro-2-[(methylamino)methyl]-7,8-dihydro-6H-spiro[[1,3]oxazolo[5,4-f]quinazoline-9,1′-cyclohexan]-7-one; 5-chloro-2-[(cyclopropylamino)methyl]-7,8-dihydro-6H-spiro[[1,3]oxazolo[5,4-f]quinazoline-9,1′-cyclohexan]-7-one; 5-chloro-2-[(piperazin-1-yl)methyl]-7,8-dihydro-6H-spiro[[1,3]oxazolo[5,4-f]quinazoline-9,1′-cyclohexan]-7-one; 5-chloro-2-[(pyrrolidin-1-yl)methyl]-7,8-dihydro-6H-spiro[[1,3]oxazolo[5,4-f]quinazoline-9,1′-cyclohexan]-7-one; 5-chloro-2-(2-methoxyethyl)-7,8-dihydro-6H-spiro[[1,3]oxazolo[5,4-f]quinazoline-9,1′-cyclohexan]-7-one; 5-chloro-2-(2-hydroxyethyl)-7,8-dihydro-6H-spiro[[1,3]oxazolo[5,4-f]quinazoline-9,1′-cyclohexan]-7-one; 5-chloro-2-{[(2-methoxyethyl)amino]methyl}-7,8-dihydro-6H-spiro[[1,3]oxazolo[5,4-f]quinazoline-9,1′-cyclohexan]-7-one; 5-chloro-2-{[(propan-2-yl)amino]methyl}-7,8-dihydro-6H-spiro[[1,3]oxazolo[5,4-f]quinazoline-9,1′-cyclohexan]-7-one; 5-chloro-2-[(ethylamino)methyl]-7,8-dihydro-6H-spiro[[1,3]oxazolo[5,4-f]quinazoline-9,1′-cyclohexan]-7-one; 5-chloro-2-{[(2-hydroxyethyl)amino]methyl}-7,8-dihydro-6H-spiro[[1,3]oxazolo[5,4-f]quinazoline-9,1′-cyclohexan]-7-one; 5-chloro-2-[(4,4-difluoropiperidin-1-yl)methyl]-7,8-dihydro-6H-spiro[[1,3]oxazolo[5,4-f]quinazoline-9,1′-cyclohexan]-7-one; 5-chloro-2-{[(2,2,2-trifluoroethyl)amino]methyl}-7,8-dihydro-6H-spiro[[1,3]oxazolo[5,4-f]quinazoline-9,1′-cyclohexan]-7-one; 5-chloro-2-{[(2-hydroxyethyl)(methyl)amino]methyl}-7,8-dihydro-6H-spiro[[1,3]oxazolo[5,4-f]quinazoline-9,1′-cyclohexan]-7-one; 5-chloro-2-[(3,3-difluoroazetidin-1-yl)methyl]-7,8-dihydro-6H-spiro[[1,3]oxazolo[5,4-f]quinazoline-9,1′-cyclohexan]-7-one; 5-chloro-2-[(3,3-difluoropyrrolidin-1-yl)methyl]-7,8-dihydro-6H-spiro[[1,3]oxazolo[5,4-f]quinazoline-9,1′-cyclohexan]-7-one; 2-[(4-acetylpiperazin-1-yl)methyl]-5-chloro-7,8-dihydro-6H-spiro[[1,3]oxazolo[5,4-f]quinazoline-9,1′-cyclohexan]-7-one; 5-chloro-2-[(4-propanoylpiperazin-1-yl)methyl]-7,8-dihydro-6H-spiro[[1,3]oxazolo[5,4-f]quinazoline-9,1′-cyclohexan]-7-one; 5-chloro-2-({2-oxa-8-azaspiro[4.5]decan-8-yl}methyl)-7,8-dihydro-6H-spiro[[1,3]oxazolo[5,4-f]quinazoline-9,1′-cyclohexan]-7-one; 5-chloro-2-{[4-(oxetan-3-yl)piperazin-1-yl]methyl}-7,8-dihydro-6H-spiro[[1,3]oxazolo[5,4-f]quinazoline-9,1′-cyclohexan]-7-one; 2-[({5-chloro-7-oxo-7,8-dihydro-6H-spiro[[1,3]oxazolo[5,4-f]quinazoline-9,1′-cyclohexan]-2-yl}methyl)amino]-N,N-dimethylacetamide; tert-butyl 2-{[({5-chloro-7-oxo-7,8-dihydro-6H-spiro[[1,3]oxazolo[5,4-f]quinazoline-9,1′-cyclohexan]-2-yl}methyl)amino]methyl}piperidine-1-carboxylate; 5-chloro-2-({[(3,5-difluoropyridin-2-yl)methyl]amino}methyl)-7,8-dihydro-6H-spiro[[1,3]oxazolo[5,4-f]quinazoline-9,1′-cyclohexan]-7-one; 5-chloro-2-({[(piperidin-2-yl)methyl]amino}methyl)-7,8-dihydro-6H-spiro[[1,3]oxazolo[5,4-f]quinazoline-9,1′-cyclohexan]-7-one; 5-chloro-2-({[2-oxo-2-(piperidin-1-yl)ethyl]amino}methyl)-7,8-dihydro-6H-spiro[[1,3]oxazolo[5,4-f]quinazoline-9,1′-cyclohexan]-7-one; 2-[({5-chloro-7-oxo-7,8-dihydro-6H-spiro[[1,3]oxazolo[5,4-f]quinazoline-9,1′-cyclohexan]-2-yl}methyl)amino]-N-ethyl-N-methylacetamide; 5-chloro-2-[(4-methyl-3-oxopiperazin-1-yl)methyl]-7,8-dihydro-6H-spiro[[1,3]oxazolo[5,4-f]quinazoline-9,1′-cyclohexan]-7-one; 5-chloro-2-[(4-ethyl-3-oxopiperazin-1-yl)methyl]-7,8-dihydro-6H-spiro[[1,3]oxazolo[5,4-f]quinazoline-9,1′-cyclohexan]-7-one; 5-chloro-2-({[(pyridin-2-yl)methyl]amino}methyl)-7,8-dihydro-6H-spiro[[1,3]oxazolo[5,4-f]quinazoline-9,1′-cyclohexan]-7-one; 5-chloro-2-({[(pyrimidin-2-yl)methyl]amino}methyl)-7,8-dihydro-6H-spiro[[1,3]oxazolo[5,4-f]quinazoline-9,1′-cyclohexan]-7-one; 5-chloro-2-({[(5-methoxypyridin-3-yl)methyl]amino}methyl)-7,8-dihydro-6H-spiro[[1,3]oxazolo[5,4-f]quinazoline-9,1′-cyclohexan]-7-one; 5-chloro-2-[(2,4-dimethyl-3-oxopiperazin-1-yl)methyl]-7,8-dihydro-6H-spiro[[1,3]oxazolo[5,4-f]quinazoline-9,1′-cyclohexan]-7-one; ethyl 1-({5-chloro-7-oxo-7,8-dihydro-6H-spiro[[1,3]oxazolo[5,4f]quinazoline-9,1′-cyclohexan]-2-yl}methyl)piperidine-4-carboxylate; 5-chloro-2-({1-oxo-2-oxa-8-azaspiro[4.5]decan-8-yl}methyl)-7,8-dihydro-6H-spiro[[1,3]oxazolo[5,4-f]quinazoline-9,1′-cyclohexan]-7-one; 2-[(4-acetylpiperidin-1-yl)methyl]-5-chloro-7,8-dihydro-6H-spiro[[1,3]oxazolo[5,4-f]quinazoline-9,1′-cyclohexan]-7-one; methyl 1-({5-chloro-7-oxo-7,8-dihydro-6H-spiro[[1,3]oxazolo[5,4f]quinazoline-9,1′-cyclohexan]-2-yl}methyl)piperidine-4-carboxylate; 5-chloro-2-({[(5-fluoropyridin-2-yl)methyl]amino}methyl)-7,8-dihydro-6H-spiro[[1,3]oxazolo[5,4-f]quinazoline-9,1′-cyclohexan]-7-one; 5-chloro-2-{[3-(2-methylpropoxy)azetidin-1-yl]methyl}-7,8-dihydro-6H-spiro[[1,3]oxazolo[5,4-f]quinazoline-9,1′-cyclohexan]-7-one; 5-chloro-2-[(4-ethoxypiperidin-1-yl)methyl]-7,8-dihydro-6H-spiro[[1,3]oxazolo[5,4-f]quinazoline-9,1′-cyclohexan]-7-one; 5-chloro-2-{[4-(cyclopropylmethoxy)piperidin-1-yl]methyl}-7,8-dihydro-6H-spiro[[1,3]oxazolo[5,4-f]quinazoline-9,1′-cyclohexan]-7-one; 5-chloro-2-{[4-hydroxy-4-(trifluoromethyl)piperidin-1-yl]methyl}-7,8-dihydro-6H-spiro[[1,3]oxazolo[5,4-f]quinazoline-9,1′-cyclohexan]-7-one; 5-chloro-2-{[4-(propan-2-yloxy)piperidin-1-yl]methyl}-7,8-dihydro-6H-spiro[[1,3]oxazolo[5,4-f]quinazoline-9,1′-cyclohexan]-7-one; 5-chloro-2-[(3-fluoroazetidin-1-yl)methyl]-7,8-dihydro-6H-spiro[[1,3]oxazolo[5,4-f]quinazoline-9,1′-cyclohexan]-7-one; 5-chloro-2-[(3-methoxyazetidin-1-yl)methyl]-7,8-dihydro-6H-spiro[[1,3]oxazolo[5,4-f]quinazoline-9,1′-cyclohexan]-7-one; 5-chloro-2-{[3-(methoxymethyl)azetidin-1-yl]methyl}-7,8-dihydro-6H-spiro[[1,3]oxazolo[5,4-f]quinazoline-9,1′-cyclohexan]-7-one; 5-chloro-2-({7-oxa-2-azaspiro[3.5]nonan-2-yl}methyl)-7,8-dihydro-6H-spiro[[1,3]oxazolo[5,4-f]quinazoline-9,1′-cyclohexan]-7-one; 5-chloro-2-[(4-methoxy-4-methylpiperidin-1-yl)methyl]-7,8-dihydro-6H-spiro[[1,3]oxazolo[5,4-f]quinazoline-9,1′-cyclohexan]-7-one; 5-chloro-2-({3-oxo-1-oxa-8-azaspiro[4.5]decan-8-yl}methyl)-7,8-dihydro-6H-spiro[[1,3]oxazolo[5,4-f]quinazoline-9,1′-cyclohexan]-7-one; 2-[({5-chloro-7-oxo-7,8-dihydro-6H-spiro[[1,3]oxazolo[5,4-f]quinazoline-9,1′-cyclohexan]-2-yl}methyl)(methyl)amino]-N,N-dimethylacetamide; 5-chloro-2-({[(6-methylpyridin-3-yl)methyl]amino}methyl)-7,8-dihydro-6H-spiro[[1,3]oxazolo[5,4-f]quinazoline-9,1′-cyclohexan]-7-one; 5-chloro-2-({methyl[(oxan-4-yl)methyl]amino}methyl)-7,8-dihydro-6H-spiro[[1,3]oxazolo[5,4-f]quinazoline-9,1′-cyclohexan]-7-one; 5-chloro-2-({methyl[(3-methyloxetan-3-yl)methyl]amino}methyl)-7,8-dihydro-6H-spiro[[1,3]oxazolo[5,4-f]quinazoline-9,1′-cyclohexan]-7-one; 5-chloro-2-({[(3-fluorooxetan-3-yl)methyl]amino}methyl)-7,8-dihydro-6H-spiro[[1,3]oxazolo[5,4-f]quinazoline-9,1′-cyclohexan]-7-one; 5-chloro-2-({[(oxan-4-yl)methyl]amino}methyl)-7,8-dihydro-6H-spiro[[1,3]oxazolo[5,4-f]quinazoline-9,1′-cyclohexan]-7-one; 5-chloro-2-({2-oxa-7-azaspiro[3.5]nonan-7-yl}methyl)-7,8-dihydro-6H-spiro[[1,3]oxazolo[5,4-f]quinazoline-9,1′-cyclohexan]-7-one; 5-chloro-2-({[(3-methyloxetan-3-yl)methyl]amino}methyl)-7,8-dihydro-6H-spiro[[1,3]oxazolo[5,4-f]quinazoline-9,1′-cyclohexan]-7-one; 5-chloro-2-({2-oxa-5-azabicyclo[2.2.1]heptan-5-yl}methyl)-7,8-dihydro-6H-spiro[[1,3]oxazolo[5,4-f]quinazoline-9,1′-cyclohexan]-7-one; 5-chloro-2-{[methyl(2,2,2-trifluoroethyl)amino]methyl}-7,8-dihydro-6H-spiro[[1,3]oxazolo[5,4-f]quinazoline-9,1′-cyclohexan]-7-one; 5-chloro-2-({[(oxetan-3-yl)methyl]amino}methyl)-7,8-dihydro-6H-spiro[[1,3]oxazolo[5,4-f]quinazoline-9,1′-cyclohexan]-7-one; 5-chloro-2-[(4-oxopiperidin-1-yl)methyl]-7,8-dihydro-6H-spiro[[1,3]oxazolo[5,4-f]quinazoline-9,1′-cyclohexan]-7-one; 5-chloro-2-({[(2-methoxyphenyl)methyl]amino}methyl)-7,8-dihydro-6H-spiro[[1,3]oxazolo[5,4-f]quinazoline-9,1′-cyclohexan]-7-one; 5-chloro-2-({[(2,4-dimethoxyphenyl)methyl]amino}methyl)-7,8-dihydro-6H-spiro[[1,3]oxazolo[5,4-f]quinazoline-9,1′-cyclohexan]-7-one; 5-chloro-2-{[4-(propan-2-yl)piperazin-1-yl]methyl}-7,8-dihydro-6H-spiro[[1,3]oxazolo[5,4-f]quinazoline-9,1′-cyclohexan]-7-one; 5-chloro-2-[(3,3,4-trimethylpiperazin-1-yl)methyl]-7,8-dihydro-6H-spiro[[1,3]oxazolo[5,4-f]quinazoline-9,1′-cyclohexan]-7-one; 5-chloro-2-{[2-(hydroxymethyl)piperidin-1-yl]methyl}-7,8-dihydro-6H-spiro[[1,3]oxazolo[5,4-f]quinazoline-9,1′-cyclohexan]-7-one; 5-chloro-2-({[1-(hydroxymethyl)cyclopentyl]amino}methyl)-7,8-dihydro-6H-spiro[[1,3]oxazolo[5,4-f]quinazoline-9,1′-cyclohexan]-7-one; 5-chloro-2-{[(2R)-2-(hydroxymethyl)pyrrolidin-1-yl]methyl}-7,8-dihydro-6H-spiro[[1,3]oxazolo[5,4-f]quinazoline-9,1′-cyclohexan]-7-one; 5-chloro-2-[(2-methylpiperidin-1-yl)methyl]-7,8-dihydro-6H-spiro[[1,3]oxazolo[5,4-f]quinazoline-9,1′-cyclohexan]-7-one; 5-chloro-2-{[2-(2-methylpropyl)morpholin-4-yl]methyl}-7,8-dihydro-6H-spiro[[1,3]oxazolo[5,4-f]quinazoline-9,1′-cyclohexan]-7-one; 5-chloro-2-{[(2-cyclobutyl-2,2-difluoroethyl)amino]methyl}-7,8-dihydro-6H-spiro[[1,3]oxazolo[5,4-f]quinazoline-9,1′-cyclohexan]-7-one; 5-chloro-2-[(3-ethylmorpholin-4-yl)methyl]-7,8-dihydro-6H-spiro[[1,3]oxazolo[5,4-f]quinazoline-9,1′-cyclohexan]-7-one; 5-chloro-2-{[(2-cyclohexyl-2-hydroxyethyl)amino]methyl}-7,8-dihydro-6H-spiro[[1,3]oxazolo[5,4-f]quinazoline-9,1′-cyclohexan]-7-one; 5-chloro-2-({6-oxa-9-azaspiro[4.5]decan-9-yl}methyl)-7,8-dihydro-6H-spiro[[1,3]oxazolo[5,4-f]quinazoline-9,1′-cyclohexan]-7-one; 5-chloro-2-({[1-(oxan-2-yl)ethyl]amino}methyl)-7,8-dihydro-6H-spiro[[1,3]oxazolo[5,4-f]quinazoline-9,1′-cyclohexan]-7-one; 5-chloro-2-({[(oxan-2-yl)methyl]amino}methyl)-7,8-dihydro-6H-spiro[[1,3]oxazolo[5,4-f]quinazoline-9,1′-cyclohexan]-7-one; 5-chloro-2-[(2-methoxyethoxy)methyl]-7,8-dihydro-6H-spiro[[1,3]oxazolo[5,4-f]quinazoline-9,1′-cyclohexan]-7-one; ethyl 2-{5-chloro-7-oxo-7,8-dihydro-6H-spiro[[1,3]oxazolo[5,4-f]quinazoline-9,1′-cyclohexan]-2-yl}acetate; 2-[(4,4-difluoropiperidin-1-yl)methyl]-5-fluoro-7,8-dihydro-6H-spiro[[1,3]oxazolo[5,4-f]quinazoline-9,1′-cyclohexan]-7-one; 5-fluoro-2-{[(2-methoxyethyl)amino]methyl}-7,8-dihydro-6H-spiro[[1,3]oxazolo[5,4-f]quinazoline-9,1′-cyclohexan]-7-one; 5-chloro-2-{[(3-methyl-2-oxobutyl)amino]methyl}-7,8-dihydro-6H-spiro[[1,3]oxazolo[5,4-f]quinazoline-9,1′-cyclohexan]-7-one; 5-fluoro-2-[(3-methoxyazetidin-1-yl)methyl]-7,8-dihydro-6H-spiro[[1,3]oxazolo[5,4-f]quinazoline-9,1′-cyclohexan]-7-one; 5-chloro-2-{[(4,4-difluorocyclohexyl)amino]methyl}-7,8-dihydro-6H-spiro[[1,3]oxazolo[5,4-f]quinazoline-9,1′-cyclohexan]-7-one; 5-chloro-2-({[(1R,4R)-4-methoxycyclohexyl]amino}methyl)-7,8-dihydro-6H-spiro[[1,3]oxazolo[5,4-f]quinazoline-9,1′-cyclohexan]-7-one; 5-chloro-2-{[(1R,4R)-2-oxa-5-azabicyclo[2.2.1]heptan-5-yl]methyl}-7,8-dihydro-6H-spiro[[1,3]oxazolo[5,4-f]quinazoline-9,1′-cyclohexan]-7-one; 5-chloro-2-{[(1S,4S)-2-oxa-5-azabicyclo[2.2.1]heptan-5-yl]methyl}-7,8-dihydro-6H-spiro[[1,3]oxazolo[5,4-f]quinazoline-9,1′-cyclohexan]-7-one; 5-chloro-2-[(3-methoxy-3-methylazetidin-1-yl)methyl]-7,8-dihydro-6H-spiro[[1,3]oxazolo[5,4-f]quinazoline-9,1′-cyclohexan]-7-one; 5-chloro-2-[(4-methoxypiperidin-1-yl)methyl]-7,8-dihydro-6H-spiro[[1,3]oxazolo[5,4-f]quinazoline-9,1′-cyclohexan]-7-one; 5-chloro-2-{[4-(dimethylamino)piperidin-1-yl]methyl}-7,8-dihydro-6H-spiro[[1,3]oxazolo[5,4-f]quinazoline-9,1′-cyclohexan]-7-one; 5-chloro-2-({5-methyl-octahydropyrrolo[3,4-c]pyrrol-2-yl}methyl)-7,8-dihydro-6H-spiro[[1,3]oxazolo[5,4-f]quinazoline-9,1′-cyclohexan]-7-one; 5-chloro-2-{[(2R)-2-(methoxymethyl)pyrrolidin-1-yl]methyl}-7,8-dihydro-6H-spiro[[1,3]oxazolo[5,4-f]quinazoline-9,1′-cyclohexan]-7-one; 5-chloro-2-{[3-(hydroxymethyl) pyrrolidin-1-yl]methyl}-7,8-dihydro-6H-spiro[[1,3]oxazolo[5,4-f]quinazoline-9,1′-cyclohexan]-7-one; 5-chloro-2-{[(3R)-3-methoxypiperidin-1-yl]methyl}-7,8-dihydro-6H-spiro[[1,3]oxazolo[5,4-f]quinazoline-9,1′-cyclohexan]-7-one; 5-chloro-2-{[(3S)-3-methoxypiperidin-1-yl]methyl}-7,8-dihydro-6H-spiro[[1,3]oxazolo[5,4-f]quinazoline-9,1′-cyclohexan]-7-one; 5-chloro-2-{[(3R)-3-ethoxypyrrolidin-1-yl]methyl}-7,8-dihydro-6H-spiro[[1,3]oxazolo[5,4-f]quinazoline-9,1′-cyclohexan]-7-one; 5-chloro-2-{[(3R)-3-(2-hydroxypropan-2-yl)pyrrolidin-1-yl]methyl}-7,8-dihydro-6H-spiro[[1,3]oxazolo[5,4-f]quinazoline-9,1′-cyclohexan]-7-one; 5-chloro-2-{[(3R)-3-hydroxypyrrolidin-1-yl]methyl}-7,8-dihydro-6H-spiro[[1,3]oxazolo[5,4-f]quinazoline-9,1′-cyclohexan]-7-one; 5-chloro-2-({[2-(dimethylamino)ethyl](methyl)amino}methyl)-7,8-dihydro-6H-spiro[[1,3]oxazolo[5,4-f]quinazoline-9,1′-cyclohexan]-7-one; 3-[({5-chloro-7-oxo-7,8-dihydro-6H-spiro[[1,3]oxazolo[5,4-f]quinazoline-9,1′-cyclohexan]-2-yl}methyl)(methyl)amino]-N,N-dimethylpropanamide; 5-chloro-2-({methyl[2-(morpholin-4-yl)ethyl]amino}methyl)-7,8-dihydro-6H-spiro[[1,3]oxazolo[5,4-f]quinazoline-9,1′-cyclohexan]-7-one 5-chloro-2-[(3-methoxypyrrolidin-1-yl)methyl]-7,8-dihydro-6H-spiro[[1,3]oxazolo[5,4-f]quinazoline-9,1′-cyclohexan]-7-one; 5-chloro-2-{[4-(2,2,2-trifluoroethyl)piperazin-1-yl]methyl}-7,8-dihydro-6H-spiro[[1,3]oxazolo[5,4-f]quinazoline-9,1′-cyclohexan]-7-one; methyl 2-[({5-chloro-7-oxo-7,8-dihydro-6H-spiro[[1,3]oxazolo[5,4f]quinazoline-9,1′-cyclohexan]-2-yl}methyl)(methyl)amino]acetate; 2-{[(8aR)-octahydropyrrolo[1,2-a]pyrazin-2-yl]methyl}-5-chloro-7,8-dihydro-6H-spiro[[1,3]oxazolo[5,4-f]quinazoline-9,1′-cyclohexan]-7-one; 5-chloro-2-{[methyl(oxan-4-yl)amino]methyl}-7,8-dihydro-6H-spiro[[1,3]oxazolo[5,4-f]quinazoline-9,1′-cyclohexan]-7-one; methyl 1-({5-chloro-7-oxo-7,8-dihydro-6H-spiro[[1,3]oxazolo[5,4-f]quinazoline-9,1′-cyclohexan]-2-yl}methyl)piperidine-3-carboxylate; methyl 2-[({5-chloro-7-oxo-7,8-dihydro-6H-spiro[[1,3]oxazolo[5,4-f]quinazoline-9,1′-cyclohexan]-2-yl}methyl)amino]acetate; 5-chloro-2-{[(3S)-3-methoxypyrrolidin-1-yl]methyl}-7,8-dihydro-6H-spiro[[1,3]oxazolo[5,4-f]quinazoline-9,1′-cyclohexan]-7-one; 5-chloro-2-{[(oxan-3-yl)amino]methyl}-7,8-dihydro-6H-spiro[[1,3]oxazolo[5,4-f]quinazoline-9,1′-cyclohexan]-7-one; 5-chloro-2-{[(3R)-3-methoxypyrrolidin-1-yl]methyl}-7,8-dihydro-6H-spiro[[1,3]oxazolo[5,4-f]quinazoline-9,1′-cyclohexan]-7-one; 5-chloro-2-{[(3S)-3-(methoxymethyl)pyrrolidin-1-yl]methyl}-7,8-dihydro-6H-spiro[[1,3]oxazolo[5,4-f]quinazoline-9,1′-cyclohexan]-7-one; 5-chloro-2-{[(3R)-3-(methoxymethyl)pyrrolidin-1-yl]methyl}-7,8-dihydro-6H-spiro[[1,3]oxazolo[5,4-f]quinazoline-9,1′-cyclohexan]-7-one; methyl (3R)-1-({5-chloro-7-oxo-7,8-dihydro-6H-spiro[[1,3]oxazolo[5,4-f]quinazoline-9,1′-cyclohexan]-2-yl}methyl)piperidine-3-carboxylate; 2-({5-chloro-7-oxo-7,8-dihydro-6H-spiro[[1,3]oxazolo[5,4-f]quinazoline-9,1′-cyclohexan]-2-yl}methoxy)-N,N-dimethylacetamide; 5-chloro-2-[(oxan-4-yloxy)methyl]-7,8-dihydro-6H-spiro[[1,3]oxazolo[5,4-f]quinazoline-9,1′-cyclohexan]-7-one; 5-chloro-2-({[2-(trifluoromethoxy)ethyl]amino}methyl)-7,8-dihydro-6H-spiro[[1,3]oxazolo[5,4-f]quinazoline-9,1′-cyclohexan]-7-one; 5-chloro-2-[(4-ethylpiperazin-1-yl)methyl]-7,8-dihydro-6H-spiro[[1,3]oxazolo[5,4-f]quinazoline-9,1′-cyclohexan]-7-one; 5-chloro-2-{[3-(dimethylamino)pyrrolidin-1-yl]methyl}-7,8-dihydro-6H-spiro[[1,3]oxazolo[5,4-f]quinazoline-9,1′-cyclohexan]-7-one; 2-({5-chloro-7-oxo-7,8-dihydro-6H-spiro[[1,3]oxazolo[5,4-f]quinazoline-9,1′-cyclohexan]-2-yl}methyl)amino]-N,N-diethylacetamide; (2S)-2-[({5-chloro-7-oxo-7,8-dihydro-6H-spiro[[1,3]oxazolo[5,4-f]quinazoline-9,1′-cyclohexan]-2-yl}methyl)amino]-N,N-dimethylpropanamide; (2R)-2-[({5-chloro-7-oxo-7,8-dihydro-6H-spiro[[1,3]oxazolo[5,4-f]quinazoline-9,1′-cyclohexan]-2-yl}methyl)amino]-N,N-dimethylpropanamide; 5-chloro-2-({[2-oxo-2-(pyrrolidin-1-yl)ethyl]amino}methyl)-7,8-dihydro-6H-spiro[[1,3]oxazolo[5,4-f]quinazoline-9,1′-cyclohexan]-7-one; 5-chloro-2-{[(oxan-4-yl)amino]methyl}-7,8-dihydro-6H-spiro[[1,3]oxazolo[5,4-f]quinazoline-9,1′-cyclohexan]-7-one; 5-chloro-2-[({3-oxabicyclo[3.1.0]hexan-6-yl}amino)methyl]-7,8-dihydro-6H-spiro[[1,3]oxazolo[5,4-f]quinazoline-9,1′-cyclohexan]-7-one; 5-chloro-2-{[(oxepan-4-yl)amino]methyl}-7,8-dihydro-6H-spiro[[1,3]oxazolo[5,4-f]quinazoline-9,1′-cyclohexan]-7-one; 5-chloro-2-({[(oxolan-3-yl)methyl]amino}methyl)-7,8-dihydro-6H-spiro[[1,3]oxazolo[5,4-f]quinazoline-9,1′-cyclohexan]-7-one; 5-chloro-2-{[(3,3-difluoro-2-hydroxypropyl)amino]methyl}-7,8-dihydro-6H-spiro[[1,3]oxazolo[5,4-f]quinazoline-9,1′-cyclohexan]-7-one; 5-chloro-2-{[(4-cyclopropyloxan-4-yl)amino]methyl}-7,8-dihydro-6H-spiro[[1,3]oxazolo[5,4-f]quinazoline-9,1′-cyclohexan]-7-one; 5-chloro-2-{[(2,6-dimethyloxan-4-yl)amino]methyl}-7,8-dihydro-6H-spiro[[1,3]oxazolo[5,4-f]quinazoline-9,1′-cyclohexan]-7-one; 5-chloro-2-({[(3S)-oxan-3-yl]amino}methyl)-7,8-dihydro-6H-spiro[[1,3]oxazolo[5,4-f]quinazoline-9,1′-cyclohexan]-7-one; 5-chloro-2-({[(3S)-oxolan-3-yl]amino}methyl)-7,8-dihydro-6H-spiro[[1,3]oxazolo[5,4-f]quinazoline-9,1′-cyclohexan]-7-one; 5-chloro-2-{[(3-methyloxolan-3-yl)amino]methyl}-7,8-dihydro-6H-spiro[[1,3]oxazolo[5,4-f]quinazoline-9,1′-cyclohexan]-7-one; 5-chloro-2-({[(3R)-oxolan-3-yl]amino}methyl)-7,8-dihydro-6H-spiro[[1,3]oxazolo[5,4-f]quinazoline-9,1′-cyclohexan]-7-one; 5-chloro-2-{[4-(2-methoxyethoxy)piperidin-1-yl]methyl}-7,8-dihydro-6H-spiro[[1,3]oxazolo[5,4-f]quinazoline-9,1′-cyclohexan]-7-one; 2-{[(8aS)-octahydropyrrolo[1,2-a]pyrazin-2-yl]methyl}-5-chloro-7,8-dihydro-6H-spiro[[1,3]oxazolo[5,4-f]quinazoline-9,1′-cyclohexan]-7-one; methyl (3R)-1-({5-chloro-7-oxo-7,8-dihydro-6H-spiro[[1,3]oxazolo[5,4-f]quinazoline-9,1′-cyclohexan]-2-yl}methyl)pyrrolidine-3-carboxylate; 5-chloro-2-{[4-(2,2-difluoroethyl)piperazin-1-yl]methyl}-7,8-dihydro-6H-spiro[[1,3]oxazolo[5,4-f]quinazoline-9,1′-cyclohexan]-7-one; 5-chloro-2-({[(3-methyloxolan-3-yl)methyl]amino}methyl)-7,8-dihydro-6H-spiro[[1,3]oxazolo[5,4-f]quinazoline-9,1′-cyclohexan]-7-one; 5-chloro-2-({[(3R)-oxan-3-yl]amino}methyl)-7,8-dihydro-6H-spiro[[1,3]oxazolo[5,4-f]quinazoline-9,1′-cyclohexan]-7-one; 5-chloro-2-[({[(3R)-oxolan-3-yl]methyl}amino)methyl]-7,8-dihydro-6H-spiro[[1,3]oxazolo[5,4-f]quinazoline-9,1′-cyclohexan]-7-one; 5-chloro-2-{[(3-fluorooxan-4-yl)amino]methyl}-7,8-dihydro-6H-spiro[[1,3]oxazolo[5,4-f]quinazoline-9,1′-cyclohexan]-7-one; 5-chloro-2-{[(2,2-difluoro-3-hydroxypropyl)amino]methyl}-7,8-dihydro-6H-spiro[[1,3]oxazolo[5,4-f]quinazoline-9,1′-cyclohexan]-7-one; 5-chloro-2-{[(2,2-difluorocyclopentyl)amino]methyl}-7,8-dihydro-6H-spiro[[1,3]oxazolo[5,4-f]quinazoline-9,1′-cyclohexan]-7-one; 5-chloro-2-({[4-(methoxymethyl)oxan-4-yl]amino}methyl)-7,8-dihydro-6H-spiro[[1,3]oxazolo[5,4-f]quinazoline-9,1′-cyclohexan]-7-one; 5-chloro-2-[({[(3S)-oxolan-3-yl]methyl}amino)methyl]-7,8-dihydro-6H-spiro[[1,3]oxazolo[5,4-f]quinazoline-9,1′-cyclohexan]-7-one; 5-chloro-2-({[(1,3-oxazol-2-yl)methyl]amino}methyl)-7,8-dihydro-6H-spiro[[1,3]oxazolo[5,4-f]quinazoline-9,1′-cyclohexan]-7-one; 5-chloro-4′,4′-difluoro-2-methyl-7,8-dihydro-6H-spiro[[1,3]oxazolo[5,4-f]quinazoline-9,1′-cyclohexan]-7-one; 5-chloro-2-{[(1-methylpiperidin-4-yl)oxy]methyl}-7,8-dihydro-6H-spiro[[1,3]oxazolo[5,4-f]quinazoline-9,1′-cyclohexan]-7-one; 2-[({5-fluoro-7-oxo-7,8-dihydro-6H-spiro[[1,3]oxazolo[5,4-f]quinazoline-9,1′-cyclohexan]-2-yl}methyl)(methyl)amino]-N,N-dimethylacetamide; 2-[({5-fluoro-7-oxo-7,8-dihydro-6H-spiro[[1,3]oxazolo[5,4-f]quinazoline-9,1′-cyclohexan]-2-yl}methyl)amino]-N,N-dimethylacetamide; 5-chloro-4′,4′-difluoro-2-{[(1S,4S)-2-oxa-5-azabicyclo[2.2.1]heptan-5-yl]methyl}-7,8-dihydro-6H-spiro[[1,3]oxazolo[5,4-f]quinazoline-9,1′-cyclohexan]-7-one; 5-chloro-4′,4′-difluoro-2-[({[(3S)-oxolan-3-yl]methyl}amino)methyl]-7,8-dihydro-6H-spiro[[1,3]oxazolo[5,4-f]quinazoline-9,1′-cyclohexan]-7-one; 5-chloro-2,4′,4′-trimethyl-7,8-dihydro-6H-spiro[[1,3]oxazolo[5,4-f]quinazoline-9,1′-cyclohexan]-7-one; 5-chloro-4′,4′-difluoro-2-({[(3S)-oxan-3-yl]amino}methyl)-7,8-dihydro-6H-spiro[[1,3]oxazolo[5,4-f]quinazoline-9,1′-cyclohexan]-7-one; 5-chloro-4′,4′-difluoro-2-[(4-methylpiperazin-1-yl)methyl]-7,8-dihydro-6H-spiro[[1,3]oxazolo[5,4-f]quinazoline-9,1′-cyclohexan]-7-one; 5-chloro-4′,4′-difluoro-2-[({[(3R)-oxolan-3-yl]methyl}amino)methyl]-7,8-dihydro-6H-spiro[[1,3]oxazolo[5,4-f]quinazoline-9,1′-cyclohexan]-7-one; 5-chloro-4′,4′-difluoro-2-({[(3S)-oxolan-3-yl]amino}methyl)-7,8-dihydro-6H-spiro[[1,3]oxazolo[5,4-f]quinazoline-9,1′-cyclohexan]-7-one; 5-chloro-4′,4′-difluoro-2-({[(3R)-oxan-3-yl]amino}methyl)-7,8-dihydro-6H-spiro[[1,3]oxazolo[5,4-f]quinazoline-9,1′-cyclohexan]-7-one; 5-chloro-2-({[(1,3-oxazol-5-yl)methyl]amino}methyl)-7,8-dihydro-6H-spiro[[1,3]oxazolo[5,4-f]quinazoline-9,1′-cyclohexan]-7-one; 5-chloro-2-({[2-(1,2-oxazol-3-yl)ethyl]amino}methyl)-7,8-dihydro-6H-spiro[[1,3]oxazolo[5,4-f]quinazoline-9,1′-cyclohexan]-7-one; 5-chloro-2-({[(1,3-oxazol-4-yl)methyl]amino}methyl)-7,8-dihydro-6H-spiro[[1,3]oxazolo[5,4-f]quinazoline-9,1′-cyclohexan]-7-one; 5-chloro-2-({[(1-methyl-1H-pyrazol-3-yl)methyl]amino}methyl)-7,8-dihydro-6H-spiro[[1,3]oxazolo[5,4-f]quinazoline-9,1′-cyclohexan]-7-one; 5-chloro-2-({[(1,2-oxazol-5-yl)methyl]amino}methyl)-7,8-dihydro-6H-spiro[[1,3]oxazolo[5,4-f]quinazoline-9,1′-cyclohexan]-7-one; and pharmaceutically acceptable salts thereof; or 5′-chloro-7′-(hydroxymethyl)-7′,8′-dihydro-6′H-spiro[cyclohexane-1,9′-furo[2,3-f]quinazoline]-7′-one; 5′-chloro-7′-[4-(2-fluoroethyl)piperazine-1-carbonyl]-7′,8′-dihydro-6′H-spiro[cyclohexane-1,9′-furo[2,3-f]quinazoline]-7′-one; N-{2-[benzyl(methyl)amino]ethyl}-5′-chloro-N-methyl-7′-oxo-7′,8′-dihydro-6′H-spiro[cyclohexane-1,9′-furo[2,3-f]quinazoline]-2′-carboxamide; 5′-chloro-2′-{[4-(propan-2-yl)piperazin-1-yl]methyl}-7′,8′-dihydro-6′H-spiro[cyclohexane-1,9′-furo[2 3-f]quinazoline]-7′-one; 5′-chloro-2′-{[methyl({[4-(propan-2-yl)-4H-1,2,4-triazol-3-yl]methyl})amino]methyl}-7′,8′-dihydro-6′H-spiro[cyclohexane-1,9′-furo[2,3-]quinazoline]-7′-one; 5′-chloro-2′-(4-methylpiperazine-1-carbonyl)-7′,8′-dihydro-6′H-spiro[cyclohexane-1,9′-furo[2,3-f]quinazoline]-7′-one; 5′-chloro-2′-(4-ethylpiperazine-1-carbonyl)-7′,8′-dihydro-6′H-spiro[cyclohexane-1,9′-furo[2,3-f]quinazoline]-7′-one; 5′-chloro-N-(2-methoxyethyl)-N-methyl-7′-oxo-7′,8′-dihydro-6′H-spiro[cyclohexane-1,9′-furo[2,3-f]quinazoline]-2′-carboxamide; 5′-chloro-2′-[4-(2-methoxyethyl)piperazine-1-carbonyl]-7′,8′-dihydro-6′H-spiro[cyclohexane-1,9′-furo[2,3-f]quinazoline]-7′-one; 5′-chloro-7′-(piperidine-1-carbonyl)-7′,8′-dihydro-6′H-spiro[cyclohexane-1,9′-furo[2,3-f]quinazoline]-7′-one; 2′-(azetidine-1-carbonyl)-5′-chloro-7′,8′-dihydro-6′H-spiro[cyclohexane-1,9′-furo[2,3-f]quinazoline]-7′-one; 5′-chloro-7′-(pyrrolidine-1-carbonyl)-7′,8′-dihydro-6′H-spiro[cyclohexane-1,9′-furo[2,3-f]quinazoline]-7′-one; 5′-chloro-7′-(morpholine-4-carbonyl)-7′,8′-dihydro-6′H-spiro[cyclohexane-1,9′-furo[2,3-f]quinazoline]-7′-one; 5′-chloro-2′-[4-(2,2,2-trifluoroethyl)piperazine-1-carbonyl]-7′,8′-dihydro-6′H-spiro[cyclohexane-1,9′-furo[2,3-f]quinazoline]-7′-one; 5′-chloro-2′-[4-(2,2-difluoroethyl)piperazine-1-carbonyl]-7′,8′-dihydro-6′H-spiro[cyclohexane-1,9′-furo[2,3-f]quinazoline]-7′-one; 5′-chloro-2′-[(1S,4S)-2-oxa-5-azabicyclo[2.2.1]heptan-5-ylmethyl]-7′,8′-dihydro-6′H-spiro[cyclohexane-1,9′-furo[2,3-f]quinazoline]-7′-one; 5′-chloro-2′-(piperidin-1-ylmethyl)-7′,8′-dihydro-6′H-spiro[cyclohexane-1,9′-furo[2,3-f]quinazoline]-7′-one; 5′-chloro-2′-(morpholin-4-ylmethyl)-7′,8′-dihydro-6′H-spiro[cyclohexane-1,9′-furo[2,3-f]quinazoline]-7′-one; 5′-chloro-2′-(pyrrolidin-1-ylmethyl)-7′,8′-dihydro-6′H-spiro[cyclohexane-1,9′-furo[2,3-f]quinazoline]-7′-one; 5′-chloro-2′-[(4-methylpiperazin-1-yl)methyl]-7′,8′-dihydro-6′H-spiro[cyclohexane-1,9′-furo[2,3-f]quinazoline]-7′-one; 5′-chloro-N-ethyl-7′-oxo-N-(propan-2-yl)-7′,8′-dihydro-6′H-spiro[cyclohexane-1,9′-furo[2,3-f]quinazoline]-2′-carboxamide; 5′-chloro-7′-oxo-N-[2-(propan-2-yloxy)ethyl]-7′,8′-dihydro-6′H-spiro[cyclohexane-1,9′-furo[2,3-f]quinazoline]-2′-carboxamide; 5′-chloro-2′-(4-methoxypiperidine-1-carbonyl)-7′,8′-dihydro-6′H-spiro[cyclohexane-1,9′-furo[2,3-f]quinazoline]-7′-one; 5′-chloro-N-[(4-fluorophenyl)methyl]-7′-oxo-7′,8′-dihydro-6′H-spiro[cyclohexane-1,9′-furo[2,3-f]quinazoline]-2′-carboxamide; 5′-chloro-N-(2-methoxyethyl)-7′-oxo-7′,8′-dihydro-6′H-spiro[cyclohexane-1,9′-furo[2,3-f]quinazoline]-7′-carboxamide; 5′-chloro-N-methyl-N-(2-methylpropyl)-7′-oxo-7′,8′-dihydro-6′H-spiro[cyclohexane-1,9′-furo[2,3-f]quinazoline]-7′-carboxamide; 5′-chloro-N-cyclopentyl-N-methyl-7′-oxo-7′,8′-dihydro-6′H-spiro[cyclohexane-1,9′-furo[2,3-f]quinazoline]-7′-carboxamide; 5′-chloro-N-(1-hydroxy-3-methylbutan-2-yl)-7′-oxo-7′,8′-dihydro-6′H-spiro[cyclohexane-1,9′-furo[2,3-f]quinazoline]-2′-carboxamide; 5′-chloro-N-(1-methylpiperidin-4-yl)-7′-oxo-7′,8′-dihydro-6′H-spiro[cyclohexane-1,9′-furo[2,3-f]quinazoline]-2′-carboxamide; 5′-chloro-N-[(2-fluorophenyl)methyl]-7′-oxo-7′,8′-dihydro-6′H-spiro[cyclohexane-1,9′-furo[2,3-f]quinazoline]-2′-carboxamide; 5′-chloro-2′-[4-(2-hydroxyethyl)piperazine-1-carbonyl]-7′,8′-dihydro-6′H-spiro[cyclohexane-1,9′-furo[2,3-f]quinazoline]-7′-one; 5′-chloro-N-(3-hydroxypropyl)-7′-oxo-7′,8′-dihydro-6′H-spiro[cyclohexane-1,9′-furo[2,3-f]quinazoline]-2′-carboxamide; 5′-chloro-N-(4-hydroxybutyl)-7′-oxo-7′,8′-dihydro-6′H-spiro[cyclohexane-1,9′-furo[2,3-f]quinazoline]-2′-carboxamide; 5′-chloro-N-(2-hydroxyethyl)-7′-oxo-N-propyl-7′,8′-dihydro-6′H-spiro[cyclohexane-1,9′-furo[2,3-f]quinazoline]-2′-carboxamide; 5′-chloro-7′-oxo-N-[2-(pyrrolidin-1-yl)ethyl]-7′,8′-dihydro-6′H-spiro[cyclohexane-1,9′-furo[2,3-f]quinazoline]-2′-carboxamide; 5′-chloro-N-[2-(dimethylamino)ethyl]-N-ethyl-7′-oxo-7′,8′-dihydro-6′H-spiro[cyclohexane-1,9′-furo[2,3-f]quinazoline]-2′-carboxamide; 5′-chloro-N-[3-(1H-imidazol-1-yl)propyl]-7′-oxo-7′,8′-dihydro-6′H-spiro[cyclohexane-1,9′-furo[2,3-f]quinazoline]-2′-carboxamide; 5′-chloro-N-(2-hydroxyethyl)-N-methyl-7′-oxo-7′,8′-dihydro-6′H-spiro[cyclohexane-1,9′-furo[2,3-f]quinazoline]-2′-carboxamide; 5′-chloro-N-(2-hydroxybutyl)-7′-oxo-7′,8′-dihydro-6′H-spiro[cyclohexane-1,9′-furo[2,3-f]quinazoline]-2′-carboxamide; 5′-chloro-N-[3-(dimethylamino)propyl]-N-methyl-7′-oxo-7′,8′-dihydro-6′H-spiro[cyclohexane-1,9′-furo[2,3-f]quinazoline]-2′-carboxamide; 2′-[(8aS)-octahydropyrrolo[1,2-a]piperazine-2-carbonyl]-5′-chloro-7′,8′-dihydro-6′H-spiro[cyclohexane-1,9′-furo[2,3-f]quinazoline]-7′-one; 5′-chloro-N-methyl-7′-oxo-N-[2-(pyridin-2-yl)ethyl]-7′,8′-dihydro-6′H-spiro[cyclohexane-1,9′-furo[2,3-f]quinazoline]-2′-carboxamide; 5′-chloro-N-(2-cyanoethyl)-N-methyl-7′-oxo-7′,8′-dihydro-6′H-spiro[cyclohexane-1,9′-furo[2,3-f]quinazoline]-2′-carboxamide; 5′-chloro-N-(2-ethoxyethyl)-7′-oxo-7′,8′-dihydro-6′H-spiro[cyclohexane-1,9′-furo[2,3-f]quinazoline]-2′-carboxamide; 5′-chloro-7′-oxo-N-(oxolan-2-ylmethyl)-7′,8′-dihydro-6′H-spiro[cyclohexane-1,9′-furo[2,3-f]quinazoline]-2′-carboxamide; 5′-chloro-N-(2-methoxyethyl)-7′-oxo-N-(propan-2-yl)-7′,8′-dihydro-6′H-spiro[cyclohexane-1,9′-furo[2,3-f]quinazoline]-2′-carboxamide; 5′-chloro-7′-oxo-N-[2-(thiophen-2-yl)ethyl]-7′,8′-dihydro-6′H-spiro[cyclohexane-1,9′-furo[2,3-f]quinazoline]-2′-carboxamide; 5′-chloro-N-cyclopentyl-7′-oxo-7′,8′-dihydro-6′H-spiro[cyclohexane-1,9′-furo[2,3-f]quinazoline]-2′-carboxamide; 5′-chloro-N-ethyl-N-(2-hydroxyethyl)-7′-oxo-7′,8′-dihydro-6′H-spiro[cyclohexane-1,9′-furo[2,3-f]quinazoline]-2′-carboxamide; 5′-chloro-2′-(3-hydroxypiperidine-1-carbonyl)-7′,8′-dihydro-6′H-spiro[cyclohexane-1,9′-furo[2,3-f]quinazoline]-7′-one; 5′-chloro-7′-oxo-N-[2-(1H-pyrrol-1-yl)ethyl]-7′,8′-dihydro-6′H-spiro[cyclohexane-1,9′-furo[2,3-f]quinazoline]-2′-carboxamide; 5′-chloro-N-methyl-N-(1-methylpyrrolidin-3-yl)-7′-oxo-7′,8′-dihydro-6′H-spiro[cyclohexane-1,9′-furo[2,3-f]quinazoline]-2′-carboxamide; 5′-chloro-N-(3-hydroxy-3-phenylpropyl)-N-methyl-7′-oxo-7′,8′-dihydro-6′H-spiro[cyclohexane-1,9′-furo[2,3-f]quinazoline]-2′-carboxamide; 5′-chloro-2′-[3-(hydroxymethyl)-3-(2-methylpropyl)piperidine-1-carbonyl]-7′,8′-dihydro-6′H-spiro[cyclohexane-1,9′-furo[2,3-f]quinazoline]-7′-one; 5′-chloro-N-[(2,3-dimethoxyphenyl)methyl]-N-methyl-7′-oxo-7′,8′-dihydro-6′H-spiro[cyclohexane-1,9′-furo[2,3-f]quinazoline]-7′-carboxamide; 5′-chloro-7′-[2-(trifluoromethyl)-5H,6H,7H,8H-imidazo [1,2-a]pyrazine-7-carbonyl]-7′,8′-dihydro-6′H-spiro[cyclohexane-1,9′-furo[2,3-f]quinazoline]-7′-one; 5′-chloro-N-[3-(morpholin-4-yl)propyl]-7′-oxo-7′,8′-dihydro-6′H-spiro[cyclohexane-1,9′-furo[2,3-f]quinazoline]-7′-carboxamide; 5′-chloro-N-[(2-methoxyphenyl)methyl]-N-methyl-7′-oxo-7′,8′-dihydro-6′H-spiro[cyclohexane-1,9′-furo[2,3-f]quinazoline]-2′-carboxamide; 5′-chloro-2′-[3-(1H-imidazol-1-ylmethyl)piperidine-1-carbonyl]-7′,8′-dihydro-6′H-spiro[cyclohexane-1,9′-furo[2,3-f]quinazoline]-7′-one; ethyl 2-[4-({5′-chloro-7′-oxo-7′,8′-dihydro-6′H-spiro[cyclohexane-1,9′-furo[2,3-f]quinazoline]-2′-yl}carbonyl)piperazin-1-yl]acetate; 5′-chloro-N-[(2,4-dimethoxyphenyl)methyl]-N-methyl-7′-oxo-7′,8′-dihydro-6′H-spiro[cyclohexane-1,9′-furo[2,3-f]quinazoline]-2′-carboxamide; 5′-chloro-N-[(3-methoxyphenyl)methyl]-N-methyl-7′-oxo-7′,8′-dihydro-6′H-spiro[cyclohexane-1,9′-furo[2,3-f]quinazoline]-2′-carboxamide; 5′-chloro-N-(2,2-dimethyloxan-4-yl)-N-ethyl-7′-oxo-7′,8′-dihydro-6′H-spiro[cyclohexane-1,9′-furo[2,3-f]quinazoline]-2′-carboxamide; 5′-chloro-N-methyl-N-[(1-methyl-1H-1,3-benzodiazol-2-yl)methyl]-7′-oxo-7′,8′-dihydro-6′H-spiro[cyclohexane-1,9′-furo[2,3-f]quinazoline]-2′-carboxamide; 5′-chloro-N-[(3,5-dimethoxyphenyl)methyl]-N-methyl-7′-oxo-7′,8′-dihydro-6′H-spiro[cyclohexane-1,9′-furo[2,3-f]quinazoline]-2′-carboxamide; 5′-chloro-2′-{5H,6H,7H, 8H-imidazo [1,2-a]pyrazine-7-carbonyl}-7′,8′-dihydro-6′H-spiro[cyclohexane-1,9′-furo[2,3-f]quinazoline]-7′-one; 5′-chloro-2′-[2-(pyridin-2-yl)pyrrolidine-1-carbonyl]-7′,8′-dihydro-6′H-spiro[cyclohexane-1,9′-furo[2,3-f]quinazoline]-7′-one; 5′-chloro-N-(2-hydroxy-2-phenylethyl)-N-methyl-7′-oxo-7′,8′-dihydro-6′H-spiro[cyclohexane-1,9′-furo[2,3-f]quinazoline]-2′-carboxamide; 5′-chloro-N-[2-(2,3-dihydro-1H-indol-1-yl)ethyl]-7′-oxo-7′,8′-dihydro-6′H-spiro[cyclohexane-1,9′-furo[2,3-f]quinazoline]-2′-carboxamide; 5′-chloro-N-[(2,3-dimethoxyphenyl)methyl]-7′-oxo-7′,8′-dihydro-6′H-spiro[cyclohexane-1,9′-furo[2,3-f]quinazoline]-2′-carboxamide; 5′-chloro-N-methyl-N-[(5-methyl-1H-1,3-benzodiazol-2-yl)methyl]-7′-oxo-7′,8′-dihydro-6′H-spiro[cyclohexane-1,9′-furo[2,3-f]quinazoline]-2′-carboxamide; 5′-chloro-N-methyl-7′-oxo-N-[3-(trimethyl-1H-pyrazol-4-yl)propyl]-7′,8′-dihydro-6′H-spiro[cyclohexane-1,9′-furo[2,3-f]quinazoline]-2′-carboxamide; 5′-chloro-2′-{octahydropyrrolo[1,2-a]piperazine-2-carbonyl}-7′,8′-dihydro-6′H-spiro[cyclohexane-1,9′-furo[2,3-f]quinazoline]-7′-one; 5′-chloro-N-(2,3-dihydro-1,4-benzodioxin-6-ylmethyl)-7′-oxo-7′,8′-dihydro-6′H-spiro[cyclohexane-1,9′-furo[2,3-f]quinazoline]-2′-carboxamide; 5′-chloro-N-[(5-cyclopropyl-1H-pyrazol-3-yl)methyl]-N-methyl-7′-oxo-7′,8′-dihydro-6′H-spiro[cyclohexane-1,9′-furo[2,3-f]quinazoline]-2′-carboxamide; 5′-chloro-N-[(2,4-dimethoxyphenyl)methyl]-7′-oxo-7′,8′-dihydro-6′H-spiro[cyclohexane-1,9′-furo[2,3-f]quinazoline]-2′-carboxamide; N-(1-benzylpyrrolidin-3-yl)-5′-chloro-7′-oxo-7′,8′-dihydro-6′H-spiro[cyclohexane-1,9′-furo[2,3-f]quinazoline]-2′-carboxamide; 5′-chloro-N-[(2,3-difluoro-4-methoxyphenyl)methyl]-7′-oxo-7′,8′-dihydro-6′H-spiro[cyclohexane-1,9′-furo[2,3-f]quinazoline]-2′-carboxamide; 5′-chloro-N-{[4-(dimethylamino)phenyl]methyl}-7′-oxo-7′,8′-dihydro-6′H-spiro[cyclohexane-1,9′-furo[2,3-f]quinazoline]-2′-carboxamide; 5′-chloro-N-[(1-methyl-1H-imidazol-2-yl)methyl]-7′-oxo-N-(propan-2-yl)-7′,8′-dihydro-6′H-spiro[cyclohexane-1,9′-furo[2,3-f]quinazoline]-2′-carboxamide; 5′-chloro-2′-[4-(pyridin-4-yl)piperazine-1-carbonyl]-7′,8′-dihydro-6′H-spiro[cyclohexane-1,9′-furo[2,3-f]quinazoline]-7′-one; N-[(5-tert-butyl-1H-pyrazol-3-yl)methyl]-5′-chloro-N-methyl-7′-oxo-7′,8′-dihydro-6′H-spiro[cyclohexane-1,9′-furo[2,3-f]quinazoline]-2′-carboxamide; 2′-(4-benzylpiperazine-1-carbonyl)-5′-chloro-7′,8′-dihydro-6′H-spiro[cyclohexane-1,9′-furo[2,3-f]quinazoline]-7′-one; 5′-chloro-N-[(1-methylpiperidin-4-yl)methyl]-7′-oxo-7′,8′-dihydro-6′H-spiro[cyclohexane-1,9′-furo[2,3-f]quinazoline]-2′-carboxamide; 5′-chloro-7′-(3-methoxypiperidine-1-carbonyl)-7′,8′-dihydro-6′H-spiro[cyclohexane-1,9′-furo[2,3-f]quinazoline]-7′-one; 5′-chloro-N-methyl-7′-oxo-N-[(trimethyl-1H-pyrazol-4-yl)methyl]-7′,8′-dihydro-6′H-spiro[cyclohexane-1,9′-furo[2,3-f]quinazoline]-2′-carboxamide; 5′-chloro-2′-[4-(pyridin-2-yl)piperazine-1-carbonyl]-7′,8′-dihydro-6′H-spiro[cyclohexane-1,9′-furo[2,3-f]quinazoline]-7′-one; 5′-chloro-N-[3-(3,5-dimethyl-1H-pyrazol-1-yl)propyl]-N-methyl-7′-oxo-7′,8′-dihydro-6′H-spiro[cyclohexane-1,9′-furo[2,3-f]quinazoline]-2′-carboxamide; 5′-chloro-2′-(4-methyl-2-phenylpiperazine-1-carbonyl)-7′,8′-dihydro-6′H-spiro[cyclohexane-1,9′-furo[2,3-f]quinazoline]-7′-one; 5′-chloro-2′-[3-(dimethylamino)piperidine-1-carbonyl]-7′,8′-dihydro-6′H-spiro[cyclohexane-1,9′-furo[2,3-f]quinazoline]-7′-one; 5′-chloro-N-[2-(morpholin-4-yl)ethyl]-7′-oxo-7′,8′-dihydro-6′H-spiro[cyclohexane-1,9′-furo[2,3-f]quinazoline]-2′-carboxamide; 5′-chloro-7′-oxo-N-[2-(pyridin-3-yloxy)ethyl]-7′,8′-dihydro-6′H-spiro[cyclohexane-1,9′-furo[2,3-f]quinazoline]-2′-carboxamide; 5′-chloro-N-[(2-fluoro-4-methoxyphenyl)methyl]-7′-oxo-7′,8′-dihydro-6′H-spiro[cyclohexane-1,9′-furo[2,3-f]quinazoline]-2′-carboxamide; 1-({5′-chloro-7′-oxo-7′,8′-dihydro-6′H-spiro[cyclohexane-1,9′-furo[2,3-f]quinazoline]-2′-yl}carbonyl)azetidine-3-carbonitrile; 5′-chloro-2′-{3-phenyl-5H,6H,7H,8H-imidazo [1,2-a]pyrazine-7-carbonyl}-7′,8′-dihydro-6′H-spiro[cyclohexane-1,9′-furo[2,3-f]quinazoline]-7′-one; 5′-chloro-7′-oxo-N-[2-(2-oxopyrrolidin-1-yl)ethyl]-7′,8′-dihydro-6′H-spiro[cyclohexane-1,9′-furo[2,3-f]quinazoline]-2′-carboxamide; 5′-chloro-7′-oxo-N-(2,2,6,6-tetramethyloxan-4-yl)-7′,8′-dihydro-6′H-spiro[cyclohexane-1,9′-furo[2,3-f]quinazoline]-2′-carboxamide; 2′-[(1R,5S)-3-azabicyclo[3.1.0]hexane-3-carbonyl]-5′-chloro-7′,8′-dihydro-6′H-spiro[cyclohexane-1,9′-furo[2,3-f]quinazoline]-7′-one; 5′-chloro-N-[2-(4-fluorophenoxy)ethyl]-7′-oxo-7′,8′-dihydro-6′H-spiro[cyclohexane-1,9′-furo[2,3-f]quinazoline]-2′-carboxamide; 5′-chloro-7′-oxo-N-[2-(1H-pyrazol-1-yl)ethyl]-7′,8′-dihydro-6′H-spiro[cyclohexane-1,9′-furo[2,3-f]quinazoline]-2′-carboxamide; 5′-chloro-N-[4-(4-hydroxypiperidin-1-yl)phenyl]-7′-oxo-7′,8′-dihydro-6′H-spiro[cyclohexane-1,9′-furo[2,3-f]quinazoline]-2′-carboxamide; 5′-chloro-N-(3,3-dimethyl-2-oxobutyl)-7′-oxo-7′,8′-dihydro-6′H-spiro[cyclohexane-1,9′-furo[2,3-f]quinazoline]-2′-carboxamide; 5′-chloro-N-(furan-2-ylmethyl)-N-methyl-7′-oxo-7′,8′-dihydro-6′H-spiro[cyclohexane-1,9′-furo[2,3-f]quinazoline]-2′-carboxamide; 5′-chloro-2′-{4-[2-(dimethylamino)ethyl]piperidine-1-carbonyl}-7′,8′-dihydro-6′H-spiro[cyclohexane-1,9′-furo[2,3-f]quinazoline]-7′-one; 5′-chloro-2′-[4-(5-chloropyridin-2-yl)piperazine-1-carbonyl]-7′,8′-dihydro-6′H-spiro[cyclohexane-1,9′-furo[2,3-f]quinazoline]-7′-one; 5′-chloro-N-[(2-methoxyphenyl)methyl]-7′-oxo-7′,8′-dihydro-6′H-spiro[cyclohexane-1,9′-furo[2,3-f]quinazoline]-2′-carboxamide; 5′-chloro-N-(2-hydroxy-2-methylpropyl)-7′-oxo-7′,8′-dihydro-6′H-spiro[cyclohexane-1,9′-furo[2,3-f]quinazoline]-2′-carboxamide; 5′-chloro-N-[(4-cyanophenyl)methyl]-7′-oxo-7′,8′-dihydro-6′H-spiro[cyclohexane-1,9′-furo[2,3-f]quinazoline]-2′-carboxamide; 5′-chloro-2′-[3-oxo-4-(propan-2-yl)piperazine-1-carbonyl]-7′,8′-dihydro-6′H-spiro[cyclohexane-1,9′-furo[2,3-f]quinazoline]-7′-one; 5′-chloro-N-{[5-(difluoromethoxy)pyridin-2-yl]methyl}-7′-oxo-7′,8′-dihydro-6′H-spiro[cyclohexane-1,9′-furo[2,3-f]quinazoline]-2′-carboxamide; 5′-chloro-2′-[4-(oxetan-3-yl)piperazine-1-carbonyl]-7′,8′-dihydro-6′H-spiro[cyclohexane-1,9′-furo[2,3-f]quinazoline]-7′-one; 5′-chloro-N-[(1R,5S)-6,6-difluorobicyclo[3.1.0]hexan-3-yl]-7′-oxo-7′,8′-dihydro-6′H-spiro[cyclohexane-1,9′-furo[2,3-f]quinazoline]-2′-carboxamide; N-[2-(2H-1,3-benzodioxol-5-yl)ethyl]-5′-chloro-7′-oxo-7′,8′-dihydro-6′H-spiro[cyclohexane-1,9′-furo[2,3-f]quinazoline]-2′-carboxamide; 5′-chloro-N-[(4-ethyl-4H-1,2,4-triazol-3-yl)methyl]-N-methyl-7′-oxo-7′,8′-dihydro-6′H-spiro[cyclohexane-1,9′-furo[2,3-f]quinazoline]-2′-carboxamide; 5′-chloro-N-[(4-cyano-3-fluorophenyl)methyl]-7′-oxo-7′,8′-dihydro-6′H-spiro[cyclohexane-1,9′-furo[2,3-f]quinazoline]-7′-carboxamide; 2-{5′-chloro-7′-oxo-7′,8′-dihydro-6′H-spiro[cyclohexane-1,9′-furo[2,3-f]quinazoline]-7′-ylformamido}-N,N-diethylacetamide; 5′-chloro-7′-({6′-methyl-5′,6′-dihydro-4′H-spiro[piperidine-4,7′-thieno[2,3-c]pyridine]-1-yl}carbonyl)-7′,8′-dihydro-6′H-spiro[cyclohexane-1,9′-furo[2,3-f]quinazoline]-7′-one; 5′-chloro-N-[(6-methylpyridin-3-yl)methyl]-7′-oxo-7′,8′-dihydro-6′H-spiro[cyclohexane-1,9′-furo[2,3-f]quinazoline]-7′-carboxamide; 5′-chloro-7′-oxo-N-[(trimethyl-1H-imidazol-2-yl)methyl]-7′,8′-dihydro-6′H-spiro[cyclohexane-1,9′-furo[2,3-f]quinazoline]-2′-carboxamide; 5′-chloro-N-[(5-methylpyridin-3-yl)methyl]-7′-oxo-7′,8′-dihydro-6′H-spiro[cyclohexane-1,9′-furo[2,3-f]quinazoline]-2′-carboxamide; 5′-chloro-N-methyl-7′-oxo-N-[(5-phenyl-1,2-oxazol-3-yl)methyl]-7′,8′-dihydro-6′H-spiro[cyclohexane-1,9′-furo[2,3-f]quinazoline]-2′-carboxamide; 2′-{6-azaspiro[2.5]octane-6-carbonyl}-5′-chloro-7′,8′-dihydro-6′H-spiro[cyclohexane-1,9′-furo[2,3-f]quinazoline]-7′-one; 5′-chloro-N-[2-(oxan-4-yl)ethyl]-7′-oxo-7′,8′-dihydro-6′H-spiro[cyclohexane-1,9′-furo[2,3-f]quinazoline]-2′-carboxamide; 5′-chloro-2′-({6′,7′-dihydro-5′H-spiro[piperidine-4,4′-thieno[3,2-c]pyridine]-1-yl}carbonyl)-7′,8′-dihydro-6′H-spiro[cyclohexane-1,9′-furo[2,3-f]quinazoline]-7′-one; 5′-chloro-N-(1-methoxy-2-methylpropan-2-yl)-7′-oxo-7′,8′-dihydro-6′H-spiro[cyclohexane-1,9′-furo[2,3-f]quinazoline]-2′-carboxamide; 5′-chloro-7′-oxo-N-[1-(pyridin-3-yl)piperidin-4-yl]-7′,8′-dihydro-6′H-spiro[cyclohexane-1,9′-furo[2,3-f]quinazoline]-2′-carboxamide; 5′-chloro-2′-({3,4-dihydrospiro[2-benzopyran-1,4′-piperidine]-1′-yl}carbonyl)-7′,8′-dihydro-6′H-spiro[cyclohexane-1,9′-furo[2,3-f]quinazoline]-7′-one; 5′-chloro-7′-oxo-N-(2-phenoxyethyl)-7′,8′-dihydro-6′H-spiro[cyclohexane-1,9′-furo[2,3-f]quinazoline]-2′-carboxamide; 3-{5′-chloro-7′-oxo-7′,8′-dihydro-6′H-spiro[cyclohexane-1,9′-furo[2,3-f]quinazoline]-2′-ylformamido}-N,N-dimethylpropanamide; 5′-chloro-N-[1-(methoxymethyl)cyclopropyl]-7′-oxo-7′,8′-dihydro-6′H-spiro[cyclohexane-1,9′-furo[2,3-f]quinazoline]-7′-carboxamide; 5′-chloro-N-[(3-fluoropyridin-4-yl)methyl]-7′-oxo-7′,8′-dihydro-6′H-spiro[cyclohexane-1,9′-furo[2,3-f]quinazoline]-7′-carboxamide; 5′-chloro-N-(cyclobutylmethyl)-7′-oxo-N-(oxolan-2-ylmethyl)-7′,8′-dihydro-6′H-spiro[cyclohexane-1,9′-furo[2,3-f]quinazoline]-7′-carboxamide; 5′-chloro-7′-[4-(furan-2-ylmethyl)piperazine-1-carbonyl]-7′,8′-dihydro-6′H-spiro[cyclohexane-1,9′-furo[2,3-f]quinazoline]-7′-one; 5′-chloro-N-[(1-methyl-1H-pyrazol-4-yl)methyl]-7′-oxo-7′,8′-dihydro-6′H-spiro[cyclohexane-1,9′-furo[2,3-f]quinazoline]-2′-carboxamide; 5′-chloro-7′-oxo-N-[1-(pyridin-2-yl)cyclopropyl]-7′,8′-dihydro-6′H-spiro[cyclohexane-1,9′-furo[2,3-f]quinazoline]-2′-carboxamide; 5′-chloro-N-(4-methyloxan-4-yl)-7′-oxo-7′,8′-dihydro-6′H-spiro[cyclohexane-1,9′-furo[2,3-f]quinazoline]-2′-carboxamide; 5′-chloro-N-{[1-(ethoxymethyl)cyclopropyl]methyl}-7′-oxo-7′,8′-dihydro-6′H-spiro[cyclohexane-1,9′-furo[2,3-f]quinazoline]-2′-carboxamide; 2′-(4-benzoylpiperazine-1-carbonyl)-5′-chloro-7′,8′-dihydro-6′H-spiro[cyclohexane-1,9′-furo[2,3-f]quinazoline]-7′-one; 2′-(4-{bicyclo[2.2.1]heptan-2-yl}piperazine-1-carbonyl)-5′-chloro-7′,8′-dihydro-6′H-spiro[cyclohexane-1,9′-furo[2,3-f]quinazoline]-7′-one; 5′-chloro-N-[(1-methyl-1H-imidazol-5-yl)methyl]-7′-oxo-7′,8′-dihydro-6′H-spiro[cyclohexane-1,9′-furo[2,3-f]quinazoline]-2′-carboxamide; 5′-chloro-7′-oxo-N-[2-oxo-2-(piperidin-1-yl)ethyl]-7′,8′-dihydro-6′H-spiro[cyclohexane-1,9′-furo[2,3-f]quinazoline]-2′-carboxamide; 5′-chloro-2′-(3-hydroxyazetidine-1-carbonyl)-7′,8′-dihydro-6′H-spiro[cyclohexane-1,9′-furo[2,3-f]quinazoline]-7′-one; 5′-chloro-7′-oxo-N-{[5-(trifluoromethyl)pyridin-2-yl]methyl}-7′,8′-dihydro-6′H-spiro[cyclohexane-1,9′-furo[2,3-f]quinazoline]-2′-carboxamide; 5′-chloro-N-[1-(oxan-4-yl)cyclopropyl]-7′-oxo-7′,8′-dihydro-6′H-spiro[cyclohexane-1,9′-furo[2,3-f]quinazoline]-2′-carboxamide; 5′-chloro-N-methyl-7′-oxo-N-(prop-2-yn-1-yl)-7′,8′-dihydro-6′H-spiro[cyclohexane-1,9′-furo[2,3-f]quinazoline]-7′-carboxamide; 5′-chloro-N,N-diethyl-7′-oxo-7′,8′-dihydro-6′H-spiro[cyclohexane-1,9′-furo[2,3-f]quinazoline]-7′-carboxamide; 5′-chloro-N-(1-methyl-2-oxopiperidin-4-yl)-7′-oxo-7′,8′-dihydro-6′H-spiro[cyclohexane-1,9′-furo[2,3-f]quinazoline]-7′-carboxamide; 5′-chloro-2′-[(1R,5S,6S)-6-(pyridin-2-yl)-3-azabicyclo[3.1.0]hexane-3-carbonyl]-7′,8′-dihydro-6′H-spiro[cyclohexane-1,9′-furo[2,3-f]quinazoline]-7′-one; 5′-chloro-N-[(6-fluoropyridin-2-yl)methyl]-7′-oxo-7′,8′-dihydro-6′H-spiro[cyclohexane-1,9′-furo[2,3-f]quinazoline]-2′-carboxamide; 5′-chloro-N-ethyl-N-methyl-7′-oxo-7′,8′-dihydro-6′H-spiro[cyclohexane-1,9′-furo[2,3-f]quinazoline]-2′-carboxamide; 5′-chloro-2′-{6,6-dimethyl-3-azabicyclo[3.1.0]hexane-3-carbonyl}-7′,8′-dihydro-6′H-spiro[cyclohexane-1,9′-furo[2,3-f]quinazoline]-7′-one; 5′-chloro-2′-{7,7-difluoro-3-azabicyclo[4.1.0]heptane-3-carbonyl}-7′,8′-dihydro-6′H-spiro[cyclohexane-1,9′-furo[2,3-f]quinazoline]-7′-one; 5′-chloro-2′-{6,6-difluoro-3-azabicyclo[3.1.0]hexane-3-carbonyl}-7′,8′-dihydro-6′H-spiro[cyclohexane-1,9′-furo[2,3-f]quinazoline]-7′-one; 5′-chloro-7′-oxo-N-(2,2,2-trifluoroethyl)-7′,8′-dihydro-6′H-spiro[cyclohexane-1,9′-furo[2,3-f]quinazoline]-2′-carboxamide; 5′-chloro-7′-oxo-N-[(5-oxopyrrolidin-2-yl)methyl]-N-(propan-2-yl)-7′,8′-dihydro-6′H-spiro[cyclohexane-1,9′-furo[2,3-f]quinazoline]-2′-carboxamide; 5′-chloro-N-[2-(2,3-dihydro-1-benzofuran-5-yl)ethyl]-7′-oxo-7′,8′-dihydro-6′H-spiro[cyclohexane-1,9′-furo[2,3-f]quinazoline]-2′-carboxamide; 5′-chloro-N-(4,4-difluorocyclohexyl)-7′-oxo-7′,8′-dihydro-6′H-spiro[cyclohexane-1,9′-furo[2,3-f]quinazoline]-2′-carboxamide; N-{bicyclo[1.1.1]pentan-1-yl}-5′-chloro-7′-oxo-7′,8′-dihydro-6′H-spiro[cyclohexane-1,9′-furo[2,3-f]quinazoline]-2′-carboxamide; 5′-chloro-N-[(1S,3R)-3-fluorocyclopentyl]-7′-oxo-7′,8′-dihydro-6′H-spiro[cyclohexane-1,9′-furo[2,3-f]quinazoline]-2′-carboxamide; 5′-chloro-N-methyl-7′-oxo-N-(prop-2-en-1-yl)-7′,8′-dihydro-6′H-spiro[cyclohexane-1,9′-furo[2,3-f]quinazoline]-7′-carboxamide; 5′-chloro-7′-oxo-N-(prop-2-yn-1-yl)-7′,8′-dihydro-6′H-spiro[cyclohexane-1,9′-furo[2,3-f]quinazoline]-7′-carboxamide; 5′-chloro-7′-{2-ethyl-1-oxo-1H,2H,5H,6H,7H,8H-pyrido[3,4-d]pyridazine-6-carbonyl}-7′,8′-dihydro-6′H-spiro[cyclohexane-1,9′-furo[2,3-]quinazoline]-7′-one; 5′-chloro-N-(1,3-dihydro-2-benzofuran-5-ylmethyl)-7′-oxo-7′,8′-dihydro-6′H-spiro[cyclohexane-1,9′-furo[2,3-f]quinazoline]-2′-carboxamide; 5′-chloro-N-[4-(methoxymethyl)oxan-4-yl]-7′-oxo-7′,8′-dihydro-6′H-spiro[cyclohexane-1,9′-furo[2,3-]quinazoline]-2′-carboxamide; 5′-chloro-N-cyclobutyl-7′-oxo-7′,8′-dihydro-6′H-spiro[cyclohexane-1,9′-furo[2,3-f]quinazoline]-2′-carboxamide; 5′-chloro-2′-{9-propanoyl-2,9-diazaspiro[5.5]undecane-2-carbonyl}-7′,8′-dihydro-6′H-spiro[cyclohexane-1,9′-furo[2,3-f]quinazoline]-7′-one; 5′-chloro-N-methyl-7′-oxo-N-(propan-2-yl)-7′,8′-dihydro-6′H-spiro[cyclohexane-1,9′-furo[2,3-f]quinazoline]-2′-carboxamide; 5′-chloro-2′-[(1R,5S,6S)-6-(2-methoxyphenyl)-3-azabicyclo[3.1.0]hexane-3-carbonyl]-7′,8′-dihydro-6′H-spiro[cyclohexane-1,9′-furo[2,3-]quinazoline]-7′-one; N-butyl-5′-chloro-7′-oxo-7′,8′-dihydro-6′H-spiro[cyclohexane-1,9′-furo[2,3-f]quinazoline]-2′-carboxamide; 5′-chloro-2′-{8-ethoxy-2H,3H,4H,5H-pyrido[3,2-f][1,4]oxazepine-4-carbonyl}-7′,8′-dihydro-6′H-spiro[cyclohexane-1,9′-furo[2,3-f]quinazoline]-7′-one; 5′-chloro-7′-oxo-N-[2-(thiophen-3-yl)ethyl]-7′,8′-dihydro-6′H-spiro[cyclohexane-1,9′-furo[2,3-f]quinazoline]-2′-carboxamide; 5′-chloro-N-(2-cyclobutyl-2,2-difluoroethyl)-7′-oxo-7′,8′-dihydro-6′H-spiro[cyclohexane-1,9′-furo[2,3-f]quinazoline]-2′-carboxamide; 5′-chloro-N-methyl-7′-oxo-N-propyl-7′,8′-dihydro-6′H-spiro[cyclohexane-1,9′-furo[2,3-f]quinazoline]-2′-carboxamide; (2R)-1-({5′-chloro-7′-oxo-7′,8′-dihydro-6′H-spiro[cyclohexane-1,9′-furo[2,3-f]quinazoline]-2′-yl}carbonyl)pyrrolidine-2-carboxylic acid; 5′-chloro-7′-[(1R,6R)-7,7-difluoro-6-methyl-3-azabicyclo[4.1.0]heptane-3-carbonyl]-7′,8′-dihydro-6′H-spiro[cyclohexane-1,9′-furo[2,3-f]quinazoline]-7′-one; 5′-chloro-N-methyl-7′-oxo-N-{[4-(propan-2-yl)-4H-1,2,4-triazol-3-yl]methyl}-7′,8′-dihydro-6′H-spiro[cyclohexane-1,9′-furo[2,3-]quinazoline]-2′-carboxamide; 5′-chloro-7′-({4-oxo-3,4-dihydrospiro[1-benzopyran-2,3′-pyrrolidine]-1′-yl}carbonyl)-7′,8′-dihydro-6′H-spiro[cyclohexane-1,9′-furo[2,3-f]quinazoline]-7′-one; 5′-chloro-N-cyclopropyl-7′-oxo-7′,8′-dihydro-6′H-spiro[cyclohexane-1,9′-furo[2,3-f]quinazoline]-7′-carboxamide; 5′-chloro-N-ethyl-7′-oxo-N-propyl-7′,8′-dihydro-6′H-spiro[cyclohexane-1,9′-furo[2,3-f]quinazoline]-2′-carboxamide; 5′-chloro-7′-oxo-N-propyl-7′,8′-dihydro-6′H-spiro[cyclohexane-1,9′-furo[2,3-f]quinazoline]-2′-carboxamide; 5′-chloro-N-methyl-7′-oxo-N-(thiophen-3-ylmethyl)-7′,8′-dihydro-6′H-spiro[cyclohexane-1,9′-furo[2,3-f]quinazoline]-2′-carboxamide; 1-({5′-chloro-7′-oxo-7′,8′-dihydro-6′H-spiro[cyclohexane-1,9′-furo[2,3-f]quinazoline]-2′-yl}carbonyl)piperidine-4-carbonitrile; 5′-chloro-2′-[3-(dimethylamino)pyrrolidine-1-carbonyl]-7′,8′-dihydro-6′H-spiro[cyclohexane-1,9′-furo[2,3-]quinazoline]-7′-one; N-benzyl-5′-chloro-N-methyl-7′-oxo-7′,8′-dihydro-6′H-spiro[cyclohexane-1,9′-furo[2,3-f]quinazoline]-2′-carboxamide; 5′-chloro-N-methyl-7′-oxo-N-(thiophen-2-ylmethyl)-7′,8′-dihydro-6′H-spiro[cyclohexane-1,9′-furo[2,3-]quinazoline]-2′-carboxamide; 5′-chloro-N-[(3-methoxyphenyl)methyl]-7′-oxo-7′,8′-dihydro-6′H-spiro[cyclohexane-1,9′-furo[2,3-f]quinazoline]-2′-carboxamide; 5′-chloro-2′-(3-hydroxypyrrolidine-1-carbonyl)-7′,8′-dihydro-6′H-spiro[cyclohexane-1,9′-furo[2,3-f]quinazoline]-7′-one; 5′-chloro-7′-oxo-N,N-bis(propan-2-yl)-7′,8′-dihydro-6′H-spiro[cyclohexane-1,9′-furo[2,3-f]quinazoline]-2′-carboxamide; 5′-chloro-N-[2-(furan-2-yl)ethyl]-7′-oxo-7′,8′-dihydro-6′H-spiro[cyclohexane-1,9′-furo[2,3-f]quinazoline]-2′-carboxamide; 5′-chloro-7′-[2-(hydroxymethyl)piperidine-1-carbonyl]-7′,8′-dihydro-6′H-spiro[cyclohexane-1,9′-furo[2,3-f]quinazoline]-7′-one; 5′-chloro-7′-[4-(propan-2-yl)piperazine-1-carbonyl]-7′,8′-dihydro-6′H-spiro[cyclohexane-1,9′-furo[2,3-f]quinazoline]-7′-one; 5′-chloro-7′-[2-(furan-2-yl)pyrrolidine-1-carbonyl]-7′,8′-dihydro-6′H-spiro[cyclohexane-1,9′-furo[2,3-f]quinazoline]-7′-one; 5′-chloro-N-(3-methylbutyl)-7′-oxo-7′,8′-dihydro-6′H-spiro[cyclohexane-1,9′-furo[2,3-f]quinazoline]-2′-carboxamide; 5′-chloro-N-(2-cyanoethyl)-N-ethyl-7′-oxo-7′,8′-dihydro-6′H-spiro[cyclohexane-1,9′-furo[2,3-f]quinazoline]-2′-carboxamide; 5′-chloro-N-methyl-7′-oxo-N-(pyridin-2-ylmethyl)-7′,8′-dihydro-6′H-spiro[cyclohexane-1,9′-furo[2,3-]quinazoline]-2′-carboxamide; 5′-chloro-7′-oxo-N-[3-(pyrrolidin-1-yl)propyl]-7′,8′-dihydro-6′H-spiro[cyclohexane-1,9′-furo[2,3-f]quinazoline]-2′-carboxamide; 5′-chloro-7′-oxo-N-(pentan-3-yl)-7′,8′-dihydro-6′H-spiro[cyclohexane-1,9′-furo[2,3-f]quinazoline]-2′-carboxamide; 2′-(azepane-1-carbonyl)-5′-chloro-7′,8′-dihydro-6′H-spiro[cyclohexane-1,9′-furo[2,3-f]quinazoline]-7′-one; 5′-chloro-N-[2-(1H-imidazol-1-yl)ethyl]-7′-oxo-7′,8′-dihydro-6′H-spiro[cyclohexane-1,9′-furo[2,3-f]quinazoline]-2′-carboxamide; 5′-chloro-2′-[(2R,6S)-2,6-dimethylmorpholine-4-carbonyl]-7′,8′-dihydro-6′H-spiro[cyclohexane-1,9′-furo[2,3-f]quinazoline]-7′-one; 5′-chloro-N-methyl-7′-oxo-N-(pyridin-3-ylmethyl)-7′,8′-dihydro-6′H-spiro[cyclohexane-1,9′-furo[2,3-]quinazoline]-2′-carboxamide; 5′-chloro-N-methyl-N-(1-methylpiperidin-4-yl)-7′-oxo-7′,8′-dihydro-6′H-spiro[cyclohexane-1,9′-furo[2,3-f]quinazoline]-2′-carboxamide; 5′-chloro-N-[(3-ethyl-1,2-oxazol-5-yl)methyl]-N-methyl-7′-oxo-7′,8′-dihydro-6′H-spiro[cyclohexane-1,9′-furo[2,3-f]quinazoline]-2′-carboxamide; 5′-chloro-N-(3-methylbutan-2-yl)-7′-oxo-7′,8′-dihydro-6′H-spiro[cyclohexane-1,9′-furo[2,3-f]quinazoline]-2′-carboxamide; 5′-chloro-7′-(3-methylpiperidine-1-carbonyl)-7′,8′-dihydro-6′H-spiro[cyclohexane-1,9′-furo[2,3-f]quinazoline]-7′-one; 5′-chloro-N-[2-(dimethylamino)ethyl]-N-methyl-7′-oxo-7′,8′-dihydro-6′H-spiro[cyclohexane-1,9′-furo[2,3-f]quinazoline]-7′-carboxamide; 5′-chloro-7′-(2,2-dimethylmorpholine-4-carbonyl)-7′,8′-dihydro-6′H-spiro[cyclohexane-1,9′-furo[2,3-f]quinazoline]-7′-one; 5′-chloro-N-[(3-hydroxyphenyl)methyl]-7′-oxo-7′,8′-dihydro-6′H-spiro[cyclohexane-1,9′-furo[2,3-f]quinazoline]-7′-carboxamide; 5′-chloro-7′-[4-(dimethylamino)piperidine-1-carbonyl]-7′,8′-dihydro-6′H-spiro[cyclohexane-1,9′-furo[2,3-f]quinazoline]-7′-one; 5′-chloro-7′-oxo-N-[3-(2-oxopyrrolidin-1-yl)propyl]-7′,8′-dihydro-6′H-spiro[cyclohexane-1,9′-furo[2,3-]quinazoline]-2′-carboxamide; 5′-chloro-2′-(4-methylpiperidine-1-carbonyl)-7′,8′-dihydro-6′H-spiro[cyclohexane-1,9′-furo[2,3-f]quinazoline]-7′-one; 5′-chloro-7′-oxo-N-(thiophen-3-ylmethyl)-7′,8′-dihydro-6′H-spiro[cyclohexane-1,9′-furo[2,3-f]quinazoline]-2′-carboxamide; 5′-chloro-N-[(3-fluorophenyl)methyl]-7′-oxo-7′,8′-dihydro-6′H-spiro[cyclohexane-1,9′-furo[2,3-f]quinazoline]-2′-carboxamide; N-benzyl-5′-chloro-N-(2-hydroxyethyl)-7′-oxo-7′,8′-dihydro-6′H-spiro[cyclohexane-1,9′-furo[2,3-f]quinazoline]-2′-carboxamide; 5′-chloro-N-[2-(3,5-dimethyl-1H-pyrazol-4-yl)ethyl]-N-methyl-7′-oxo-7′,8′-dihydro-6′H-spiro[cyclohexane-1,9′-furo[2,3-f]quinazoline]-2′-carboxamide; 5′-chloro-2′-(4-cyclohexylpiperazine-1-carbonyl)-7′,8′-dihydro-6′H-spiro[cyclohexane-1,9′-furo[2,3-f]quinazoline]-7′-one; 5′-chloro-2′-(4-hydroxy-4-phenylpiperidine-1-carbonyl)-7′,8′-dihydro-6′H-spiro[cyclohexane-1,9′-furo[2,3-f]quinazoline]-7′-one; 5′-chloro-2′-[4-(oxolane-2-carbonyl)piperazine-1-carbonyl]-7′,8′-dihydro-6′H-spiro[cyclohexane-1,9′-furo[2,3-f]quinazoline]-7′-one; 5′-chloro-2′-{4-[(dimethylamino)methyl]piperidine-1-carbonyl}-7′,8′-dihydro-6′H-spiro[cyclohexane-1,9′-furo[2,3-f]quinazoline]-7′-one; 1-({5′-chloro-7′-oxo-7′,8′-dihydro-6′H-spiro[cyclohexane-1,9′-furo[2,3-f]quinazoline]-2′-yl}carbonyl)-N-methylpiperidine-4-carboxamide; 5′-chloro-N-[2-(4-methoxyphenyl)ethyl]-7′-oxo-7′,8′-dihydro-6′H-spiro[cyclohexane-1,9′-furo[2,3-f]quinazoline]-7′-carboxamide; 5′-chloro-N-[2-(dimethyl-1,2-oxazol-4-yl)ethyl]-N-methyl-7′-oxo-7′,8′-dihydro-6′H-spiro[cyclohexane-1,9′-furo[2,3-f]quinazoline]-7′-carboxamide; 5′-chloro-2′-[4-(1,3-thiazol-2-yl)piperazine-1-carbonyl]-7′,8′-dihydro-6′H-spiro[cyclohexane-1,9′-furo[2,3-f]quinazoline]-7′-one; 5′-chloro-N-{[4-(1H-imidazol-1-yl)phenyl]methyl}-N-methyl-7′-oxo-7′,8′-dihydro-6′H-spiro[cyclohexane-1,9′-furo[2,3-f]quinazoline]-2′-carboxamide; 2′-(4-tert-butylpiperazine-1-carbonyl)-5′-chloro-7′,8′-dihydro-6′H-spiro[cyclohexane-1,9′-furo[2,3-f]quinazoline]-7′-one; 5′-chloro-N-[2-(3-methoxyphenyl)ethyl]-7′-oxo-7′,8′-dihydro-6′H-spiro[cyclohexane-1,9′-furo[2,3-f]quinazoline]-2′-carboxamide; 5′-chloro-2′-[4-(pyrrolidin-1-yl)piperidine-1-carbonyl]-7′,8′-dihydro-6′H-spiro[cyclohexane-1,9′-furo[2,3-f]quinazoline]-7′-one; 5′-chloro-N-(2,3-dihydro-1,4-benzodioxin-2-ylmethyl)-7′-oxo-7′,8′-dihydro-6′H-spiro[cyclohexane-1,9′-furo[2,3-f]quinazoline]-2′-carboxamide; 5′-chloro-2′-[4-(morpholin-4-yl)piperidine-1-carbonyl]-7′,8′-dihydro-6′H-spiro[cyclohexane-1,9′-furo[2,3-f]quinazoline]-7′-one; 5′-chloro-2′-[4-(3-methoxyphenyl)piperazine-1-carbonyl]-7′,8′-dihydro-6′H-spiro[cyclohexane-1,9′-furo[2,3-f]quinazoline]-7′-one; 5′-chloro-7′-oxo-N-[1-(propan-2-yl)piperidin-4-yl]-7′,8′-dihydro-6′H-spiro[cyclohexane-1,9′-furo[2,3-f]quinazoline]-2′-carboxamide; 5′-chloro-N-(1-hydroxy-3-phenylpropan-2-yl)-7′-oxo-7′,8′-dihydro-6′H-spiro[cyclohexane-1,9′-furo[2,3-f]quinazoline]-2′-carboxamide; N-(2H-1,3-benzodioxol-5-ylmethyl)-5′-chloro-N-methyl-7′-oxo-7′,8′-dihydro-6′H-spiro[cyclohexane-1,9′-furo[2,3-f]quinazoline]-2′-carboxamide; methyl 3-[1-({5′-chloro-7′-oxo-7′,8′-dihydro-6′H-spiro[cyclohexane-1,9′-furo[2,3-f]quinazoline]-2′-yl}carbonyl)piperidin-2-yl]propanoate; 5′-chloro-7′-(4-cyclopentylpiperazine-1-carbonyl)-7′,8′-dihydro-6′H-spiro[cyclohexane-1,9′-furo[2,3-f]quinazoline]-7′-one; 5′-chloro-7′-[4-(2-methylpropanoyl)piperazine-1-carbonyl]-7′,8′-dihydro-6′H-spiro[cyclohexane-1,9′-furo[2,3-f]quinazoline]-7′-one; N-benzyl-5′-chloro-N-(3-hydroxypropyl)-7′-oxo-7′,8′-dihydro-6′H-spiro[cyclohexane-1,9′-furo[2,3-f]quinazoline]-7′-carboxamide; 5′-chloro-N-(2-methoxyethyl)-7′-oxo-N-(thiophen-2-ylmethyl)-7′,8′-dihydro-6′H-spiro[cyclohexane-1,9′-furo[2,3-f]quinazoline]-2′-carboxamide; 5′-chloro-N-[2-(dimethylamino)ethyl]-7′-oxo-7′,8′-dihydro-6′H-spiro[cyclohexane-1,9′-furo[2,3-f]quinazoline]-2′-carboxamide; 5′-chloro-2′-{5H,6H,7H-pyrrolo [3,4-b]pyridine-6-carbonyl}-7′,8′-dihydro-6′H-spiro[cyclohexane-1,9′-furo[2,3-f]quinazoline]-7′-one; 5′-chloro-N-cyclohexyl-N-(2-hydroxyethyl)-7′-oxo-7′,8′-dihydro-6′H-spiro[cyclohexane-1,9′-furo[2,3-f]quinazoline]-2′-carboxamide; 5′-chloro-N-[2-(dimethyl-1,3-thiazol-2-yl)ethyl]-7′-oxo-7′,8′-dihydro-6′H-spiro[cyclohexane-1,9′-furo[2,3-f]quinazoline]-2′-carboxamide; 5′-chloro-2′-({[(3R,4R)-3-methoxyoxan-4-yl]amino}methyl)-7′,8′-dihydro-6′H-spiro[cyclohexane-1,9′-furo[2,3-f]quinazoline]-7′-one; 5′-chloro-2′-{[2-(2-hydroxyethyl)piperidin-1-yl]methyl}-7′,8′-dihydro-6′H-spiro[cyclohexane-1,9′-furo[2,3-f]quinazoline]-7′-one; 5′-chloro-2′-[(3-oxopiperazin-1-yl)methyl]-7′,8′-dihydro-6′H-spiro[cyclohexane-1,9′-furo[2,3-f]quinazoline]-7′-one; 5′-chloro-2′-({[(1-methyl-1H-pyrazol-3-yl)methyl]amino}methyl)-7′,8′-dihydro-6′H-spiro[cyclohexane-1,9′-furo[2,3-f]quinazoline]-7′-one; 5′-chloro-2′-{[(2-methoxyethyl)(propan-2-yl)amino]methyl}-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}piperidine-4-carbonitrile; 5′-chloro-2′-{[(2-hydroxyethyl)(propan-2-yl)amino]methyl}-7′,8′-dihydro-6′H-spiro[cyclohexane-1,9′-furo[2,3-f]quinazoline]-7′-one; 5′-chloro-2′-{[4-(pyridin-4-yl)piperazin-1-yl]methyl}-7′,8′-dihydro-6′H-spiro[cyclohexane-1,9′-furo[2,3-f]quinazoline]-7′-one; 5′-chloro-2′-({[2-(1H-pyrazol-1-yl)ethyl]amino}methyl)-7′,8′-dihydro-6′H-spiro[cyclohexane-1,9′-furo[2,3-f]quinazoline]-7′-one; 5′-chloro-2′-{[(oxan-4-yl)amino]methyl}-7′,8′-dihydro-6′H-spiro[cyclohexane-1,9′-furo[2,3-f]quinazoline]-7′-one; 2′-[(8aS)-octahydropyrrolo[1,2-a]piperazin-2-ylmethyl]-5′-chloro-7′,8′-dihydro-6′H-spiro[cyclohexane-1,9′-furo[2,3-f]quinazoline]-7′-one; 5′-chloro-2′-{[(oxolan-2-ylmethyl)amino]methyl}-7′,8′-dihydro-6′H-spiro[cyclohexane-1,9′-furo[2,3-f]quinazoline]-7′-one; 3-({5′-chloro-7′-oxo-7′,8′-dihydro-6′H-spiro[cyclohexane-1,9′-furo[2,3-f]quinazoline]-2′-ylmethyl}(ethyl)amino)propanenitrile; 5′-chloro-2′-{[(pyridin-2-ylmethyl)amino]methyl}-7′,8′-dihydro-6′H-spiro[cyclohexane-1,9′-furo[2,3-f]quinazoline]-7′-one; 5′-chloro-2′-{[2-(hydroxymethyl)pyrrolidin-1-yl]methyl}-7′,8′-dihydro-6′H-spiro[cyclohexane-1,9′-furo[2,3-f]quinazoline]-7′-one; 5′-chloro-2′-{[(2-methanesulfonylethyl)amino]methyl}-7′,8′-dihydro-6′H-spiro[cyclohexane-1,9′-furo[2,3-f]quinazoline]-7′-one; 2′-{[benzyl(2-hydroxyethyl)amino]methyl}-5′-chloro-7′,8′-dihydro-6′H-spiro[cyclohexane-1,9′-furo[2,3-f]quinazoline]-7′-one; 5′-chloro-2′-{[(furan-2-ylmethyl)(methyl)amino]methyl}-7′,8′-dihydro-6′H-spiro[cyclohexane-1,9′-furo[2,3-f]quinazoline]-7′-one; 5′-chloro-2′-[(4-methoxypiperidin-1-yl)methyl]-7′,8′-dihydro-6′H-spiro[cyclohexane-1,9′-furo[2,3-f]quinazoline]-7′-one; 2′-[(4-acetyl-1,4-diazepan-1-yl)methyl]-5′-chloro-7′,8′-dihydro-6′H-spiro[cyclohexane-1,9′-furo[2,3-f]quinazoline]-7′-one; 5′-chloro-2′-{[4-(2-hydroxyethyl)piperazin-1-yl]methyl}-7′,8′-dihydro-6′H-spiro[cyclohexane-1,9′-furo[2,3-f]quinazoline]-7′-one; 5′-chloro-2′-({4-[(dimethylamino)methyl]piperidin-1-yl}methyl)-7′,8′-dihydro-6′H-spiro[cyclohexane-1,9′-furo[2,3-f]quinazoline]-7′-one; 5′-chloro-7′-{[4-(oxolane-2-carbonyl)piperazin-1-yl]methyl}-7′,8′-dihydro-6′H-spiro[cyclohexane-1,9′-furo[2,3-f]quinazoline]-7′-one; 5′-chloro-2′-({[(5-oxopyrrolidin-2-yl)methyl](propan-2-yl)amino}methyl)-7′,8′-dihydro-6′H-spiro[cyclohexane-1,9′-furo[2,3-f]quinazoline]-7′-one; 5′-chloro-2′-{5H,6H,7H,8H-imidazo [1,2-a]pyrazin-7-ylmethyl}-7′,8′-dihydro-6′H-spiro[cyclohexane-1,9′-furo[2,3-f]quinazoline]-7′-one; 5′-chloro-2′-({[3-(2-oxopyrrolidin-1-yl)propyl]amino}methyl)-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}-N-methylpiperidine-4-carboxamide; 5′-chloro-2′-{[(3-hydroxypropyl)(methyl)amino]methyl}-7′,8′-dihydro-6′H-spiro[cyclohexane-1,9′-furo[2,3-f]quinazoline]-7′-one; 5′-chloro-2′-[(4-hydroxy-4-phenylpiperidin-1-yl)methyl]-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}piperidine-3-carboxamide; 3-({5′-chloro-7′-oxo-7′,8′-dihydro-6′H-spiro[cyclohexane-1,9′-furo[2,3-f]quinazoline]-2′-ylmethyl}(methyl)amino)-N-methylpropanamide; 5′-chloro-2′-[{(1R,5S,6S)-6-(hydroxymethyl)-3-azabicyclo[3.1.0]hexan-3-yl]methyl}-7′,8′-dihydro-6′H-spiro[cyclohexane-1,9′-furo[2,3-f]quinazoline]-7′-one; 5′-chloro-2′-{[ethyl(2-hydroxyethyl)amino]methyl}-7′,8′-dihydro-6′H-spiro[cyclohexane-1,9′-furo[2,3-f]quinazoline]-7′-one; 5′-chloro-2′-{[(2-hydroxyethyl)(methyl)amino]methyl}-7′,8′-dihydro-6′H-spiro[cyclohexane-1,9′-furo[2,3-f]quinazoline]-7′-one; 5′-chloro-2′-[(3-hydroxypiperidin-1-yl)methyl]-7′,8′-dihydro-6′H-spiro[cyclohexane-1,9′-furo[2,3-f]quinazoline]-7′-one; 5′-chloro-2′-{[4-(2-hydroxyethyl)piperidin-1-yl]methyl}-7′,8′-dihydro-6′H-spiro[cyclohexane-1,9′-furo[2,3-f]quinazoline]-7′-one; 5′-chloro-2′-{[(2R,6S)-2,6-dimethylmorpholin-4-yl]methyl}-7′,8′-dihydro-6′H-spiro[cyclohexane-1,9′-furo[2,3-f]quinazoline]-7′-one; 5′-chloro-2′-({[(4-ethyl-4H-1,2,4-triazol-3-yl)methyl](methyl)amino}methyl)-7′,8′-dihydro-6′H-spiro[cyclohexane-1,9′-furo[2,3-f]quinazoline]-7′-one; 5′-chloro-7′-{2-oxa-6-azaspiro[3.5]nonan-6-ylmethyl}-7′,8′-dihydro-6′H-spiro[cyclohexane-1,9′-furo[2,3-f]quinazoline]-7′-one; 5′-chloro-7′-{[ethyl(propan-2-yl)amino]methyl}-7′,8′-dihydro-6′H-spiro[cyclohexane-1,9′-furo[2,3-f]quinazoline]-7′-one; 5′-chloro-2′-({[(5-cyclopropyl-1H-pyrazol-3-yl)methyl](methyl)amino}methyl)-7′,8′-dihydro-6′H-spiro[cyclohexane-1,9′-furo[2,3-f]quinazoline]-7′-one; 5′-chloro-2′-({[3-(dimethylamino)propyl](methyl)amino}methyl)-7′,8′-dihydro-6′H-spiro[cyclohexane-1,9′-furo[2,3-f]quinazoline]-7′-one; 5′-chloro-2′-{[3-(dimethylamino)pyrrolidin-1-yl]methyl}-7′,8′-dihydro-6′H-spiro[cyclohexane-1,9′-furo[2,3-f]quinazoline]-7′-one; 2-(4-{5′-chloro-7′-oxo-7′,8′-dihydro-6′H-spiro[cyclohexane-1,9′-furo[2,3-f]quinazoline]-2′-ylmethyl}piperazin-1-yl)pyridine-3-carbonitrile; 5′-chloro-2′-{[3-(1H-imidazol-1-ylmethyl)piperidin-1-yl]methyl}-7′,8′-dihydro-6′H-spiro[cyclohexane-1,9′-furo[2,3-f]quinazoline]-7′-one; 5′-chloro-2′-{[4-hydroxy-4-(thiophen-2-yl)piperidin-1-yl]methyl}-7′,8′-dihydro-6′H-spiro[cyclohexane-1,9′-furo[2,3-f]quinazoline]-7′-one; 5′-chloro-2′-{[4-(2-methoxyethyl)piperazin-1-yl]methyl}-7′,8′-dihydro-6′H-spiro[cyclohexane-1,9′-furo[2,3-f]quinazoline]-7′-one; 5′-chloro-2′-{[methyl(pyridin-3-ylmethyl)amino]methyl}-7′,8′-dihydro-6′H-spiro[cyclohexane-1,9′-furo[2,3-f]quinazoline]-7′-one; 4-{5′-chloro-7′-oxo-7′,8′-dihydro-6′H-spiro[cyclohexane-1,9′-furo[2,3-f]quinazoline]-2′-ylmethyl}-thiomorpholine-1,1-dione; 5′-chloro-2′-{[(4-hydroxy-2-methylbutan-2-yl)amino]methyl}-7′,8′-dihydro-6′H-spiro[cyclohexane-1,9′-furo[2,3-f]quinazoline]-7′-one; 4-{5′-chloro-7′-oxo-7′,8′-dihydro-6′H-spiro[cyclohexane-1,9′-furo[2,3-f]quinazoline]-2′-ylmethyl}piperazine-1-carbaldehyde; 5′-chloro-2′-{[4-(morpholin-4-yl)piperidin-1-yl]methyl}-7′,8′-dihydro-6′H-spiro[cyclohexane-1,9′-furo[2,3-f]quinazoline]-7′-one; 4-{5′-chloro-7′-oxo-7′,8′-dihydro-6′H-spiro[cyclohexane-1,9′-furo[2,3-f]quinazoline]-2′-ylmethyl}-N,N-dimethylpiperazine-1-carboxamide; 5′-chloro-2′-[({[4-(propan-2-yl)-4H-1,2,4-triazol-3-yl]methyl}amino)methyl]-7′,8′-dihydro-6′H-spiro[cyclohexane-1,9′-furo[2,3-f]quinazoline]-7′-one; 5′-chloro-2′-{[3-(trifluoromethyl)-5H,6H,7H,8H-[1,2,4]triazolo[4,3-a]pyrazin-7-yl]methyl}-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}azetidine-3-carbonitrile; 4-({5′-chloro-7′-oxo-7′,8′-dihydro-6′H-spiro[cyclohexane-1,9′-furo[2,3-f]quinazoline]-2′-ylmethyl}amino)-thiane-1,1-dione; 5′-chloro-2′-[(4-methanesulfonylpiperazin-1-yl)methyl]-7′,8′-dihydro-6′H-spiro[cyclohexane-1,9′-furo[2,3-f]quinazoline]-7′-one; ethyl 7-{5′-chloro-7′-oxo-7′,8′-dihydro-6′H-spiro[cyclohexane-1,9′-furo[2,3-f]quinazoline]-2′-ylmethyl}-5H,6H,7H,8H-imidazo[1,2-a]pyrazine-2-carboxylate; 2′-{6-azaspiro[2.5]octan-6-ylmethyl}-5′-chloro-7′,8′-dihydro-6′H-spiro[cyclohexane-1,9′-furo[2,3-f]quinazoline]-7′-one; 5′-chloro-2′-{[(2,2,2-trifluoroethyl)amino]methyl}-7′,8′-dihydro-6′H-spiro[cyclohexane-1,9′-furo[2,3-f]quinazoline]-7′-one; 5′-chloro-2′-{octahydropyrrolo[1,2-a]piperazin-2-ylmethyl}-7′,8′-dihydro-6′H-spiro[cyclohexane-1,9′-furo[2,3-f]quinazoline]-7′-one; 5′-chloro-2′-{[4-(furan-2-ylmethyl)piperazin-1-yl]methyl}-7′,8′-dihydro-6′H-spiro[cyclohexane-1,9′-furo[2,3-f]quinazoline]-7′-one; 5′-chloro-2′-{[methyl(pro pan-2-yl)amino]methyl}-7′,8′-dihydro-6′H-spiro[cyclohexane-1,9′-furo[2,3-f]quinazoline]-7′-one; 3-({5′-chloro-7′-oxo-7′,8′-dihydro-6′H-spiro[cyclohexane-1,9′-furo[2,3-f]quinazoline]-2′-ylmethyl}amino)-thiolane-1,1-dione; 5′-chloro-2′-{[4-(pyridin-3-ylmethyl)piperazin-1-yl]methyl}-7′,8′-dihydro-6′H-spiro[cyclohexane-1,9′-furo[2,3-f]quinazoline]-7′-one; 5′-chloro-2′-[(4-methyl-1,4-diazepan-1-yl)methyl]-7′,8′-dihydro-6′H-spiro[cyclohexane-1,9′-furo[2,3-f]quinazoline]-7′-one; 5′-chloro-7′-{[4-(dimethylamino)piperidin-1-yl]methyl}-7′,8′-dihydro-6′H-spiro[cyclohexane-1,9′-furo[2,3-f]quinazoline]-7′-one; 5′-chloro-7′-({methyl[(trimethyl-1H-pyrazol-4-yl)methyl]amino}methyl)-7′,8′-dihydro-6′H-spiro[cyclohexane-1,9′-furo[2,3-f]quinazoline]-7′-one; 5′-chloro-7′-{[3-oxo-4-(propan-2-yl)piperazin-1-yl]methyl}-7′,8′-dihydro-6′H-spiro[cyclohexane-1,9′-furo[2,3-f]quinazoline]-7′-one; 5′-chloro-7′-{[(2,2-dimethyloxan-4-yl)(ethyl)amino]methyl}-7′,8′-dihydro-6′H-spiro[cyclohexane-1,9′-furo[2,3-f]quinazoline]-7′-one; 5′-chloro-2′-{[4-(pyrrolidine-1-carbonyl)piperazin-1-yl]methyl}-7′,8′-dihydro-6′H-spiro[cyclohexane-1,9′-furo[2,3-f]quinazoline]-7′-one; 5′-chloro-2′-{[(1,3-oxazol-5-ylmethyl)amino]methyl}-7′,8′-dihydro-6′H-spiro[cyclohexane-1,9′-furo[2,3-f]quinazoline]-7′-one; 5′-chloro-2′-[(propylamino)methyl]-7′,8′-dihydro-6′H-spiro[cyclohexane-1,9′-furo[2,3-f]quinazoline]-7′-one; 5′-chloro-2′-({[(1-methyl-1H-pyrazol-5-yl)methyl]amino}methyl)-7′,8′-dihydro-6′H-spiro[cyclohexane-1,9′-furo[2,3-f]quinazoline]-7′-one; 5′-chloro-2′-{[4-(oxetan-3-yl)piperazin-1-yl]methyl}-7′,8′-dihydro-6′H-spiro[cyclohexane-1,9′-furo[2,3-f]quinazoline]-7′-one; 5′-chloro-2′-({methyl[2-(pyridin-2-yl)ethyl]amino}methyl)-7′,8′-dihydro-6′H-spiro[cyclohexane-1,9′-furo[2,3-f]quinazoline]-7′-one; 5′-chloro-2′-{[(2-methoxyethyl)(methyl)amino]methyl}-7′,8′-dihydro-6′H-spiro[cyclohexane-1,9′-furo[2,3-f]quinazoline]-7′-one; 5′-chloro-2′-{5H,6H,7H-pyrrolo [3,4-b]pyridin-6-ylmethyl}-7′,8′-dihydro-6′H-spiro[cyclohexane-1,9′-furo[2,3-f]quinazoline]-7′-one; 5′-chloro-2′-({[2-(3,5-dimethyl-1H-pyrazol-4-yl)ethyl](methyl)amino}methyl)-7′,8′-dihydro-6′H-spiro[cyclohexane-1,9′-furo[2,3-f]quinazoline]-7′-one; 5′-chloro-2′-({[2-(dimethylamino)ethyl](ethyl)amino}methyl)-7′,8′-dihydro-6′H-spiro[cyclohexane-1,9′-furo[2,3-f]quinazoline]-7′-one; 5′-chloro-2′-[(3-hydroxypyrrolidin-1-yl)methyl]-7′,8′-dihydro-6′H-spiro[cyclohexane-1,9′-furo[2,3-f]quinazoline]-7′-one; 5′-chloro-7′-[({[6-(morpholin-4-yl)pyridin-3-yl]methyl}amino)methyl]-7′,8′-dihydro-6′H-spiro[cyclohexane-1,9′-furo[2,3-f]quinazoline]-7′-one; 5′-chloro-7′-{[methyl(propyl)amino]methyl}-7′,8′-dihydro-6′H-spiro[cyclohexane-1,9′-furo[2,3-f]quinazoline]-7′-one; 5′-chloro-2′-({[3-(3,5-dimethyl-1H-pyrazol-1-yl)propyl](methyl)amino}methyl)-7′,8′-dihydro-6′H-spiro[cyclohexane-1,9′-furo[2,3-f]quinazoline]-7′-one; 5′-chloro-7′-[(2,2-dimethylmorpholin-4-yl)methyl]-7′,8′-dihydro-6′H-spiro[cyclohexane-1,9′-furo[2,3-f]quinazoline]-7′-one; 5′-chloro-2′-[(4-ethylpiperazin-1-yl)methyl]-7′,8′-dihydro-6′H-spiro[cyclohexane-1,9′-furo[2,3-f]quinazoline]-7′-one; 5′-chloro-2′-({[(3-ethyl-1,2-oxazol-5-yl)methyl](methyl)amino}methyl)-7′,8′-dihydro-6′H-spiro[cyclohexane-1,9′-furo[2,3-f]quinazoline]-7′-one; 5′-chloro-2′-{[methyl(pyridin-2-ylmethyl)amino]methyl}-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}piperidine-4-carboxamide; 5′-chloro-2′-({[2-(2-oxopyrrolidin-1-yl)ethyl]amino}methyl)-7′,8′-dihydro-6′H-spiro[cyclohexane-1,9′-furo[2,3-f]quinazoline]-7′-one; 5′-chloro-2′-[({[1-(ethoxymethyl)cyclopropyl]methyl}amino)methyl]-7′,8′-dihydro-6′H-spiro[cyclohexane-1,9′-furo[2,3-f]quinazoline]-7′-one; 5′-chloro-2′-({[2-(dimethyl-1,2-oxazol-4-yl)ethyl](methyl)amino}methyl)-7′,8′-dihydro-6′H-spiro[cyclohexane-1,9′-furo[2,3-f]quinazoline]-7′-one; N-(1-{5′-chloro-7′-oxo-7′,8′-dihydro-6′H-spiro[cyclohexane-1,9′-furo[2,3-f]quinazoline]-2′-ylmethyl}piperidin-4-yl)acetamide; 5′-chloro-2′-{[2-(hydroxymethyl)piperidin-1-yl]methyl}-7′,8′-dihydro-6′H-spiro[cyclohexane-1,9′-furo[2,3-f]quinazoline]-7′-one; 5′-chloro-2′-{[methyl(1-methylpiperidin-4-yl)amino]methyl}-7′,8′-dihydro-6′H-spiro[cyclohexane-1,9′-furo[2,3-f]quinazoline]-7′-one; 5′-chloro-2′-({6,6-dimethyl-3-azabicyclo[3.1.0]hexan-3-yl}methyl)-7′,8′-dihydro-6′H-spiro[cyclohexane-1,9′-furo[2,3-f]quinazoline]-7′-one; 5′-chloro-2′-({[(1-methyl-1H-imidazol-2-yl)methyl]amino}methyl)-7′,8′-dihydro-6′H-spiro[cyclohexane-1,9′-furo[2,3-f]quinazoline]-7′-one; 5′-chloro-7′-{[3-(hydroxymethyl) piperidin-1-yl]methyl}-7′,8′-dihydro-6′H-spiro[cyclohexane-1,9′-furo[2,3-f]quinazoline]-7′-one; 5′-chloro-7′-{[(2-hydroxyethyl)(propyl)amino]methyl}-7′,8′-dihydro-6′H-spiro[cyclohexane-1,9′-furo[2,3-f]quinazoline]-7′-one; ethyl 2-({5′-chloro-7′-oxo-7′,8′-dihydro-6′H-spiro[cyclohexane-1,9′-furo[2,3-f]quinazoline]-2′-ylmethyl}(methyl)amino)acetate; 2′-[(3-aminopyrrolidin-1-yl)methyl]-5′-chloro-7′,8′-dihydro-6′H-spiro[cyclohexane-1,9′-furo[2,3-f]quinazoline]-7′-one; N-{2-({5′-chloro-7′-oxo-7′,8′-dihydro-6′H-spiro[cyclohexane-1,9′-furo[2,3-f]quinazoline]-2′-ylmethyl}amino)ethyl}acetamide; 5′-chloro-2′-{[4-(hydroxymethyl)piperidin-1-yl]methyl}-7′,8′-dihydro-6′H-spiro[cyclohexane-1,9′-furo[2,3-f]quinazoline]-7′-one; 5′-chloro-2′-{[(piperidin-3-yl)amino]methyl}-7′,8′-dihydro-6′H-spiro[cyclohexane-1,9′-furo[2,3-f]quinazoline]-7′-one; 5′-chloro-2′-{[(pyrrolidin-3-yl)amino]methyl}-7′,8′-dihydro-6′H-spiro[cyclohexane-1,9′-furo[2,3-f]quinazoline]-7′-one; 2′-benzoyl-5′-chloro-7′,8′-dihydro-6′H-spiro[cyclohexane-1,9′-furo[2,3-f]quinazoline]-7′-one; 5′-chloro-2′-(pyridine-4-carbonyl)-7′,8′-dihydro-6′H-spiro[cyclohexane-1,9′-furo[2,3-f]quinazoline]-7′-one; 2′-acetyl-5′-chloro-7′,8′-dihydro-6′H-spiro[cyclohexane-1,9′-furo[2,3-]quinazoline]-7′-one; 5′-chloro-2′-({[(3S,4R)-3-methoxyoxan-4-yl]amino}methyl)-7′,8′-dihydro-6′H-spiro[cyclohexane-1,9′-furo[2,3-f]quinazoline]-7′-one; 5′-chloro-2′-{[(3-fluorooxan-4-yl)amino]methyl}-7′,8′-dihydro-6′H-spiro[cyclohexane-1,9′-furo[2,3-f]quinazoline]-7′-one; 5′-chloro-2′-({[(3R)-oxolan-3-yl]amino}methyl)-7′,8′-dihydro-6′H-spiro[cyclohexane-1,9′-furo[2,3-f]quinazoline]-7′-one; 5′-chloro-2′-({[(3S)-oxolan-3-yl]amino}methyl)-7′,8′-dihydro-6′H-spiro[cyclohexane-1,9′-furo[2,3-f]quinazoline]-7′-one; 5′-chloro-7′-({[(3R,4S)-3-methoxyoxan-4-yl]amino}methyl)-7′,8′-dihydro-6′H-spiro[cyclohexane-1,9′-furo[2,3-f]quinazoline]-7′-one; 5′-chloro-7′-({[(3S,4R)-3-methoxyoxan-4-yl]amino}methyl)-7′,8′-dihydro-6′H-spiro[cyclohexane-1,9′-furo[2,3-f]quinazoline]-7′-one; 5′-chloro-7′-{[(2-methoxyethyl)amino]methyl}-7′,8′-dihydro-6′H-spiro[cyclohexane-1,9′-furo[2,3-f]quinazoline]-7′-one; 5′-chloro-2′-{[(oxetan-3-yl)amino]methyl}-7′,8′-dihydro-6′H-spiro[cyclohexane-1,9′-furo[2,3-f]quinazoline]-7′-one; and pharmaceutically acceptable salts thereof.

In one embodiment, a pharmaceutical composition comprises a compound or pharmaceutically acceptable salt thereof of the formula (1), and a pharmaceutically acceptable carrier.

In one embodiment, a method of treating inhibiting or relieving a disorder or ameliorating a symptom of a disorder that includes having an aberrant or dysregulated signaling pathway mediated by PDE7 in a subject, the method comprises administering a compound of the formula (1), or pharmaceutically acceptable salt thereof, to a subject in need thereof.

In one embodiment, PDE7 inhibitor compounds useful in the methods of the invention have the formula (1) or are selected from the group consisting of: 5-chloro-2-[(1S,4S)-2-oxa-5-azabicyclo[2.2.1]heptan-5-ylmethyl]-7,8-dihydro-6H-spiro[[1,3]oxazolo[5,4-f]quinazoline-9,1′-cyclohexane]-7-one; 5-chloro-2-[(3-methoxy-3-methylazetidin-1-yl)methyl]-7,8-dihydro-6H-spiro[[1,3]oxazolo[5,4-f]quinazoline-9,1′-cyclohexane]-7-one; 5-chloro-2-{[3-(hydroxymethyl)pyrrolidin-1-yl]methyl}-7,8-dihydro-6H-spiro[[1,3]oxazolo[5,4-f]quinazoline-9,1′-cyclohexane]-7-one; 5-chloro-2-({[2-(dimethylamino)ethyl](methyl)amino}methyl)-7,8-dihydro-6H-spiro[[1,3]oxazolo[5,4-f]quinazoline-9,1′-cyclohexane]-7-one; 5-chloro-2-{[methyl(oxan-4-yl)amino]methyl}-7,8-dihydro-6H-spiro[[1,3]oxazolo[5,4-f]quinazoline-9,1′-cyclohexane]-7-one; and pharmaceutically acceptable salts thereof.

In one embodiment, PDE7 inhibitor compounds useful in the methods of the invention have the formulas:

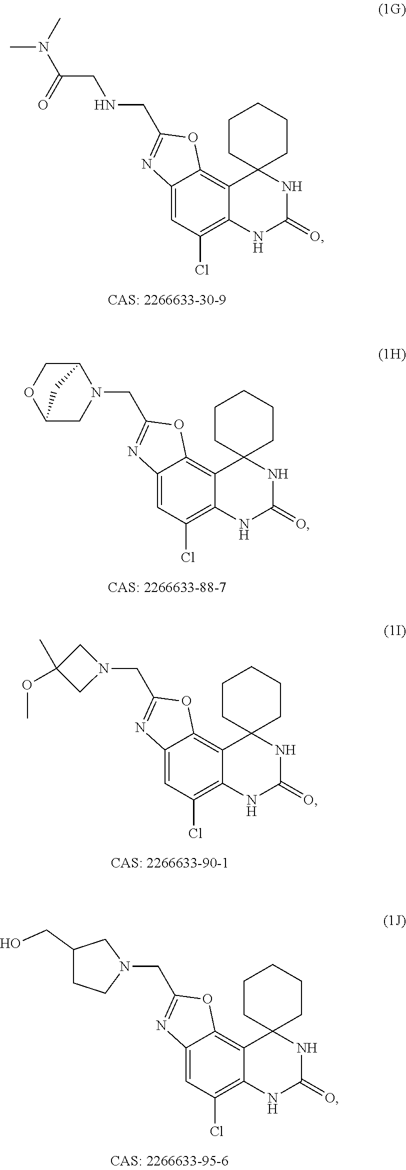 [SMILES: CN(C)C(=O)CNCc1nc2cc(Cl)c3c(c2o1)C1(CCCCC1)NC(=O)N3 ; O=C1Nc2c(Cl)cc3nc(CN4C[C@@H]5C[C@H]4CO5)oc3c2C2(CCCCC2)N1 ; COC1(C)CN(Cc2nc3cc(Cl)c4c(c3o2)C2(CCCCC2)NC(=O)N4)C1 ; O=C1Nc2c(Cl)cc3nc(CN4CCC(CO)C4)oc3c2C2(CCCCC2)N1]

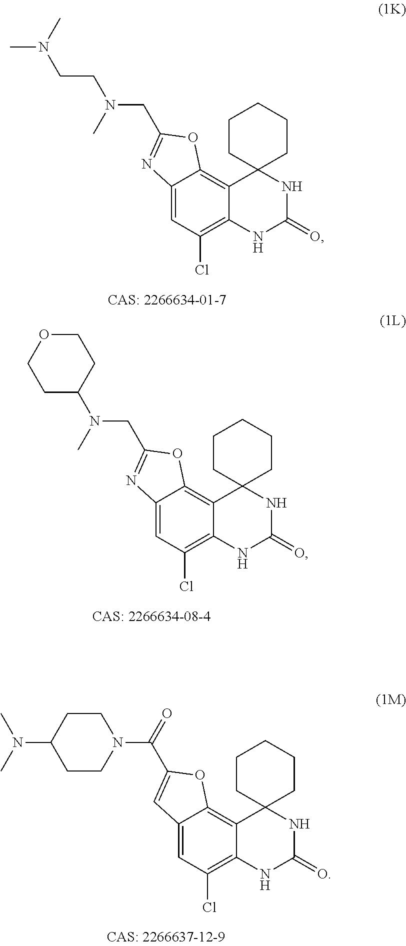 [SMILES: CN(C)CCN(C)Cc1nc2cc(Cl)c3c(c2o1)C1(CCCCC1)NC(=O)N3 ; CN(Cc1nc2cc(Cl)c3c(c2o1)C1(CCCCC1)NC(=O)N3)C1CCOCC1 ; CN(C)C1CCN(C(=O)c2cc3cc(Cl)c4c(c3o2)C2(CCCCC2)NC(=O)N4)CC1]

More specific embodiments comprise a pharmaceutical composition comprising a pharmaceutically acceptable salt and a pharmaceutically acceptable carrier.

The preparation of the above compounds is described in U.S. Pat. No. 11,685,745 and WO 2019/014305.

The invention provides a method of treating diseases associated with chronic fatigue (DACF) by administering it to a patient in need thereof an amount of a PDE7 inhibitory agent effective to inhibit the enzymatic activity of PDE7, wherein such inhibition of PDE7 enzymatic activity is the principal therapeutic mode of action of the PDE7 inhibitor in the treatment of DACF.

For each of the PDE7 inhibitory chemical compounds useful in the method of the present invention, all possible stereoisomers and geometric isomers are included. The compounds include not only racemic compounds, but also the optically active isomers. When a PDE7 inhibitory agent is desired as a single enantiomer, it can be obtained either by resolution of the final product or by stereospecific synthesis from either isomerically pure starting material or use of a chiral auxiliary reagent, for example, see Ma, Z., et al., Tetrahedron: Asymmetry 8(6):883-888, 1997. Resolution of the final product, an intermediate, or a starting material can be achieved by any suitable method known in the art. Additionally, in situations where tautomers of the compounds are possible, the present invention is intended to include all tautomeric forms of the compounds.

The PDE7 inhibitory agents that contain acidic moieties can form pharmaceutically acceptable salts with suitable cations. Suitable pharmaceutically acceptable cations include alkali metal (e.g., sodium or potassium) and alkaline earth metal (e.g., calcium or magnesium) cations. The pharmaceutically acceptable salts of the PDE7 inhibitory agents, which contain a basic center, are acid addition salts formed with pharmaceutically acceptable acids. Examples include the hydrochloride, hydro bromide, sulfate or bisulfate, phosphate or hydrogen phosphate, acetate, benzoate, succinate, fumarate, maleate, lactate, citrate, tartarate, gluconate, methanefulgonate, bezenesulphonate, and p-toluenesulphonate salts. In light of the foregoing, any reference to compounds useful in the method of the invention appearing herein is intended to include PDE7 inhibitory agents, as well as pharmaceutically acceptable salts and solvates thereof.

The compounds of the present invention can be therapeutically administered as the neat chemical, but it is preferable to administer the PDE7 inhibitory agents as a pharmaceutical composition or formulation, by way of example, as a tablet or capsule. Accordingly, the present invention further provides for pharmaceutical compositions or formulations comprising a PDE7 inhibitory agent, or pharmaceutically acceptable salts thereof, together with one or more pharmaceutically acceptable carriers and, optionally, other therapeutic and/or prophylactic ingredients. Suitable carriers are compatible with the other ingredients of the formulation and not deleterious to the recipient thereof. Compounds of the present invention may also be carried in a delivery system to provide for sustained release or enhanced uptake or activity of the compound, such as a liposomal or hydrogel system for injection, a microparticle, nanopartical, or micelle system for oral or parenteral delivery, or a staged capsule system for oral delivery.

### EXAMPLES

The following examples describe the use of some compounds in accordance with the invention. These examples are not limiting and serve only to illustrate the present invention.

### Example 1—PDE7 Enzymatic Assay and IC₅₀ Determination

The ability of compounds to inhibit PDE7 may be measured using the following assay protocal: Assay methods for IC₅₀ determination of PDE7 and variants involve as enzyme source human recombinant insect Sf9 cells. The substrate is 0.1 M FAM-cAMP and the vehicle is 1.0% DMSO or 1.0% PEG 400. Pre-incubation time and temperature are 15 minutes at 25° C. and incubation time and temperature are 30 minutes at 25° C. The incubation buffer consists of 10 mM Tris-HCl, pH 7.2, 10 mM MgCl₂, 0.05% NaN₃, and 0.1% phosphate-free BSA. The quantitation method used is spectrofluorimetric quantitation of Fluorescein-AMP-IMAP. IBMX reference standards are run as an integral part of each assay to ensure the validity of the results obtained. The IC₅₀ is the concentration of the compound (inhibitor) tested in the assay which makes it possible to reduce the enzymatic activity of PDE7 by 50%. IC₅₀ values are finally determined by a non-linear, least squares regression analysis using MathlQ™ (ID Business Solutions Ltd., UK).

The compounds to be tested as PDE7 inhibitors were initially dissolved in vehicle at a concentration of 30 mM. These solutions were tested as the highest test concentration and then further diluted in cascade always in a ratio of 1:6 to obtain solutions with the desired lower test concentrations. Each assay was carried out in duplicate.

As illustrative and nonlimiting examples, the following compounds in accordance with the invention inhibit PDE7 with the IC₅₀ values indicated below and show their advantage as therapeutically active substances:

| | | IC₅₀ | |
|---|---|---|---|
| Formula | CAS number | PDE7A (nM) | IC₅₀ PDE7B (nM) |
| No. 1G | 2266633-30-9 | 5.57 | 18.8 |
| No. 1H | 2266633-88-7 | | 10-100 |
| No. 1I | 2266633-90-1 | | 10-100 |
| No. 1J | 2266633-95-6 | | 10-100 |
| No. 1K | 2266634-01-7 | | 100-1,000 |
| No. 1L | 2266634-08-4 | | 10-100 |
| No. 1M | 2266637-12-9 | 28 | 58 |
| Dipyridamole | 58-32-2 | 42,000 | |
| IBMX | 28822-58-4 | 10,000 | |

### Example 2—Ex Vivo Model on More Residual Muscle Force (Less Fatigue) Via Na⁺/K⁺ ATPase Activation

### Introduction of the Model

The claim of this patent is that PDE7 inhibitors can improve disturbed body function and symptoms of ME/CFS, as an example of diseases associated with chronic fatigue (DACF). Muscular fatigue and loss of force (e.g., loss of handgrip strength) is a hallmark in ME/CFS patients. A main target of PDE7 inhibition is the skeletal muscle whose energetic function is deeply disturbed in ME/CFS and which is presumed to be very important in the symptomatology of ME/CFS. Insufficient stimulation of the Na⁺/K⁺ ATPase plays a key role in the development of the disturbances by a rise in intramuscular sodium that finally leads to calcium overload. The latter causes a deep functional energetic disturbance or damage. The isolated rat muscle either soleus muscle or extensor digitorum longus muscle is a well-characterized and well-validated model for the study of the function of the Na⁺/K⁺ ATPase. The isolated rat soleus muscle is particularly appropriate for experiments ex vivo as it is very thin so that there is no need for blood supply. It has been demonstrated under different experimental conditions that cAMP stimulates the Na⁺/K⁺ ATPase via protein kinase A (PKA) to reduce the decline in contractile force that occurs after measures such as carbachol application or high KCl concentration (12.5 mM) or by worsening of the energetic situation during electrical stimulation. In the experimental setting used, the high KCl concentration buffer causes partial depolarization that leads to sodium influx to disturb and to decrease excitability to subsequently reduce muscle force. Stimulation of Na⁺/K⁺ ATPase lowers intracellular sodium levels towards physiological levels to restore excitability and muscular force during electrical stimulation. Prevention or diminution of a loss of muscular force in this procedure indicates effective stimulation of Na⁺/K⁺ ATPase by a test compound with the described mode of action of PDE7-inhibition.

The decline in force in the soleus muscle can be considered as muscular fatigue and related to the loss of hand grip muscular strength in ME/CFS patients. As the phosphodiesterase isoenzyme subtype 7 (PDE7) is the predominant PDE isoenzyme in skeletal muscle to raise cAMP this ex vivo model is suited for the testing the effect of PDE7 inhibitors and in particular their effect on force generation disturbed by mechanisms that worsen excitability or the energetic situation. Since the isoenzyme variant PDE7A shows a much higher expression in skeletal muscle than that of the variant PDE7B, higher inhibitory activity of the PDE7-inhibitor on PDE7A compared with PDE7B is desirable. High KCl in a buffer (12.5 mM) was used to cause a decline in contractile force. Electrical stimulations after adding the high KCl buffer show a decline in contractile force to values close to 13% of the initial force which is considered as muscular fatigue. High level activity of Na⁺/K⁺ ATPase stimulated by cAMP can counteract the decrease in excitability and the loss of force. Drugs like salbutamol preserve contractile force and muscular fatigue when compared with the control situation. As PDE7 is the predominant phosphodiesterase isoenzyme in skeletal muscle, PDE7 inhibition is expected to strongly enhance the effect of salbutamol by inhibiting the degradation of cAMP, whose production is stimulated by salbutamol via adenylyl cyclase. The experiments with the PDE7 inhibitors were performed in the presence of the B2-adrenergic agonist salbutamol to generate a certain level of cAMP whose degradation was then inhibited by the PDE7 inhibitor to still considerably raise its levels.

### Method

Muscle preparation and incubation: Female Sprague Dawley rats of 5 weeks of age were used. After isoflurane anesthesia and cervical dislocation the intact soleus muscles of the animals were dissected. The soleus muscles were mounted at resting length in a vertical position between two field stimulation electrodes (thick copper wires) in organ bathes containing 15 mL incubation medium. The lower end of the muscle was fixed to a clip fixed to the bottom of the organ bath, the upper end of the muscle was fixed to an isometric force transducer (Force transducer K300 Hugo Sachs) by a clip allowing measurement of isometric contractions, and a tension of 100 mN was applied.

Force measurement: The experimental set-up used for force measurements allowed for the simultaneous recordings from 6 muscles in separate incubation chambers in parallel. Force was recorded and stored with a Notocord HEM data acquisition system. The standard incubation medium was a Krebs-Ringer (KR) bicarbonate buffer (pH 7.4) containing (in mM): 120.1 NaCl, 25.1 NaHCO₃, 4.7 KCl, 1.2 KH₂PO₄, 1.2 MgSO₄, 1.3 CaCl₂ and 5 glucose. Incubation took place at 30° C. The buffer was continuously gassed with a mixture of 95% O₂ and 5% CO₂. Muscles were equilibrated with a mixture of 95% O₂ and 5% CO₂ in the standard medium for 30 minutes (min) before stimulation.

Electrical stimulation protocol: After an equilibration period of 30 min muscles were field-stimulated to deliver optimal force at 83 Hz, 2-3 ms pulses, 12 V (biphasic), 0.2-0.5 s train width with a Hugo Sachs stimulator (HSE). After optimizing for the stimulation protocol the muscles were stimulated every 10 min for 30 min in the buffer indicated above to obtain the control values of force before weakening the contractile force by the depolarizing effect of KCl at 12.5 mM in the buffer.

Investigation of test compounds: At time zero (TO) the buffer described above was exchanged against a buffer containing KCl 12.5 mM. Stimulations were then performed every 5 min for 30 min. The high KCl buffer caused a strong loss of force in controls. The test compounds were added in a volume of 0.1 mL to the organ baths containing 15 mL buffer solution already 10 min before exchanging (10 min before TO) the initial buffer for the new buffer with high KCl of 12.5 mM and they were newly added again to the new high KCl buffer after the exchange. Two control groups were included. A group with high KCl 12.5 mM buffer only and a group with high KCl 12.5 mM plus salbutamol 1 μM. PDE7 inhibitors were always tested in the presence of salbutamol 1 M.

### Evaluation

The depolarizing effect of high KCl 12.5 mM caused a strong loss of force in controls. An effective drug would show a lower loss of contractile force, thus preserve contractile force. The residual contractile force after 30 min in high KCl buffer was chosen as the efficacy parameter and related to the last value of stimulated force in the initial buffer in the same organ bath. The percentages of residual force were compared between the different groups and used for statistical calculation. PDE7 inhibitor drugs were tested in the presence of salbutamol 1 M and the effects were compared with those of the salbutamol 1 M control group.

### Results

As illustrative and nonlimiting examples, the following compounds in accordance with the invention were tested according to the above protocol and delayed the rat soleus muscle force decline as indicated below, thus, showing their advantage as therapeutically active substances:

| | | | | Mean residual |
|---|---|---|---|---|
| | | | | contractile force |
| | CAS | | | in % (SEM in %); |
| Formula | number/name | Concentration | | number (n); p-value |
| Control | Salbutamol | 1 | μM | 26.6 (3.7); n = 31 |
| group | (alone) | | | |
| No. 1G | 2266633-30-9 | 16.8 | nM | 60.5 (3.0); n = 6; p < 0.01 |
| No. 1M | 2266637-12-9 | 0.3 | μM | 73.7 (9.6); n = 4; p < 0.05 |
| Dipyr- | 58-32-2 | 30 | μM | 46.3 (6.3); n = 15; |
| idamole | | | | p < 0.01 |

Soleus muscle force strongly declined under the depolarizing effect of a high extracellular potassium concentration (12.5 mM) to 13.6±1.9% (mean±SEM, n=30) mean residual contractile force compared to the force at baseline at normal potassium ion concentration.

Results for control group and compounds are shown in the table above as means with standard error of mean (SEM). Stimulation by salbutamol (1 M) moderately diminished the loss of contractile force. The effect of salbutamol 10 M alone leaving a mean of 33.6% (SEM: 7.3) of residual contractile force at n=9 was only moderately stronger than that of salbutamol 1 μM. PDE7 inhibitors were investigated in the presence of salbutamol 1 M to generate cAMP whose concentration could then be augmented by the inhibition of its hydrolyzation by PDE7 inhibition.

The PDE7 inhibitors were therefore evaluated against the effect of salbutamol 1 M and tested for statistical significance. PDE inhibition by the pan-PDE inhibitor dipyridamole (high concentration) plus moderate dose of salbutamol (1 μM) shows an even numerically stronger effect than salbutamol alone at the high dose of 10 M and so did the effective PDE7 inhibitors. The specific PDE7 inhibitors with formula No. 1 G (2266633-30-9) and No. 1 M (2266637-12-9) were more active than dipyridamole.

### CONCLUSION

PDE7 inhibitors, in particular PDE7A inhibitors, reduce muscular fatigue as shown by a preservation of contractile force under conditions of reduced excitability by sodium overload caused by the depolarizing effect of high KCl buffer. The mechanism is via restoring excitability by activation of the Na⁺/K⁺ ATPase. Based on these actions PDE7 inhibitors can treat muscular fatigue and loss of force.

Moreover, due to the finding that stimulation of the Na⁺/K⁺ ATPase lowers intracellular sodium—high intramuscular sodium causes calcium overload via the reverse mode of the sodium-calcium-exchanger (NCX) to cause muscular damage and mitochondrial dysfunction—PDE7 inhibitors can treat and prevent mitochondrial dysfunction and the deleterious secondary consequences of energy deprivation. This deprivation otherwise results in the physiological stimulation of the production of vasoactive mediators from skeletal muscle, which are normally physiologically meant to raise muscular blood flow. These mediators are then excessively produced due to the very poor energetic situation and therefore released into the systemic blood stream (spillover). Thus, these mediators with algesic, hyperalgesic, spasmogenic and microvascular leakage-inducing properties can reach every organ to cause a variety of symptoms of diseases associated with chronic fatigue. These include pain, edema and spasms, which are prevented by treatment involving PDE7 inhibition in the form of an indirect effect.

1. Substance with phosphodiesterase-7 inhibitory activity (PDE7 inhibitors) for use as active ingredient in a therapeutic agent with phosphodiesterase-7 inhibitory activity for the treatment and prevention of chronic fatigue, exhaustion and/or exertional intolerance and for the treatment and prevention of diseases that are associated with chronic fatigue, exhaustion and/or exertional intolerance.

2. Substance as claimed in claim 1, wherein diseases that are associated with chronic fatigue, exhaustion and/or exertional intolerance are diseases diagnosed as Myalgic Encephalomyelitis/Chronic Fatigue Syndrome (ME/CFS), systemic exertional intolerance, exertional intolerance, Long COVID complaints, post-Covid-19 syndrome (PCS), post-acute Covid syndrome (PACS), Post-Acute Sequelae of SARS-CoV-2 Infection (PASC), post-vaccination syndrome (post-vac-syndrome) after COVID-19 vaccinations (Long Vax) and vaccinations against other germs, virus and pathogenic agents, and postinfectious fatigue after viral, bacterial, or fungal infections, in particular ME/CFS of non-infectious, non-inflammatory and non-immunological cause.

3. Substance as claimed in claim 1, wherein chronic fatigue and exhaustion are symptoms of or are associated with cancer (cancer-related fatigue), fibromyalgia, Ehlers-Danlos syndrome, Marfan syndrome, Gulf War illness, the autoimmune diseases Rheumatoid Arthritis, ANCA vasculitis and Sjögren's syndrome, and other autoimmune diseases with fatigue and exhaustion as debilitating symptoms.

4. The substance as claimed in one or more of claims 1 to 3, wherein the PDE7 inhibitory material is a material that inhibits the enzymatic activity of PDE7 (PDE7A, PDE7B, or PDE7A and PDE7B) at an IC₅₀<50 μM, preferably at an IC₅₀ 1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.

5. The substance as claimed in one or more of claims 1 to 4, wherein the PDE7 inhibitory material is a material that inhibits the enzymatic activity of PDE7A, preferably the PDE7 inhibitory material is a material that inhibits the enzymatic activity of PDE7A at an IC₅₀<50 μM, preferably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.

6. The substance as claimed in one or more of claims 1, 2, 3, 4, and 5, wherein the PDE7 inhibitory agent stimulates the Na⁺/K⁺-ATPase and the NCLX in skeletal muscle and improves blood flow to skeletal muscles and brain and inhibits neuroinflammation by raising cAMP.

7. The substance as claimed in one or more of claims 1 to 6, wherein the PDE7 inhibitory agent or pharmaceutically acceptable salt thereof and a pharmaceutically acceptable carrier are present in a pharmaceutical preparation, preferably in the form of an oral drug, intravenous, subcutaneous, intramuscular, pharyngeal, and nasal administration.

8. The substance as claimed in one or more of claims 1 to 7, wherein the PDE7 inhibitory agent is selected from the formula (1): 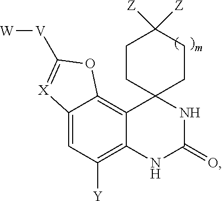
or pharmaceutically acceptable salt thereof, wherein, X is —CH or —N; Y is selected from the group consisting of: —H, halo, and —C₁₋₄alkyl; each Z is independently selected from the group consisting of: —H, -halo, and —C₁₋₄alkyl; V is selected from the group consisting of: a bond, —(CH₂)ₘ—, —(CH₂)ₘO(CH₂)ₙ—, —(CH₂)ₘO(CH₂)ₙC(O)—, —(CH₂)_{M}N[(CH₂)_{N}R^{1A}]—, —(CH₂)ₘC(O)O—, —(CH₂)ₘC(O)—, —(CH₂)ₘC(O)N[(CH₂)ₙR^{1A}]—, -L¹-L²-, -L¹-L²-L³-, -L¹-L²-L³-L⁴-, and -L¹-L⁵; each m is independently 0, 1, 2 or 3; each n is independently 0, 1, 2 or 3; L¹ is —C(O)(CH₂)ₘ—, —[C(R^{1A})₂]ₘ— or —(CH₂)ₘ—; L² is —N[(CH₂)ₙR^{1A}]— or —N((CH₂)ₙR^{1A})(CH₂)ₚ—; L³ is selected from the group consisting of —(CH₂)ₘ—C₃₋₇cycloalkyl, —[C((CH₂)ₙR^{1B})₂]ₘ[C((CH₂)ₙR^{1C})₂]ₙ—, —[N[(CH₂)ₘR^{1A}](CH₂)ₙ]—, —[(CH₂)ₘN((CH₂)ₙR^{1A})(CH₂)ₚ], —[(CH₂)ₘC(O)N((CH₂)ₙR^{1B})(CH₂)ₚ]—, —(CH₂)ₘC(O)—, —[C(R^{1A})₂]ₘ— and —[(CH₂)ₚO]—; L⁴ is selected from the group consisting of —(CH₂)ₘC(O)—, —(CH₂)ₘO—, —C₁₋₆alkyl, —C₃₋₇cycloalkyl, heteroaryl, aryl, 3-10 membered heterocycloalkyl, and —CH(OH)—, said —C₁₋₆alkyl, —C₃₋₇cycloalkyl, heteroaryl, aryl, and 3-10 membered heterocycloalkyl each optionally substituted with one to four R^{2A}; L⁵ is selected from the group consisting of —C(O)(CH₂)ₘN[(CH₂)ₙR^{1B}]—, —C(O)(CH₂)ₘ—, —CH(OH)— and —(CH₂)ₘC(O)O—; each p is independently 0, 1, 2 or 3; each R^{1A} is independently selected from the group consisting of: —H, —OH, —CN, halo, —C₁₋₆alkyl, —C₂₋₆alkenyl, —C₂₋₆alkynyl, —C₃₋₇cycloalkyl, aryl, heteroaryl, 3-15 membered heterocycloalkyl, —C₁₋₆haloalkyl, and —C₁₋₆alkoxy, said —C₃₋₇cycloalkyl, aryl, heteroaryl, 3-15 membered heterocycloalkyl, —C₁₋₆haloalkyl, and —C₁₋₆alkoxy each optionally substituted with one to four R^{2A}; each R^{1B} is independently selected from the group consisting of: —H, —OH, —CN, halo, —C₁₋₆alkyl, —C₂₋₆alkenyl, —C₂₋₆alkynyl, —C₃₋₇cycloalkyl, aryl, heteroaryl, 3-15 membered heterocycloalkyl, —C₁₋₆haloalkyl, and —C₁₋₆alkoxy, said —C₃₋₇cycloalkyl, aryl, heteroaryl, 3-15 membered heterocycloalkyl, —C₁₋₆haloalkyl, and —C₁₋₆alkoxy each optionally substituted with one to four R^{2A}; each R^{1C} is independently selected from the group consisting of: —H, —OH, —CN, halo, —C₁₋₆alkyl, —C₂₋₆alkenyl, —C₂₋₆alkynyl, —C₃₋₇cycloalkyl, aryl, heteroaryl, 3-15 membered heterocycloalkyl, —C₁₋₆haloalkyl, and —C₁₋₆alkoxy, said —C₃₋₇cycloalkyl, aryl, heteroaryl, 3-15 membered heterocycloalkyl, —C₁₋₆haloalkyl, and —C₁₋₆alkoxy each optionally substituted with one to four R^{2A}; W is selected from the group consisting of: —H, -halo, —OH, —CN, —C₁₋₆alkyl, —C₁₋₆haloalkyl, aryl, heteroaryl, 3-15 membered heterocycloalkyl, —C₃₋₇cycloalkyl, —SO₂C₁₋₆alkyl, —(CH₂)ₘR^{1A}, —(CH₂)ₘN(R^{1B})₂, —(CH₂)ₘO(CH₂)ₙR^{1A}, —(CH₂)ₘS(CH₂)ₙR^{1A}, —(CH₂)ₘC(O)C₁₋₆alkyl, —(CH₂)ₘC(O)heterocycloalkyl, —(CH₂)ₘC(O)OH, —(CH₂)ₘC(O)OC₁₋₆alkyl, —(CH₂)ₘC(O)NH₂, —(CH₂)ₘC(O)N(C₁₋₄alkyl)₂, and —(CH₂)ₘC(O)NH(C₁₋₄alkyl), said aryl, heteroaryl, 3-15 membered heterocycloalkyl, and —C₃₋₇cycloalkyl, each optionally substituted with one to five R^{3A}; each R^{2A} is independently selected from the group consisting of: halo, —CN, ═O, —OH, —SO₂C₁₋₆alkyl, —C₁₋₆alkyl, —C₁₋₆alkoxy, —C₁₋₆haloalkyl, aryl, heteroaryl, 3-15 membered heterocycloalkyl, —C₃₋₇cycloalkyl, —(CH₂)ₘN(C₁₋₄alkyl)₂, —OCH₂(CH₂)ₘR^{2AA}, —CH₂(CH₂)ₘR^{2AA}, —C₁₋₆alkyl-OH, —C₁₋₆haloalkylOH, —C₁₋₆haloalkyl-C₃₋₇cycloalkyl, —C₂₋₆alkenyl, —C₂₋₆alkynyl, —C(O)C(CH₃)₃, —OC₃₋₇cycloalkyl, —C(O)C₁₋₆alkyl, —C(O)aryl, —C(O)heterocycloalkyl, —C(O)OC₁₋₆alkyl, —C(O)R^{2AA}, —NHC(O)R^{2AA}, —C(O)NH(C₁₋₄alkyl), —C(O)N(C₁₋₄alkyl)₂, —CH₂CH(OH)C₃₋₇cycloalkyl, —C(CH₃)₂OH, —N(R^{2AA})₂, —C(CH₃)₂CH₂OCH₃, —CH(CH₃)C(O)N(C₁₋₄alkyl)₂, and —CH₂CF₂C₃₋₇cycloalkyl, said aryl, heteroaryl, 3-15 membered heterocycloalkyl, and —C₃₋₇cycloalkyl, each optionally substituted with one to five substituents each independently selected from the group consisting of: —CN, ═O, —OH, —SO₂C₁₋₆alkyl, —C₁₋₆alkyl, —C₁₋₆alkoxy, —C₁₋₆haloalkyl, aryl, heteroaryl, 3-15 membered heterocycloalkyl, —C₃₋₇cycloalkyl, —N(C₁₋₄alkyl)₂, —NH(C₁₋₄alkyl), —C₂₋₆alkenyl, —C₂₋₆alkynyl, —C(O)C(CH₃)₃, —OC₃₋₇cycloalkyl, —C(O)C₁₋₆alkyl, —C(O)aryl, —C(O)heterocycloalkyl, —C(O)OC₁₋₆alkyl, —C(O)N(C₁₋₄alkyl)₂, —CH₂CH(OH)C₃₋₇cycloalkyl, and —C(CH₃)₂OH; each R^{3A} is independently selected from the group consisting of: halo, —CN, ═O, —OH, —SO₂C₁₋₆alkyl, —C₁₋₆alkyl, —C₁₋₆alkoxy, —C₁₋₆haloalkyl, aryl, heteroaryl, 3-15 membered heterocycloalkyl, —C₃₋₇cycloalkyl, —(CH₂)ₘN(C₁₋₄alkyl)₂, —OCH₂(CH₂)ₘR^{3AA}, —CH₂(CH₂)ₘR^{3AA}, —C₁₋₆alkyl-OH, —C₁₋₆haloalkylOH, —C₁₋₆haloalkyl-C₃₋₇cycloalkyl, —C₂₋₆alkenyl, —C₂₋₆alkynyl, —C(O)C(CH₃)₃, —OC₃₋₇cycloalkyl, —C(O)C₁₋₆alkyl, —C(O)aryl, —C(O)heterocycloalkyl, —C(O)OC₁₋₆alkyl, —C(O)R^{3AA}, —NHC(O)R^{3AA}, —C(O)NH(C₁₋₄alkyl), —C(O)N(C₁₋₄alkyl)₂, —CH₂CH(OH)C₃₋₇cycloalkyl, —C(CH₃)₂OH, —N(R^{3AA})₂, —C(CH₃)₂CH₂OCH₃, —CH(CH₃)C(O)N(C₁₋₄alkyl)₂, and —CH₂CF₂C₃₋₇cycloalkyl, said aryl, heteroaryl, 3-15 membered heterocycloalkyl, and —C₃₋₇cycloalkyl, each optionally substituted with one to five substituents each independently selected from the group consisting of: —CN, ═O, —OH, —SO₂C₁₋₆alkyl, —C₁₋₆alkyl, —C₁₋₆alkoxy, —C₁₋₆haloalkyl, aryl, heteroaryl, 3-15 membered heterocycloalkyl, —C₃₋₇cycloalkyl, —N(C₁₋₄alkyl)₂, —NH(C₁₋₄alkyl), —C₂₋₆alkenyl, —C₂₋₆alkynyl, —C(O)C(CH₃)₃, —OC₃₋₇cycloalkyl, —C(O)C₁₋₆alkyl, —C(O)aryl, —C(O)heterocycloalkyl, —C(O)OC₁₋₆alkyl, —C(O)N(C₁₋₄alkyl)₂, —CH₂CH(OH)C₃₋₇cycloalkyl, and —C(CH₃)₂OH; each R^{2AA} is independently selected from the group consisting of: —H, —OH, —SO₂C₁₋₆alkyl, halo, —CN, —C₁₋₆alkoxy, —C₁₋₆haloalkyl, —N(C₁₋₄alkyl)₂, —NH(C₁₋₄alkyl), —NH₂, aryl, heteroaryl, 3-15 membered heterocycloalkyl, —C₂₋₆alkenyl, —C₂₋₆alkynyl, —C₃₋₇cycloalkyl, —C(O)C₁₋₆alkyl, —C(O)heterocycloalkyl, —C(O)OC₁₋₆alkyl, —C(O)N(C₁₋₄alkyl)₂, and —C(O)aryl; and each R^{3A} is independently selected from the group consisting of: —H, —OH, —SO₂C₁₋₆alkyl, halo, —CN, —C₁₋₆alkoxy, —C₁₋₆haloalkyl, —N(C₁₋₄alkyl)₂, —NH(C₁₋₄alkyl), —NH₂, aryl, heteroaryl, 3-15 membered heterocycloalkyl, —C₂₋₆alkenyl, —C₂₋₆alkynyl, —C₃₋₇cycloalkyl, —C(O)C₁₋₆alkyl, —C(O)heterocycloalkyl, —C(O)OC₁₋₆alkyl, —C(O)N(C₁₋₄alkyl)₂, and —C(O)aryl.

9. The substance as claimed in one or more of claims 1 to 8, wherein the PDE7 inhibitory agent is a member selected from the group consisting of substances comprising spiro derivatives; benzoxazole derivates; benzofuran derivates; methylene-substituted 7,8-dihydro-6H-spiro[[1,3]oxazolo[5,4-f]quinazoline-9,1′-cyclohexane]-7-one derivatives; acylated 7,8-dihydro-6H-spiro[[1,3]oxazolo[5,4-f]quinazoline-9,1′-cyclohexane]-7-one derivatives; methylene-substituted 7,8-dihydro-6H-spiro[[1,3]furo[5,4-f]quinazoline-9,1′-cyclohexane]-7-one derivatives; and acylated 7,8-dihydro-6H-spiro[[1,3]furo[5,4-f]quinazoline-9,1′-cyclohexane]-7-one derivatives.

10. The substance as claimed in one or more of claims 1 to 9, wherein the PDE7 inhibitory agent is a compound selected from the group consisting of: methyl 5-chloro-7-oxo-7,8-dihydro-6H-spiro[[1,3]oxazolo[5,4-f]quinazoline-9,1′-cyclohexane]-2-carboxylate; 5-chloro-7-oxo-7,8-dihydro-6H-spiro[[1,3]oxazolo[5,4-f]quinazoline-9,1′-cyclohexane]-2-carboxylic acid; 5-chloro-2-(chloromethyl)-7,8-dihydro-6H-spiro[[1,3]oxazolo[5,4-f]quinazoline-9,1′-cyclohexane]-7-one; 5-chloro-2-{[(2-methoxyethyl)amino]methyl}-7,8-dihydro-6H-spiro[[1,3]oxazolo[5,4-f]quinazoline-9,1′-cyclohexane]-7-one; 5-chloro-2-[(2-methoxyethoxy)methyl]-7,8-dihydro-6H-spiro[[1,3]oxazolo[5,4-f]quinazoline-9,1′-cyclohexane]-7-one; ethyl 2-{5-chloro-7-oxo-7,8-dihydro-6H-spiro[[1,3]oxazolo[5,4f]quinazoline-9,1′-cyclohexane]-2-yl}acetate; 5-chloro-2-(4-methoxypiperidine-1-carbonyl)-7,8-dihydro-6H-spiro[[1,3]oxazolo[5,4-f]quinazoline-9,1′-cyclohexane]-7-one; 5-fluoro-2-[(3-methoxyazetidin-1-yl)methyl]-7,8-dihydro-6H-spiro[[1,3]oxazolo[5,4-f]quinazoline-9,1′-cyclohexane]-7-one; methyl 5-chloro-4′,4′-difluoro-7-oxo-7,8-dihydro-6H-spiro[[1,3]oxazolo[5,4-f]quinazoline-9,1′-cyclohexane]-2-carboxylate; 5-chloro-4′,4′-difluoro-2-[(4-methylpiperazin-1-yl)methyl]-7,8-dihydro-6H-spiro[[1,3]oxazolo[5,4-f]quinazoline-9,1′-cyclohexane]-7-one; 5-chloro-4′,4′-difluoro-2-methyl-7,8-dihydro-6H-spiro[[1,3]oxazolo[5,4-f]quinazoline-9,1′-cyclohexane]-7-one; 5-chloro-2,4′,4′-trimethyl-7,8-dihydro-6H-spiro[[1,3]oxazolo[5,4-f]quinazoline-9,1′-cyclohexane]-7-one; 5-chloro-7,8-dihydro-6H-spiro[[1,3]oxazolo[5,4-f]quinazoline-9,1′-cyclohexane]-7-one; 5-chloro-2-methyl-7,8-dihydro-6H-spiro[[1,3]oxazolo[5,4-f]quinazoline-9,1′-cyclohexane]-7-one; 5-chloro-2-ethyl-7,8-dihydro-6H-spiro[[1,3]oxazolo[5,4-f]quinazoline-9,1′-cyclohexane]-7-one; 5-chloro-2-propyl-7,8-dihydro-6H-spiro[[1,3]oxazolo[5,4-f]quinazoline-9,1′-cyclohexane]-7-one; 5-chloro-2-(propan-2-yl)-7,8-dihydro-6H-spiro[[1,3]oxazolo[5,4-f]quinazoline-9,1′-cyclohexane]-7-one; 5-chloro-2-cyclopropyl-7,8-dihydro-6H-spiro[[1,3]oxazolo[5,4-f]quinazoline-9,1′-cyclohexane]-7-one; 5-chloro-2-(methoxymethyl)-7,8-dihydro-6H-spiro[[1,3]oxazolo[5,4-f]quinazoline-9,1′-cyclohexane]-7-one; 5-chloro-2-(hydroxymethyl)-7,8-dihydro-6H-spiro[[1,3]oxazolo[5,4-f]quinazoline-9,1′-cyclohexane]-7-one; 5-chloro-2-(2-methoxyethyl)-7,8-dihydro-6H-spiro[[1,3]oxazolo[5,4-f]quinazoline-9,1′-cyclohexane]-7-one; 5-chloro-2-(2-hydroxyethyl)-7,8-dihydro-6H-spiro[[1,3]oxazolo[5,4-f]quinazoline-9,1′-cyclohexane]-7-one; N-[5-chloro-7-oxo-7,8-dihydro-6H-spiro[[1,3]oxazolo[5,4-f]quinazoline-9,1′-cyclohexane]-2-yl]acetamide; 2-[bis(2-methoxyethyl)amino]-5-chloro-7,8-dihydro-6H-spiro[[1,3]oxazolo[5,4-f]quinazoline-9,1′-cyclohexane]-7-one; 5-chloro-7-oxo-7,8-dihydro-6H-spiro[[1,3]oxazolo[5,4-f]quinazoline-9,1′-cyclohexane]-2-carboxamide; 5-chloro-2-[(dimethylamino)methyl]-7,8-dihydro-6H-spiro[[1,3]oxazolo[5,4-f]quinazoline-9,1′-cyclohexane]-7-one; 5-chloro-2-[(4-methylpiperazin-1-yl)methyl]-7,8-dihydro-6H-spiro[[1,3]oxazolo[5,4-f]quinazoline-9,1′-cyclohexane]-7-one; 5-chloro-2-(morpholin-4-ylmethyl)-7,8-dihydro-6H-spiro[[1,3]oxazolo[5,4-f]quinazoline-9,1′-cyclohexane]-7-one; 5-chloro-2-[(4-hydroxypiperidin-1-yl)methyl]-7,8-dihydro-6H-spiro[[1,3]oxazolo[5,4-f]quinazoline-9,1′-cyclohexane]-7-one; 5-chloro-2-[(methylamino)methyl]-7,8-dihydro-6H-spiro[[1,3]oxazolo[5,4-f]quinazoline-9,1′-cyclohexane]-7-one; 5-chloro-2-[(cyclopropylamino)methyl]-7,8-dihydro-6H-spiro[[1,3]oxazolo[5,4-f]quinazoline-9,1′-cyclohexane]-7-one; 5-chloro-2-(piperazin-1-ylmethyl)-7,8-dihydro-6H-spiro[[1,3]oxazolo[5,4-f]quinazoline-9,1′-cyclohexane]-7-one; 5-chloro-2-(pyrrolidin-1-ylmethyl)-7,8-dihydro-6H-spiro[[1,3]oxazolo[5,4-f]quinazoline-9,1′-cyclohexane]-7-one; 5-chloro-2-{[(propan-2-yl)amino]methyl}-7,8-dihydro-6H-spiro[[1,3]oxazolo[5,4-f]quinazoline-9,1′-cyclohexane]-7-one; 5-chloro-2-[(ethylamino)methyl]-7,8-dihydro-6H-spiro[[1,3]oxazolo[5,4-f]quinazoline-9,1′-cyclohexane]-7-one; 5-chloro-2-{[(2-hydroxyethyl)amino]methyl}-7,8-dihydro-6H-spiro[[1,3]oxazolo[5,4-f]quinazoline-9,1′-cyclohexane]-7-one; 5-chloro-2-[(4,4-difluoropiperidin-1-yl)methyl]-7,8-dihydro-6H-spiro[[1,3]oxazolo[5,4-f]quinazoline-9,1′-cyclohexane]-7-one; 5-chloro-2-{[(2,2,2-trifluoroethyl)amino]methyl}-7,8-dihydro-6H-spiro[[1,3]oxazolo[5,4-f]quinazoline-9,1′-cyclohexane]-7-one; 5-chloro-2-{[(2-hydroxyethyl)(methyl)amino]methyl}-7,8-dihydro-6H-spiro[[1,3]oxazolo[5,4-f]quinazoline-9,1′-cyclohexane]-7-one; 5-chloro-2-[(3,3-difluoroazetidin-1-yl)methyl]-7,8-dihydro-6H-spiro[[1,3]oxazolo[5,4-f]quinazoline-9,1′-cyclohexane]-7-one; 5-chloro-2-[(3,3-difluoropyrrolidin-1-yl)methyl]-7,8-dihydro-6H-spiro[[1,3]oxazolo[5,4-f]quinazoline-9,1′-cyclohexane]-7-one; 2-[(4-acetylpiperazin-1-yl)methyl]-5-chloro-7,8-dihydro-6H-spiro[[1,3]oxazolo[5,4-f]quinazoline-9,1′-cyclohexane]-7-one; 5-chloro-2-[(4-propanoylpiperazin-1-yl)methyl]-7,8-dihydro-6H-spiro[[1,3]oxazolo[5,4-f]quinazoline-9,1′-cyclohexane]-7-one; 5-chloro-2-{2-oxa-8-azaspiro[4.5]decan-8-ylmethyl}-7,8-dihydro-6H-spiro[[1,3]oxazolo[5,4-f]quinazoline-9,1′-cyclohexane]-7-one; 5-chloro-2-{[4-(oxetan-3-yl)piperazin-1-yl]methyl}-7,8-dihydro-6H-spiro[[1,3]oxazolo[5,4-f]quinazoline-9,1′-cyclohexane]-7-one; 2-({5-chloro-7-oxo-7,8-dihydro-6H-spiro[[1,3]oxazolo[5,4-f]quinazoline-9,1′-cyclohexane]-2-ylmethyl}amino)-N,N-dimethylacetamide; tert-butyl 2-[({5-chloro-7-oxo-7,8-dihydro-6H-spiro[[1,3]oxazolo[5,4-f]quinazoline-9,1′-cyclohexane]-2-ylmethyl}amino)methyl]piperidine-1-carboxylate; 5-chloro-2-({[(3,5-difluoropyridin-2-yl)methyl]amino}methyl)-7,8-dihydro-6H-spiro[[1,3]oxazolo[5,4-f]quinazoline-9,1′-cyclohexane]-7-one; 5-chloro-2-{[(piperidin-2-ylmethyl)amino]methyl}-7,8-dihydro-6H-spiro[[1,3]oxazolo[5,4-f]quinazoline-9,1′-cyclohexane]-7-one; 5-chloro-2-({[2-oxo-2-(piperidin-1-yl)ethyl]amino}methyl)-7,8-dihydro-6H-spiro[[1,3]oxazolo[5,4-f]quinazoline-9,1′-cyclohexane]-7-one; 2-({5-chloro-7-oxo-7,8-dihydro-6H-spiro[[1,3]oxazolo[5,4-f]quinazoline-9,1′-cyclohexane]-2-ylmethyl}amino)-N-ethyl-N-methylacetamide; 5-chloro-2-[(4-methyl-3-oxopiperazin-1-yl)methyl]-7,8-dihydro-6H-spiro[[1,3]oxazolo[5,4-f]quinazoline-9,1′-cyclohexane]-7-one; 5-chloro-2-[(4-ethyl-3-oxopiperazin-1-yl)methyl]-7,8-dihydro-6H-spiro[[1,3]oxazolo[5,4-f]quinazoline-9,1′-cyclohexane]-7-one; 5-chloro-2-{[(pyridin-2-ylmethyl)amino]methyl}-7,8-dihydro-6H-spiro[[1,3]oxazolo[5,4-f]quinazoline-9,1′-cyclohexane]-7-one; 5-chloro-2-{[(pyrimidin-2-ylmethyl)amino]methyl}-7,8-dihydro-6H-spiro[[1,3]oxazolo[5,4-f]quinazoline-9,1′-cyclohexane]-7-one; 5-chloro-2-({[(5-methoxypyridin-3-yl)methyl]amino}methyl)-7,8-dihydro-6H-spiro[[1,3]oxazolo[5,4-f]quinazoline-9,1′-cyclohexane]-7-one; 5-chloro-2-[(2,4-dimethyl-3-oxopiperazin-1-yl)methyl]-7,8-dihydro-6H-spiro[[1,3]oxazolo[5,4-f]quinazoline-9,1′-cyclohexane]-7-one; ethyl 1-{5-chloro-7-oxo-7,8-dihydro-6H-spiro[[1,3]oxazolo[5,4-f]quinazoline-9,1′-cyclohexane]-2-ylmethyl}piperidine-4-carboxylate; 5-chloro-2-({1-oxo-2-oxa-8-azaspiro[4.5]decan-8-yl}methyl)-7,8-dihydro-6H-spiro[[1,3]oxazolo[5,4-f]quinazoline-9,1′-cyclohexane]-7-one; 2-[(4-acetylpiperidin-1-yl)methyl]-5-chloro-7,8-dihydro-6H-spiro[[1,3]oxazolo[5,4-f]quinazoline-9,1′-cyclohexane]-7-one; methyl 1-{5-chloro-7-oxo-7,8-dihydro-6H-spiro[[1,3]oxazolo[5,4-f]quinazoline-9,1′-cyclohexane]-2-ylmethyl}piperidine-4-carboxylate; 5-chloro-2-({[(5-fluoropyridin-2-yl)methyl]amino}methyl)-7,8-dihydro-6H-spiro[[1,3]oxazolo[5,4-f]quinazoline-9,1′-cyclohexane]-7-one; 5-chloro-2-{[3-(2-methylpropoxy)azetidin-1-yl]methyl}-7,8-dihydro-6H-spiro[[1,3]oxazolo[5,4-f]quinazoline-9,1′-cyclohexane]-7-one; 5-chloro-2-[(4-ethoxypiperidin-1-yl)methyl]-7,8-dihydro-6H-spiro[[1,3]oxazolo[5,4-f]quinazoline-9,1′-cyclohexane]-7-one; 5-chloro-2-{[4-(cyclopropylmethoxy)piperidin-1-yl]methyl}-7,8-dihydro-6H-spiro[[1,3]oxazolo[5,4-f]quinazoline-9,1′-cyclohexane]-7-one; 5-chloro-2-{[4-hydroxy-4-(trifluoromethyl)piperidin-1-yl]methyl}-7,8-dihydro-6H-spiro[[1,3]oxazolo[5,4-f]quinazoline-9,1′-cyclohexane]-7-one; 5-chloro-2-{[4-(propan-2-yloxy)piperidin-1-yl]methyl}-7,8-dihydro-6H-spiro[[1,3]oxazolo[5,4-f]quinazoline-9,1′-cyclohexane]-7-one; 5-chloro-2-[(3-fluoroazetidin-1-yl)methyl]-7,8-dihydro-6H-spiro[[1,3]oxazolo[5,4-f]quinazoline-9,1′-cyclohexane]-7-one; 5-chloro-2-[(3-methoxyazetidin-1-yl)methyl]-7,8-dihydro-6H-spiro[[1,3]oxazolo[5,4-f]quinazoline-9,1′-cyclohexane]-7-one; 5-chloro-2-{[3-(methoxymethyl)azetidin-1-yl]methyl}-7,8-dihydro-6H-spiro[[1,3]oxazolo[5,4-f]quinazoline-9,1′-cyclohexane]-7-one; 5-chloro-2-{7-oxa-2-azaspiro[3.5]nonan-2-ylmethyl}-7,8-dihydro-6H-spiro[[1,3]oxazolo[5,4-f]quinazoline-9,1′-cyclohexane]-7-one; 5-chloro-2-[(4-methoxy-4-methylpiperidin-1-yl)methyl]-7,8-dihydro-6H-spiro[[1,3]oxazolo[5,4-f]quinazoline-9,1′-cyclohexane]-7-one; 5-chloro-2-({3-oxo-1-oxa-8-azaspiro[4.5]decan-8-yl}methyl)-7,8-dihydro-6H-spiro[[1,3]oxazolo[5,4-f]quinazoline-9,1′-cyclohexane]-7-one; 2-({5-chloro-7-oxo-7,8-dihydro-6H-spiro[[1,3]oxazolo[5,4-f]quinazoline-9,1′-cyclohexane]-2-ylmethyl}(methyl)amino)-N,N-dimethylacetamide; 5-chloro-2-({[(6-methylpyridin-3-yl)methyl]amino}methyl)-7,8-dihydro-6H-spiro[[1,3]oxazolo[5,4-f]quinazoline-9,1′-cyclohexane]-7-one; 5-chloro-2-{[methyl(oxan-4-ylmethyl)amino]methyl}-7,8-dihydro-6H-spiro[[1,3]oxazolo[5,4-f]quinazoline-9,1′-cyclohexane]-7-one; 5-chloro-2-({methyl[(3-methyloxetan-3-yl)methyl]amino}methyl)-7,8-dihydro-6H-spiro[[1,3]oxazolo[5,4-f]quinazoline-9,1′-cyclohexane]-7-one; 5-chloro-2-({[(3-fluorooxetan-3-yl)methyl]amino}methyl)-7,8-dihydro-6H-spiro[[1,3]oxazolo[5,4-f]quinazoline-9,1′-cyclohexane]-7-one; 5-chloro-2-{[(oxan-4-ylmethyl)amino]methyl}-7,8-dihydro-6H-spiro[[1,3]oxazolo[5,4-f]quinazoline-9,1′-cyclohexane]-7-one; 5-chloro-2-{2-oxa-7-azaspiro[3.5]nonan-7-ylmethyl}-7,8-dihydro-6H-spiro[[1,3]oxazolo[5,4-f]quinazoline-9,1′-cyclohexane]-7-one; 5-chloro-2-({[(3-methyloxetan-3-yl)methyl]amino}methyl)-7,8-dihydro-6H-spiro[[1,3]oxazolo[5,4-f]quinazoline-9,1′-cyclohexane]-7-one; 5-chloro-2-{2-oxa-5-azabicyclo[2.2.1]heptan-5-ylmethyl}-7,8-dihydro-6H-spiro[[1,3]oxazolo[5,4-f]quinazoline-9,1′-cyclohexane]-7-one; 5-chloro-2-{[methyl(2,2,2-trifluoroethyl)amino]methyl}-7,8-dihydro-6H-spiro[[1,3]oxazolo[5,4-f]quinazoline-9,1′-cyclohexane]-7-one; 5-chloro-2-{[(oxetan-3-ylmethyl)amino]methyl}-7,8-dihydro-6H-spiro[[1,3]oxazolo[5,4-f]quinazoline-9,1′-cyclohexane]-7-one; 5-chloro-2-[(4-oxopiperidin-1-yl)methyl]-7,8-dihydro-6H-spiro[[1,3]oxazolo[5,4-f]quinazoline-9,1′-cyclohexane]-7-one; 5-chloro-2-({[(2-methoxyphenyl)methyl]amino}methyl)-7,8-dihydro-6H-spiro[[1,3]oxazolo[5,4-f]quinazoline-9,1′-cyclohexane]-7-one; 5-chloro-2-({[(2,4-dimethoxyphenyl)methyl]amino}methyl)-7,8-dihydro-6H-spiro[[1,3]oxazolo[5,4-f]quinazoline-9,1′-cyclohexane]-7-one; 5-chloro-2-{[4-(propan-2-yl)piperazin-1-yl]methyl}-7,8-dihydro-6H-spiro[[1,3]oxazolo[5,4-f]quinazoline-9,1′-cyclohexane]-7-one; 5-chloro-2-[(3,3,4-trimethylpiperazin-1-yl)methyl]-7,8-dihydro-6H-spiro[[1,3]oxazolo[5,4-f]quinazoline-9,1′-cyclohexane]-7-one; 5-chloro-2-{[2-(hydroxymethyl)piperidin-1-yl]methyl}-7,8-dihydro-6H-spiro[[1,3]oxazolo[5,4-f]quinazoline-9,1′-cyclohexane]-7-one; 5-chloro-2-({[1-(hydroxymethyl)cyclopentyl]amino}methyl)-7,8-dihydro-6H-spiro[[1,3]oxazolo[5,4-f]quinazoline-9,1′-cyclohexane]-7-one; 5-chloro-2-{[(2R)-2-(hydroxymethyl)pyrrolidin-1-yl]methyl}-7,8-dihydro-6H-spiro[[1,3]oxazolo[5,4-f]quinazoline-9,1′-cyclohexane]-7-one; 5-chloro-2-[(2-methylpiperidin-1-yl)methyl]-7,8-dihydro-6H-spiro[[1,3]oxazolo[5,4-f]quinazoline-9,1′-cyclohexane]-7-one; 5-chloro-2-{[2-(2-methylpropyl)morpholin-4-yl]methyl}-7,8-dihydro-6H-spiro[[1,3]oxazolo[5,4-f]quinazoline-9,1′-cyclohexane]-7-one; 5-chloro-2-{[(2-cyclobutyl-2,2-difluoroethyl)amino]methyl}-7,8-dihydro-6H-spiro[[1,3]oxazolo[5,4-f]quinazoline-9,1′-cyclohexane]-7-one; 5-chloro-2-[(3-ethylmorpholin-4-yl)methyl]-7,8-dihydro-6H-spiro[[1,3]oxazolo[5,4-f]quinazoline-9,1′-cyclohexane]-7-one; 5-chloro-2-{[(2-cyclohexyl-2-hydroxyethyl)amino]methyl}-7,8-dihydro-6H-spiro[[1,3]oxazolo[5,4-f]quinazoline-9,1′-cyclohexane]-7-one; 5-chloro-2-{6-oxa-9-azaspiro[4.5]decan-9-ylmethyl}-7,8-dihydro-6H-spiro[[1,3]oxazolo[5,4-f]quinazoline-9,1′-cyclohexane]-7-one; 5-chloro-2-({[1-(oxan-2-yl)ethyl]amino}methyl)-7,8-dihydro-6H-spiro[[1,3]oxazolo[5,4-f]quinazoline-9,1′-cyclohexane]-7-one; 5-chloro-2-{[(oxan-2-ylmethyl)amino]methyl}-7,8-dihydro-6H-spiro[[1,3]oxazolo[5,4-f]quinazoline-9,1′-cyclohexane]-7-one; 5-chloro-2-{[(3-methyl-2-oxobutyl)amino]methyl}-7,8-dihydro-6H-spiro[[1,3]oxazolo[5,4-f]quinazoline-9,1′-cyclohexane]-7-one; 5-chloro-2-{[(4,4-difluorocyclohexyl)amino]methyl}-7,8-dihydro-6H-spiro[[1,3]oxazolo[5,4-f]quinazoline-9,1′-cyclohexane]-7-one; 5-chloro-2-({[(1r,4r)-4-methoxycyclohexyl]amino}methyl)-7,8-dihydro-6H-spiro[[1,3]oxazolo[5,4-f]quinazoline-9,1′-cyclohexane]-7-one; 5-chloro-2-[(1R,4R)-2-oxa-5-azabicyclo[2.2.1]heptan-5-ylmethyl]-7,8-dihydro-6H-spiro[[1,3]oxazolo[5,4-f]quinazoline-9,1′-cyclohexane]-7-one; 5-chloro-2-[(1S,4S)-2-oxa-5-azabicyclo[2.2.1]heptan-5-ylmethyl]-7,8-dihydro-6H-spiro[[1,3]oxazolo[5,4-f]quinazoline-9,1′-cyclohexane]-7-one; 5-chloro-2-[(3-methoxy-3-methylazetidin-1-yl)methyl]-7,8-dihydro-6H-spiro[[1,3]oxazolo[5,4-f]quinazoline-9,1′-cyclohexane]-7-one; 5-chloro-2-[(4-methoxypiperidin-1-yl)methyl]-7,8-dihydro-6H-spiro[[1,3]oxazolo[5,4-f]quinazoline-9,1′-cyclohexane]-7-one; 5-chloro-2-{[4-(dimethylamino)piperidin-1-yl]methyl}-7,8-dihydro-6H-spiro[[1,3]oxazolo[5,4-f]quinazoline-9,1′-cyclohexane]-7-one; 5-chloro-2-({5-methyl-octahydropyrrolo[3,4-c]pyrrol-2-yl}methyl)-7,8-dihydro-6H-spiro[[1,3]oxazolo[5,4-f]quinazoline-9,1′-cyclohexane]-7-one; 5-chloro-2-{[(2R)-2-(methoxymethyl)pyrrolidin-1-yl]methyl}-7,8-dihydro-6H-spiro[[1,3]oxazolo[5,4-f]quinazoline-9,1′-cyclohexane]-7-one; 5-chloro-2-{[3-(hydroxymethyl)pyrrolidin-1-yl]methyl}-7,8-dihydro-6H-spiro[[1,3]oxazolo[5,4-f]quinazoline-9,1′-cyclohexane]-7-one; 5-chloro-2-{[(3R)-3-methoxypiperidin-1-yl]methyl}-7,8-dihydro-6H-spiro[[1,3]oxazolo[5,4-f]quinazoline-9,1′-cyclohexane]-7-one; 5-chloro-2-{[(3S)-3-methoxypiperidin-1-yl]methyl}-7,8-dihydro-6H-spiro[[1,3]oxazolo[5,4-f]quinazoline-9,1′-cyclohexane]-7-one; 5-chloro-2-{[(3R)-3-ethoxypyrrolidin-1-yl]methyl}-7,8-dihydro-6H-spiro[[1,3]oxazolo[5,4-f]quinazoline-9,1′-cyclohexane]-7-one; 5-chloro-2-{[(3R)-3-(2-hydroxypropan-2-yl)pyrrolidin-1-yl]methyl}-7,8-dihydro-6H-spiro[[1,3]oxazolo[5,4-f]quinazoline-9,1′-cyclohexane]-7-one; 5-chloro-2-{[(3R)-3-hydroxypyrrolidin-1-yl]methyl}-7,8-dihydro-6H-spiro[[1,3]oxazolo[5,4-f]quinazoline-9,1′-cyclohexane]-7-one; 5-chloro-2-({[2-(dimethylamino)ethyl](methyl)amino}methyl)-7,8-dihydro-6H-spiro[[1,3]oxazolo[5,4-f]quinazoline-9,1′-cyclohexane]-7-one; 3-({5-chloro-7-oxo-7,8-dihydro-6H-spiro[[1,3]oxazolo[5,4-f]quinazoline-9,1′-cyclohexane]-2-ylmethyl}(methyl)amino)-N,N-dimethylpropanamide; 5-chloro-2-({methyl[2-(morpholin-4-yl)ethyl]amino}methyl)-7,8-dihydro-6H-spiro[[1,3]oxazolo[5,4-f]quinazoline-9,1′-cyclohexane]-7-one; 5-chloro-2-[(3-methoxypyrrolidin-1-yl)methyl]-7,8-dihydro-6H-spiro[[1,3]oxazolo[5,4-f]quinazoline-9,1′-cyclohexane]-7-one; 5-chloro-2-{[4-(2,2,2-trifluoroethyl)piperazin-1-yl]methyl}-7,8-dihydro-6H-spiro[[1,3]oxazolo[5,4-f]quinazoline-9,1′-cyclohexane]-7-one; methyl 2-({5-chloro-7-oxo-7,8-dihydro-6H-spiro[[1,3]oxazolo[5,4-f]quinazoline-9,1′-cyclohexane]-2-ylmethyl}(methyl)amino)acetate; 2-[(8aR)-octahydropyrrolo[1,2-a]piperazin-2-ylmethyl]-5-chloro-7,8-dihydro-6H-spiro[[1,3]oxazolo[5,4-f]quinazoline-9,1′-cyclohexane]-7-one; 5-chloro-2-{[methyl(oxan-4-yl)amino]methyl}-7,8-dihydro-6H-spiro[[1,3]oxazolo[5,4-f]quinazoline-9,1′-cyclohexane]-7-one; methyl 1-{5-chloro-7-oxo-7,8-dihydro-6H-spiro[[1,3]oxazolo[5,4-f]quinazoline-9,1′-cyclohexane]-2-ylmethyl}piperidine-3-carboxylate; methyl 2-({5-chloro-7-oxo-7,8-dihydro-6H-spiro[[1,3]oxazolo[5,4-f]quinazoline-9,1′-cyclohexane]-2-ylmethyl}amino)acetate; 5-chloro-2-{[(3S)-3-methoxypyrrolidin-1-yl]methyl}-7,8-dihydro-6H-spiro[[1,3]oxazolo[5,4-f]quinazoline-9,1′-cyclohexane]-7-one; 5-chloro-2-{[(oxan-3-yl)amino]methyl}-7,8-dihydro-6H-spiro[[1,3]oxazolo[5,4-f]quinazoline-9,1′-cyclohexane]-7-one; 5-chloro-2-{[(3R)-3-methoxypyrrolidin-1-yl]methyl}-7,8-dihydro-6H-spiro[[1,3]oxazolo[5,4-f]quinazoline-9,1′-cyclohexane]-7-one; 5-chloro-2-{[(3S)-3-(methoxymethyl)pyrrolidin-1-yl]methyl}-7,8-dihydro-6H-spiro[[1,3]oxazolo[5,4-f]quinazoline-9,1′-cyclohexane]-7-one; 5-chloro-2-{[(3R)-3-(methoxymethyl)pyrrolidin-1-yl]methyl}-7,8-dihydro-6H-spiro[[1,3]oxazolo[5,4-f]quinazoline-9,1′-cyclohexane]-7-one; methyl (3R)-1-{5-chloro-7-oxo-7,8-dihydro-6H-spiro[[1,3]oxazolo[5,4-f]quinazoline-9,1′-cyclohexane]-2-ylmethyl}piperidine-3-carboxylate; 5-chloro-2-({[2-(trifluoromethoxy)ethyl]amino}methyl)-7,8-dihydro-6H-spiro[[1,3]oxazolo[5,4-f]quinazoline-9,1′-cyclohexane]-7-one; 5-chloro-2-[(4-ethylpiperazin-1-yl)methyl]-7,8-dihydro-6H-spiro[[1,3]oxazolo[5,4-f]quinazoline-9,1′-cyclohexane]-7-one; 5-chloro-2-{[3-(dimethylamino)pyrrolidin-1-yl]methyl}-7,8-dihydro-6H-spiro[[1,3]oxazolo[5,4-f]quinazoline-9,1′-cyclohexane]-7-one; 2-({5-chloro-7-oxo-7,8-dihydro-6H-spiro[[1,3]oxazolo[5,4-f]quinazoline-9,1′-cyclohexane]-2-ylmethyl}amino)-N,N-diethylacetamide; (2S)-2-({5-chloro-7-oxo-7,8-dihydro-6H-spiro[[1,3]oxazolo[5,4-f]quinazoline-9,1′-cyclohexane]-2-ylmethyl}amino)-N,N-dimethylpropanamide; (2R)-2-({5-chloro-7-oxo-7,8-dihydro-6H-spiro[[1,3]oxazolo[5,4-f]quinazoline-9,1′-cyclohexane]-2-ylmethyl}amino)-N,N-dimethylpropanamide; 5-chloro-2-({[2-oxo-2-(pyrrolidin-1-yl)ethyl]amino}methyl)-7,8-dihydro-6H-spiro[[1,3]oxazolo[5,4-f]quinazoline-9,1′-cyclohexane]-7-one; 5-chloro-2-{[(oxan-4-yl)amino]methyl}-7,8-dihydro-6H-spiro[[1,3]oxazolo[5,4-f]quinazoline-9,1′-cyclohexane]-7-one; 5-chloro-2-[({3-oxabicyclo[3.1.0]hexan-6-yl}amino)methyl]-7,8-dihydro-6H-spiro[[1,3]oxazolo[5,4-f]quinazoline-9,1′-cyclohexane]-7-one; 5-chloro-2-{[(oxepan-4-yl)amino]methyl}-7,8-dihydro-6H-spiro[[1,3]oxazolo[5,4-f]quinazoline-9,1′-cyclohexane]-7-one; 5-chloro-2-{[(oxolan-3-ylmethyl)amino]methyl}-7,8-dihydro-6H-spiro[[1,3]oxazolo[5,4-f]quinazoline-9,1′-cyclohexane]-7-one; 5-chloro-2-{[(3,3-difluoro-2-hydroxypropyl)amino]methyl}-7,8-dihydro-6H-spiro[[1,3]oxazolo[5,4-f]quinazoline-9,1′-cyclohexane]-7-one; 5-chloro-2-{[(4-cyclopropyloxan-4-yl)amino]methyl}-7,8-dihydro-6H-spiro[[1,3]oxazolo[5,4-f]quinazoline-9,1′-cyclohexane]-7-one; 5-chloro-2-{[(2,6-dimethyloxan-4-yl)amino]methyl}-7,8-dihydro-6H-spiro[[1,3]oxazolo[5,4-f]quinazoline-9,1′-cyclohexane]-7-one; 5-chloro-2-({[(3S)-oxan-3-yl]amino}methyl)-7,8-dihydro-6H-spiro[[1,3]oxazolo[5,4-f]quinazoline-9,1′-cyclohexane]-7-one; 5-chloro-2-({[(3S)-oxolan-3-yl]amino}methyl)-7,8-dihydro-6H-spiro[[1,3]oxazolo[5,4-f]quinazoline-9,1′-cyclohexane]-7-one; 5-chloro-2-{[(3-methyloxolan-3-yl)amino]methyl}-7,8-dihydro-6H-spiro[[1,3]oxazolo[5,4-f]quinazoline-9,1′-cyclohexane]-7-one; 5-chloro-2-({[(3R)-oxolan-3-yl]amino}methyl)-7,8-dihydro-6H-spiro[[1,3]oxazolo[5,4-f]quinazoline-9,1′-cyclohexane]-7-one; 5-chloro-2-{[4-(2-methoxyethoxy)piperidin-1-yl]methyl}-7,8-dihydro-6H-spiro[[1,3]oxazolo[5,4-f]quinazoline-9,1′-cyclohexane]-7-one; 2-[(8aS)-octahydropyrrolo[1,2-a]piperazin-2-ylmethyl]-5-chloro-7,8-dihydro-6H-spiro[[1,3]oxazolo[5,4-f]quinazoline-9,1′-cyclohexane]-7-one; methyl (3R)-1-{5-chloro-7-oxo-7,8-dihydro-6H-spiro[[1,3]oxazolo[5,4-f]quinazoline-9,1′-cyclohexane]-2-ylmethyl}pyrrolidine-3-carboxylate; 5-chloro-2-{[4-(2,2-difluoroethyl)piperazin-1-yl]methyl}-7,8-dihydro-6H-spiro[[1,3]oxazolo[5,4-f]quinazoline-9,1′-cyclohexane]-7-one; 5-chloro-2-({[(3-methyloxolan-3-yl)methyl]amino}methyl)-7,8-dihydro-6H-spiro[[1,3]oxazolo[5,4-f]quinazoline-9,1′-cyclohexane]-7-one; 5-chloro-2-({[(3R)-oxan-3-yl]amino}methyl)-7,8-dihydro-6H-spiro[[1,3]oxazolo[5,4-f]quinazoline-9,1′-cyclohexane]-7-one; 5-chloro-2-({[(3R)-oxolan-3-ylmethyl]amino}methyl)-7,8-dihydro-6H-spiro[[1,3]oxazolo[5,4-f]quinazoline-9,1′-cyclohexane]-7-one; 5-chloro-2-{[(3-fluorooxan-4-yl)amino]methyl}-7,8-dihydro-6H-spiro[[1,3]oxazolo[5,4-f]quinazoline-9,1′-cyclohexane]-7-one; 5-chloro-2-{[(2,2-difluoro-3-hydroxypropyl)amino]methyl}-7,8-dihydro-6H-spiro[[1,3]oxazolo[5,4-f]quinazoline-9,1′-cyclohexane]-7-one; 5-chloro-2-{[(2,2-difluorocyclopentyl)amino]methyl}-7,8-dihydro-6H-spiro[[1,3]oxazolo[5,4-f]quinazoline-9,1′-cyclohexane]-7-one; 5-chloro-2-({[4-(methoxymethyl)oxan-4-yl]amino}methyl)-7,8-dihydro-6H-spiro[[1,3]oxazolo[5,4-f]quinazoline-9,1′-cyclohexane]-7-one; 5-chloro-2-({[(3S)-oxolan-3-ylmethyl]amino}methyl)-7,8-dihydro-6H-spiro[[1,3]oxazolo[5,4-f]quinazoline-9,1′-cyclohexane]-7-one; 5-chloro-4′,4′-difluoro-2-[(1S,4S)-2-oxa-5-azabicyclo[2.2.1]heptan-5-ylmethyl]-7,8-dihydro-6H-spiro[[1,3]oxazolo[5,4-f]quinazoline-9,1′-cyclohexane]-7-one; 5-chloro-4′,4′-difluoro-2-({[(3S)-oxolan-3-ylmethyl]amino}methyl)-7,8-dihydro-6H-spiro[[1,3]oxazolo[5,4-f]quinazoline-9,1′-cyclohexane]-7-one; 5-chloro-2-{[(1,3-oxazol-2-ylmethyl)amino]methyl}-7,8-dihydro-6H-spiro[[1,3]oxazolo[5,4-f]quinazoline-9,1′-cyclohexane]-7-one; 5-chloro-2-{[(1,3-oxazol-5-ylmethyl)amino]methyl}-7,8-dihydro-6H-spiro[[1,3]oxazolo[5,4-f]quinazoline-9,1′-cyclohexane]-7-one; 5-chloro-2-({[2-(1,2-oxazol-3-yl)ethyl]amino}methyl)-7,8-dihydro-6H-spiro[[1,3]oxazolo[5,4-f]quinazoline-9,1′-cyclohexane]-7-one; 5-chloro-2-{[(1,3-oxazol-4-ylmethyl)amino]methyl}-7,8-dihydro-6H-spiro[[1,3]oxazolo[5,4-f]quinazoline-9,1′-cyclohexane]-7-one; 5-chloro-2-({[(1-methyl-1H-pyrazol-3-yl)methyl]amino}methyl)-7,8-dihydro-6H-spiro[[1,3]oxazolo[5,4-f]quinazoline-9,1′-cyclohexane]-7-one; 5-chloro-2-{[(1,2-oxazol-5-ylmethyl)amino]methyl}-7,8-dihydro-6H-spiro[[1,3]oxazolo[5,4-f]quinazoline-9,1′-cyclohexane]-7-one; 2-{5-chloro-7-oxo-7,8-dihydro-6H-spiro[[1,3]oxazolo[5,4-f]quinazoline-9,1′-cyclohexane]-2-ylmethoxy}-N,N-dimethylacetamide; 5-chloro-2-[(oxan-4-yloxy)methyl]-7,8-dihydro-6H-spiro[[1,3]oxazolo[5,4-f]quinazoline-9,1′-cyclohexane]-7-one; 5-chloro-2-{[(1-methylpiperidin-4-yl)oxy]methyl}-7,8-dihydro-6H-spiro[[1,3]oxazolo[5,4-f]quinazoline-9,1′-cyclohexane]-7-one; 5-chloro-N-[2-(dimethylamino)ethyl]-7-oxo-7,8-dihydro-6H-spiro[[1,3]oxazolo[5,4-f]quinazoline-9,1′-cyclohexane]-2-carboxamide; 5-chloro-2-(morpholine-4-carbonyl)-7,8-dihydro-6H-spiro[[1,3]oxazolo[5,4-f]quinazoline-9,1′-cyclohexane]-7-one; 5-chloro-N,N-dimethyl-7-oxo-7,8-dihydro-6H-spiro[[1,3]oxazolo[5,4-f]quinazoline-9,1′-cyclohexane]-2-carboxamide; 5-chloro-2-(4-methylpiperazine-1-carbonyl)-7,8-dihydro-6H-spiro[[1,3]oxazolo[5,4-f]quinazoline-9,1′-cyclohexane]-7-one; 5-chloro-N-(2-methoxyethyl)-7-oxo-7,8-dihydro-6H-spiro[[1,3]oxazolo[5,4-f]quinazoline-9,1′-cyclohexane]-2-carboxamide; 5-chloro-2-[(1S,4S)-2-oxa-5-azabicyclo[2.2.1]heptane-5-carbonyl]-7,8-dihydro-6H-spiro[[1,3]oxazolo[5,4-f]quinazoline-9,1′-cyclohexane]-7-one; 5-chloro-N-(2-methoxyethyl)-N-methyl-7-oxo-7,8-dihydro-6H-spiro[[1,3]oxazolo[5,4-f]quinazoline-9,1′-cyclohexane]-2-carboxamide; 5-chloro-N-methyl-N-(oxan-4-ylmethyl)-7-oxo-7,8-dihydro-6H-spiro[[1,3]oxazolo[5,4-f]quinazoline-9,1′-cyclohexane]-2-carboxamide; 5-chloro-2-[(3R)-3-ethoxypyrrolidine-1-carbonyl]-7,8-dihydro-6H-spiro[[1,3]oxazolo[5,4-f]quinazoline-9,1′-cyclohexane]-7-one; 2-{1-5-chloro-7-oxo-7,8-dihydro-6H-spiro[[1,3]oxazolo[5,4-f]quinazoline-9,1′-cyclohexane]-2-yl-N-methylformamido}-N,N-dimethylacetamide; 3-{1-5-chloro-7-oxo-7,8-dihydro-6H-spiro[[1,3]oxazolo[5,4-f]quinazoline-9,1′-cyclohexane]-2-yl-N-methylformamido}-N,N-dimethylpropanamide; 5-chloro-2-[(2S)-2-(methoxymethyl)pyrrolidine-1-carbonyl]-7,8-dihydro-6H-spiro[[1,3]oxazolo[5,4-f]quinazoline-9,1′-cyclohexane]-7-one; methyl 2-{1-5-chloro-7-oxo-7,8-dihydro-6H-spiro[[1,3]oxazolo[5,4-f]quinazoline-9,1′-cyclohexane]-2-yl-N-methylformamido}acetate; 5-chloro-N-methyl-N-[2-(methylamino)ethyl]-7-oxo-7,8-dihydro-6H-spiro[[1,3]oxazolo[5,4-f]quinazoline-9,1′-cyclohexane]-2-carboxamide; 5-chloro-N-[2-(dimethylamino)ethyl]-N-methyl-7-oxo-7,8-dihydro-6H-spiro[[1,3]oxazolo[5,4-f]quinazoline-9,1′-cyclohexane]-2-carboxamide; 5-chloro-2-[4-(dimethylamino)piperidine-1-carbonyl]-7,8-dihydro-6H-spiro[[1,3]oxazolo[5,4-f]quinazoline-9,1′-cyclohexane]-7-one; 5-chloro-2-[4-(2,2,2-trifluoroethyl)piperazine-1-carbonyl]-7,8-dihydro-6H-spiro[[1,3]oxazolo[5,4-f]quinazoline-9,1′-cyclohexane]-7-one; 5-chloro-2-[4-(2-methoxyethyl)piperazine-1-carbonyl]-7,8-dihydro-6H-spiro[[1,3]oxazolo[5,4-f]quinazoline-9,1′-cyclohexane]-7-one; 2-[(8aR)-octahydropyrrolo[1,2-a]piperazine-2-carbonyl]-5-chloro-7,8-dihydro-6H-spiro[[1,3]oxazolo[5,4-f]quinazoline-9,1′-cyclohexane]-7-one; 5-chloro-N-methyl-N-(oxan-4-yl)-7-oxo-7,8-dihydro-6H-spiro[[1,3]oxazolo[5,4-f]quinazoline-9,1′-cyclohexane]-2-carboxamide; 5-chloro-2-(3-methoxypyrrolidine-1-carbonyl)-7,8-dihydro-6H-spiro[[1,3]oxazolo[5,4-f]quinazoline-9,1′-cyclohexane]-7-one; 5-chloro-2-[(3R)-3-methoxypiperidine-1-carbonyl]-7,8-dihydro-6H-spiro[[1,3]oxazolo[5,4-f]quinazoline-9,1′-cyclohexane]-7-one; 5-chloro-N-(oxan-3-yl)-7-oxo-7,8-dihydro-6H-spiro[[1,3]oxazolo[5,4-f]quinazoline-9,1′-cyclohexane]-2-carboxamide; 5-chloro-2-[(3S)-3-methoxypyrrolidine-1-carbonyl]-7,8-dihydro-6H-spiro[[1,3]oxazolo[5,4-f]quinazoline-9,1′-cyclohexane]-7-one; 5-chloro-2-[(3R)-3-(methoxymethyl)pyrrolidine-1-carbonyl]-7,8-dihydro-6H-spiro[[1,3]oxazolo[5,4-f]quinazoline-9,1′-cyclohexane]-7-one; 5-chloro-2-[(3S)-3-(methoxymethyl)pyrrolidine-1-carbonyl]-7,8-dihydro-6H-spiro[[1,3]oxazolo[5,4-f]quinazoline-9,1′-cyclohexane]-7-one; 5-chloro-7-oxo-N-[2-(trifluoromethoxy)ethyl]-7,8-dihydro-6H-spiro[[1,3]oxazolo[5,4-f]quinazoline-9,1′-cyclohexane]-2-carboxamide; 5-chloro-2-[(3R)-3-methoxypyrrolidine-1-carbonyl]-7,8-dihydro-6H-spiro[[1,3]oxazolo[5,4-f]quinazoline-9,1′-cyclohexane]-7-one; 5-chloro-2-(4-ethylpiperazine-1-carbonyl)-7,8-dihydro-6H-spiro[[1,3]oxazolo[5,4-f]quinazoline-9,1′-cyclohexane]-7-one; 5-chloro-2-[4-(propan-2-yl)piperazine-1-carbonyl]-7,8-dihydro-6H-spiro[[1,3]oxazolo[5,4-f]quinazoline-9,1′-cyclohexane]-7-one; 5-chloro-2-[4-(2,2-difluoroethyl)piperazine-1-carbonyl]-7,8-dihydro-6H-spiro[[1,3]oxazolo[5,4-f]quinazoline-9,1′-cyclohexane]-7-one; 2-[(8aS)-octahydropyrrolo[1,2-a]piperazine-2-carbonyl]-5-chloro-7,8-dihydro-6H-spiro[[1,3]oxazolo[5,4-f]quinazoline-9,1′-cyclohexane]-7-one; 5-chloro-2-(piperazine-1-carbonyl)-7,8-dihydro-6H-spiro[[1,3]oxazolo[5,4-f]quinazoline-9,1′-cyclohexane]-7-one; 5-chloro-7-oxo-N-[(3R)-oxolan-3-ylmethyl]-7,8-dihydro-6H-spiro[[1,3]oxazolo[5,4-f]quinazoline-9,1′-cyclohexane]-2-carboxamide; 5-chloro-2-[4-(2-fluoroethyl)piperazine-1-carbonyl]-7,8-dihydro-6H-spiro[[1,3]oxazolo[5,4-f]quinazoline-9,1′-cyclohexane]-7-one; 5-chloro-N-(2,2-difluorocyclopentyl)-7-oxo-7,8-dihydro-6H-spiro[[1,3]oxazolo[5,4-f]quinazoline-9,1′-cyclohexane]-2-carboxamide; 5-chloro-N-[4-(methoxymethyl)oxan-4-yl]-7-oxo-7,8-dihydro-6H-spiro[[1,3]oxazolo[5,4-f]quinazoline-9,1′-cyclohexane]-2-carboxamide; 5-chloro-N-(3-fluorooxan-4-yl)-7-oxo-7,8-dihydro-6H-spiro[[1,3]oxazolo[5,4-f]quinazoline-9,1′-cyclohexane]-2-carboxamide; 5-chloro-N-(2,2-difluoro-3-hydroxypropyl)-7-oxo-7,8-dihydro-6H-spiro[[1,3]oxazolo[5,4-f]quinazoline-9,1′-cyclohexane]-2-carboxamide; 5-chloro-7-oxo-N-[(3S)-oxolan-3-ylmethyl]-7,8-dihydro-6H-spiro[[1,3]oxazolo[5,4-f]quinazoline-9,1′-cyclohexane]-2-carboxamide; 5-chloro-7-oxo-N-[(3S)-oxolan-3-yl]-7,8-dihydro-6H-spiro[[1,3]oxazolo[5,4-f]quinazoline-9,1′-cyclohexane]-2-carboxamide; 5-chloro-7-oxo-N-[(3R)-oxolan-3-yl]-7,8-dihydro-6H-spiro[[1,3]oxazolo[5,4-f]quinazoline-9,1′-cyclohexane]-2-carboxamide; 2-[(8aS)-octahydropyrrolo[1,2-a]piperazine-2-carbonyl]-5-chloro-4′,4′-difluoro-7,8-dihydro-6H-spiro[[1,3]oxazolo[5,4-f]quinazoline-9,1′-cyclohexane]-7-one; 2-[(4,4-difluoropiperidin-1-yl)methyl]-5-fluoro-7,8-dihydro-6H-spiro[[1,3]oxazolo[5,4-f]quinazoline-9,1′-cyclohexane]-7-one; 5-fluoro-2-{[(2-methoxyethyl)amino]methyl}-7,8-dihydro-6H-spiro[[1,3]oxazolo[5,4-f]quinazoline-9,1′-cyclohexane]-7-one; 2-({5-fluoro-7-oxo-7,8-dihydro-6H-spiro[[1,3]oxazolo[5,4-f]quinazoline-9,1′-cyclohexane]-2-ylmethyl}(methyl)amino)-N,N-dimethylacetamide; 2-({5-fluoro-7-oxo-7,8-dihydro-6H-spiro[[1,3]oxazolo[5,4-f]quinazoline-9,1′-cyclohexane]-2-ylmethyl}amino)-N,N-dimethylacetamide; 5-chloro-4′,4′-difluoro-2-({[(3S)-oxan-3-yl]amino}methyl)-7,8-dihydro-6H-spiro[[1,3]oxazolo[5,4-f]quinazoline-9,1′-cyclohexane]-7-one; 5-chloro-4′,4′-difluoro-2-({[(3R)-oxolan-3-ylmethyl]amino}methyl)-7,8-dihydro-6H-spiro[[1,3]oxazolo[5,4-f]quinazoline-9,1′-cyclohexane]-7-one; 5-chloro-4′,4′-difluoro-2-({[(3S)-oxolan-3-yl]amino}methyl)-7,8-dihydro-6H-spiro[[1,3]oxazolo[5,4-f]quinazoline-9,1′-cyclohexane]-7-one; 5-chloro-4′,4′-difluoro-2-({[(3R)-oxan-3-yl]amino}methyl)-7,8-dihydro-6H-spiro[[1,3]oxazolo[5,4-f]quinazoline-9,1′-cyclohexane]-7-one; and pharmaceutically acceptable salts thereof; or
a compound selected from the group consisting of methyl 5-chloro-7-oxo-7,8-dihydro-6H-spiro[[1,3]oxazolo[5,4-f]quinazoline-9,1′-cyclohexane]-2-carboxylate; 5-chloro-7-oxo-7,8-dihydro-6H-spiro[[1,3]oxazolo[5,4-f]quinazoline-9,1′-cyclohexane]-2-carboxamide; 5-chloro-N-[2-(dimethylamino)ethyl]-7-oxo-7,8-dihydro-6H-spiro[[1,3]oxazolo[5,4-f]quinazoline-9,1′-cyclohexane]-2-carboxamide; 5-chloro-2-(morpholine-4-carbonyl)-7,8-dihydro-6H-spiro[[1,3]oxazolo[5,4-f]quinazoline-9,1′-cyclohexane]-7-one; 5-chloro-7-oxo-7,8-dihydro-6H-spiro[[1,3]oxazolo[5,4-f]quinazoline-9,1′-cyclohexane]-2-carboxylic acid; 5-chloro-N,N-dimethyl-7-oxo-7,8-dihydro-6H-spiro[[1,3]oxazolo[5,4-f]quinazoline-9,1′-cyclohexane]-2-carboxamide; 5-chloro-2-(4-methylpiperazine-1-carbonyl)-7,8-dihydro-6H-spiro[[1,3]oxazolo[5,4-f]quinazoline-9,1′-cyclohexane]-7-one; 5-chloro-2-(4-methoxypiperidine-1-carbonyl)-7,8-dihydro-6H-spiro[[1,3]oxazolo[5,4-f]quinazoline-9,1′-cyclohexane]-7-one; 5-chloro-N-(2-methoxyethyl)-7-oxo-7,8-dihydro-6H-spiro[[1,3]oxazolo[5,4-f]quinazoline-9,1′-cyclohexane]-2-carboxamide; 5-chloro-2-[(1 S,4S)-2-oxa-5-azabicyclo[2.2.1]heptane-5-carbonyl]-7,8-dihydro-6H-spiro[[1,3]oxazolo[5,4-f]quinazoline-9,1′-cyclohexane]-7-one; 5-chloro-N-(2-methoxyethyl)-N-methyl-7-oxo-7,8-dihydro-6H-spiro[[1,3]oxazolo[5,4-f]quinazoline-9,1′-cyclohexane]-2-carboxamide; 5-chloro-N-methyl-N-(oxan-4-ylmethyl)-7-oxo-7,8-dihydro-6H-spiro[[1,3]oxazolo[5,4-f]quinazoline-9,1′-cyclohexane]-2-carboxamide; 5-chloro-2-[(3R)-3-ethoxypyrrolidine-1-carbonyl]-7,8-dihydro-6H-spiro[[1,3]oxazolo[5,4-f]quinazoline-9,1′-cyclohexane]-7-one; 2-{1-5-chloro-7-oxo-7,8-dihydro-6H-spiro[[1,3]oxazolo[5,4-f]quinazoline-9,1′-cyclohexane]-2-yl-N-methylformamido}-N,N-dimethylacetamide; 3-{1-5-chloro-7-oxo-7,8-dihydro-6H-spiro[[1,3]oxazolo[5,4-f]quinazoline-9,1′-cyclohexane]-2-yl-N-methylformamido}-N,N-dimethylpropanamide; 5-chloro-2-[(2S)-2-(methoxymethyl)pyrrolidine-1-carbonyl]-7,8-dihydro-6H-spiro[[1,3]oxazolo[5,4-f]quinazoline-9,1′-cyclohexane]-7-one; methyl 2-{1-5-chloro-7-oxo-7,8-dihydro-6H-spiro[[1,3]oxazolo[5,4-f]quinazoline-9,1′-cyclohexane]-2-yl-N-methylformamido}acetate; 5-chloro-N-methyl-N-[2-(methylamino)ethyl]-7-oxo-7,8-dihydro-6H-spiro[[1,3]oxazolo[5,4-f]quinazoline-9,1′-cyclohexane]-2-carboxamide; 5-chloro-N-[2-(dimethylamino)ethyl]-N-methyl-7-oxo-7,8-dihydro-6H-spiro[[1,3]oxazolo[5,4-f]quinazoline-9,1′-cyclohexane]-2-carboxamide; 5-chloro-2-[4-(dimethylamino)piperidine-1-carbonyl]-7,8-dihydro-6H-spiro[[1,3]oxazolo[5,4-f]quinazoline-9,1′-cyclohexane]-7-one; 5-chloro-2-[4-(2,2,2-trifluoroethyl)piperazine-1-carbonyl]-7,8-dihydro-6H-spiro[[1,3]oxazolo[5,4-f]quinazoline-9,1′-cyclohexane]-7-one; 5-chloro-2-[4-(2-methoxyethyl)piperazine-1-carbonyl]-7,8-dihydro-6H-spiro[[1,3]oxazolo[5,4-f]quinazoline-9,1′-cyclohexane]-7-one; 2-[(8aR)-octahydropyrrolo[1,2-a]piperazine-2-carbonyl]-5-chloro-7,8-dihydro-6H-spiro[[1,3]oxazolo[5,4-f]quinazoline-9,1′-cyclohexane]-7-one; 5-chloro-N-methyl-N-(oxan-4-yl)-7-oxo-7,8-dihydro-6H-spiro[[1,3]oxazolo[5,4-f]quinazoline-9,1′-cyclohexane]-2-carboxamide; 5-chloro-2-(3-methoxypyrrolidine-1-carbonyl)-7,8-dihydro-6H-spiro[[1,3]oxazolo[5,4-f]quinazoline-9,1′-cyclohexane]-7-one; 5-chloro-2-[(3R)-3-methoxypiperidine-1-carbonyl]-7,8-dihydro-6H-spiro[[1,3]oxazolo[5,4-f]quinazoline-9,1′-cyclohexane]-7-one; 5-chloro-N-(oxan-3-yl)-7-oxo-7,8-dihydro-6H-spiro[[1,3]oxazolo[5,4-f]quinazoline-9,1′-cyclohexane]-2-carboxamide; 5-chloro-2-[(3S)-3-methoxypyrrolidine-1-carbonyl]-7,8-dihydro-6H-spiro[[1,3]oxazolo[5,4-f]quinazoline-9,1′-cyclohexane]-7-one; 5-chloro-2-[(3R)-3-(methoxymethyl)pyrrolidine-1-carbonyl]-7,8-dihydro-6H-spiro[[1,3]oxazolo[5,4-f]quinazoline-9,1′-cyclohexane]-7-one; 5-chloro-2-[(3S)-3-(methoxymethyl)pyrrolidine-1-carbonyl]-7,8-dihydro-6H-spiro[[1,3]oxazolo[5,4-f]quinazoline-9,1′-cyclohexane]-7-one; 5-chloro-7-oxo-N-[2-(trifluoromethoxy)ethyl]-7,8-dihydro-6H-spiro[[1,3]oxazolo[5,4-f]quinazoline-9,1′-cyclohexane]-2-carboxamide; 5-chloro-2-[(3R)-3-methoxypyrrolidine-1-carbonyl]-7,8-dihydro-6H-spiro[[1,3]oxazolo[5,4-f]quinazoline-9,1′-cyclohexane]-7-one; 5-chloro-2-(4-ethylpiperazine-1-carbonyl)-7,8-dihydro-6H-spiro[[1,3]oxazolo[5,4-f]quinazoline-9,1′-cyclohexane]-7-one; 5-chloro-2-[4-(propan-2-yl)piperazine-1-carbonyl]-7,8-dihydro-6H-spiro[[1,3]oxazolo[5,4-f]quinazoline-9,1′-cyclohexane]-7-one; 5-chloro-2-[4-(2,2-difluoroethyl)piperazine-1-carbonyl]-7,8-dihydro-6H-spiro[[1,3]oxazolo[5,4-f]quinazoline-9,1′-cyclohexane]-7-one; 2-[(8aS)-octahydropyrrolo[1,2-a]piperazine-2-carbonyl]-5-chloro-7,8-dihydro-6H-spiro[[1,3]oxazolo[5,4-f]quinazoline-9,1′-cyclohexane]-7-one; 5-chloro-2-(piperazine-1-carbonyl)-7,8-dihydro-6H-spiro[[1,3]oxazolo[5,4-f]quinazoline-9,1′-cyclohexane]-7-one; 5-chloro-7-oxo-N-[(3R)-oxolan-3-ylmethyl]-7,8-dihydro-6H-spiro[[1,3]oxazolo[5,4-f]quinazoline-9,1′-cyclohexane]-2-carboxamide; 5-chloro-2-[4-(2-fluoroethyl)piperazine-1-carbonyl]-7,8-dihydro-6H-spiro[[1,3]oxazolo[5,4-f]quinazoline-9,1′-cyclohexane]-7-one; 5-chloro-N-(2,2-difluorocyclopentyl)-7-oxo-7,8-dihydro-6H-spiro[[1,3]oxazolo[5,4-f]quinazoline-9,1′-cyclohexane]-2-carboxamide; 5-chloro-N-[4-(methoxymethyl)oxan-4-yl]-7-oxo-7,8-dihydro-6H-spiro[[1,3]oxazolo[5,4-f]quinazoline-9,1′-cyclohexane]-2-carboxamide; 5-chloro-N-(3-fluorooxan-4-yl)-7-oxo-7,8-dihydro-6H-spiro[[1,3]oxazolo[5,4-f]quinazoline-9,1′-cyclohexane]-2-carboxamide; 5-chloro-N-(2,2-difluoro-3-hydroxypropyl)-7-oxo-7,8-dihydro-6H-spiro[[1,3]oxazolo[5,4-f]quinazoline-9,1′-cyclohexane]-2-carboxamide; 5-chloro-7-oxo-N-[(3S)-oxolan-3-ylmethyl]-7,8-dihydro-6H-spiro[[1,3]oxazolo[5,4-f]quinazoline-9,1′-cyclohexane]-2-carboxamide; 5-chloro-7-oxo-N-[(3S)-oxolan-3-yl]-7,8-dihydro-6H-spiro[[1,3]oxazolo[5,4-f]quinazoline-9,1′-cyclohexane]-2-carboxamide; 5-chloro-7-oxo-N-[(3R)-oxolan-3-yl]-7,8-dihydro-6H-spiro[[1,3]oxazolo[5,4-f]quinazoline-9,1′-cyclohexane]-2-carboxamide; methyl 5-chloro-4′,4′-difluoro-7-oxo-7,8-dihydro-6H-spiro[[1,3]oxazolo[5,4-f]quinazoline-9,1′-cyclohexane]-2-carboxylate; 2-[(8aS)-octahydropyrrolo[1,2-a]piperazine-2-carbonyl]-5-chloro-4′,4′-difluoro-7,8-dihydro-6H-spiro[[1,3]oxazolo[5,4-f]quinazoline-9,1′-cyclohexane]-7-one; and pharmaceutically acceptable salts thereof; or
a compound selected from the group consisting of: 5-chloro-2-methyl-7,8-dihydro-6H-spiro[[1,3]oxazolo[5,4-f]quinazoline-9,1′-cyclohexan]-7-one; 5-chloro-2-(chloromethyl)-7,8-dihydro-6H-spiro[[1,3]oxazolo[5,4-f]quinazoline-9,1′-cyclohexan]-7-one; 5-chloro-2-(methoxymethyl)-7,8-dihydro-6H-spiro[[1,3]oxazolo[5,4-f]quinazoline-9,1′-cyclohexan]-7-one; 5-chloro-2-(hydroxymethyl)-7,8-dihydro-6H-spiro[[1,3]oxazolo[5,4-f]quinazoline-9,1′-cyclohexan]-7-one; 5-chloro-2-propyl-7,8-dihydro-6H-spiro[[1,3]oxazolo[5,4-f]quinazoline-9,1′-cyclohexan]-7-one; 5-chloro-2-ethyl-7,8-dihydro-6H-spiro[[1,3]oxazolo[5,4-f]quinazoline-9,1′-cyclohexan]-7-one; 5-chloro-2-[(dimethylamino)methyl]-7,8-dihydro-6H-spiro[[1,3]oxazolo[5,4-f]quinazoline-9,1′-cyclohexan]-7-one; 5-chloro-2-[(4-methylpiperazin-1-yl)methyl]-7,8-dihydro-6H-spiro[[1,3]oxazolo[5,4-f]quinazoline-9,1′-cyclohexan]-7-one; 5-chloro-2-[(morpholin-4-yl)methyl]-7,8-dihydro-6H-spiro[[1,3]oxazolo[5,4-f]quinazoline-9,1′-cyclohexan]-7-one; 5-chloro-2-[(4-hydroxypiperidin-1-yl)methyl]-7,8-dihydro-6H-spiro[[1,3]oxazolo[5,4-f]quinazoline-9,1′-cyclohexan]-7-one; 5-chloro-2-[(methylamino)methyl]-7,8-dihydro-6H-spiro[[1,3]oxazolo[5,4-f]quinazoline-9,1′-cyclohexan]-7-one; 5-chloro-2-[(cyclopropylamino)methyl]-7,8-dihydro-6H-spiro[[1,3]oxazolo[5,4-f]quinazoline-9,1′-cyclohexan]-7-one; 5-chloro-2-[(piperazin-1-yl)methyl]-7,8-dihydro-6H-spiro[[1,3]oxazolo[5,4-f]quinazoline-9,1′-cyclohexan]-7-one; 5-chloro-2-[(pyrrolidin-1-yl)methyl]-7,8-dihydro-6H-spiro[[1,3]oxazolo[5,4-f]quinazoline-9,1′-cyclohexan]-7-one; 5-chloro-2-(2-methoxyethyl)-7,8-dihydro-6H-spiro[[1,3]oxazolo[5,4-f]quinazoline-9,1′-cyclohexan]-7-one; 5-chloro-2-(2-hydroxyethyl)-7,8-dihydro-6H-spiro[[1,3]oxazolo[5,4-f]quinazoline-9,1′-cyclohexan]-7-one; 5-chloro-2-{[(2-methoxyethyl)amino]methyl}-7,8-dihydro-6H-spiro[[1,3]oxazolo[5,4-f]quinazoline-9,1′-cyclohexan]-7-one; 5-chloro-2-{[(propan-2-yl)amino]methyl}-7,8-dihydro-6H-spiro[[1,3]oxazolo[5,4-f]quinazoline-9,1′-cyclohexan]-7-one; 5-chloro-2-[(ethylamino)methyl]-7,8-dihydro-6H-spiro[[1,3]oxazolo[5,4-f]quinazoline-9,1′-cyclohexan]-7-one; 5-chloro-2-{[(2-hydroxyethyl)amino]methyl}-7,8-dihydro-6H-spiro[[1,3]oxazolo[5,4-f]quinazoline-9,1′-cyclohexan]-7-one; 5-chloro-2-[(4,4-difluoropiperidin-1-yl)methyl]-7,8-dihydro-6H-spiro[[1,3]oxazolo[5,4-f]quinazoline-9,1′-cyclohexan]-7-one; 5-chloro-2-{[(2,2,2-trifluoroethyl)amino]methyl}-7,8-dihydro-6H-spiro[[1,3]oxazolo[5,4-f]quinazoline-9,1′-cyclohexan]-7-one; 5-chloro-2-{[(2-hydroxyethyl)(methyl)amino]methyl}-7,8-dihydro-6H-spiro[[1,3]oxazolo[5,4-f]quinazoline-9,1′-cyclohexan]-7-one; 5-chloro-2-[(3,3-difluoroazetidin-1-yl)methyl]-7,8-dihydro-6H-spiro[[1,3]oxazolo[5,4-f]quinazoline-9,1′-cyclohexan]-7-one; 5-chloro-2-[(3,3-difluoropyrrolidin-1-yl)methyl]-7,8-dihydro-6H-spiro[[1,3]oxazolo[5,4-f]quinazoline-9,1′-cyclohexan]-7-one; 2-[(4-acetylpiperazin-1-yl)methyl]-5-chloro-7,8-dihydro-6H-spiro[[1,3]oxazolo[5,4-f]quinazoline-9,1′-cyclohexan]-7-one; 5-chloro-2-[(4-propanoylpiperazin-1-yl)methyl]-7,8-dihydro-6H-spiro[[1,3]oxazolo[5,4-f]quinazoline-9,1′-cyclohexan]-7-one; 5-chloro-2-({2-oxa-8-azaspiro[4.5]decan-8-yl}methyl)-7,8-dihydro-6H-spiro[[1,3]oxazolo[5,4-f]quinazoline-9,1′-cyclohexan]-7-one; 5-chloro-2-{[4-(oxetan-3-yl)piperazin-1-yl]methyl}-7,8-dihydro-6H-spiro[[1,3]oxazolo[5,4-f]quinazoline-9,1′-cyclohexan]-7-one; 2-[({5-chloro-7-oxo-7,8-dihydro-6H-spiro[[1,3]oxazolo[5,4-f]quinazoline-9,1′-cyclohexan]-2-yl}methyl)amino]-N,N-dimethylacetamide; tert-butyl 2-{[({5-chloro-7-oxo-7,8-dihydro-6H-spiro[[1,3]oxazolo[5,4-f]quinazoline-9,1′-cyclohexan]-2-yl}methyl)amino]methyl}piperidine-1-carboxylate; 5-chloro-2-({[(3,5-difluoropyridin-2-yl)methyl]amino}methyl)-7,8-dihydro-6H-spiro[[1,3]oxazolo[5,4-f]quinazoline-9,1′-cyclohexan]-7-one; 5-chloro-2-({[(piperidin-2-yl)methyl]amino}methyl)-7,8-dihydro-6H-spiro[[1,3]oxazolo[5,4-f]quinazoline-9,1′-cyclohexan]-7-one; 5-chloro-2-({[2-oxo-2-(piperidin-1-yl)ethyl]amino}methyl)-7,8-dihydro-6H-spiro[[1,3]oxazolo[5,4-f]quinazoline-9,1′-cyclohexan]-7-one; 2-[({5-chloro-7-oxo-7,8-dihydro-6H-spiro[[1,3]oxazolo[5,4-f]quinazoline-9,1′-cyclohexan]-2-yl}methyl)amino]-N-ethyl-N-methylacetamide; 5-chloro-2-[(4-methyl-3-oxopiperazin-1-yl)methyl]-7,8-dihydro-6H-spiro[[1,3]oxazolo[5,4-f]quinazoline-9,1′-cyclohexan]-7-one; 5-chloro-2-[(4-ethyl-3-oxopiperazin-1-yl)methyl]-7,8-dihydro-6H-spiro[[1,3]oxazolo[5,4-f]quinazoline-9,1′-cyclohexan]-7-one; 5-chloro-2-({[(pyridin-2-yl)methyl]amino}methyl)-7,8-dihydro-6H-spiro[[1,3]oxazolo[5,4-f]quinazoline-9,1′-cyclohexan]-7-one; 5-chloro-2-({[(pyrimidin-2-yl)methyl]amino}methyl)-7,8-dihydro-6H-spiro[[1,3]oxazolo[5,4-f]quinazoline-9,1′-cyclohexan]-7-one; 5-chloro-2-({[(5-methoxypyridin-3-yl)methyl]amino}methyl)-7,8-dihydro-6H-spiro[[1,3]oxazolo[5,4-f]quinazoline-9,1′-cyclohexan]-7-one; 5-chloro-2-[(2,4-dimethyl-3-oxopiperazin-1-yl)methyl]-7,8-dihydro-6H-spiro[[1,3]oxazolo[5,4-f]quinazoline-9,1′-cyclohexan]-7-one; ethyl 1-({5-chloro-7-oxo-7,8-dihydro-6H-spiro[[1,3]oxazolo[5,4f]quinazoline-9,1′-cyclohexan]-2-yl}methyl)piperidine-4-carboxylate; 5-chloro-2-({1-oxo-2-oxa-8-azaspiro[4.5]decan-8-yl}methyl)-7,8-dihydro-6H-spiro[[1,3]oxazolo[5,4-f]quinazoline-9,1′-cyclohexan]-7-one; 2-[(4-acetylpiperidin-1-yl)methyl]-5-chloro-7,8-dihydro-6H-spiro[[1,3]oxazolo[5,4-f]quinazoline-9,1′-cyclohexan]-7-one; methyl 1-({5-chloro-7-oxo-7,8-dihydro-6H-spiro[[1,3]oxazolo[5,4f]quinazoline-9,1′-cyclohexan]-2-yl}methyl)piperidine-4-carboxylate; 5-chloro-2-({[(5-fluoropyridin-2-yl)methyl]amino}methyl)-7,8-dihydro-6H-spiro[[1,3]oxazolo[5,4-f]quinazoline-9,1′-cyclohexan]-7-one; 5-chloro-2-{[3-(2-methylpropoxy)azetidin-1-yl]methyl}-7,8-dihydro-6H-spiro[[1,3]oxazolo[5,4-f]quinazoline-9,1′-cyclohexan]-7-one; 5-chloro-2-[(4-ethoxypiperidin-1-yl)methyl]-7,8-dihydro-6H-spiro[[1,3]oxazolo[5,4-f]quinazoline-9,1′-cyclohexan]-7-one; 5-chloro-2-{[4-(cyclopropylmethoxy)piperidin-1-yl]methyl}-7,8-dihydro-6H-spiro[[1,3]oxazolo[5,4-f]quinazoline-9,1′-cyclohexan]-7-one; 5-chloro-2-{[4-hydroxy-4-(trifluoromethyl) piperidin-1-yl]methyl}-7,8-dihydro-6H-spiro[[1,3]oxazolo[5,4-f]quinazoline-9,1′-cyclohexan]-7-one; 5-chloro-2-{[4-(propan-2-yloxy)piperidin-1-yl]methyl}-7,8-dihydro-6H-spiro[[1,3]oxazolo[5,4-f]quinazoline-9,1′-cyclohexan]-7-one; 5-chloro-2-[(3-fluoroazetidin-1-yl)methyl]-7,8-dihydro-6H-spiro[[1,3]oxazolo[5,4-f]quinazoline-9,1′-cyclohexan]-7-one; 5-chloro-2-[(3-methoxyazetidin-1-yl)methyl]-7,8-dihydro-6H-spiro[[1,3]oxazolo[5,4-f]quinazoline-9,1′-cyclohexan]-7-one; 5-chloro-2-{[3-(methoxymethyl)azetidin-1-yl]methyl}-7,8-dihydro-6H-spiro[[1,3]oxazolo[5,4-f]quinazoline-9,1′-cyclohexan]-7-one; 5-chloro-2-({7-oxa-2-azaspiro[3.5]nonan-2-yl}methyl)-7,8-dihydro-6H-spiro[[1,3]oxazolo[5,4-f]quinazoline-9,1′-cyclohexan]-7-one; 5-chloro-2-[(4-methoxy-4-methylpiperidin-1-yl)methyl]-7,8-dihydro-6H-spiro[[1,3]oxazolo[5,4-f]quinazoline-9,1′-cyclohexan]-7-one; 5-chloro-2-({3-oxo-1-oxa-8-azaspiro[4.5]decan-8-yl}methyl)-7,8-dihydro-6H-spiro[[1,3]oxazolo[5,4-f]quinazoline-9,1′-cyclohexan]-7-one; 2-[({5-chloro-7-oxo-7,8-dihydro-6H-spiro[[1,3]oxazolo[5,4-f]quinazoline-9,1′-cyclohexan]-2-yl}methyl)(methyl)amino]-N,N-dimethylacetamide; 5-chloro-2-({[(6-methylpyridin-3-yl)methyl]amino}methyl)-7,8-dihydro-6H-spiro[[1,3]oxazolo[5,4-f]quinazoline-9,1′-cyclohexan]-7-one; 5-chloro-2-({methyl[(oxan-4-yl)methyl]amino}methyl)-7,8-dihydro-6H-spiro[[1,3]oxazolo[5,4-f]quinazoline-9,1′-cyclohexan]-7-one; 5-chloro-2-({methyl[(3-methyloxetan-3-yl)methyl]amino}methyl)-7,8-dihydro-6H-spiro[[1,3]oxazolo[5,4-f]quinazoline-9,1′-cyclohexan]-7-one; 5-chloro-2-({[(3-fluorooxetan-3-yl)methyl]amino}methyl)-7,8-dihydro-6H-spiro[[1,3]oxazolo[5,4-f]quinazoline-9,1′-cyclohexan]-7-one; 5-chloro-2-({[(oxan-4-yl)methyl]amino}methyl)-7,8-dihydro-6H-spiro[[1,3]oxazolo[5,4-f]quinazoline-9,1′-cyclohexan]-7-one; 5-chloro-2-({2-oxa-7-azaspiro[3.5]nonan-7-yl}methyl)-7,8-dihydro-6H-spiro[[1,3]oxazolo[5,4-f]quinazoline-9,1′-cyclohexan]-7-one; 5-chloro-2-({[(3-methyloxetan-3-yl)methyl]amino}methyl)-7,8-dihydro-6H-spiro[[1,3]oxazolo[5,4-f]quinazoline-9,1′-cyclohexan]-7-one; 5-chloro-2-({2-oxa-5-azabicyclo[2.2.1]heptan-5-yl}methyl)-7,8-dihydro-6H-spiro[[1,3]oxazolo[5,4-f]quinazoline-9,1′-cyclohexan]-7-one; 5-chloro-2-{[methyl(2,2,2-trifluoroethyl)amino]methyl}-7,8-dihydro-6H-spiro[[1,3]oxazolo[5,4-f]quinazoline-9,1′-cyclohexan]-7-one; 5-chloro-2-({[(oxetan-3-yl)methyl]amino}methyl)-7,8-dihydro-6H-spiro[[1,3]oxazolo[5,4-f]quinazoline-9,1′-cyclohexan]-7-one; 5-chloro-2-[(4-oxopiperidin-1-yl)methyl]-7,8-dihydro-6H-spiro[[1,3]oxazolo[5,4-f]quinazoline-9,1′-cyclohexan]-7-one; 5-chloro-2-({[(2-methoxyphenyl)methyl]amino}methyl)-7,8-dihydro-6H-spiro[[1,3]oxazolo[5,4-f]quinazoline-9,1′-cyclohexan]-7-one; 5-chloro-2-({[(2,4-dimethoxyphenyl)methyl]amino}methyl)-7,8-dihydro-6H-spiro[[1,3]oxazolo[5,4-f]quinazoline-9,1′-cyclohexan]-7-one; 5-chloro-2-{[4-(propan-2-yl)piperazin-1-yl]methyl}-7,8-dihydro-6H-spiro[[1,3]oxazolo[5,4-f]quinazoline-9,1′-cyclohexan]-7-one; 5-chloro-2-[(3,3,4-trimethylpiperazin-1-yl)methyl]-7,8-dihydro-6H-spiro[[1,3]oxazolo[5,4-f]quinazoline-9,1′-cyclohexan]-7-one; 5-chloro-2-{[2-(hydroxymethyl)piperidin-1-yl]methyl}-7,8-dihydro-6H-spiro[[1,3]oxazolo[5,4-f]quinazoline-9,1′-cyclohexan]-7-one; 5-chloro-2-({[1-(hydroxymethyl)cyclopentyl]amino}methyl)-7,8-dihydro-6H-spiro[[1,3]oxazolo[5,4-f]quinazoline-9,1′-cyclohexan]-7-one; 5-chloro-2-{[(2R)-2-(hydroxymethyl)pyrrolidin-1-yl]methyl}-7,8-dihydro-6H-spiro[[1,3]oxazolo[5,4-f]quinazoline-9,1′-cyclohexan]-7-one; 5-chloro-2-[(2-methylpiperidin-1-yl)methyl]-7,8-dihydro-6H-spiro[[1,3]oxazolo[5,4-f]quinazoline-9,1′-cyclohexan]-7-one; 5-chloro-2-{[2-(2-methylpropyl)morpholin-4-yl]methyl}-7,8-dihydro-6H-spiro[[1,3]oxazolo[5,4-f]quinazoline-9,1′-cyclohexan]-7-one; 5-chloro-2-{[(2-cyclobutyl-2,2-difluoroethyl)amino]methyl}-7,8-dihydro-6H-spiro[[1,3]oxazolo[5,4-f]quinazoline-9,1′-cyclohexan]-7-one; 5-chloro-2-[(3-ethylmorpholin-4-yl)methyl]-7,8-dihydro-6H-spiro[[1,3]oxazolo[5,4-f]quinazoline-9,1′-cyclohexan]-7-one; 5-chloro-2-{[(2-cyclohexyl-2-hydroxyethyl)amino]methyl}-7,8-dihydro-6H-spiro[[1,3]oxazolo[5,4-f]quinazoline-9,1′-cyclohexan]-7-one; 5-chloro-2-({6-oxa-9-azaspiro[4.5]decan-9-yl}methyl)-7,8-dihydro-6H-spiro[[1,3]oxazolo[5,4-f]quinazoline-9,1′-cyclohexan]-7-one; 5-chloro-2-({[1-(oxan-2-yl)ethyl]amino}methyl)-7,8-dihydro-6H-spiro[[1,3]oxazolo[5,4-f]quinazoline-9,1′-cyclohexan]-7-one; 5-chloro-2-({[(oxan-2-yl)methyl]amino}methyl)-7,8-dihydro-6H-spiro[[1,3]oxazolo[5,4-f]quinazoline-9,1′-cyclohexan]-7-one; 5-chloro-2-[(2-methoxyethoxy)methyl]-7,8-dihydro-6H-spiro[[1,3]oxazolo[5,4-f]quinazoline-9,1′-cyclohexan]-7-one; ethyl 2-{5-chloro-7-oxo-7,8-dihydro-6H-spiro[[1,3]oxazolo[5,4-f]quinazoline-9,1′-cyclohexan]-2-yl}acetate; 2-[(4,4-difluoropiperidin-1-yl)methyl]-5-fluoro-7,8-dihydro-6H-spiro[[1,3]oxazolo[5,4-f]quinazoline-9,1′-cyclohexan]-7-one; 5-fluoro-2-{[(2-methoxyethyl)amino]methyl}-7,8-dihydro-6H-spiro[[1,3]oxazolo[5,4-f]quinazoline-9,1′-cyclohexan]-7-one; 5-chloro-2-{[(3-methyl-2-oxobutyl)amino]methyl}-7,8-dihydro-6H-spiro[[1,3]oxazolo[5,4-f]quinazoline-9,1′-cyclohexan]-7-one; 5-fluoro-2-[(3-methoxyazetidin-1-yl)methyl]-7,8-dihydro-6H-spiro[[1,3]oxazolo[5,4-f]quinazoline-9,1′-cyclohexan]-7-one; 5-chloro-2-{[(4,4-difluorocyclohexyl)amino]methyl}-7,8-dihydro-6H-spiro[[1,3]oxazolo[5,4-f]quinazoline-9,1′-cyclohexan]-7-one; 5-chloro-2-({[(1R,4R)-4-methoxycyclohexyl]amino}methyl)-7,8-dihydro-6H-spiro[[1,3]oxazolo[5,4-f]quinazoline-9,1′-cyclohexan]-7-one; 5-chloro-2-{[(1R,4R)-2-oxa-5-azabicyclo[2.2.1]heptan-5-yl]methyl}-7,8-dihydro-6H-spiro[[1,3]oxazolo[5,4-f]quinazoline-9,1′-cyclohexan]-7-one; 5-chloro-2-{[(1S,4S)-2-oxa-5-azabicyclo[2.2.1]heptan-5-yl]methyl}-7,8-dihydro-6H-spiro[[1,3]oxazolo[5,4-f]quinazoline-9,1′-cyclohexan]-7-one; 5-chloro-2-[(3-methoxy-3-methylazetidin-1-yl)methyl]-7,8-dihydro-6H-spiro[[1,3]oxazolo[5,4-f]quinazoline-9,1′-cyclohexan]-7-one; 5-chloro-2-[(4-methoxypiperidin-1-yl)methyl]-7,8-dihydro-6H-spiro[[1,3]oxazolo[5,4-f]quinazoline-9,1′-cyclohexan]-7-one; 5-chloro-2-{[4-(dimethylamino)piperidin-1-yl]methyl}-7,8-dihydro-6H-spiro[[1,3]oxazolo[5,4-f]quinazoline-9,1′-cyclohexan]-7-one; 5-chloro-2-({5-methyl-octahydropyrrolo[3,4-c]pyrrol-2-yl}methyl)-7,8-dihydro-6H-spiro[[1,3]oxazolo[5,4-f]quinazoline-9,1′-cyclohexan]-7-one; 5-chloro-2-{[(2R)-2-(methoxymethyl)pyrrolidin-1-yl]methyl}-7,8-dihydro-6H-spiro[[1,3]oxazolo[5,4-f]quinazoline-9,1′-cyclohexan]-7-one; 5-chloro-2-{[3-(hydroxymethyl)pyrrolidin-1-yl]methyl}-7,8-dihydro-6H-spiro[[1,3]oxazolo[5,4-f]quinazoline-9,1′-cyclohexan]-7-one; 5-chloro-2-{[(3R)-3-methoxypiperidin-1-yl]methyl}-7,8-dihydro-6H-spiro[[1,3]oxazolo[5,4-f]quinazoline-9,1′-cyclohexan]-7-one; 5-chloro-2-{[(3S)-3-methoxypiperidin-1-yl]methyl}-7,8-dihydro-6H-spiro[[1,3]oxazolo[5,4-f]quinazoline-9,1′-cyclohexan]-7-one; 5-chloro-2-{[(3R)-3-ethoxypyrrolidin-1-yl]methyl}-7,8-dihydro-6H-spiro[[1,3]oxazolo[5,4-f]quinazoline-9,1′-cyclohexan]-7-one; 5-chloro-2-{[(3R)-3-(2-hydroxypropan-2-yl)pyrrolidin-1-yl]methyl}-7,8-dihydro-6H-spiro[[1,3]oxazolo[5,4-f]quinazoline-9,1′-cyclohexan]-7-one; 5-chloro-2-{[(3R)-3-hydroxypyrrolidin-1-yl]methyl}-7,8-dihydro-6H-spiro[[1,3]oxazolo[5,4-f]quinazoline-9,1′-cyclohexan]-7-one; 5-chloro-2-({[2-(dimethylamino)ethyl](methyl)amino}methyl)-7,8-dihydro-6H-spiro[[1,3]oxazolo[5,4-f]quinazoline-9,1′-cyclohexan]-7-one; 3-[({5-chloro-7-oxo-7,8-dihydro-6H-spiro[[1,3]oxazolo[5,4-f]quinazoline-9,1′-cyclohexan]-2-yl}methyl)(methyl)amino]-N,N-dimethylpropanamide; 5-chloro-2-({methyl[2-(morpholin-4-yl)ethyl]amino}methyl)-7,8-dihydro-6H-spiro[[1,3]oxazolo[5,4-f]quinazoline-9,1′-cyclohexan]-7-one 5-chloro-2-[(3-methoxypyrrolidin-1-yl)methyl]-7,8-dihydro-6H-spiro[[1,3]oxazolo[5,4-f]quinazoline-9,1′-cyclohexan]-7-one; 5-chloro-2-{[4-(2,2,2-trifluoroethyl)piperazin-1-yl]methyl}-7,8-dihydro-6H-spiro[[1,3]oxazolo[5,4-f]quinazoline-9,1′-cyclohexan]-7-one; methyl 2-[({5-chloro-7-oxo-7,8-dihydro-6H-spiro[[1,3]oxazolo[5,4f]quinazoline-9,1′-cyclohexan]-2-yl}methyl)(methyl)amino]acetate; 2-{[(8aR)-octahydropyrrolo[1,2-a]pyrazin-2-yl]methyl}-5-chloro-7,8-dihydro-6H-spiro[[1,3]oxazolo[5,4-f]quinazoline-9,1′-cyclohexan]-7-one; 5-chloro-2-{[methyl(oxan-4-yl)amino]methyl}-7,8-dihydro-6H-spiro[[1,3]oxazolo[5,4-f]quinazoline-9,1′-cyclohexan]-7-one; methyl 1-({5-chloro-7-oxo-7,8-dihydro-6H-spiro[[1,3]oxazolo[5,4-f]quinazoline-9,1′-cyclohexan]-2-yl}methyl)piperidine-3-carboxylate; methyl 2-[({5-chloro-7-oxo-7,8-dihydro-6H-spiro[[1,3]oxazolo[5,4-f]quinazoline-9,1′-cyclohexan]-2-yl}methyl)amino]acetate; 5-chloro-2-{[(3S)-3-methoxypyrrolidin-1-yl]methyl}-7,8-dihydro-6H-spiro[[1,3]oxazolo[5,4-f]quinazoline-9,1′-cyclohexan]-7-one; 5-chloro-2-{[(oxan-3-yl)amino]methyl}-7,8-dihydro-6H-spiro[[1,3]oxazolo[5,4-f]quinazoline-9,1′-cyclohexan]-7-one; 5-chloro-2-{[(3R)-3-methoxypyrrolidin-1-yl]methyl}-7,8-dihydro-6H-spiro[[1,3]oxazolo[5,4-f]quinazoline-9,1′-cyclohexan]-7-one; 5-chloro-2-{[(3S)-3-(methoxymethyl)pyrrolidin-1-yl]methyl}-7,8-dihydro-6H-spiro[[1,3]oxazolo[5,4-f]quinazoline-9,1′-cyclohexan]-7-one; 5-chloro-2-{[(3R)-3-(methoxymethyl)pyrrolidin-1-yl]methyl}-7,8-dihydro-6H-spiro[[1,3]oxazolo[5,4-f]quinazoline-9,1′-cyclohexan]-7-one; methyl (3R)-1-({5-chloro-7-oxo-7,8-dihydro-6H-spiro[[1,3]oxazolo[5,4-f]quinazoline-9,1′-cyclohexan]-2-yl}methyl)piperidine-3-carboxylate; 2-({5-chloro-7-oxo-7,8-dihydro-6H-spiro[[1,3]oxazolo[5,4-f]quinazoline-9,1′-cyclohexan]-2-yl}methoxy)-N,N-dimethylacetamide; 5-chloro-2-[(oxan-4-yloxy)methyl]-7,8-dihydro-6H-spiro[[1,3]oxazolo[5,4-f]quinazoline-9,1′-cyclohexan]-7-one; 5-chloro-2-({[2-(trifluoromethoxy)ethyl]amino}methyl)-7,8-dihydro-6H-spiro[[1,3]oxazolo[5,4-f]quinazoline-9,1′-cyclohexan]-7-one; 5-chloro-2-[(4-ethylpiperazin-1-yl)methyl]-7,8-dihydro-6H-spiro[[1,3]oxazolo[5,4-f]quinazoline-9,1′-cyclohexan]-7-one; 5-chloro-2-{[3-(dimethylamino)pyrrolidin-1-yl]methyl}-7,8-dihydro-6H-spiro[[1,3]oxazolo[5,4-f]quinazoline-9,1′-cyclohexan]-7-one; 2-({5-chloro-7-oxo-7,8-dihydro-6H-spiro[[1,3]oxazolo[5,4-f]quinazoline-9,1′-cyclohexan]-2-yl}methyl)amino]-N,N-diethylacetamide; (2S)-2-[({5-chloro-7-oxo-7,8-dihydro-6H-spiro[[1,3]oxazolo[5,4-f]quinazoline-9,1′-cyclohexan]-2-yl}methyl)amino]-N,N-dimethylpropanamide; (2R)-2-[({5-chloro-7-oxo-7,8-dihydro-6H-spiro[[1,3]oxazolo[5,4-f]quinazoline-9,1′-cyclohexan]-2-yl}methyl)amino]-N,N-dimethylpropanamide; 5-chloro-2-({[2-oxo-2-(pyrrolidin-1-yl)ethyl]amino}methyl)-7,8-dihydro-6H-spiro[[1,3]oxazolo[5,4-f]quinazoline-9,1′-cyclohexan]-7-one; 5-chloro-2-{[(oxan-4-yl)amino]methyl}-7,8-dihydro-6H-spiro[[1,3]oxazolo[5,4-f]quinazoline-9,1′-cyclohexan]-7-one; 5-chloro-2-[({3-oxabicyclo[3.1.0]hexan-6-yl}amino)methyl]-7,8-dihydro-6H-spiro[[1,3]oxazolo[5,4-f]quinazoline-9,1′-cyclohexan]-7-one; 5-chloro-2-{[(oxepan-4-yl)amino]methyl}-7,8-dihydro-6H-spiro[[1,3]oxazolo[5,4-f]quinazoline-9,1′-cyclohexan]-7-one; 5-chloro-2-({[(oxolan-3-yl)methyl]amino}methyl)-7,8-dihydro-6H-spiro[[1,3]oxazolo[5,4-f]quinazoline-9,1′-cyclohexan]-7-one; 5-chloro-2-{[(3,3-difluoro-2-hydroxypropyl)amino]methyl}-7,8-dihydro-6H-spiro[[1,3]oxazolo[5,4-f]quinazoline-9,1′-cyclohexan]-7-one; 5-chloro-2-{[(4-cyclopropyloxan-4-yl)amino]methyl}-7,8-dihydro-6H-spiro[[1,3]oxazolo[5,4-f]quinazoline-9,1′-cyclohexan]-7-one; 5-chloro-2-{[(2,6-dimethyloxan-4-yl)amino]methyl}-7,8-dihydro-6H-spiro[[1,3]oxazolo[5,4-f]quinazoline-9,1′-cyclohexan]-7-one; 5-chloro-2-({[(3S)-oxan-3-yl]amino}methyl)-7,8-dihydro-6H-spiro[[1,3]oxazolo[5,4-f]quinazoline-9,1′-cyclohexan]-7-one; 5-chloro-2-({[(3S)-oxolan-3-yl]amino}methyl)-7,8-dihydro-6H-spiro[[1,3]oxazolo[5,4-f]quinazoline-9,1′-cyclohexan]-7-one; 5-chloro-2-{[(3-methyloxolan-3-yl)amino]methyl}-7,8-dihydro-6H-spiro[[1,3]oxazolo[5,4-f]quinazoline-9,1′-cyclohexan]-7-one; 5-chloro-2-({[(3R)-oxolan-3-yl]amino}methyl)-7,8-dihydro-6H-spiro[[1,3]oxazolo[5,4-f]quinazoline-9,1′-cyclohexan]-7-one; 5-chloro-2-{[4-(2-methoxyethoxy)piperidin-1-yl]methyl}-7,8-dihydro-6H-spiro[[1,3]oxazolo[5,4-f]quinazoline-9,1′-cyclohexan]-7-one; 2-{[(8aS)-octahydropyrrolo[1,2-a]pyrazin-2-yl]methyl}-5-chloro-7,8-dihydro-6H-spiro[[1,3]oxazolo[5,4-f]quinazoline-9,1′-cyclohexan]-7-one; methyl (3R)-1-({5-chloro-7-oxo-7,8-dihydro-6H-spiro[[1,3]oxazolo[5,4-f]quinazoline-9,1′-cyclohexan]-2-yl}methyl)pyrrolidine-3-carboxylate; 5-chloro-2-{[4-(2,2-difluoroethyl)piperazin-1-yl]methyl}-7,8-dihydro-6H-spiro[[1,3]oxazolo[5,4-f]quinazoline-9,1′-cyclohexan]-7-one; 5-chloro-2-({[(3-methyloxolan-3-yl)methyl]amino}methyl)-7,8-dihydro-6H-spiro[[1,3]oxazolo[5,4-f]quinazoline-9,1′-cyclohexan]-7-one; 5-chloro-2-({[(3R)-oxan-3-yl]amino}methyl)-7,8-dihydro-6H-spiro[[1,3]oxazolo[5,4-f]quinazoline-9,1′-cyclohexan]-7-one; 5-chloro-2-[({[(3R)-oxolan-3-yl]methyl}amino)methyl]-7,8-dihydro-6H-spiro[[1,3]oxazolo[5,4-f]quinazoline-9,1′-cyclohexan]-7-one; 5-chloro-2-{[(3-fluorooxan-4-yl)amino]methyl}-7,8-dihydro-6H-spiro[[1,3]oxazolo[5,4-f]quinazoline-9,1′-cyclohexan]-7-one; 5-chloro-2-{[(2,2-difluoro-3-hydroxypropyl)amino]methyl}-7,8-dihydro-6H-spiro[[1,3]oxazolo[5,4-f]quinazoline-9,1′-cyclohexan]-7-one; 5-chloro-2-{[(2,2-difluorocyclopentyl)amino]methyl}-7,8-dihydro-6H-spiro[[1,3]oxazolo[5,4-f]quinazoline-9,1′-cyclohexan]-7-one; 5-chloro-2-({[4-(methoxymethyl)oxan-4-yl]amino}methyl)-7,8-dihydro-6H-spiro[[1,3]oxazolo[5,4-f]quinazoline-9,1′-cyclohexan]-7-one; 5-chloro-2-[({[(3S)-oxolan-3-yl]methyl}amino)methyl]-7,8-dihydro-6H-spiro[[1,3]oxazolo[5,4-f]quinazoline-9,1′-cyclohexan]-7-one; 5-chloro-2-({[(1,3-oxazol-2-yl)methyl]amino}methyl)-7,8-dihydro-6H-spiro[[1,3]oxazolo[5,4-f]quinazoline-9,1′-cyclohexan]-7-one; 5-chloro-4′,4′-difluoro-2-methyl-7,8-dihydro-6H-spiro[[1,3]oxazolo[5,4-f]quinazoline-9,1′-cyclohexan]-7-one; 5-chloro-2-{[(1-methylpiperidin-4-yl)oxy]methyl}-7,8-dihydro-6H-spiro[[1,3]oxazolo[5,4-f]quinazoline-9,1′-cyclohexan]-7-one; 2-[({5-fluoro-7-oxo-7,8-dihydro-6H-spiro[[1,3]oxazolo[5,4-f]quinazoline-9,1′-cyclohexan]-2-yl}methyl)(methyl)amino]-N,N-dimethylacetamide; 2-[({5-fluoro-7-oxo-7,8-dihydro-6H-spiro[[1,3]oxazolo[5,4-f]quinazoline-9,1′-cyclohexan]-2-yl}methyl)amino]-N,N-dimethylacetamide; 5-chloro-4′,4′-difluoro-2-{[(1S,4S)-2-oxa-5-azabicyclo[2.2.1]heptan-5-yl]methyl}-7,8-dihydro-6H-spiro[[1,3]oxazolo[5,4-f]quinazoline-9,1′-cyclohexan]-7-one; 5-chloro-4′,4′-difluoro-2-[({[(3S)-oxolan-3-yl]methyl}amino)methyl]-7,8-dihydro-6H-spiro[[1,3]oxazolo[5,4-f]quinazoline-9,1′-cyclohexan]-7-one; 5-chloro-2,4′,4′-trimethyl-7,8-dihydro-6H-spiro[[1,3]oxazolo[5,4-f]quinazoline-9,1′-cyclohexan]-7-one; 5-chloro-4′,4′-difluoro-2-({[(3S)-oxan-3-yl]amino}methyl)-7,8-dihydro-6H-spiro[[1,3]oxazolo[5,4-f]quinazoline-9,1′-cyclohexan]-7-one; 5-chloro-4′,4′-difluoro-2-[(4-methylpiperazin-1-yl)methyl]-7,8-dihydro-6H-spiro[[1,3]oxazolo[5,4-f]quinazoline-9,1′-cyclohexan]-7-one; 5-chloro-4′,4′-difluoro-2-[({[(3R)-oxolan-3-yl]methyl}amino)methyl]-7,8-dihydro-6H-spiro[[1,3]oxazolo[5,4-f]quinazoline-9,1′-cyclohexan]-7-one; 5-chloro-4′,4′-difluoro-2-({[(3S)-oxolan-3-yl]amino}methyl)-7,8-dihydro-6H-spiro[[1,3]oxazolo[5,4-f]quinazoline-9,1′-cyclohexan]-7-one; 5-chloro-4′,4′-difluoro-2-({[(3R)-oxan-3-yl]amino}methyl)-7,8-dihydro-6H-spiro[[1,3]oxazolo[5,4-f]quinazoline-9,1′-cyclohexan]-7-one; 5-chloro-2-({[(1,3-oxazol-5-yl)methyl]amino}methyl)-7,8-dihydro-6H-spiro[[1,3]oxazolo[5,4-f]quinazoline-9,1′-cyclohexan]-7-one; 5-chloro-2-({[2-(1,2-oxazol-3-yl)ethyl]amino}methyl)-7,8-dihydro-6H-spiro[[1,3]oxazolo[5,4-f]quinazoline-9,1′-cyclohexan]-7-one; 5-chloro-2-({[(1,3-oxazol-4-yl)methyl]amino}methyl)-7,8-dihydro-6H-spiro[[1,3]oxazolo[5,4-f]quinazoline-9,1′-cyclohexan]-7-one; 5-chloro-2-({[(1-methyl-1H-pyrazol-3-yl)methyl]amino}methyl)-7,8-dihydro-6H-spiro[[1,3]oxazolo[5,4-f]quinazoline-9,1′-cyclohexan]-7-one; 5-chloro-2-({[(1,2-oxazol-5-yl)methyl]amino}methyl)-7,8-dihydro-6H-spiro[[1,3]oxazolo[5,4-f]quinazoline-9,1′-cyclohexan]-7-one; and pharmaceutically acceptable salts thereof; or 5′-chloro-7′-(hydroxymethyl)-7′,8′-dihydro-6′H-spiro[cyclohexane-1,9′-furo[2,3-f]quinazoline]-7′-one; 5′-chloro-7′-[4-(2-fluoroethyl)piperazine-1-carbonyl]-7′,8′-dihydro-6′H-spiro[cyclohexane-1,9′-furo[2,3-f]quinazoline]-7′-one; N-{2-[benzyl(methyl)amino]ethyl}-5′-chloro-N-methyl-7′-oxo-7′,8′-dihydro-6′H-spiro[cyclohexane-1,9′-furo[2,3-f]quinazoline]-2′-carboxamide; 5′-chloro-2′-{[4-(propan-2-yl)piperazin-1-yl]methyl}-7′,8′-dihydro-6′H-spiro[cyclohexane-1,9′-furo[2 3-f]quinazoline]-7′-one; 5′-chloro-2′-{[methyl({[4-(propan-2-yl)-4H-1,2,4-triazol-3-yl]methyl})amino]methyl}-7′,8′-dihydro-6′H-spiro[cyclohexane-1,9′-furo[2,3-]quinazoline]-7′-one; 5′-chloro-2′-(4-methylpiperazine-1-carbonyl)-7′,8′-dihydro-6′H-spiro[cyclohexane-1,9′-furo[2,3-f]quinazoline]-7′-one; 5′-chloro-2′-(4-ethylpiperazine-1-carbonyl)-7′,8′-dihydro-6′H-spiro[cyclohexane-1,9′-furo[2,3-f]quinazoline]-7′-one; 5′-chloro-N-(2-methoxyethyl)-N-methyl-7′-oxo-7′,8′-dihydro-6′H-spiro[cyclohexane-1,9′-furo[2,3-f]quinazoline]-2′-carboxamide; 5′-chloro-2′-[4-(2-methoxyethyl)piperazine-1-carbonyl]-7′,8′-dihydro-6′H-spiro[cyclohexane-1,9′-furo[2,3-f]quinazoline]-7′-one; 5′-chloro-7′-(piperidine-1-carbonyl)-7′,8′-dihydro-6′H-spiro[cyclohexane-1,9′-furo[2,3-f]quinazoline]-7′-one; 2′-(azetidine-1-carbonyl)-5′-chloro-7′,8′-dihydro-6′H-spiro[cyclohexane-1,9′-furo[2,3-f]quinazoline]-7′-one; 5′-chloro-7′-(pyrrolidine-1-carbonyl)-7′,8′-dihydro-6′H-spiro[cyclohexane-1,9′-furo[2,3-f]quinazoline]-7′-one; 5′-chloro-7′-(morpholine-4-carbonyl)-7′,8′-dihydro-6′H-spiro[cyclohexane-1,9′-furo[2,3-f]quinazoline]-7′-one; 5′-chloro-2′-[4-(2,2,2-trifluoroethyl)piperazine-1-carbonyl]-7′,8′-dihydro-6′H-spiro[cyclohexane-1,9′-furo[2,3-f]quinazoline]-7′-one; 5′-chloro-2′-[4-(2,2-difluoroethyl)piperazine-1-carbonyl]-7′,8′-dihydro-6′H-spiro[cyclohexane-1,9′-furo[2,3-f]quinazoline]-7′-one; 5′-chloro-2′-[(1S,4S)-2-oxa-5-azabicyclo[2.2.1]heptan-5-ylmethyl]-7′,8′-dihydro-6′H-spiro[cyclohexane-1,9′-furo[2,3-f]quinazoline]-7′-one; 5′-chloro-2′-(piperidin-1-ylmethyl)-7′,8′-dihydro-6′H-spiro[cyclohexane-1,9′-furo[2,3-f]quinazoline]-7′-one; 5′-chloro-2′-(morpholin-4-ylmethyl)-7′,8′-dihydro-6′H-spiro[cyclohexane-1,9′-furo[2,3-f]quinazoline]-7′-one; 5′-chloro-2′-(pyrrolidin-1-ylmethyl)-7′,8′-dihydro-6′H-spiro[cyclohexane-1,9′-furo[2,3-f]quinazoline]-7′-one; 5′-chloro-2′-[(4-methylpiperazin-1-yl)methyl]-7′,8′-dihydro-6′H-spiro[cyclohexane-1,9′-furo[2,3-f]quinazoline]-7′-one; 5′-chloro-N-ethyl-7′-oxo-N-(propan-2-yl)-7′,8′-dihydro-6′H-spiro[cyclohexane-1,9′-furo[2,3-f]quinazoline]-2′-carboxamide; 5′-chloro-7′-oxo-N-[2-(propan-2-yloxy)ethyl]-7′,8′-dihydro-6′H-spiro[cyclohexane-1,9′-furo[2,3-f]quinazoline]-2′-carboxamide; 5′-chloro-2′-(4-methoxypiperidine-1-carbonyl)-7′,8′-dihydro-6′H-spiro[cyclohexane-1,9′-furo[2,3-f]quinazoline]-7′-one; 5′-chloro-N-[(4-fluorophenyl)methyl]-7′-oxo-7′,8′-dihydro-6′H-spiro[cyclohexane-1,9′-furo[2,3-f]quinazoline]-2′-carboxamide; 5′-chloro-N-(2-methoxyethyl)-7′-oxo-7′,8′-dihydro-6′H-spiro[cyclohexane-1,9′-furo[2,3-f]quinazoline]-7′-carboxamide; 5′-chloro-N-methyl-N-(2-methylpropyl)-7′-oxo-7′,8′-dihydro-6′H-spiro[cyclohexane-1,9′-furo[2,3-f]quinazoline]-7′-carboxamide; 5′-chloro-N-cyclopentyl-N-methyl-7′-oxo-7′,8′-dihydro-6′H-spiro[cyclohexane-1,9′-furo[2,3-f]quinazoline]-7′-carboxamide; 5′-chloro-N-(1-hydroxy-3-methylbutan-2-yl)-7′-oxo-7′,8′-dihydro-6′H-spiro[cyclohexane-1,9′-furo[2,3-f]quinazoline]-2′-carboxamide; 5′-chloro-N-(1-methylpiperidin-4-yl)-7′-oxo-7′,8′-dihydro-6′H-spiro[cyclohexane-1,9′-furo[2,3-f]quinazoline]-2′-carboxamide; 5′-chloro-N-[(2-fluorophenyl)methyl]-7′-oxo-7′,8′-dihydro-6′H-spiro[cyclohexane-1,9′-furo[2,3-f]quinazoline]-2′-carboxamide; 5′-chloro-2′-[4-(2-hydroxyethyl)piperazine-1-carbonyl]-7′,8′-dihydro-6′H-spiro[cyclohexane-1,9′-furo[2,3-f]quinazoline]-7′-one; 5′-chloro-N-(3-hydroxypropyl)-7′-oxo-7′,8′-dihydro-6′H-spiro[cyclohexane-1,9′-furo[2,3-f]quinazoline]-2′-carboxamide; 5′-chloro-N-(4-hydroxybutyl)-7′-oxo-7′,8′-dihydro-6′H-spiro[cyclohexane-1,9′-furo[2,3-f]quinazoline]-2′-carboxamide; 5′-chloro-N-(2-hydroxyethyl)-7′-oxo-N-propyl-7′,8′-dihydro-6′H-spiro[cyclohexane-1,9′-furo[2,3-f]quinazoline]-2′-carboxamide; 5′-chloro-7′-oxo-N-[2-(pyrrolidin-1-yl)ethyl]-7′,8′-dihydro-6′H-spiro[cyclohexane-1,9′-furo[2,3-f]quinazoline]-2′-carboxamide; 5′-chloro-N-[2-(dimethylamino)ethyl]-N-ethyl-7′-oxo-7′,8′-dihydro-6′H-spiro[cyclohexane-1,9′-furo[2,3-f]quinazoline]-2′-carboxamide; 5′-chloro-N-[3-(1H-imidazol-1-yl)propyl]-7′-oxo-7′,8′-dihydro-6′H-spiro[cyclohexane-1,9′-furo[2,3-f]quinazoline]-2′-carboxamide; 5′-chloro-N-(2-hydroxyethyl)-N-methyl-7′-oxo-7′,8′-dihydro-6′H-spiro[cyclohexane-1,9′-furo[2,3-f]quinazoline]-2′-carboxamide; 5′-chloro-N-(2-hydroxybutyl)-7′-oxo-7′,8′-dihydro-6′H-spiro[cyclohexane-1,9′-furo[2,3-f]quinazoline]-2′-carboxamide; 5′-chloro-N-[3-(dimethylamino)propyl]-N-methyl-7′-oxo-7′,8′-dihydro-6′H-spiro[cyclohexane-1,9′-furo[2,3-f]quinazoline]-2′-carboxamide; 2′-[(8aS)-octahydropyrrolo[1,2-a]piperazine-2-carbonyl]-5′-chloro-7′,8′-dihydro-6′H-spiro[cyclohexane-1,9′-furo[2,3-f]quinazoline]-7′-one; 5′-chloro-N-methyl-7′-oxo-N-[2-(pyridin-2-yl)ethyl]-7′,8′-dihydro-6′H-spiro[cyclohexane-1,9′-furo[2,3-f]quinazoline]-2′-carboxamide; 5′-chloro-N-(2-cyanoethyl)-N-methyl-7′-oxo-7′,8′-dihydro-6′H-spiro[cyclohexane-1,9′-furo[2,3-f]quinazoline]-2′-carboxamide; 5′-chloro-N-(2-ethoxyethyl)-7′-oxo-7′,8′-dihydro-6′H-spiro[cyclohexane-1,9′-furo[2,3-f]quinazoline]-2′-carboxamide; 5′-chloro-7′-oxo-N-(oxolan-2-ylmethyl)-7′,8′-dihydro-6′H-spiro[cyclohexane-1,9′-furo[2,3-f]quinazoline]-2′-carboxamide; 5′-chloro-N-(2-methoxyethyl)-7′-oxo-N-(propan-2-yl)-7′,8′-dihydro-6′H-spiro[cyclohexane-1,9′-furo[2,3-f]quinazoline]-2′-carboxamide; 5′-chloro-7′-oxo-N-[2-(thiophen-2-yl)ethyl]-7′,8′-dihydro-6′H-spiro[cyclohexane-1,9′-furo[2,3-f]quinazoline]-2′-carboxamide; 5′-chloro-N-cyclopentyl-7′-oxo-7′,8′-dihydro-6′H-spiro[cyclohexane-1,9′-furo[2,3-f]quinazoline]-2′-carboxamide; 5′-chloro-N-ethyl-N-(2-hydroxyethyl)-7′-oxo-7′,8′-dihydro-6′H-spiro[cyclohexane-1,9′-furo[2,3-f]quinazoline]-2′-carboxamide; 5′-chloro-2′-(3-hydroxypiperidine-1-carbonyl)-7′,8′-dihydro-6′H-spiro[cyclohexane-1,9′-furo[2,3-f]quinazoline]-7′-one; 5′-chloro-7′-oxo-N-[2-(1H-pyrrol-1-yl)ethyl]-7′,8′-dihydro-6′H-spiro[cyclohexane-1,9′-furo[2,3-f]quinazoline]-2′-carboxamide; 5′-chloro-N-methyl-N-(1-methylpyrrolidin-3-yl)-7′-oxo-7′,8′-dihydro-6′H-spiro[cyclohexane-1,9′-furo[2,3-f]quinazoline]-2′-carboxamide; 5′-chloro-N-(3-hydroxy-3-phenylpropyl)-N-methyl-7′-oxo-7′,8′-dihydro-6′H-spiro[cyclohexane-1,9′-furo[2,3-f]quinazoline]-2′-carboxamide; 5′-chloro-2′-[3-(hydroxymethyl)-3-(2-methylpropyl)piperidine-1-carbonyl]-7′,8′-dihydro-6′H-spiro[cyclohexane-1,9′-furo[2,3-f]quinazoline]-7′-one; 5′-chloro-N-[(2,3-dimethoxyphenyl)methyl]-N-methyl-7′-oxo-7′,8′-dihydro-6′H-spiro[cyclohexane-1,9′-furo[2,3-f]quinazoline]-7′-carboxamide; 5′-chloro-7′-[2-(trifluoromethyl)-5H,6H,7H,8H-imidazo [1,2-a]pyrazine-7-carbonyl]-7′,8′-dihydro-6′H-spiro[cyclohexane-1,9′-furo[2,3-f]quinazoline]-7′-one; 5′-chloro-N-[3-(morpholin-4-yl)propyl]-7′-oxo-7′,8′-dihydro-6′H-spiro[cyclohexane-1,9′-furo[2,3-f]quinazoline]-7′-carboxamide; 5′-chloro-N-[(2-methoxyphenyl)methyl]-N-methyl-7′-oxo-7′,8′-dihydro-6′H-spiro[cyclohexane-1,9′-furo[2,3-f]quinazoline]-2′-carboxamide; 5′-chloro-2′-[3-(1H-imidazol-1-ylmethyl)piperidine-1-carbonyl]-7′,8′-dihydro-6′H-spiro[cyclohexane-1,9′-furo[2,3-f]quinazoline]-7′-one; ethyl 2-[4-({5′-chloro-7′-oxo-7′,8′-dihydro-6′H-spiro[cyclohexane-1,9′-furo[2,3-f]quinazoline]-2′-yl}carbonyl)piperazin-1-yl]acetate; 5′-chloro-N-[(2,4-dimethoxyphenyl)methyl]-N-methyl-7′-oxo-7′,8′-dihydro-6′H-spiro[cyclohexane-1,9′-furo[2,3-f]quinazoline]-2′-carboxamide; 5′-chloro-N-[(3-methoxyphenyl)methyl]-N-methyl-7′-oxo-7′,8′-dihydro-6′H-spiro[cyclohexa ne-1,9′-furo[2,3-f]quinazoline]-2′-carboxamide; 5′-chloro-N-(2,2-dimethyloxan-4-yl)-N-ethyl-7′-oxo-7′,8′-dihydro-6′H-spiro[cyclohexane-1,9′-furo[2,3-f]quinazoline]-2′-carboxamide; 5′-chloro-N-methyl-N-[(1-methyl-1H-1,3-benzodiazol-2-yl)methyl]-7′-oxo-7′,8′-dihydro-6′H-spiro[cyclohexane-1,9′-furo[2,3-f]quinazoline]-2′-carboxamide; 5′-chloro-N-[(3,5-dimethoxyphenyl)methyl]-N-methyl-7′-oxo-7′,8′-dihydro-6′H-spiro[cyclohexane-1,9′-furo[2,3-f]quinazoline]-2′-carboxamide; 5′-chloro-2′-{5H,6H,7H, 8H-imidazo [1,2-a]pyrazine-7-carbonyl}-7′,8′-dihydro-6′H-spiro[cyclohexane-1,9′-furo[2,3-f]quinazoline]-7′-one; 5′-chloro-2′-[2-(pyridin-2-yl)pyrrolidine-1-carbonyl]-7′,8′-dihydro-6′H-spiro[cyclohexane-1,9′-furo[2,3-f]quinazoline]-7′-one; 5′-chloro-N-(2-hydroxy-2-phenylethyl)-N-methyl-7′-oxo-7′,8′-dihydro-6′H-spiro[cyclohexane-1,9′-furo[2,3-f]quinazoline]-2′-carboxamide; 5′-chloro-N-[2-(2,3-dihydro-1H-indol-1-yl)ethyl]-7′-oxo-7′,8′-dihydro-6′H-spiro[cyclohexane-1,9′-furo[2,3-f]quinazoline]-2′-carboxamide; 5′-chloro-N-[(2,3-dimethoxyphenyl)methyl]-7′-oxo-7′,8′-dihydro-6′H-spiro[cyclohexane-1,9′-furo[2,3-f]quinazoline]-2′-carboxamide; 5′-chloro-N-methyl-N-[(5-methyl-1H-1,3-benzodiazol-2-yl)methyl]-7′-oxo-7′,8′-dihydro-6′H-spiro[cyclohexane-1,9′-furo[2,3-f]quinazoline]-2′-carboxamide; 5′-chloro-N-methyl-7′-oxo-N-[3-(trimethyl-1H-pyrazol-4-yl)propyl]-7′,8′-dihydro-6′H-spiro[cyclohexane-1,9′-furo[2,3-f]quinazoline]-2′-carboxamide; 5′-chloro-2′-{octahydropyrrolo[1,2-a]piperazine-2-carbonyl}-7′,8′-dihydro-6′H-spiro[cyclohexane-1,9′-furo[2,3-f]quinazoline]-7′-one; 5′-chloro-N-(2,3-dihydro-1,4-benzodioxin-6-ylmethyl)-7′-oxo-7′,8′-dihydro-6′H-spiro[cyclohexane-1,9′-furo[2,3-f]quinazoline]-2′-carboxamide; 5′-chloro-N-[(5-cyclopropyl-1H-pyrazol-3-yl)methyl]-N-methyl-7′-oxo-7′,8′-dihydro-6′H-spiro[cyclohexane-1,9′-furo[2,3-f]quinazoline]-2′-carboxamide; 5′-chloro-N-[(2,4-dimethoxyphenyl)methyl]-7′-oxo-7′,8′-dihydro-6′H-spiro[cyclohexane-1,9′-furo[2,3-f]quinazoline]-2′-carboxamide; N-(1-benzylpyrrolidin-3-yl)-5′-chloro-7′-oxo-7′,8′-dihydro-6′H-spiro[cyclohexane-1,9′-furo[2,3-f]quinazoline]-2′-carboxamide; 5′-chloro-N-[(2,3-difluoro-4-methoxyphenyl)methyl]-7′-oxo-7′,8′-dihydro-6′H-spiro[cyclohexane-1,9′-furo[2,3-f]quinazoline]-2′-carboxamide; 5′-chloro-N-{[4-(dimethylamino)phenyl]methyl}-7′-oxo-7′,8′-dihydro-6′H-spiro[cyclohexane-1,9′-furo[2,3-f]quinazoline]-2′-carboxamide; 5′-chloro-N-[(1-methyl-1H-imidazol-2-yl)methyl]-7′-oxo-N-(propan-2-yl)-7′,8′-dihydro-6′H-spiro[cyclohexane-1,9′-furo[2,3-f]quinazoline]-2′-carboxamide; 5′-chloro-2′-[4-(pyridin-4-yl)piperazine-1-carbonyl]-7′,8′-dihydro-6′H-spiro[cyclohexane-1,9′-furo[2,3-f]quinazoline]-7′-one; N-[(5-tert-butyl-1H-pyrazol-3-yl)methyl]-5′-chloro-N-methyl-7′-oxo-7′,8′-dihydro-6′H-spiro[cyclohexane-1,9′-furo[2,3-f]quinazoline]-2′-carboxamide; 2′-(4-benzylpiperazine-1-carbonyl)-5′-chloro-7′,8′-dihydro-6′H-spiro[cyclohexane-1,9′-furo[2,3-f]quinazoline]-7′-one; 5′-chloro-N-[(1-methylpiperidin-4-yl)methyl]-7′-oxo-7′,8′-dihydro-6′H-spiro[cyclohexane-1,9′-furo[2,3-f]quinazoline]-2′-carboxamide; 5′-chloro-7′-(3-methoxypiperidine-1-carbonyl)-7′,8′-dihydro-6′H-spiro[cyclohexane-1,9′-furo[2,3-f]quinazoline]-7′-one; 5′-chloro-N-methyl-7′-oxo-N-[(trimethyl-1H-pyrazol-4-yl)methyl]-7′,8′-dihydro-6′H-spiro[cyclohexane-1,9′-furo[2,3-f]quinazoline]-2′-carboxamide; 5′-chloro-2′-[4-(pyridin-2-yl)piperazine-1-carbonyl]-7′,8′-dihydro-6′H-spiro[cyclohexane-1,9′-furo[2,3-f]quinazoline]-7′-one; 5′-chloro-N-[3-(3,5-dimethyl-1H-pyrazol-1-yl)propyl]-N-methyl-7′-oxo-7′,8′-dihydro-6′H-spiro[cyclohexane-1,9′-furo[2,3-f]quinazoline]-2′-carboxamide; 5′-chloro-2′-(4-methyl-2-phenylpiperazine-1-carbonyl)-7′,8′-dihydro-6′H-spiro[cyclohexane-1,9′-furo[2,3-f]quinazoline]-7′-one; 5′-chloro-2′-[3-(dimethylamino)piperidine-1-carbonyl]-7′,8′-dihydro-6′H-spiro[cyclohexane-1,9′-furo[2,3-f]quinazoline]-7′-one; 5′-chloro-N-[2-(morpholin-4-yl)ethyl]-7′-oxo-7′,8′-dihydro-6′H-spiro[cyclohexane-1,9′-furo[2,3-f]quinazoline]-2′-carboxamide; 5′-chloro-7′-oxo-N-[2-(pyridin-3-yloxy)ethyl]-7′,8′-dihydro-6′H-spiro[cyclohexane-1,9′-furo[2,3-f]quinazoline]-2′-carboxamide; 5′-chloro-N-[(2-fluoro-4-methoxyphenyl)methyl]-7′-oxo-7′,8′-dihydro-6′H-spiro[cyclohexane-1,9′-furo[2,3-f]quinazoline]-2′-carboxamide; 1-({5′-chloro-7′-oxo-7′,8′-dihydro-6′H-spiro[cyclohexane-1,9′-furo[2,3-f]quinazoline]-2′-yl}carbonyl)azetidine-3-carbonitrile; 5′-chloro-2′-{3-phenyl-5H,6H,7H,8H-imidazo [1,2-a]pyrazine-7-carbonyl}-7′,8′-dihydro-6′H-spiro[cyclohexane-1,9′-furo[2,3-f]quinazoline]-7′-one; 5′-chloro-7′-oxo-N-[2-(2-oxopyrrolidin-1-yl)ethyl]-7′,8′-dihydro-6′H-spiro[cyclohexane-1,9′-furo[2,3-f]quinazoline]-2′-carboxamide; 5′-chloro-7′-oxo-N-(2,2,6,6-tetramethyloxan-4-yl)-7′,8′-dihydro-6′H-spiro[cyclohexane-1,9′-furo[2,3-f]quinazoline]-2′-carboxamide; 2′-[(1R,5S)-3-azabicyclo[3.1.0]hexane-3-carbonyl]-5′-chloro-7′,8′-dihydro-6′H-spiro[cyclohexane-1,9′-furo[2,3-f]quinazoline]-7′-one; 5′-chloro-N-[2-(4-fluorophenoxy)ethyl]-7′-oxo-7′,8′-dihydro-6′H-spiro[cyclohexane-1,9′-furo[2,3-f]quinazoline]-2′-carboxamide; 5′-chloro-7′-oxo-N-[2-(1H-pyrazol-1-yl)ethyl]-7′,8′-dihydro-6′H-spiro[cyclohexane-1,9′-furo[2,3-f]quinazoline]-2′-carboxamide; 5′-chloro-N-[4-(4-hydroxypiperidin-1-yl)phenyl]-7′-oxo-7′,8′-dihydro-6′H-spiro[cyclohexane-1,9′-furo[2,3-f]quinazoline]-2′-carboxamide; 5′-chloro-N-(3,3-dimethyl-2-oxobutyl)-7′-oxo-7′,8′-dihydro-6′H-spiro[cyclohexane-1,9′-furo[2,3-f]quinazoline]-2′-carboxamide; 5′-chloro-N-(furan-2-ylmethyl)-N-methyl-7′-oxo-7′,8′-dihydro-6′H-spiro[cyclohexane-1,9′-furo[2,3-f]quinazoline]-2′-carboxamide; 5′-chloro-2′-{4-[2-(dimethylamino)ethyl]piperidine-1-carbonyl}-7′,8′-dihydro-6′H-spiro[cyclohexane-1,9′-furo[2,3-f]quinazoline]-7′-one; 5′-chloro-2′-[4-(5-chloropyridin-2-yl)piperazine-1-carbonyl]-7′,8′-dihydro-6′H-spiro[cyclohexane-1,9′-furo[2,3-f]quinazoline]-7′-one; 5′-chloro-N-[(2-methoxyphenyl)methyl]-7′-oxo-7′,8′-dihydro-6′H-spiro[cyclohexane-1,9′-furo[2,3-f]quinazoline]-2′-carboxamide; 5′-chloro-N-(2-hydroxy-2-methylpropyl)-7′-oxo-7′,8′-dihydro-6′H-spiro[cyclohexane-1,9′-furo[2,3-f]quinazoline]-2′-carboxamide; 5′-chloro-N-[(4-cyanophenyl)methyl]-7′-oxo-7′,8′-dihydro-6′H-spiro[cyclohexane-1,9′-furo[2,3-f]quinazoline]-2′-carboxamide; 5′-chloro-2′-[3-oxo-4-(propan-2-yl)piperazine-1-carbonyl]-7′,8′-dihydro-6′H-spiro[cyclohexane-1,9′-furo[2,3-f]quinazoline]-7′-one; 5′-chloro-N-{[5-(difluoromethoxy)pyridin-2-yl]methyl}-7′-oxo-7′,8′-dihydro-6′H-spiro[cyclohexane-1,9′-furo[2,3-f]quinazoline]-2′-carboxamide; 5′-chloro-2′-[4-(oxetan-3-yl)piperazine-1-carbonyl]-7′,8′-dihydro-6′H-spiro[cyclohexane-1,9′-furo[2,3-f]quinazoline]-7′-one; 5′-chloro-N-[(1R,5S)-6,6-difluorobicyclo[3.1.0]hexan-3-yl]-7′-oxo-7′,8′-dihydro-6′H-spiro[cyclohexane-1,9′-furo[2,3-f]quinazoline]-2′-carboxamide; N-[2-(2H-1,3-benzodioxol-5-yl)ethyl]-5′-chloro-7′-oxo-7′,8′-dihydro-6′H-spiro[cyclohexane-1,9′-furo[2,3-f]quinazoline]-2′-carboxamide; 5′-chloro-N-[(4-ethyl-4H-1,2,4-triazol-3-yl)methyl]-N-methyl-7′-oxo-7′,8′-dihydro-6′H-spiro[cyclohexane-1,9′-furo[2,3-f]quinazoline]-2′-carboxamide; 5′-chloro-N-[(4-cyano-3-fluorophenyl)methyl]-7′-oxo-7′,8′-dihydro-6′H-spiro[cyclohexane-1,9′-furo[2,3-f]quinazoline]-7′-carboxamide; 2-{5′-chloro-7′-oxo-7′,8′-dihydro-6′H-spiro[cyclohexane-1,9′-furo[2,3-f]quinazoline]-7′-ylformamido}-N,N-diethylacetamide; 5′-chloro-7′-({6′-methyl-5′,6′-dihydro-4′H-spiro[piperidine-4,7′-thieno[2,3-c]pyridine]-1-yl}carbonyl)-7′,8′-dihydro-6′H-spiro[cyclohexane-1,9′-furo[2,3-f]quinazoline]-7′-one; 5′-chloro-N-[(6-methylpyridin-3-yl)methyl]-7′-oxo-7′,8′-dihydro-6′H-spiro[cyclohexane-1,9′-furo[2,3-f]quinazoline]-7′-carboxamide; 5′-chloro-7′-oxo-N-[(trimethyl-1H-imidazol-2-yl)methyl]-7′,8′-dihydro-6′H-spiro[cyclohexane-1,9′-furo[2,3-f]quinazoline]-2′-carboxamide; 5′-chloro-N-[(5-methylpyridin-3-yl)methyl]-7′-oxo-7′,8′-dihydro-6′H-spiro[cyclohexane-1,9′-furo[2,3-f]quinazoline]-2′-carboxamide; 5′-chloro-N-methyl-7′-oxo-N-[(5-phenyl-1,2-oxazol-3-yl)methyl]-7′,8′-dihydro-6′H-spiro[cyclohexane-1,9′-furo[2,3-f]quinazoline]-2′-carboxamide; 2′-{6-azaspiro[2.5]octane-6-carbonyl}-5′-chloro-7′,8′-dihydro-6′H-spiro[cyclohexane-1,9′-furo[2,3-f]quinazoline]-7′-one; 5′-chloro-N-[2-(oxan-4-yl)ethyl]-7′-oxo-7′,8′-dihydro-6′H-spiro[cyclohexane-1,9′-furo[2,3-f]quinazoline]-2′-carboxamide; 5′-chloro-2′-({6′,7′-dihydro-5′H-spiro[piperidine-4,4′-thieno[3,2-c]pyridine]-1-yl}carbonyl)-7′,8′-dihydro-6′H-spiro[cyclohexane-1,9′-furo[2,3-f]quinazoline]-7′-one; 5′-chloro-N-(1-methoxy-2-methylpropan-2-yl)-7′-oxo-7′,8′-dihydro-6′H-spiro[cyclohexane-1,9′-furo[2,3-f]quinazoline]-2′-carboxamide; 5′-chloro-7′-oxo-N-[1-(pyridin-3-yl)piperidin-4-yl]-7′,8′-dihydro-6′H-spiro[cyclohexane-1,9′-furo[2,3-f]quinazoline]-2′-carboxamide; 5′-chloro-2′-({3,4-dihydrospiro[2-benzopyran-1,4′-piperidine]-1′-yl}carbonyl)-7′,8′-dihydro-6′H-spiro[cyclohexane-1,9′-furo[2,3-f]quinazoline]-7′-one; 5′-chloro-7′-oxo-N-(2-phenoxyethyl)-7′,8′-dihydro-6′H-spiro[cyclohexane-1,9′-furo[2,3-f]quinazoline]-2′-carboxamide; 3-{5′-chloro-7′-oxo-7′,8′-dihydro-6′H-spiro[cyclohexane-1,9′-furo[2,3-f]quinazoline]-2′-ylformamido}-N,N-dimethylpropanamide; 5′-chloro-N-[1-(methoxymethyl)cyclopropyl]-7′-oxo-7′,8′-dihydro-6′H-spiro[cyclohexane-1,9′-furo[2,3-f]quinazoline]-7′-carboxamide; 5′-chloro-N-[(3-fluoropyridin-4-yl)methyl]-7′-oxo-7′,8′-dihydro-6′H-spiro[cyclohexane-1,9′-furo[2,3-f]quinazoline]-7′-carboxamide; 5′-chloro-N-(cyclobutylmethyl)-7′-oxo-N-(oxolan-2-ylmethyl)-7′,8′-dihydro-6′H-spiro[cyclohexane-1,9′-furo[2,3-f]quinazoline]-7′-carboxamide; 5′-chloro-7′-[4-(furan-2-ylmethyl)piperazine-1-carbonyl]-7′,8′-dihydro-6′H-spiro[cyclohexane-1,9′-furo[2,3-f]quinazoline]-7′-one; 5′-chloro-N-[(1-methyl-1H-pyrazol-4-yl)methyl]-7′-oxo-7′,8′-dihydro-6′H-spiro[cyclohexane-1,9′-furo[2,3-f]quinazoline]-2′-carboxamide; 5′-chloro-7′-oxo-N-[1-(pyridin-2-yl)cyclopropyl]-7′,8′-dihydro-6′H-spiro[cyclohexane-1,9′-furo[2,3-f]quinazoline]-2′-carboxamide; 5′-chloro-N-(4-methyloxan-4-yl)-7′-oxo-7′,8′-dihydro-6′H-spiro[cyclohexane-1,9′-furo[2,3-f]quinazoline]-2′-carboxamide; 5′-chloro-N-{[1-(ethoxymethyl)cyclopropyl]methyl}-7′-oxo-7′,8′-dihydro-6′H-spiro[cyclohexane-1,9′-furo[2,3-f]quinazoline]-2′-carboxamide; 2′-(4-benzoylpiperazine-1-carbonyl)-5′-chloro-7′,8′-dihydro-6′H-spiro[cyclohexane-1,9′-furo[2,3-f]quinazoline]-7′-one; 2′-(4-{bicyclo[2.2.1]heptan-2-yl}piperazine-1-carbonyl)-5′-chloro-7′,8′-dihydro-6′H-spiro[cyclohexane-1,9′-furo[2,3-f]quinazoline]-7′-one; 5′-chloro-N-[(1-methyl-1H-imidazol-5-yl)methyl]-7′-oxo-7′,8′-dihydro-6′H-spiro[cyclohexane-1,9′-furo[2,3-f]quinazoline]-2′-carboxamide; 5′-chloro-7′-oxo-N-[2-oxo-2-(piperidin-1-yl)ethyl]-7′,8′-dihydro-6′H-spiro[cyclohexane-1,9′-furo[2,3-f]quinazoline]-2′-carboxamide; 5′-chloro-2′-(3-hydroxyazetidine-1-carbonyl)-7′,8′-dihydro-6′H-spiro[cyclohexane-1,9′-furo[2,3-f]quinazoline]-7′-one; 5′-chloro-7′-oxo-N-{[5-(trifluoromethyl)pyridin-2-yl]methyl}-7′,8′-dihydro-6′H-spiro[cyclohexane-1,9′-furo[2,3-f]quinazoline]-2′-carboxamide; 5′-chloro-N-[1-(oxan-4-yl)cyclopropyl]-7′-oxo-7′,8′-dihydro-6′H-spiro[cyclohexane-1,9′-furo[2,3-f]quinazoline]-2′-carboxamide; 5′-chloro-N-methyl-7′-oxo-N-(prop-2-yn-1-yl)-7′,8′-dihydro-6′H-spiro[cyclohexane-1,9′-furo[2,3-f]quinazoline]-7′-carboxamide; 5′-chloro-N,N-diethyl-7′-oxo-7′,8′-dihydro-6′H-spiro[cyclohexane-1,9′-furo[2,3-f]quinazoline]-7′-carboxamide; 5′-chloro-N-(1-methyl-2-oxopiperidin-4-yl)-7′-oxo-7′,8′-dihydro-6′H-spiro[cyclohexane-1,9′-furo[2,3-f]quinazoline]-7′-carboxamide; 5′-chloro-2′-[(1R,5S,6S)-6-(pyridin-2-yl)-3-azabicyclo[3.1.0]hexane-3-carbonyl]-7′,8′-dihydro-6′H-spiro[cyclohexane-1,9′-furo[2,3-f]quinazoline]-7′-one; 5′-chloro-N-[(6-fluoropyridin-2-yl)methyl]-7′-oxo-7′,8′-dihydro-6′H-spiro[cyclohexane-1,9′-furo[2,3-f]quinazoline]-2′-carboxamide; 5′-chloro-N-ethyl-N-methyl-7′-oxo-7′,8′-dihydro-6′H-spiro[cyclohexane-1,9′-furo[2,3-f]quinazoline]-2′-carboxamide; 5′-chloro-2′-{6,6-dimethyl-3-azabicyclo[3.1.0]hexane-3-carbonyl}-7′,8′-dihydro-6′H-spiro[cyclohexane-1,9′-furo[2,3-f]quinazoline]-7′-one; 5′-chloro-2′-{7,7-difluoro-3-azabicyclo[4.1.0]heptane-3-carbonyl}-7′,8′-dihydro-6′H-spiro[cyclohexane-1,9′-furo[2,3-f]quinazoline]-7′-one; 5′-chloro-2′-{6,6-difluoro-3-azabicyclo[3.1.0]hexane-3-carbonyl}-7′,8′-dihydro-6′H-spiro[cyclohexane-1,9′-furo[2,3-f]quinazoline]-7′-one; 5′-chloro-7′-oxo-N-(2,2,2-trifluoroethyl)-7′,8′-dihydro-6′H-spiro[cyclohexane-1,9′-furo[2,3-f]quinazoline]-2′-carboxamide; 5′-chloro-7′-oxo-N-[(5-oxopyrrolidin-2-yl)methyl]-N-(propan-2-yl)-7′,8′-dihydro-6′H-spiro[cyclohexane-1,9′-furo[2,3-f]quinazoline]-2′-carboxamide; 5′-chloro-N-[2-(2,3-dihydro-1-benzofuran-5-yl)ethyl]-7′-oxo-7′,8′-dihydro-6′H-spiro[cyclohexane-1,9′-furo[2,3-f]quinazoline]-2′-carboxamide; 5′-chloro-N-(4,4-difluorocyclohexyl)-7′-oxo-7′,8′-dihydro-6′H-spiro[cyclohexane-1,9′-furo[2,3-f]quinazoline]-2′-carboxamide; N-{bicyclo[1.1.1]pentan-1-yl}-5′-chloro-7′-oxo-7′,8′-dihydro-6′H-spiro[cyclohexane-1,9′-furo[2,3-f]quinazoline]-2′-carboxamide; 5′-chloro-N-[(1S,3R)-3-fluorocyclopentyl]-7′-oxo-7′,8′-dihydro-6′H-spiro[cyclohexane-1,9′-furo[2,3-f]quinazoline]-2′-carboxamide; 5′-chloro-N-methyl-7′-oxo-N-(prop-2-en-1-yl)-7′,8′-dihydro-6′H-spiro[cyclohexane-1,9′-furo[2,3-f]quinazoline]-7′-carboxamide; 5′-chloro-7′-oxo-N-(prop-2-yn-1-yl)-7′,8′-dihydro-6′H-spiro[cyclohexane-1,9′-furo[2,3-f]quinazoline]-7′-carboxamide; 5′-chloro-7′-{2-ethyl-1-oxo-1H,2H,5H,6H,7H,8H-pyrido[3,4-d]pyridazine-6-carbonyl}-7′,8′-dihydro-6′H-spiro[cyclohexane-1,9′-furo[2,3-]quinazoline]-7′-one; 5′-chloro-N-(1,3-dihydro-2-benzofuran-5-ylmethyl)-7′-oxo-7′,8′-dihydro-6′H-spiro[cyclohexane-1,9′-furo[2,3-f]quinazoline]-2′-carboxamide; 5′-chloro-N-[4-(methoxymethyl)oxan-4-yl]-7′-oxo-7′,8′-dihydro-6′H-spiro[cyclohexane-1,9′-furo[2,3-]quinazoline]-2′-carboxamide; 5′-chloro-N-cyclobutyl-7′-oxo-7′,8′-dihydro-6′H-spiro[cyclohexane-1,9′-furo[2,3-f]quinazoline]-2′-carboxamide; 5′-chloro-2′-{9-propanoyl-2,9-diazaspiro[5.5]undecane-2-carbonyl}-7′,8′-dihydro-6′H-spiro[cyclohexane-1,9′-furo[2,3-f]quinazoline]-7′-one; 5′-chloro-N-methyl-7′-oxo-N-(propan-2-yl)-7′,8′-dihydro-6′H-spiro[cyclohexane-1,9′-furo[2,3-f]quinazoline]-2′-carboxamide; 5′-chloro-2′-[(1R,5S,6S)-6-(2-methoxyphenyl)-3-azabicyclo[3.1.0]hexane-3-carbonyl]-7′,8′-dihydro-6′H-spiro[cyclohexane-1,9′-furo[2,3-]quinazoline]-7′-one; N-butyl-5′-chloro-7′-oxo-7′,8′-dihydro-6′H-spiro[cyclohexane-1,9′-furo[2,3-f]quinazoline]-2′-carboxamide; 5′-chloro-2′-{8-ethoxy-2H,3H,4H,5H-pyrido[3,2-f][1,4]oxazepine-4-carbonyl}-7′,8′-dihydro-6′H-spiro[cyclohexane-1,9′-furo[2,3-f]quinazoline]-7′-one; 5′-chloro-7′-oxo-N-[2-(thiophen-3-yl)ethyl]-7′,8′-dihydro-6′H-spiro[cyclohexane-1,9′-furo[2,3-f]quinazoline]-2′-carboxamide; 5′-chloro-N-(2-cyclobutyl-2,2-difluoroethyl)-7′-oxo-7′,8′-dihydro-6′H-spiro[cyclohexane-1,9′-furo[2,3-f]quinazoline]-2′-carboxamide; 5′-chloro-N-methyl-7′-oxo-N-propyl-7′,8′-dihydro-6′H-spiro[cyclohexane-1,9′-furo[2,3-f]quinazoline]-2′-carboxamide; (2R)-1-({5′-chloro-7′-oxo-7′,8′-dihydro-6′H-spiro[cyclohexane-1,9′-furo[2,3-f]quinazoline]-2′-yl}carbonyl)pyrrolidine-2-carboxylic acid; 5′-chloro-7′-[(1R,6R)-7,7-difluoro-6-methyl-3-azabicyclo[4.1.0]heptane-3-carbonyl]-7′,8′-dihydro-6′H-spiro[cyclohexane-1,9′-furo[2,3-f]quinazoline]-7′-one; 5′-chloro-N-methyl-7′-oxo-N-{[4-(propan-2-yl)-4H-1,2,4-triazol-3-yl]methyl}-7′,8′-dihydro-6′H-spiro[cyclohexane-1,9′-furo[2,3-]quinazoline]-2′-carboxamide; 5′-chloro-7′-({4-oxo-3,4-dihydrospiro[1-benzopyran-2,3′-pyrrolidine]-1′-yl}carbonyl)-7′,8′-dihydro-6′H-spiro[cyclohexane-1,9′-furo[2,3-f]quinazoline]-7′-one; 5′-chloro-N-cyclopropyl-7′-oxo-7′,8′-dihydro-6′H-spiro[cyclohexane-1,9′-furo[2,3-f]quinazoline]-7′-carboxamide; 5′-chloro-N-ethyl-7′-oxo-N-propyl-7′,8′-dihydro-6′H-spiro[cyclohexane-1,9′-furo[2,3-f]quinazoline]-2′-carboxamide; 5′-chloro-7′-oxo-N-propyl-7′,8′-dihydro-6′H-spiro[cyclohexane-1,9′-furo[2,3-f]quinazoline]-2′-carboxamide; 5′-chloro-N-methyl-7′-oxo-N-(thiophen-3-ylmethyl)-7′,8′-dihydro-6′H-spiro[cyclohexane-1,9′-furo[2,3-f]quinazoline]-2′-carboxamide; 1-({5′-chloro-7′-oxo-7′,8′-dihydro-6′H-spiro[cyclohexane-1,9′-furo[2,3-f]quinazoline]-2′-yl}carbonyl)piperidine-4-carbonitrile; 5′-chloro-2′-[3-(dimethylamino)pyrrolidine-1-carbonyl]-7′,8′-dihydro-6′H-spiro[cyclohexane-1,9′-furo[2,3-]quinazoline]-7′-one; N-benzyl-5′-chloro-N-methyl-7′-oxo-7′,8′-dihydro-6′H-spiro[cyclohexane-1,9′-furo[2,3-f]quinazoline]-2′-carboxamide; 5′-chloro-N-methyl-7′-oxo-N-(thiophen-2-ylmethyl)-7′,8′-dihydro-6′H-spiro[cyclohexane-1,9′-furo[2,3-]quinazoline]-2′-carboxamide; 5′-chloro-N-[(3-methoxyphenyl)methyl]-7′-oxo-7′,8′-dihydro-6′H-spiro[cyclohexane-1,9′-furo[2,3-f]quinazoline]-2′-carboxamide; 5′-chloro-2′-(3-hydroxypyrrolidine-1-carbonyl)-7′,8′-dihydro-6′H-spiro[cyclohexane-1,9′-furo[2,3-f]quinazoline]-7′-one; 5′-chloro-7′-oxo-N,N-bis(propan-2-yl)-7′,8′-dihydro-6′H-spiro[cyclohexane-1,9′-furo[2,3-f]quinazoline]-2′-carboxamide; 5′-chloro-N-[2-(furan-2-yl)ethyl]-7′-oxo-7′,8′-dihydro-6′H-spiro[cyclohexane-1,9′-furo[2,3-f]quinazoline]-2′-carboxamide; 5′-chloro-7′-[2-(hydroxymethyl) piperidine-1-carbonyl]-7′,8′-dihydro-6′H-spiro[cyclohexane-1,9′-furo[2,3-f]quinazoline]-7′-one; 5′-chloro-7′-[4-(propan-2-yl)piperazine-1-carbonyl]-7′,8′-dihydro-6′H-spiro[cyclohexane-1,9′-furo[2,3-f]quinazoline]-7′-one; 5′-chloro-7′-[2-(furan-2-yl)pyrrolidine-1-carbonyl]-7′,8′-dihydro-6′H-spiro[cyclohexane-1,9′-furo[2,3-f]quinazoline]-7′-one; 5′-chloro-N-(3-methylbutyl)-7′-oxo-7′,8′-dihydro-6′H-spiro[cyclohexane-1,9′-furo[2,3-f]quinazoline]-2′-carboxamide; 5′-chloro-N-(2-cyanoethyl)-N-ethyl-7′-oxo-7′,8′-dihydro-6′H-spiro[cyclohexane-1,9′-furo[2,3-f]quinazoline]-2′-carboxamide; 5′-chloro-N-methyl-7′-oxo-N-(pyridin-2-ylmethyl)-7′,8′-dihydro-6′H-spiro[cyclohexane-1,9′-furo[2,3-]quinazoline]-2′-carboxamide; 5′-chloro-7′-oxo-N-[3-(pyrrolidin-1-yl)propyl]-7′,8′-dihydro-6′H-spiro[cyclohexane-1,9′-furo[2,3-f]quinazoline]-2′-carboxamide; 5′-chloro-7′-oxo-N-(pentan-3-yl)-7′,8′-dihydro-6′H-spiro[cyclohexane-1,9′-furo[2,3-f]quinazoline]-2′-carboxamide; 2′-(azepane-1-carbonyl)-5′-chloro-7′,8′-dihydro-6′H-spiro[cyclohexane-1,9′-furo[2,3-f]quinazoline]-7′-one; 5′-chloro-N-[2-(1H-imidazol-1-yl)ethyl]-7′-oxo-7′,8′-dihydro-6′H-spiro[cyclohexane-1,9′-furo[2,3-f]quinazoline]-2′-carboxamide; 5′-chloro-2′-[(2R,6S)-2,6-dimethylmorpholine-4-carbonyl]-7′,8′-dihydro-6′H-spiro[cyclohexane-1,9′-furo[2,3-f]quinazoline]-7′-one; 5′-chloro-N-methyl-7′-oxo-N-(pyridin-3-ylmethyl)-7′,8′-dihydro-6′H-spiro[cyclohexane-1,9′-furo[2,3-]quinazoline]-2′-carboxamide; 5′-chloro-N-methyl-N-(1-methylpiperidin-4-yl)-7′-oxo-7′,8′-dihydro-6′H-spiro[cyclohexane-1,9′-furo[2,3-f]quinazoline]-2′-carboxamide; 5′-chloro-N-[(3-ethyl-1,2-oxazol-5-yl)methyl]-N-methyl-7′-oxo-7′,8′-dihydro-6′H-spiro[cyclohexane-1,9′-furo[2,3-f]quinazoline]-2′-carboxamide; 5′-chloro-N-(3-methylbutan-2-yl)-7′-oxo-7′,8′-dihydro-6′H-spiro[cyclohexane-1,9′-furo[2,3-f]quinazoline]-2′-carboxamide; 5′-chloro-7′-(3-methylpiperidine-1-carbonyl)-7′,8′-dihydro-6′H-spiro[cyclohexane-1,9′-furo[2,3-f]quinazoline]-7′-one; 5′-chloro-N-[2-(dimethylamino)ethyl]-N-methyl-7′-oxo-7′,8′-dihydro-6′H-spiro[cyclohexane-1,9′-furo[2,3-f]quinazoline]-7′-carboxamide; 5′-chloro-7′-(2,2-dimethylmorpholine-4-carbonyl)-7′,8′-dihydro-6′H-spiro[cyclohexane-1,9′-furo[2,3-f]quinazoline]-7′-one; 5′-chloro-N-[(3-hydroxyphenyl)methyl]-7′-oxo-7′,8′-dihydro-6′H-spiro[cyclohexane-1,9′-furo[2,3-f]quinazoline]-7′-carboxamide; 5′-chloro-7′-[4-(dimethylamino)piperidine-1-carbonyl]-7′,8′-dihydro-6′H-spiro[cyclohexane-1,9′-furo[2,3-f]quinazoline]-7′-one; 5′-chloro-7′-oxo-N-[3-(2-oxopyrrolidin-1-yl)propyl]-7′,8′-dihydro-6′H-spiro[cyclohexane-1,9′-furo[2,3-]quinazoline]-2′-carboxamide; 5′-chloro-2′-(4-methylpiperidine-1-carbonyl)-7′,8′-dihydro-6′H-spiro[cyclohexane-1,9′-furo[2,3-f]quinazoline]-7′-one; 5′-chloro-7′-oxo-N-(thiophen-3-ylmethyl)-7′,8′-dihydro-6′H-spiro[cyclohexane-1,9′-furo[2,3-f]quinazoline]-2′-carboxamide; 5′-chloro-N-[(3-fluorophenyl)methyl]-7′-oxo-7′,8′-dihydro-6′H-spiro[cyclohexane-1,9′-furo[2,3-f]quinazoline]-2′-carboxamide; N-benzyl-5′-chloro-N-(2-hydroxyethyl)-7′-oxo-7′,8′-dihydro-6′H-spiro[cyclohexane-1,9′-furo[2,3-f]quinazoline]-2′-carboxamide; 5′-chloro-N-[2-(3,5-dimethyl-1H-pyrazol-4-yl)ethyl]-N-methyl-7′-oxo-7′,8′-dihydro-6′H-spiro[cyclohexane-1,9′-furo[2,3-f]quinazoline]-2′-carboxamide; 5′-chloro-2′-(4-cyclohexylpiperazine-1-carbonyl)-7′,8′-dihydro-6′H-spiro[cyclohexane-1,9′-furo[2,3-f]quinazoline]-7′-one; 5′-chloro-2′-(4-hydroxy-4-phenylpiperidine-1-carbonyl)-7′,8′-dihydro-6′H-spiro[cyclohexane-1,9′-furo[2,3-f]quinazoline]-7′-one; 5′-chloro-2′-[4-(oxolane-2-carbonyl)piperazine-1-carbonyl]-7′,8′-dihydro-6′H-spiro[cyclohexane-1,9′-furo[2,3-f]quinazoline]-7′-one; 5′-chloro-2′-{4-[(dimethylamino)methyl]piperidine-1-carbonyl}-7′,8′-dihydro-6′H-spiro[cyclohexane-1,9′-furo[2,3-f]quinazoline]-7′-one; 1-({5′-chloro-7′-oxo-7′,8′-dihydro-6′H-spiro[cyclohexane-1,9′-furo[2,3-f]quinazoline]-2′-yl}carbonyl)-N-methylpiperidine-4-carboxamide; 5′-chloro-N-[2-(4-methoxyphenyl)ethyl]-7′-oxo-7′,8′-dihydro-6′H-spiro[cyclohexane-1,9′-furo[2,3-f]quinazoline]-7′-carboxamide; 5′-chloro-N-[2-(dimethyl-1,2-oxazol-4-yl)ethyl]-N-methyl-7′-oxo-7′,8′-dihydro-6′H-spiro[cyclohexane-1,9′-furo[2,3-f]quinazoline]-7′-carboxamide; 5′-chloro-2′-[4-(1,3-thiazol-2-yl)piperazine-1-carbonyl]-7′,8′-dihydro-6′H-spiro[cyclohexane-1,9′-furo[2,3-f]quinazoline]-7′-one; 5′-chloro-N-{[4-(1H-imidazol-1-yl)phenyl]methyl}-N-methyl-7′-oxo-7′,8′-dihydro-6′H-spiro[cyclohexane-1,9′-furo[2,3-f]quinazoline]-2′-carboxamide; 2′-(4-tert-butylpiperazine-1-carbonyl)-5′-chloro-7′,8′-dihydro-6′H-spiro[cyclohexane-1,9′-furo[2,3-f]quinazoline]-7′-one; 5′-chloro-N-[2-(3-methoxyphenyl)ethyl]-7′-oxo-7′,8′-dihydro-6′H-spiro[cyclohexane-1,9′-furo[2,3-f]quinazoline]-2′-carboxamide; 5′-chloro-2′-[4-(pyrrolidin-1-yl)piperidine-1-carbonyl]-7′,8′-dihydro-6′H-spiro[cyclohexane-1,9′-furo[2,3-f]quinazoline]-7′-one; 5′-chloro-N-(2,3-dihydro-1,4-benzodioxin-2-ylmethyl)-7′-oxo-7′,8′-dihydro-6′H-spiro[cyclohexane-1,9′-furo[2,3-f]quinazoline]-2′-carboxamide; 5′-chloro-2′-[4-(morpholin-4-yl)piperidine-1-carbonyl]-7′,8′-dihydro-6′H-spiro[cyclohexane-1,9′-furo[2,3-f]quinazoline]-7′-one; 5′-chloro-2′-[4-(3-methoxyphenyl)piperazine-1-carbonyl]-7′,8′-dihydro-6′H-spiro[cyclohexane-1,9′-furo[2,3-f]quinazoline]-7′-one; 5′-chloro-7′-oxo-N-[1-(propan-2-yl)piperidin-4-yl]-7′,8′-dihydro-6′H-spiro[cyclohexane-1,9′-furo[2,3-f]quinazoline]-2′-carboxamide; 5′-chloro-N-(1-hydroxy-3-phenylpropan-2-yl)-7′-oxo-7′,8′-dihydro-6′H-spiro[cyclohexane-1,9′-furo[2,3-f]quinazoline]-2′-carboxamide; N-(2H-1,3-benzodioxol-5-ylmethyl)-5′-chloro-N-methyl-7′-oxo-7′,8′-dihydro-6′H-spiro[cyclohexane-1,9′-furo[2,3-f]quinazoline]-2′-carboxamide; methyl 3-[1-({5′-chloro-7′-oxo-7′,8′-dihydro-6′H-spiro[cyclohexane-1,9′-furo[2,3-f]quinazoline]-2′-yl}carbonyl)piperidin-2-yl]propanoate; 5′-chloro-7′-(4-cyclopentylpiperazine-1-carbonyl)-7′,8′-dihydro-6′H-spiro[cyclohexane-1,9′-furo[2,3-f]quinazoline]-7′-one; 5′-chloro-7′-[4-(2-methylpropanoyl)piperazine-1-carbonyl]-7′,8′-dihydro-6′H-spiro[cyclohexane-1,9′-furo[2,3-f]quinazoline]-7′-one; N-benzyl-5′-chloro-N-(3-hydroxypropyl)-7′-oxo-7′,8′-dihydro-6′H-spiro[cyclohexane-1,9′-furo[2,3-f]quinazoline]-7′-carboxamide; 5′-chloro-N-(2-methoxyethyl)-7′-oxo-N-(thiophen-2-ylmethyl)-7′,8′-dihydro-6′H-spiro[cyclohexane-1,9′-furo[2,3-f]quinazoline]-2′-carboxamide; 5′-chloro-N-[2-(dimethylamino)ethyl]-7′-oxo-7′,8′-dihydro-6′H-spiro[cyclohexane-1,9′-furo[2,3-f]quinazoline]-2′-carboxamide; 5′-chloro-2′-{5H,6H,7H-pyrrolo [3,4-b]pyridine-6-carbonyl}-7′,8′-dihydro-6′H-spiro[cyclohexane-1,9′-furo[2,3-f]quinazoline]-7′-one; 5′-chloro-N-cyclohexyl-N-(2-hydroxyethyl)-7′-oxo-7′,8′-dihydro-6′H-spiro[cyclohexane-1,9′-furo[2,3-f]quinazoline]-2′-carboxamide; 5′-chloro-N-[2-(dimethyl-1,3-thiazol-2-yl)ethyl]-7′-oxo-7′,8′-dihydro-6′H-spiro[cyclohexane-1,9′-furo[2,3-f]quinazoline]-2′-carboxamide; 5′-chloro-2′-({[(3R,4R)-3-methoxyoxan-4-yl]amino}methyl)-7′,8′-dihydro-6′H-spiro[cyclohexane-1,9′-furo[2,3-f]quinazoline]-7′-one; 5′-chloro-2′-{[2-(2-hydroxyethyl)piperidin-1-yl]methyl}-7′,8′-dihydro-6′H-spiro[cyclohexane-1,9′-furo[2,3-f]quinazoline]-7′-one; 5′-chloro-2′-[(3-oxopiperazin-1-yl)methyl]-7′,8′-dihydro-6′H-spiro[cyclohexane-1,9′-furo[2,3-f]quinazoline]-7′-one; 5′-chloro-2′-({[(1-methyl-1H-pyrazol-3-yl)methyl]amino}methyl)-7′,8′-dihydro-6′H-spiro[cyclohexane-1,9′-furo[2,3-f]quinazoline]-7′-one; 5′-chloro-2′-{[(2-methoxyethyl)(propan-2-yl)amino]methyl}-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}piperidine-4-carbonitrile; 5′-chloro-2′-{[(2-hydroxyethyl)(propan-2-yl)amino]methyl}-7′,8′-dihydro-6′H-spiro[cyclohexane-1,9′-furo[2,3-f]quinazoline]-7′-one; 5′-chloro-2′-{[4-(pyridin-4-yl)piperazin-1-yl]methyl}-7′,8′-dihydro-6′H-spiro[cyclohexane-1,9′-furo[2,3-f]quinazoline]-7′-one; 5′-chloro-2′-({[2-(1H-pyrazol-1-yl)ethyl]amino}methyl)-7′,8′-dihydro-6′H-spiro[cyclohexane-1,9′-furo[2,3-f]quinazoline]-7′-one; 5′-chloro-2′-{[(oxan-4-yl)amino]methyl}-7′,8′-dihydro-6′H-spiro[cyclohexane-1,9′-furo[2,3-f]quinazoline]-7′-one; 2′-[(8aS)-octahydropyrrolo[1,2-a]piperazin-2-ylmethyl]-5′-chloro-7′,8′-dihydro-6′H-spiro[cyclohexane-1,9′-furo[2,3-f]quinazoline]-7′-one; 5′-chloro-2′-{[(oxolan-2-ylmethyl)amino]methyl}-7′,8′-dihydro-6′H-spiro[cyclohexane-1,9′-furo[2,3-f]quinazoline]-7′-one; 3-({5′-chloro-7′-oxo-7′,8′-dihydro-6′H-spiro[cyclohexane-1,9′-furo[2,3-f]quinazoline]-2′-ylmethyl}(ethyl)amino)propanenitrile; 5′-chloro-2′-{[(pyridin-2-ylmethyl)amino]methyl}-7′,8′-dihydro-6′H-spiro[cyclohexane-1,9′-furo[2,3-f]quinazoline]-7′-one; 5′-chloro-2′-{[2-(hydroxymethyl)pyrrolidin-1-yl]methyl}-7′,8′-dihydro-6′H-spiro[cyclohexane-1,9′-furo[2,3-f]quinazoline]-7′-one; 5′-chloro-2′-{[(2-methanesulfonylethyl)amino]methyl}-7′,8′-dihydro-6′H-spiro[cyclohexane-1,9′-furo[2,3-f]quinazoline]-7′-one; 2′-{[benzyl(2-hydroxyethyl)amino]methyl}-5′-chloro-7′,8′-dihydro-6′H-spiro[cyclohexane-1,9′-furo[2,3-f]quinazoline]-7′-one; 5′-chloro-2′-{[(furan-2-ylmethyl)(methyl)amino]methyl}-7′,8′-dihydro-6′H-spiro[cyclohexane-1,9′-furo[2,3-f]quinazoline]-7′-one; 5′-chloro-2′-[(4-methoxypiperidin-1-yl)methyl]-7′,8′-dihydro-6′H-spiro[cyclohexane-1,9′-furo[2,3-f]quinazoline]-7′-one; 2′-[(4-acetyl-1,4-diazepan-1-yl)methyl]-5′-chloro-7′,8′-dihydro-6′H-spiro[cyclohexane-1,9′-furo[2,3-f]quinazoline]-7′-one; 5′-chloro-2′-{[4-(2-hydroxyethyl)piperazin-1-yl]methyl}-7′,8′-dihydro-6′H-spiro[cyclohexane-1,9′-furo[2,3-f]quinazoline]-7′-one; 5′-chloro-2′-({4-[(dimethylamino)methyl]piperidin-1-yl}methyl)-7′,8′-dihydro-6′H-spiro[cyclohexane-1,9′-furo[2,3-f]quinazoline]-7′-one; 5′-chloro-7′-{[4-(oxolane-2-carbonyl)piperazin-1-yl]methyl}-7′,8′-dihydro-6′H-spiro[cyclohexane-1,9′-furo[2,3-f]quinazoline]-7′-one; 5′-chloro-2′-({[(5-oxopyrrolidin-2-yl)methyl](propan-2-yl)amino}methyl)-7′,8′-dihydro-6′H-spiro[cyclohexane-1,9′-furo[2,3-f]quinazoline]-7′-one; 5′-chloro-2′-{5H,6H,7H,8H-imidazo [1,2-a]pyrazin-7-ylmethyl}-7′,8′-dihydro-6′H-spiro[cyclohexane-1,9′-furo[2,3-f]quinazoline]-7′-one; 5′-chloro-2′-({[3-(2-oxopyrrolidin-1-yl)propyl]amino}methyl)-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}-N-methylpiperidine-4-carboxamide; 5′-chloro-2′-{[(3-hydroxypropyl)(methyl)amino]methyl}-7′,8′-dihydro-6′H-spiro[cyclohexane-1,9′-furo[2,3-f]quinazoline]-7′-one; 5′-chloro-2′-[(4-hydroxy-4-phenylpiperidin-1-yl)methyl]-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}piperidine-3-carboxamide; 3-({5′-chloro-7′-oxo-7′,8′-dihydro-6′H-spiro[cyclohexane-1,9′-furo[2,3-f]quinazoline]-2′-ylmethyl}(methyl)amino)-N-methylpropanamide; 5′-chloro-2′-[{(1R,5S,6S)-6-(hydroxymethyl)-3-azabicyclo[3.1.0]hexan-3-yl]methyl}-7′,8′-dihydro-6′H-spiro[cyclohexane-1,9′-furo[2,3-f]quinazoline]-7′-one; 5′-chloro-2′-{[ethyl(2-hydroxyethyl)amino]methyl}-7′,8′-dihydro-6′H-spiro[cyclohexane-1,9′-furo[2,3-f]quinazoline]-7′-one; 5′-chloro-2′-{[(2-hydroxyethyl)(methyl)amino]methyl}-7′,8′-dihydro-6′H-spiro[cyclohexane-1,9′-furo[2,3-f]quinazoline]-7′-one; 5′-chloro-2′-[(3-hydroxypiperidin-1-yl)methyl]-7′,8′-dihydro-6′H-spiro[cyclohexane-1,9′-furo[2,3-f]quinazoline]-7′-one; 5′-chloro-2′-{[4-(2-hydroxyethyl)piperidin-1-yl]methyl}-7′,8′-dihydro-6′H-spiro[cyclohexane-1,9′-furo[2,3-f]quinazoline]-7′-one; 5′-chloro-2′-{[(2R,6S)-2,6-dimethylmorpholin-4-yl]methyl}-7′,8′-dihydro-6′H-spiro[cyclohexane-1,9′-furo[2,3-f]quinazoline]-7′-one; 5′-chloro-2′-({[(4-ethyl-4H-1,2,4-triazol-3-yl)methyl](methyl)amino}methyl)-7′,8′-dihydro-6′H-spiro[cyclohexane-1,9′-furo[2,3-f]quinazoline]-7′-one; 5′-chloro-7′-{2-oxa-6-azaspiro[3.5]nonan-6-ylmethyl}-7′,8′-dihydro-6′H-spiro[cyclohexane-1,9′-furo[2,3-f]quinazoline]-7′-one; 5′-chloro-7′-{[ethyl(propan-2-yl)amino]methyl}-7′,8′-dihydro-6′H-spiro[cyclohexane-1,9′-furo[2,3-f]quinazoline]-7′-one; 5′-chloro-2′-({[(5-cyclopropyl-1H-pyrazol-3-yl)methyl](methyl)amino}methyl)-7′,8′-dihydro-6′H-spiro[cyclohexane-1,9′-furo[2,3-f]quinazoline]-7′-one; 5′-chloro-2′-({[3-(dimethylamino)propyl](methyl)amino}methyl)-7′,8′-dihydro-6′H-spiro[cyclohexane-1,9′-furo[2,3-f]quinazoline]-7′-one; 5′-chloro-2′-{[3-(dimethylamino)pyrrolidin-1-yl]methyl}-7′,8′-dihydro-6′H-spiro[cyclohexane-1,9′-furo[2,3-f]quinazoline]-7′-one; 2-(4-{5′-chloro-7′-oxo-7′,8′-dihydro-6′H-spiro[cyclohexane-1,9′-furo[2,3-f]quinazoline]-2′-ylmethyl}piperazin-1-yl)pyridine-3-carbonitrile; 5′-chloro-2′-{[3-(1H-imidazol-1-ylmethyl)piperidin-1-yl]methyl}-7′,8′-dihydro-6′H-spiro[cyclohexane-1,9′-furo[2,3-f]quinazoline]-7′-one; 5′-chloro-2′-{[4-hydroxy-4-(thiophen-2-yl)piperidin-1-yl]methyl}-7′,8′-dihydro-6′H-spiro[cyclohexane-1,9′-furo[2,3-f]quinazoline]-7′-one; 5′-chloro-2′-{[4-(2-methoxyethyl)piperazin-1-yl]methyl}-7′,8′-dihydro-6′H-spiro[cyclohexane-1,9′-furo[2,3-f]quinazoline]-7′-one; 5′-chloro-2′-{[methyl(pyridin-3-ylmethyl)amino]methyl}-7′,8′-dihydro-6′H-spiro[cyclohexane-1,9′-furo[2,3-f]quinazoline]-7′-one; 4-{5′-chloro-7′-oxo-7′,8′-dihydro-6′H-spiro[cyclohexane-1,9′-furo[2,3-f]quinazoline]-2′-ylmethyl}-thiomorpholine-1,1-dione; 5′-chloro-2′-{[(4-hydroxy-2-methylbutan-2-yl)amino]methyl}-7′,8′-dihydro-6′H-spiro[cyclohexane-1,9′-furo[2,3-f]quinazoline]-7′-one; 4-{5′-chloro-7′-oxo-7′,8′-dihydro-6′H-spiro[cyclohexane-1,9′-furo[2,3-f]quinazoline]-2′-ylmethyl}piperazine-1-carbaldehyde; 5′-chloro-2′-{[4-(morpholin-4-yl)piperidin-1-yl]methyl}-7′,8′-dihydro-6′H-spiro[cyclohexane-1,9′-furo[2,3-f]quinazoline]-7′-one; 4-{5′-chloro-7′-oxo-7′,8′-dihydro-6′H-spiro[cyclohexane-1,9′-furo[2,3-f]quinazoline]-2′-ylmethyl}-N,N-dimethylpiperazine-1-carboxamide; 5′-chloro-2′-[({[4-(propan-2-yl)-4H-1,2,4-triazol-3-yl]methyl}amino)methyl]-7′,8′-dihydro-6′H-spiro[cyclohexane-1,9′-furo[2,3-f]quinazoline]-7′-one; 5′-chloro-2′-{[3-(trifluoromethyl)-5H,6H,7H,8H-[1,2,4]triazolo[4,3-a]pyrazin-7-yl]methyl}-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}azetidine-3-carbonitrile; 4-({5′-chloro-7′-oxo-7′,8′-dihydro-6′H-spiro[cyclohexane-1,9′-furo[2,3-f]quinazoline]-2′-ylmethyl}amino)-thiane-1,1-dione; 5′-chloro-2′-[(4-methanesulfonylpiperazin-1-yl)methyl]-7′,8′-dihydro-6′H-spiro[cyclohexane-1,9′-furo[2,3-f]quinazoline]-7′-one; ethyl 7-{5′-chloro-7′-oxo-7′,8′-dihydro-6′H-spiro[cyclohexane-1,9′-furo[2,3-f]quinazoline]-2′-ylmethyl}-5H,6H,7H,8H-imidazo[1,2-a]pyrazine-2-carboxylate; 2′-{6-azaspiro[2.5]octan-6-ylmethyl}-5′-chloro-7′,8′-dihydro-6′H-spiro[cyclohexane-1,9′-furo[2,3-f]quinazoline]-7′-one; 5′-chloro-2′-{[(2,2,2-trifluoroethyl)amino]methyl}-7′,8′-dihydro-6′H-spiro[cyclohexane-1,9′-furo[2,3-f]quinazoline]-7′-one; 5′-chloro-2′-{octahydropyrrolo[1,2-a]piperazin-2-ylmethyl}-7′,8′-dihydro-6′H-spiro[cyclohexane-1,9′-furo[2,3-f]quinazoline]-7′-one; 5′-chloro-2′-{[4-(furan-2-ylmethyl)piperazin-1-yl]methyl}-7′,8′-dihydro-6′H-spiro[cyclohexane-1,9′-furo[2,3-f]quinazoline]-7′-one; 5′-chloro-2′-{[methyl(pro pan-2-yl)amino]methyl}-7′,8′-dihydro-6′H-spiro[cyclohexane-1,9′-furo[2,3-f]quinazoline]-7′-one; 3-({5′-chloro-7′-oxo-7′,8′-dihydro-6′H-spiro[cyclohexane-1,9′-furo[2,3-f]quinazoline]-2′-ylmethyl}amino)-thiolane-1,1-dione; 5′-chloro-2′-{[4-(pyridin-3-ylmethyl)piperazin-1-yl]methyl}-7′,8′-dihydro-6′H-spiro[cyclohexane-1,9′-furo[2,3-f]quinazoline]-7′-one; 5′-chloro-2′-[(4-methyl-1,4-diazepan-1-yl)methyl]-7′,8′-dihydro-6′H-spiro[cyclohexane-1,9′-furo[2,3-f]quinazoline]-7′-one; 5′-chloro-7′-{[4-(dimethylamino)piperidin-1-yl]methyl}-7′,8′-dihydro-6′H-spiro[cyclohexane-1,9′-furo[2,3-f]quinazoline]-7′-one; 5′-chloro-7′-({methyl[(trimethyl-1H-pyrazol-4-yl)methyl]amino}methyl)-7′,8′-dihydro-6′H-spiro[cyclohexane-1,9′-furo[2,3-f]quinazoline]-7′-one; 5′-chloro-7′-{[3-oxo-4-(propan-2-yl)piperazin-1-yl]methyl}-7′,8′-dihydro-6′H-spiro[cyclohexane-1,9′-furo[2,3-f]quinazoline]-7′-one; 5′-chloro-7′-{[(2,2-dimethyloxan-4-yl)(ethyl)amino]methyl}-7′,8′-dihydro-6′H-spiro[cyclohexane-1,9′-furo[2,3-f]quinazoline]-7′-one; 5′-chloro-2′-{[4-(pyrrolidine-1-carbonyl)piperazin-1-yl]methyl}-7′,8′-dihydro-6′H-spiro[cyclohexane-1,9′-furo[2,3-f]quinazoline]-7′-one; 5′-chloro-2′-{[(1,3-oxazol-5-ylmethyl)amino]methyl}-7′,8′-dihydro-6′H-spiro[cyclohexane-1,9′-furo[2,3-f]quinazoline]-7′-one; 5′-chloro-2′-[(propylamino)methyl]-7′,8′-dihydro-6′H-spiro[cyclohexane-1,9′-furo[2,3-f]quinazoline]-7′-one; 5′-chloro-2′-({[(1-methyl-1H-pyrazol-5-yl)methyl]amino}methyl)-7′,8′-dihydro-6′H-spiro[cyclohexane-1,9′-furo[2,3-f]quinazoline]-7′-one; 5′-chloro-2′-{[4-(oxetan-3-yl)piperazin-1-yl]methyl}-7′,8′-dihydro-6′H-spiro[cyclohexane-1,9′-furo[2,3-f]quinazoline]-7′-one; 5′-chloro-2′-({methyl[2-(pyridin-2-yl)ethyl]amino}methyl)-7′,8′-dihydro-6′H-spiro[cyclohexane-1,9′-furo[2,3-f]quinazoline]-7′-one; 5′-chloro-2′-{[(2-methoxyethyl)(methyl)amino]methyl}-7′,8′-dihydro-6′H-spiro[cyclohexane-1,9′-furo[2,3-f]quinazoline]-7′-one; 5′-chloro-2′-{5H,6H,7H-pyrrolo [3,4-b]pyridin-6-ylmethyl}-7′,8′-dihydro-6′H-spiro[cyclohexane-1,9′-furo[2,3-f]quinazoline]-7′-one; 5′-chloro-2′-({[2-(3,5-dimethyl-1H-pyrazol-4-yl)ethyl](methyl)amino}methyl)-7′,8′-dihydro-6′H-spiro[cyclohexane-1,9′-furo[2,3-f]quinazoline]-7′-one; 5′-chloro-2′-({[2-(dimethylamino)ethyl](ethyl)amino}methyl)-7′,8′-dihydro-6′H-spiro[cyclohexane-1,9′-furo[2,3-f]quinazoline]-7′-one; 5′-chloro-2′-[(3-hydroxypyrrolidin-1-yl)methyl]-7′,8′-dihydro-6′H-spiro[cyclohexane-1,9′-furo[2,3-f]quinazoline]-7′-one; 5′-chloro-7′-[({[6-(morpholin-4-yl)pyridin-3-yl]methyl}amino)methyl]-7′,8′-dihydro-6′H-spiro[cyclohexane-1,9′-furo[2,3-f]quinazoline]-7′-one; 5′-chloro-7′-{[methyl(propyl)amino]methyl}-7′,8′-dihydro-6′H-spiro[cyclohexane-1,9′-furo[2,3-f]quinazoline]-7′-one; 5′-chloro-2′-({[3-(3,5-dimethyl-1H-pyrazol-1-yl)propyl](methyl)amino}methyl)-7′,8′-dihydro-6′H-spiro[cyclohexane-1,9′-furo[2,3-f]quinazoline]-7′-one; 5′-chloro-7′-[(2,2-dimethylmorpholin-4-yl)methyl]-7′,8′-dihydro-6′H-spiro[cyclohexane-1,9′-furo[2,3-f]quinazoline]-7′-one; 5′-chloro-2′-[(4-ethylpiperazin-1-yl)methyl]-7′,8′-dihydro-6′H-spiro[cyclohexane-1,9′-furo[2,3-f]quinazoline]-7′-one; 5′-chloro-2′-({[(3-ethyl-1,2-oxazol-5-yl)methyl](methyl)amino}methyl)-7′,8′-dihydro-6′H-spiro[cyclohexane-1,9′-furo[2,3-f]quinazoline]-7′-one; 5′-chloro-2′-{[methyl(pyridin-2-ylmethyl)amino]methyl}-7′,8′-dihydro-6′H-spiro[cyclohexane-1,9′-furo[2,3-f]quinazoline]-7′-one; 1-{5′-chloro-7′-oxo-7′,8′-dihydro-6′H-spiro[cyclohexane-1,9′-furo[2,3-f]quinazoline]-2′-ylmethyl}piperidine-4-carboxamide; 5′-chloro-2′-({[2-(2-oxopyrrolidin-1-yl)ethyl]amino}methyl)-7′,8′-dihydro-6′H-spiro[cyclohexane-1,9′-furo[2,3-f]quinazoline]-7′-one; 5′-chloro-2′-[({[1-(ethoxymethyl)cyclopropyl]methyl}amino)methyl]-7′,8′-dihydro-6′H-spiro[cyclohexane-1,9′-furo[2,3-f]quinazoline]-7′-one; 5′-chloro-2′-({[2-(dimethyl-1,2-oxazol-4-yl)ethyl](methyl)amino}methyl)-7′,8′-dihydro-6′H-spiro[cyclohexane-1,9′-furo[2,3-f]quinazoline]-7′-one; N-(1-{5′-chloro-7′-oxo-7′,8′-dihydro-6′H-spiro[cyclohexane-1,9′-furo[2,3-f]quinazoline]-2′-ylmethyl}piperidin-4-yl)acetamide; 5′-chloro-2′-{[2-(hydroxymethyl)piperidin-1-yl]methyl}-7′,8′-dihydro-6′H-spiro[cyclohexane-1,9′-furo[2,3-f]quinazoline]-7′-one; 5′-chloro-2′-{[methyl(1-methylpiperidin-4-yl)amino]methyl}-7′,8′-dihydro-6′H-spiro[cyclohexane-1,9′-furo[2,3-f]quinazoline]-7′-one; 5′-chloro-2′-({6,6-dimethyl-3-azabicyclo[3.1.0]hexan-3-yl}methyl)-7′,8′-dihydro-6′H-spiro[cyclohexane-1,9′-furo[2,3-f]quinazoline]-7′-one; 5′-chloro-2′-({[(1-methyl-1H-imidazol-2-yl)methyl]amino}methyl)-7′,8′-dihydro-6′H-spiro[cyclohexane-1,9′-furo[2,3-f]quinazoline]-7′-one; 5′-chloro-7′-{[3-(hydroxymethyl)piperidin-1-yl]methyl}-7′,8′-dihydro-6′H-spiro[cyclohexane-1,9′-furo[2,3-f]quinazoline]-7′-one; 5′-chloro-7′-{[(2-hydroxyethyl)(propyl)amino]methyl}-7′,8′-dihydro-6′H-spiro[cyclohexane-1,9′-furo[2,3-f]quinazoline]-7′-one; ethyl 2-({5′-chloro-7′-oxo-7′,8′-dihydro-6′H-spiro[cyclohexane-1,9′-furo[2,3-f]quinazoline]-2′-ylmethyl}(methyl)amino)acetate; 2′-[(3-aminopyrrolidin-1-yl)methyl]-5′-chloro-7′,8′-dihydro-6′H-spiro[cyclohexane-1,9′-furo[2,3-f]quinazoline]-7′-one; N-{2-({5′-chloro-7′-oxo-7′,8′-dihydro-6′H-spiro[cyclohexane-1,9′-furo[2,3-f]quinazoline]-2′-ylmethyl}amino)ethyl}acetamide; 5′-chloro-2′-{[4-(hydroxymethyl)piperidin-1-yl]methyl}-7′,8′-dihydro-6′H-spiro[cyclohexane-1,9′-furo[2,3-f]quinazoline]-7′-one; 5′-chloro-2′-{[(piperidin-3-yl)amino]methyl}-7′,8′-dihydro-6′H-spiro[cyclohexane-1,9′-furo[2,3-f]quinazoline]-7′-one; 5′-chloro-2′-{[(pyrrolidin-3-yl)amino]methyl}-7′,8′-dihydro-6′H-spiro[cyclohexane-1,9′-furo[2,3-f]quinazoline]-7′-one; 2′-benzoyl-5′-chloro-7′,8′-dihydro-6′H-spiro[cyclohexane-1,9′-furo[2,3-f]quinazoline]-7′-one; 5′-chloro-2′-(pyridine-4-carbonyl)-7′,8′-dihydro-6′H-spiro[cyclohexane-1,9′-furo[2,3-f]quinazoline]-7′-one; 2′-acetyl-5′-chloro-7′,8′-dihydro-6′H-spiro[cyclohexane-1,9′-furo[2,3-]quinazoline]-7′-one; 5′-chloro-2′-({[(3S,4R)-3-methoxyoxan-4-yl]amino}methyl)-7′,8′-dihydro-6′H-spiro[cyclohexane-1,9′-furo[2,3-f]quinazoline]-7′-one; 5′-chloro-2′-{[(3-fluorooxan-4-yl)amino]methyl}-7′,8′-dihydro-6′H-spiro[cyclohexane-1,9′-furo[2,3-f]quinazoline]-7′-one; 5′-chloro-2′-({[(3R)-oxolan-3-yl]amino}methyl)-7′,8′-dihydro-6′H-spiro[cyclohexane-1,9′-furo[2,3-f]quinazoline]-7′-one; 5′-chloro-2′-({[(3S)-oxolan-3-yl]amino}methyl)-7′,8′-dihydro-6′H-spiro[cyclohexane-1,9′-furo[2,3-f]quinazoline]-7′-one; 5′-chloro-7′-({[(3R,4S)-3-methoxyoxan-4-yl]amino}methyl)-7′,8′-dihydro-6′H-spiro[cyclohexane-1,9′-furo[2,3-f]quinazoline]-7′-one; 5′-chloro-7′-({[(3S,4R)-3-methoxyoxan-4-yl]amino}methyl)-7′,8′-dihydro-6′H-spiro[cyclohexane-1,9′-furo[2,3-f]quinazoline]-7′-one; 5′-chloro-7′-{[(2-methoxyethyl)amino]methyl}-7′,8′-dihydro-6′H-spiro[cyclohexane-1,9′-furo[2,3-f]quinazoline]-7′-one; 5′-chloro-2′-{[(oxetan-3-yl)amino]methyl}-7′,8′-dihydro-6′H-spiro[cyclohexane-1,9′-furo[2,3-f]quinazoline]-7′-one; and pharmaceutically acceptable salts thereof.

11. The substance as claimed in claim 8, wherein the inhibitory agent is a member selected from the group consisting of compounds with the CAS Registry Numbers assigned by the Chemical Abstracts Service (CAS) of 2266633-30-9; 2266633-88-7; 2266633-90-1; 2266633-95-6; 2266634-01-7; 2266634-08-4; and 2266637-12-9.

12. A compound of claim 8 selected from the group consisting of: 5-chloro-2-[(1S,4S)-2-oxa-5-azabicyclo[2.2.1]heptan-5-ylmethyl]-7,8-dihydro-6H-spiro[[1,3]oxazolo[5,4-f]quinazoline-9,1′-cyclohexane]-7-one; 5-chloro-2-[(3-methoxy-3-methylazetidin-1-yl)methyl]-7,8-dihydro-6H-spiro[[1,3]oxazolo[5,4-f]quinazoline-9,1′-cyclohexane]-7-one; 5-chloro-2-{[3-(hydroxymethyl)pyrrolidin-1-yl]methyl}-7,8-dihydro-6H-spiro[[1,3]oxazolo[5,4-f]quinazoline-9,1′-cyclohexane]-7-one; 5-chloro-2-({[2-(dimethylamino)ethyl](methyl)amino}methyl)-7,8-dihydro-6H-spiro[[1,3]oxazolo[5,4-f]quinazoline-9,1′-cyclohexane]-7-one; 5-chloro-2-{[methyl(oxan-4-yl)amino]methyl}-7,8-dihydro-6H-spiro[[1,3]oxazolo[5,4-f]quinazoline-9,1′-cyclohexane]-7-one; and pharmaceutically acceptable salts thereof.

13. A pharmaceutical composition, characterized in that it comprises at least one PDE7 inhibitory agent as claimed in one or more of claims 1 to 12.

14. The pharmaceutical composition as claimed in claim 13, characterized in that it comprises (i) at least one PDE7 inhibitory agent according to claims 8 and 9, or a pharmaceutically acceptable salt, solvate, hydrate, co-crystal, polymorph, ester, ether, enantiomer, prodrug, or metabolite thereof, and at least one pharmaceutically acceptable diluent or excipient, or (ii) at least one compound of the formulas according to claims 8, 9, 10, 11 and 12, or a pharmaceutically acceptable salt, solvate, hydrate, co-crystal, polymorph, ester, ether, enantiomer, prodrug, or metabolite thereof, and at least one pharmaceutically acceptable diluent or excipient.

15. Use of a compound with phosphodiesterase-7 inhibitory activity (PDE7 inhibitors) for the manufacture of a medicament for the treatment and prevention of chronic fatigue, exhaustion and/or exertional intolerance, preferably said PDE7 inhibitory compound inhibits the enzymatic activity of PDE7 (PDE7A, PDE7B, or PDE7A and PDE7B) at an IC₅₀<50 μM, preferrably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.
